(19) United States
(12) Patent Application Publication
(10) Pub. No.: US 2010/0040606 A1
(43) Pub. Date: Feb. 18, 2010

(54) Recombinant polyclonal antibody for treatment of respiratory syncytial virus infections

(75) Inventors: Johan Lantto, Lund (SE); Lucilla Steinaa, Horsholm (DK); Klaus Koefoed, Copenhagen (DK)

Correspondence Address: STERNE, KESSLER, GOLDSTEIN & FOX P.L.L.C., 1100 NEW YORK AVENUE, N.W., WASHINGTON, DC, 20005, US

(73) Assignee: SYMPHOGEN A/S, Lyngby (DK)

(21) Appl. No.: 11/792,927

(22) PCT Filed: Mar. 6, 2007
(86) PCT No.: PCT/DK2007/000113

§ 371 Date: Sep. 21, 2009

Related U.S. Application Data
(60) Provisional application No. 60/874,716, filed on Dec. 14, 2006.

(30) Foreign Application Priority Data
Mar. 6, 2006 (DK) PA 2006 00324

(51) Int. Cl. A61K 39/395; C07K 16/08; C12P 19/34; C12N 5/00; A61P 31/12; A61K 39/42; C07H 21/00; C12N 15/63

(52) U.S. Cl. 424/133.1; 530/387.3; 435/91.2; 435/325; 530/389.4; 424/159.1; 536/23.53; 435/320.1

(57) ABSTRACT

Disclosed are novel polyclonal antibodies, which target respiratory syncyticilal virus (RSV), and novel high affinity antibody molecules reactive with RSV. The polyclonal antibodies may comprise antibody molecules which are reactive with both RSV protein F and RSV protein G, and preferably the polyclonal antibodies target a variety of epitopes on these proteins. The single antibody molecules of the invention are shown to exhibit affinities which provide for dissociation constants as low as in the picomolar range. Also disclosed are methods of producing the antibodies of the invention as well as methods of their use in treatment for RSV infection.

### FIELD OF THE INVENTION

The present invention relates to a recombinant polyclonal antibody for prevention, treatment or amelioration of one or more symptoms associated with respiratory syncytial virus infections. The invention also relates to polyclonal expression cell lines producing anti-RSV recombinant polyclonal antibody (anti-RSV rpAb). Further, the application describes diagnostic and pharmacological compositions comprising anti-RSV rpAb and use in prevention, treatment or amelioration of one or more symptoms associated with a RSV infection.

### BACKGROUND OF THE INVENTION

Respiratory syncytial virus (RSV) is a major cause for lower respiratory tract disease in infants and small children. Premature infants and children with an underlying health problem such as chronic lung disease or congenital heart disease are at the greatest risk for serious illness such as bronchiolitis and pneumonia following RSV infection. Recently, RSV was also recognized as an important pathogen in certain high-risk adults, such as immunocompromised adults, particularly bone marrow transplant recipients, elderly individuals and individuals with chronic pulmonary disease.

Human RSV is a member of the Pneumovirus subfamily of the family Paramyxoviridae, and exists as an A and B subtype. RSV is an enveloped, non-segmented, negative-sense RNA virus. The viral genome codes for at least 11 proteins of which three are the envelope associated proteins, F (fusion glycoprotein), G (receptor-binding glycoprotein), and SH (small hydrophobic protein). The envelope proteins are present on the viral surface, and to some extent also on the surface of infected cells. The F protein promotes fusion of the viral and cell membranes, thereby allowing penetration of the viral RNA into the cell cytoplasm. The F protein consists of two disulfide-linked subunits, F₁ and F₂, produced by proteolytical cleavage of an inactive, N-glycosylated precursor of 574 amino acids. The G protein is a type II trans-membrane glycoprotein of 289-299 amino acids (depending on the virus strain). The precursor form is 32 kDa, which matures to a protein of 80-90 kDa upon addition of both N- and O-linked oligosaccharides. The RSV G protein is responsible for the attachment of virions to the target cells. In addition to the membrane-bound form of the G protein, a truncated, soluble form is also produced. It has been suggested that the function of this is to redirect the immune response away from the virus and infected cells. Further it has been shown that the G protein is associated with a number of pro-inflammatory effects such as modification of chemokine and cytokine expression as well as leukocyte recruitment. The SH protein is a protein of 64-65 amino acids that is present in very low amounts on the surface of purified RSV particles, but is abundantly expressed on the surface of RSV-infected cells. The function of the SH protein has not been defined, but it is possible that it may aid virus protein transport through the Golgi complex (Rixon et al 2004, J. Gen. Virol. 85:1153-1165). Blocking the function of the G and F proteins is believed to be relevant in prevention of RSV infection.

The prevention and treatment of RSV infection has received considerable attention during the last decades, and include vaccine development, antiviral compounds (Ribavirin approved for treatment), antisense drugs, RNA interference (RNAi) technology and antibody products such as immunoglobulin and monoclonal antibodies (all reviewed in Maggon and Barik, 2004, Rev. med. Virol. 14:149-168). Of these approaches, the intravenous immunoglobulin, RSV-IVIG, and the monoclonal antibody, Palivizumab, have been approved for RSV prophylaxis in high-risk children.

Immunoglobulin products such as RSV-IVIG (RespiGam) are, however, known to have several drawbacks such as low specific activity resulting in need for injection of large volumes, which is difficult in children with limited venous access due to prior intensive therapy. Further, there is also the risk of transmission of viral diseases from serum-derived immunoglobulin products, as well as problems with batch-to-batch variations. Finally, it is difficult to obtain sufficient donors to meet the needs for hyperimmune RSV immunoglobulin production, since only approximately 8% of normal donors have RSV neutralizing antibody titers that are high enough.

Monoclonal antibodies against the F protein or the G protein have been shown to have neutralizing effect in vitro and prophylactic effects in vivo (e.g. Beeler and Coelingh 1989. J. Virol. 63:2941-50; Garcia-Barreno et al. 1989. J. Virol. 63:925-32; Taylor et al. 1984. Immunology 52: 137-142; Walsh et al. 1984, Infection and Immunity 43:756-758; U.S. Pat. No. 5,842,307 and U.S. Pat. No. 6,818,216). Today the monoclonal antibody Palivizumab has almost substituted the use of RSV-IVIG completely. Neutralization assays show that Palivizumab and RSV-IVIG perform equally well against RSV subtype B, whereas Palivizumab perform better against subtype A (Johnson et al. 1997. J. Infect. Dis. 176:1215-24.). However, despite the good neutralizing and prophylactic effects of monoclonal antibodies as illustrated by products like Palivizumab and Numax, these may also be associated with certain drawbacks due to the nature of the RSV virus.

RSV exists in two distinct antigenic groups or subtypes, A and B. Most of the RSV proteins are highly conserved between the two subgroups, with the F protein showing 91% amino acid similarity. However, the G protein displays extensive sequence variability, with only 53% amino acid similarity between the A and B subgroups (Sullender 2000. Clin. Microbiol. Rev. 13:1-15). Most of the proteins also show some limited intra subgroup variation, except for the G protein, which differs by up to 20% within subgroup A and 9% within subgroup B on amino acid level. The A and B virus subtypes co-circulate in most RSV epidemics, with the relative frequency varying between different years. Thus, a monoclonal antibody must be carefully selected such that it is capable of neutralizing both subtypes as well as intra subtype variations.

In addition to the issue of the two RSV subtypes and intra-subtype diversity, human RSV, like most RNA viruses, has the capacity of undergoing rapid mutations under selective pressure. The selection of RSV escape mutants in vitro using mAb is well documented (e.g. Garcia-Barreno et al. 1989. J. Virol. 63:925-32). Importantly, it was recently discovered that Palivizumab also selects for escape mutants, in vitro as well as in vivo, and that some of the isolated mutants are completely resistant to Palivizumab prophylaxis in cotton rats (Zhao and Sullender 2005. J. Virol. 79:3962-8 and Zhao et al. 2004. J. Infect. Dis. 190:1941-6.). Further, wild type RSV strains that are intrinsically resistant to Palivizumab may also exist, as demonstrated by the failure of the murine antibody, which Palivizumab originates from, to neutralize one clinical isolate (Beeler and Coelingh 1989. J. Virol. 63:2941-50). Furthermore, one apparently resistant virus has also been identified following Palivizumab prophylaxis in immunocompetent cotton rats (Johnson et al. 1997. J. Infect. Dis. 176:1215-24). Thus, under certain conditions, the use of a single, monospecific antibody may not be adequate or sufficient for the treatment of RSV disease, since escape mutants exist or may develop over time as a result of treatment.

A further consideration in relation to the utility of the RSV-IVIG and Palivizumab is the dose needed for efficient treatment. Serum concentrations of greater than 30 μg/ml have been shown to be necessary to reduce pulmonary RSV replication by 100 fold in the cotton rat model of RSV infection. For RSV-IVIG a monthly dose of 750 mg total protein/kg administrated intravenously was effective in reducing the incidence of RSV hospitalization in high-risk children, whereas for Palivizumab monthly intramuscular doses of 15 mg/kg proved effective. However, the administration of multiple intravenous or intramuscular large doses is inconvenient for the patient, and impedes the broad use of these products for the prophylaxis and treatment of the large group of adults at risk for RSV infection.

Thus, a need exists for an antibody product which is not dependent on the donor availability, and which binds immunospecifically to one or more RSV antigens covering subtypes A and B as well as any escape mutants arising due to virus mutations, is highly potent, have an improved pharmacokinetic profile, and thus have an overall improved therapeutic profile, and therefore requires less frequent administration and/or administration of a lower dose.

### DISCLOSURE OF CONTRIBUTION

It is therefore the objective of the present invention to provide a highly potent alternative anti-RSV immunoglobulin product which is produced recombinantly and shows reactivity to subtypes A and B of the respiratory syncytial virus as well as to multiple epitopes on at least one of the major surface antigens to limit the possibility of escape mutations.

The invention also has as an objective to provide novel human anti-RSV antibody molecules as well as derivatives thereof, where the antibody molecules or derivatives exhibit improved characteristics over existing monoclonal anti-RSV antibodies and antibody derivatives.

### DESCRIPTION OF THE INVENTION

The use of a polyclonal antibody composition targeting multiple epitopes on RSV is expected to minimize the development of escape mutants and can also provide protection against diverse, naturally circulating viruses. In contrast to serum-derived RSV-IVIG, a polyclonal antibody of the present invention does not contain antibody molecules, which bind to non-RSV antigens.

The present invention provides a polyclonal anti-RSV antibody. Preferably, the polyclonal anti-RSV antibody is obtained from cells which do not naturally produce antibodies. Such an antibody is termed a recombinant polyclonal antibody (rpAb). An anti-RSV rpAb of the present invention is directed against multiple epitopes on the F or G protein. In particular an anti-RSV rpAb which is directed against multiple epitopes on both the G and F proteins is preferred. Preferably, G protein epitopes belonging to the conserved group and potentially also the subtype-specific group and the strain-specific group are covered by the anti-RSV rpAb. Further, antibodies with reactivity against the third envelope protein, small hydrophobic (SH) protein is a desired component of an anti-RSV rpAb of the present invention.

Further, the present invention provides pharmaceutical compositions where the active ingredient is an anti-RSV polyclonal antibody, as well as uses of such compositions for the prevention, amelioration or treatment of RSV infections.

The present invention further provides procedures for mirroring the humoral immune response raised upon infection with RSV, by isolating the original V_{H} and V_{L} gene pairs from such challenged individuals, and producing antibodies maintaining this original paring.

### DEFINITIONS

The term “antibody” describes a functional component of serum and is often referred to either as a collection of molecules (antibodies or immunoglobulin) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or on distinct, different antigens). Each antibody molecule has a unique structure that enables it to bind specifically to its corresponding antigen, and all natural antibody molecules have the same overall basic structure of two identical light chains and two identical heavy chains. Antibodies are also known collectively as immunoglobulin. The terms antibody or antibodies as used herein is used in the broadest sense and covers intact antibodies, chimeric, humanized, fully human and single chain antibodies, as well as binding fragments of antibodies, such as Fab, Fv fragments or scFv fragments, as well as multimeric forms such as dimeric IgA molecules or pentavalent IgM. In some instances, the present application uses the term “synthetic or semi-synthetic antibody analogue”, which specifically refers to non-naturally occurring molecules which exhibit antibody characteristics (by exhibiting specific binding to RSV antigens) and includes CDRs from naturally occurring antibodies—such analogues are e.g. represented by scFv fragments, diabodies etc, but could e.g. also be seemingly naturally occurring antibodies which are engineered to include the CDRs (e.g. by grafing techniques known in the art) from an anti-RSV antibody molecule disclosed herein—for instance, such an antibody analogue could comprise CDRs disclosed herein incorporated into an antibody molecule of another animal species or into a different antibody isotype or class from the same species.

The term “anti-RSV recombinant polyclonal antibody” or “anti-RSV rpAb” describes a composition of recombinantly produced diverse antibody molecules, where the individual members are capable of binding to at least one epitope on a respiratory syncytial virus, and where the polyclonal composition as a whole is capable of neutralizing RSV. Preferably, an anti-RSV rpAb composition neutralizes both RSV subtype A and B. Even more preferred the anti-RSV rpAb further comprise binding reactivity towards the G and F protein. Preferably, the composition is produced from a single polyclonal manufacturing cell line.

The term “cognate V_{H} and V_{L} coding pair” describes an original pair of V_{H} and V_{L} coding sequences contained within or derived from the same cell. Thus, a cognate V_{H} and V_{L} pair represents the V_{H} and V_{L} pairing originally present in the donor from which such a cell is derived. The term “an antibody expressed from a V_{H} and V_{L} coding pair” indicates that an antibody or an antibody fragment is produced from a vector, plasmid or similar containing the V_{H} and V_{L} coding sequence. When a cognate V_{H} and V_{L} coding pair is expressed, either as a complete antibody or as a stable fragment thereof, they preserve the binding affinity and specificity of the antibody originally expressed from the cell they are derived from. A library of cognate pairs is also termed a repertoire or collection of cognate pairs, and may be kept individually or pooled.

The terms “a distinct member of a recombinant polyclonal antibody” denotes an individual antibody molecule of the recombinant polyclonal antibody composition, comprising one or more stretches within the variable regions, which are characterized by differences in the amino acid sequence compared to the other individual members of the polyclonal protein. These stretches are in particular located in the CDR1, CDR2 and CDR 3 regions.

The term “epitope” is commonly used to describe a proportion of a larger molecule or a part of a larger molecule (e.g. antigen or antigenic site) having antigenic or immunogenic activity in an animal, preferably a mammal, and most preferably in a human. An epitope having immunogenic activity is a portion of a larger molecule that elicits an antibody response in an animal. An epitope having antigenic activity is a portion of a larger molecule to which an antibody immunospecifically binds as determined by any method well known in the art, for example, by the immunoassays described herein. Antigenic epitopes need not necessarily be immunogenic. An antigen is a substance to which an antibody or antibody fragment immunospecifically binds, e.g. toxin, virus, bacteria, proteins or DNA. An antigen or antigenic site often has more than one epitope, unless they are very small, and is often capable of stimulating an immune response. Antibodies binding to different epitopes on the same antigen can have varying effects on the activity of the antigen they bind depending on the location of the epitope. An antibody binding to an epitope in an active site of the antigen may block the function of the antigen completely, whereas another antibody binding at a different epitope may have no or little effect on the activity of the antigen alone. Such antibodies may however still activate complement and thereby result in the elimination of the antigen, and may result in synergistic effects when combined with one or more antibodies binding at different epitopes on the same antigen. In the present invention the larger molecule which the epitope is a proportion of is preferably a proportion of an RSV polypeptide. Antigens of the present invention are preferably RSV associated proteins, polypeptides or fragments thereof to which an antibody or antibody fragment immunospecifically binds. A RSV associated antigen may also be an analog or derivative of a RSV polypeptide or fragment thereof to which an antibody or antibody fragment immunospecifically binds.

The term “fully human” used for example in relation to DNA, RNA or protein sequences describes sequences which are between 98 to 100% human.

The term “immunoglobulin” commonly is used as a collective designation of the mixture of antibodies found in blood or serum, but may also be used to designate a mixture of antibodies derived from other sources.

The term “mirrors the humoral immune response” when used in relation to a polyclonal antibody refers to an antibody composition where the nucleic acid sequences encoding the individual antibody members are derived from a donor with an increased frequency of plasma cells producing anti-RSV specific antibodies. Such a donor may either be recovering from a RSV infection, has had close contact with an RSV infected individual, or has been subject to RSV vaccination (for examples of RSV vaccines see for example Maggon and Barik, 2004, Rev. med. Virol. 14:149-168). In order to mirror the affinity and specificity of antibodies raised in a donor upon infection or challenge, the sequences encoding the variable heavy chain (V_{H}) and the variable light chain (V_{L}) should be maintained in the gene pairs or combinations originally present in the donor (cognate pairs) when they are isolated. In order to mirror the diversity of a humoral immune response in a donor all the sequences encoding antibodies which bind to RSV are selected based on a screening procedure. The isolated sequences are analyzed with respect to diversity of the variable regions, in particular the CDR regions, but also with respect to the V_{H} and V_{L} family. Based on these analyses a population of cognate pairs representing the overall diversity of the RSV binding antibodies are selected. Such a polyclonal antibody typically have at least 5, 10, 20, 30, 40, 50, 100, 1000 or 104 distinct members.

A composition is said to be “pharmacologically acceptable” if its administration can be tolerated by a recipient patient—the same of course applies to excipients, vehicles carriers and diluents being part of a composition.

The term “polyclonal antibody” describes a composition of different (diverse) antibody molecules which is capable of binding to or reacting with several different specific antigenic determinants/epitopes on the same or on different antigens, where each individual antibody in the composition is capable of reacting with a particular epitope. Usually, the variability of a polyclonal antibody is located in the so-called variable regions of the polyclonal antibody, in particular in the CDR1, CDR2 and CDR3 regions. In the present invention a polyclonal antibody may either be produced in one pot from a polyclonal cell line, or it may be a mixture of different polyclonal antibodies. A mixture of monoclonal antibodies is not as such considered a polyclonal antibody, since they are produced in individual batches and not necessarily from the same cell line which will result in e.g. post translational modification differences. However, if a mixture of monoclonal antibodies provide the same antigen/epitope coverage as a polyclonal antibody of the present invention it will be considered as an equivalent of the polyclonal antibody. When stating that a member of a polyclonal antibody specifically binds to or has specific reactivity against an antigen/antigenic site/epitope, it is herein meant that the binding constant is below 100 nM, preferably below 10 nM, even more preferred below 1 nM.

The term “recombinant antibody” is used to describe an antibody molecule or several molecules that is/are expressed from a cell or cell line transfected with an expression vector comprising the coding sequence of the antibody which is not naturally associated with the cell. If the antibody molecules in a recombinant antibody composition are diverse or different, the term “recombinant polyclonal antibody” or “rpAb” applies in accordance with the definition of a polyclonal antibody.

The term “recombinant polyclonal cell line” or “polyclonal cell line” refers to a mixture/population of protein expressing cells that are transfected with a repertoire of variant nucleic acid sequences (e.g. a repertoire of antibody encoding nucleic acid sequences), which are not naturally associated with the transfected cells. Preferably, the transfection is performed such that the individual cells, which together constitute the recombinant polyclonal cell line, each carry a transcriptionally active copy of a single distinct nucleic acid sequence of interest, which encodes one member of the recombinant polyclonal antibody of interest. Even more preferred, only a single copy of the distinct nucleic acid sequence is integrated at a specific site in the genome. The cells constituting the recombinant polyclonal cell line are selected for their ability to retain the integrated copy (copies) of the distinct nucleic acid sequence of interest, for example by antibiotic selection. Cells which can constitute such a polyclonal cell line can be for example bacteria, fungi, eukaryotic cells, such as yeast, insect cells, plant cells or mammalian cells, especially immortal mammalian cell lines such as CHO cells, COS cells, BHK cells, myeloma cells (e.g., Sp2/0 cells, NS0), NIH 3T3, YB2/0 and immortalized human cells, such as HeLa cells, HEK 293 cells, or PER.C6.

The terms “sequences encoding V_{H} and V_{L} pairs” or “V_{H} and V_{L} encoding sequence pairs” indicate nucleic acid molecules, where each molecule comprise a sequence that code for the expression of a variable heavy chain and a variable light chain, such that these can be expressed as a pair from the nucleic acid molecule if suitable promoter and/or IRES regions are present and operably linked to the sequences. The nucleic acid molecule may also code for part of the constant regions or the complete constant region of the heavy chain and/or the light chain, allowing for the expression of a Fab fragment, a full-length antibody or other antibody fragments if suitable promoter and/or IRES regions are present and operably linked to the sequences.

A recombinant polyclonal antibody is said to be administered in a “therapeutically effective amount” if the amount administered is physiologically significant, e.g. prevents or attenuates an RSV infection in an animal or human.

### DESCRIPTION OF THE DRAWINGS

FIG. 1: (A) Alignment of the amino acid sequences of the whole G protein from the prototypic strains, Long (subtype A) and 18537 (subtype B). The signal/trans-membrane region is boxed with a dotted line. The two variable domains between amino acid 101-133 and 208-299 as identified by Cane et al. 1991 J. Gen. Virol. 72:2091-2096 are identified with an underline. The central fragment of the G protein has been expressed as a fusion protein in E. coli and is boxed in black. The 2 amino acid sequences are set forth in SEQ ID NOs: 711 (subtype A) and 712 (Subtype B). (B) Alignment of the central fragment, as indicated in (A). The location of the 13-aa conserved region (a.a. residue 164-176) and the G protein cystein-rich region (GCRR) are indicated with brackets. The disulphide bridges in the GCRR (identical for both subtypes) are indicated with square brackets. The 2 amino acid sequences are set forth in SEQ ID NOs: 713 (Subtype A) and 714 (subtype B).

FIG. 2: Schematic outline of the multiplex overlap-extension RT-PCR (A) and the cloning steps (B). (A) Two sets of primers, CH+VH 1-8 and VK1-6+CK1, specific for V_{H} and Vκ gene families, respectively, were used for the first PCR step. A homologous region between the V_{H} or Vκ primers results in the generation of an overlap PCR product. In the second step this product is amplified in the nested PCR. The primers also include recognition sites for restriction enzymes that facilitate cloning. (B) The generated cognate linked V_{H} and Vκ coding pairs are pooled and inserted into a mammalian IgG expression vector (e.g. FIG. 3) by the use of the flanking XhoI and NotI restriction sites. Subsequently a bi-directional promoter is inserted into the AscI-NheI restriction site between the linked V_{H} and Vκ coding sequences to facilitate expression of full length antibodies. PCR primers used are indicated by horizontal arrows. CH1: heavy chain constant domain 1, CL: constant domain, LC: light chain; Ab: antibody; P1-P2: bi-directional promoters.

FIG. 3: Schematic presentation of a mammalian full-length antibody expression vector 00-VP-530. The vector comprises the following elements: Amp and Amp pro=ampicillin resistance gene and its promoter. pUC origen=pUC origin of replication. P1=mammalian promoter driving the expression of the light chain. P2=mammalian promoter driving the expression of the heavy chain. Leader IGHV=genomic human heavy chain leader. VH=heavy chain variable region encoding sequence. IgG1=Sequence encoding genomic immunoglobulin isotype G1 heavy chain constant region. Rabbit B-globin A=rabbit beta-globin polyA sequence. Kappa leader=sequence encoding for murine kappa leader. LC=Sequence of light chain encoding sequence. SV40 term=Simian virus 40 terminator sequence. FRT=A Flp recognition target site. Neo=neomycin resistance gene. SV40 poly A=Simian virus 40 poly A signal sequence

FIG. 4: Characterization of the epitope specificity of antibody obtained from clone 801 (Ab801) using Biacore analysis. Antibody 801 binding was tested in pair-wise competition for binding to protein F, using three antibodies, 9c5 (2), 133-h (3) and Palivizumab (4), which bind to antigenic site F1, C and II, respectively. The reference cell illustrates binding to protein F of uncompeted Ab801 (1). Injection times of the four antibodies are indicated by an arrow. The response is indicated in relative resonance units (RU). The long double headed arrow indicates the magnitude of the uncompeted response and the short double headed arrow indicates the magnitude of the 9c5 inhibited response.

FIG. 5: Shows results from in vitro neutralization of RSV subtype A and B strains. Dilutions of anti-F antibody mixtures were tested for their ability to neutralize RSV Long (Panel A) and RSV B1 (Panel B) strains. Antibody mixture, anti-F(I), obtained from clones 810, 818, 819, 825 and 827 is shown as triangles (▴) and antibody mixture, anti-F(II), obtained from clones 735, 800, 810, 818, 819, 825, 827, 863, 880, 884 and 894 is shown as squares (▪). Palivizumab is shown as diamonds (♦), and an isotype-matched negative control (anti-Rhesus D) antibody is shown as circles (). The absorbance was measured at 490 nm and correlates with RSV replication.

FIG. 6: Shows results from an in vitro RSV fusion inhibition assay. Dilutions of antibody mixtures were tested for their ability to neutralize RSV B1 strain. Antibody mixture, anti-F(I)G, obtained from clones 810, 818, 819, 825, 827, 793, 796, 838, 841, 856 and 888 is shown as open squares (□) and antibody mixture, anti-F(II)G, obtained from clones 735, 800, 810, 818, 819, 825, 827, 863, 880, 884, 894, 793, 796, 838, 841, 856 and 888 is shown as open triangles (Δ). Palivizumab is shown as diamonds (♦). The absorbance was measured at 490 nm and correlates with RSV replication.

FIG. 7: Shows results from an in vitro neutralization of RSV by combinations of anti-G antibody clones as measured by the PRNT in the presence of active complement. Dilutions of individual antibody compositions (described in Table 8) were incubated with RSV strain Long in the presence of rabbit complement and afterwards allowed to infect HEp-2 cells. After 24 hours of incubation, the degree of infection was detected using immunodetection of RSV-specific plaques. Anti-RSV rpAb 13 is shown as open triangles (Δ), anti-RSV rpAb 35 as triangles (▴), anti-RSV rpAb 36 as squares (▪), anti-RSV rpAb 41 as circles () and anti-RSV rpAb 45 as open squares (□). Data are presented as % infection compared to control±SD.

### DETAILED DESCRIPTION OF THE INVENTION

### Target Antigens and Polyclonal Antibody Compositions

A polyclonal antibody of the present invention is composed of a number of distinct antibody molecules in the same composition. Each molecule is selected based on its ability to bind an RSV associated antigen. A polyclonal antibody of the present invention comprises binding reactivity corresponding to the compiled binding reactivity of the distinct antibody molecules constituting the polyclonal antibody composition.

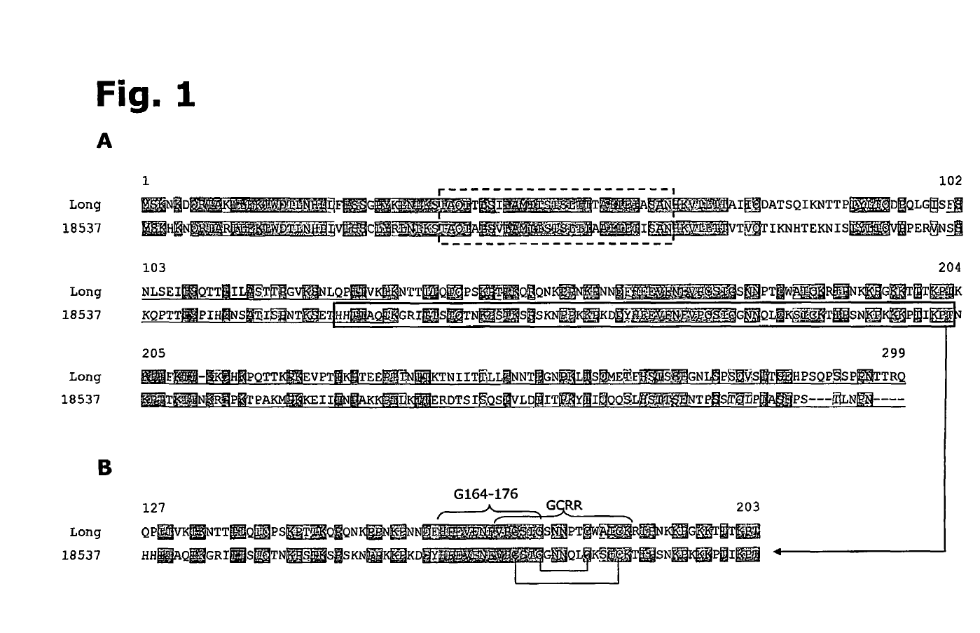

An anti-RSV polyclonal antibody of the present invention preferably comprise a compiled binding reactivity against both the G and F proteins and even more preferred against multiple epitopes to minimize the risk of development of escape mutants and achieve highest possible neutralizing capacity. At least five major antigenic sites that are recognized by neutralizing antibodies have been identified on the F protein (Lopez et al. 1998. J. Virol. 72:6922-8). All the antigenic sites have been mapped to the F₁ chain, and include site I, II, IV, V and VI, where site I and II also may be termed B and A, respectively. Site II is located in a protease-resistant region in the N-terminal segment, and sites IV, V and VI in the C-terminal end of the cystein-rich region of the protein. Site I is located in the middle of this cystein cluster. A further antigenic site on the F protein is site C in which the epitope F2 including amino acid positions 241 and 242 is located. Additionally, there are monoclonal antibodies binding to an antigenic site termed F1, comprising the epitopes termed F1a, F1b and F1c. Currently this antigenic site has not been mapped to a particular site on the F protein. The majority of these sites/epitopes give rise to broadly neutralizing antibodies, but some antibodies specific for antigenic site I have been shown to be subtype A-specific. Antibodies binding to site I also have a marginal effect in virus neutralization. The epitope recognized by Palivizumab is located in antigenic site II as judged by the localization of the selected escape mutations in amino acid position 272 (Zhao et al. 2004. J. Infect. Dis. 190:1941-6). Furthermore, three types of epitopes have been identified on the G protein: i) conserved epitopes that are present in all RSV strains, ii) group-specific epitopes that are present in all viruses belonging to the same subtype, and iii) strain-specific or variable epitopes that are present only in a subset of strains belong to the same subtype. The conserved and group-specific epitopes have been mapped to the central part of the G protein containing a cluster of four cysteins (amino acid residue 173, 176, 182 and 186) and a short amino acid segment (residues 164-176) of identical sequence among all human RSV isolates. The cystein cluster is held by disulfide bounds between position 173-183 and 176-182 and constitutes the central part of the G protein cysteine-rich region (GCRR) ranging from amino acid residue 171-187, thereby the GCRR is overlapping with the 13 amino acid conserved region. The G glycoprotein appears to play a role in both induction of protective immunity and disease pathogenesis. For example, studies in mice have shown that the G glycoprotein primes for a Th2 CD4+ T cell response, characterized by production of IL-4, IL-5, IL-13 and pulmonary eosinophilia. Eosinophil recruitment and activation are promoted by several factors, such as IL-4 and IL-5. Further, expression of RSV G protein during acute infection in mice has been associated with a modified innate immune response characterized by decreased Th1 cytokine expression (e.g., IL-2 and gamma interferon), altered chemokine mRNA expression (e.g., MIP-1 alpha, MIP-1 beta, MIP-2, IP-10, MCP-1), and decreased NK cell trafficking to the infected lung. In particular the GCRR has been shown to play an important role in modulating the innate inflammatory response, thereby potentially delaying RSV clearance (Polack et al. 2005. PNAS 102:8996-9001). The GCRR comprise a CX3C motif at amino acid positions 182 to 186. Reduction in respiratory rates in RSV infected mice has been shown to be associated with the CX3C motif, since antibodies against this motif abolish the reduction in the respiratory rates (Tripp et al. 2003. J. Virol. 77:6580-6584 and US 2004/0009177 (application Ser. No. 10/420,387)). The strain-specific epitopes are preferentially localized in the variable C-terminal third of the G polypeptide, although a strain-specific epitope has been mapped to a variable region N-terminal to the cysteine cluster in the G protein ectodomain (Martinez et al. 1997. J. Gen. Virol. 78:2419-29). FIG. 1 shows an alignment of the G proteins from the Long strain (subtype A) and the 18537 strain (subtype B), indicating the various regions of the G protein. Generally, monoclonal anti-G protein antibodies have marginal effects on RSV neutralization. However, it has been reported that mixtures of monoclonal anti-G antibodies enhance neutralization of RSV in vitro as well as in vivo (Walsh et al. 1989. J. Gen. Virol. 70:2953-61 and Martinez and Melero 1998 J. Gen. Virol. 79:2215-20). The greatest effect of combining monoclonal anti-G antibodies is apparently achieved when the antibodies bind different epitopes, although a fraction of the virus still remained resistant to neutralization. Further, it has been shown that combinations of two different anti-F antibodies with different epitope specificities as well as combinations of one anti-F and one anti-G specific antibody showed an enhanced in vitro neutralizing effect on RSV (Anderson et al. 1988. J. Virol. 62: 4232-4238). Some of the advantages obtained by mixing monoclonal antibodies seem to be due to the individual properties of the monoclonal antibodies, such as an antagonistic effect, e.g. by blockage of the active site. Other effects seem to be synergistic for reasons that currently are not understood.

The mechanisms of RSV neutralization are complex and not completely understood. The large number of different epitopes, conserved, subtype specific as well as strain specific epitopes, identified on the F and G proteins alone, as well as the potential generation of escape mutants suggests that a wide spectrum of antibody specificities is needed to address all the neutralization mechanisms that may play a role in the prevention of RSV infection. Thus, it would be very difficult, in a rational way, to select the mixture of monoclonal antibodies that is capable of preventing RSV infection with RSV strain of both subtype A and B, as well as escape mutants and new strains arising from the RSV strains known today.

An aspect of the present invention is to provide a polyclonal anti-RSV antibody with a considerable diversity and broad anti-RSV specificity. The polyclonal anti-RSV antibody of the present invention is not dependent on the donor availability at the time of production and the batch to batch variation is considerably lower than observed for donor-derived anti-RSV immunoglobulin products (e.g. RSV IVIG). In a polyclonal anti-RSV antibody of the present in invention all the individual antibody members are capable of binding a RSV associated antigen and the polyclonal antibody is capable of neutralizing RSV subtype A and B. It is preferred that each distinct antibody of the polyclonal antibody binds an epitope which is not bound by any of the other members of the polyclonal antibody. A polyclonal anti-RSV antibody of the present invention will bind to RSV antigens in a multivalent manner, which usually results in synergistic neutralization, improved phagocytosis of infected cells by macrophages and improved antibody-dependent cellular cytotoxicity (ADCC) against infected cells as well as increased complement activation. Further, a polyclonal antibody of the present invention is not “diluted” by non-binding protein which is the case for RSV IVIG, where a dose of 750 mg total protein/kg is needed to be efficient. The percentage of RSV-specific antibodies within the 750 mg total protein is not known, but it is not likely to constitute more than maximally 1%, and most likely less. Thus, when the in vitro potency of Palivizumab was estimated to be 25-30 times higher than that of RSV IVIG (Johnson et al. 1997. J. Infect. Dis. 176:1215-24), this is offset by a reduced specific activity of the RSV IVIG. Thus, if only 1% of the immunoglobulin molecules contained in the RSV-IVIG are specific for RSV, then the active dose of the RSV-IVIG polyclonal antibody is only 7.5 mg/kg which is lower than that of the monoclonal antibody Palivizumab.

For these reasons a recombinant polyclonal RSV-specific antibody of the present invention is expected to be significantly more potent than a monoclonal antibody, and it will therefore be possible to administer a smaller dose of a polyclonal antibody of the present invention, compared to the effective doses of Palivizumab and RSV IVIG. Thus, a polyclonal anti-RSV antibody of the present invention is also considered suitable for the prophylaxis and treatment of high-risk adults, in particular bone marrow transplant recipients, elderly individuals and individuals with chronic pulmonary disease. A further advantage of a polyclonal anti-RSV antibody of the present invention, is that the concentration of the individual antibody members is significantly lower than the concentration of a monoclonal antibody (even if the dose used is the same), hence the possibility that the individual antibody will be recognized as foreign by the immune system of the individual under treatment is decreased, and even if one individual antibody is eliminated by an immune response in the patient, this is not likely to affect the neutralizing capability or the clearance rate of the polyclonal anti-RSV antibody, since the remaining antibody members remain intact.

An embodiment of the present invention is a recombinant polyclonal anti-RSV antibody capable of neutralizing RSV subtype A and B, and where said polyclonal antibody comprises distinct antibody members which in union specifically binds at least three different epitopes on at least one RSV envelope protein. Preferably, the F protein is bound specifically by at least three distinct antibody members, and said epitopes are preferably located at different antigenic sites.

A further embodiment of the present invention is a recombinant polyclonal anti-RSV antibody capable of neutralizing RSV subtype A and B, and where said polyclonal antibody comprises distinct antibody members which in union provide specific reactivity against at least two RSV envelope proteins. The two envelope proteins can be selected from the RSV G protein, RSV F protein and RSV SH protein. Preferably, the polyclonal anti-RSV antibody of the present invention comprises anti-G and anti-F reactivity. The anti-G and anti-F reactivity of such a polyclonal antibody is preferably comprised of at least two distinct anti-G antibodies and at least one distinct anti-F antibody. Preferably, at least three distinct antibodies bind to different epitopes, thereby covering at least three different epitopes, and together the antibodies are capable of neutralizing RSV subtype A and subtype B strains equally well. Even more preferred the anti-G and anti-F reactivity of a polyclonal anti-RSV antibody of the present invention is comprised of any combination of the anti-G and anti-F reactivities described below. Most preferred a polyclonal anti-RSV antibody of the present invention is comprised of anti-G and anti-F reactivity against all the antigenic sites/epitopes mentioned below. To obtain the broadest specificity possible of a polyclonal anti-RSV antibody of the present invention, it is desired that one or more, preferably all the antigenic sites are covered by more than one distinct antibody. Consequently, it is preferred that several epitopes on the same antigen or antigenic site are bound by distinct members of a polyclonal antibody of the present invention.

With respect to the anti-G reactivity of a polyclonal anti-RSV antibody of the present invention, this reactivity is preferably directed against conserved epitopes. Even more preferred the anti-G reactivity is comprised of a first anti-G antibody capable of specifically binding a conserved epitope on the G-protein, and a second anti-G antibody capable of specifically binding the G protein cysteine-rich region (GCRR) The polyclonal anti-RSV antibody preferably comprise at least two distinct anti-G antibodies, where at least one first antibody is capable of specifically binding a conserved epitope on the G-protein, and at least one second antibody is capable of specifically binding a different conserved epitope or a group-specific epitope recognizing either with subtype A or subtype B. Preferably, the polyclonal antibody comprise at least three distinct anti-G antibodies where the first antibody is capable of specifically binding a conserved epitope on the G-protein, and the second antibody is capable of specifically binding a G protein of subtype A and the third antibody is capable of specifically binding a G protein of subtype B. The G protein cysteine-rich region (GCRR) partially overlaps with the upstream 13 amino acid region where the conserved epitopes are located and a region where the group specific epitopes are located. Thus, antibodies capable of specifically binding a conserved epitope as well as group specific antibodies may bind the GCRR if the epitope that they recognize is located in the GCRR. Preferably, at least one of the distinct antibodies characterized by their binding to a conserved epitope or a strain specific epitope also recognizes the GCRR. Antibodies binding to the CX3C motif of the GCRR are especially preferred from a virus neutralization point of view. However, antibodies binding to CX3C motifs may also bind a number of other unrelated human antigens, such as fractalkine and other human CX3C chemokines and thus produce undesired side-effects meaning that it will be a rational approach to test such antibodies for cross-reactivity (e.g. as demonstrated for certain antibodies in the examples) and later to test the same antibodies in suitable model systems. At any rate, it will always be necessary to test a given pharmaceutical, such as an antibody of the present invention, in a clinical trial before it can be established with a degree of certainty that side effects are absent, minor or at least acceptable. In addition to the conserved and group-specific anti-G reactivity additional anti-G reactivity directed against strain specific epitopes may also be comprised in the polyclonal anti-RSV antibody of the present invention. Strain-specific anti-G reactivity directed against the most abundant strain-specific epitopes present on virus strains which have resulted in RSV infection within the last five years is preferred. In the current invention strain-specific epitopes are understood as epitopes which only are present on a limited number of RSV strains. The addition of group-specific and/or strain specific anti-G antibodies can provide additional diversity to an anti-RSV antibody of the present invention, and may induce synergy when combined with antibodies with reactivity to the conserved region of the G protein. Preferably, the anti-G antibodies of the present invention neutralize RSV directly, block entry of the virus into the cell, prevent cell migration, inhibit inflammatory responses and/or prevent syncytia formation.

With respect to the anti-F reactivity of a polyclonal anti-RSV antibody of the present invention, this reactivity is preferably directed against at least one epitope on one or more of the antigenic sites I, II, IV, V, VI, C or F1. In further embodiments of the present invention at least two, three, four, five, six or all these antigenic sites/epitopes are covered by distinct antibodies in a polyclonal anti-RSV antibody of the present invention. Preferably, the anti-F antibodies of the present invention neutralize RSV directly and/or block entry of the virus into the cell and/or prevent syncytia formation.

In polyclonal anti-RSV antibody compositions of the present invention where the composition does not comprise binding reactivity directed against all the antigenic sites on the F protein, the presence of at least one distinct anti-F antibody which specifically binds an epitope of antigenic site II is preferred. Even more preferred the site II-specific anti-F antibody binds to the same epitope or antigenic site as the antibody Palivizumab. In addition to the site II-specific antibodies one or more distinct site IV-specific anti-F antibodies are desired, such an antibody preferably binds to the same epitope as RSVF2-5.

Subtype-specific anti-F antibodies are also known in the art. However, since the F protein shows 91% amino acid similarity between the two subgroups A and B, the subtype-specific anti-F antibodies are less abundant than for anti-G antibodies. Such strain-specific anti-F antibodies will, however, contribute to obtaining as broad specificity as possible, and are therefore also desired components of a polyclonal anti-RSV antibody of the present invention.

In addition to the RSV G and F protein antigens mentioned above, the RS virus expresses a third envelope protein, the small hydrophobic (SH) protein. Hyperimmune sera raised against peptides from the SH proteins have been shown to be unable to neutralize RSV in vitro (Akerlind-Stopner et al. 1993 J. Med. Virol. 40:112-120). However, since the protein is mainly expressed on infected cells, we believe that antibodies against the SH protein will have an effect on fusion inhibition and potentially be relevant for in vivo protection against RSV infections. This is supported by the fact that RSV strains lacking the SH gene replicate 10-fold less efficient in the upper respiratory tract (Bukreyev et al. 1997 J. Virol. 71:8973-82).

An additional embodiment of the present invention is a polyclonal anti-RSV antibody capable of neutralizing RSV subtype A and B and comprising anti-SH reactivity, and anti-G or anti-F reactivity. The C-terminus ranging from amino acid 41 to 64/65 (subtype A/B) of the SH protein is exposed on the cell surface. Hence, anti-SH reactivity against an epitope located in this area is desired. The C-terminus of the SH protein varies from subtype A and B, and it is therefore desired to include anti-SH reactivity against both subtype A and B in a polyclonal antibody of the present invention. This SH reactivity can be provided by at least two distinct anti-SH antibodies where the first antibody is capable of specifically binding SH subtype A and the second antibody is capable of specifically binding SH subtype B.

In one embodiment of the present invention a polyclonal anti-RSV antibody comprises specific reactivity against SH subtype A and/or B as well as specific reactivity against the G protein. The reactivity against the G protein can be composed of any of the reactivities mentioned above.

In an alternative embodiment the specific reactivity against SH subtype A and/or B can be combined with any of the anti-F reactivities described in the above to constitute a polyclonal anti-RSV antibody.

In a preferred embodiment of the present invention a polyclonal anti-RSV antibody comprises reactivity against all three of the envelope proteins, F, G and SH.

The reactivity comprised in a polyclonal anti-RSV antibody of the present invention may constitute any possible combination of distinct antibodies with specific binding reactivity against the antigens/antigenic sites and/or epitopes summarized in Table 1, as long as the combination is capable of neutralizing RSV subtype A and B. Preferably, the combination contain reactivity against at least two RSV envelope proteins.

Preferably, the individual distinct antibody members of a polyclonal antibody according to the present invention, have neutralizing and/or anti-inflammatory properties on their own. Antibodies without these particular properties may however also play a role in RSV clearance for example through complement activation.

TABLE 1
Summary of RSV associated antigens, antigenic sites and epitopes

| Antigen | Antigenic site/epitope |
|---|---|
| F Protein | Antigenic site I |
| | Antigenic site II |
| | Antigenic site IV |
| | Antigenic site V |
| | Antigenic site VI |
| | Antigenic site C |
| | F1 epitope |
| G Protein | Conserved region (a.a. 164-176) |
| | Subtype A specific |
| | Subtype B specific |
| | GCRR (a.a. 171-187) |
| | (conserved as well as strain specific) |
| | CX3C motif (a.a. 182-186) |
| | Strain specific |
| SH protein | Subtype A |
| | Subtype B |

Preferably, a polyclonal antibody of the present invention is produced as a single batch or a few batches from a polyclonal cell line which is not naturally expressing antibody molecules (also termed recombinant polyclonal antibody expression). One of the advantages of producing a recombinant polyclonal antibody compared to mixing monoclonal antibodies, is the ability to produce an unlimited number of distinct antibody molecules at the same time (at a cost similar to that of producing a single monoclonal antibody). Thus, it is possible to include antibodies with reactivity towards a large number of RSV associated antigens, without increasing the cost of the end product significantly. In particular with a target as complex as the RSV, where the biology is not completely understood, individual antibodies which have not been shown to neutralize or protect against RSV alone, may when combined with other antibodies induce a synergistic effect. Thus, it can be an advantage to include distinct antibodies, in addition to those described above, in a polyclonal antibody composition, where the only criterion is that the individual antibody binds to an RSV associated antigen (e.g. assessed by binding to RSV infected cells). Preferably all the polyclonal anti-RSV antibody compositions described above are recombinant polyclonal anti-RSV antibody (anti-RSV rpAb) compositions.

One way to acquire potentially relevant antibodies that bind RSV target antigens which have not been verified as relevant antigens, but nonetheless may be so, is to generate a polyclonal antibody composition which is composed of individual antibodies raised by the immune response of a donor which has been infected with RSV (full immune response). In addition to obtaining antibodies representing a full immune response against RSV, a positive selection for antibodies binding to antigens that are likely to be of particular relevance in the protection, neutralization, and/or elimination of RSV infections, can be performed. Further, if antibodies to a particular antigen, antigenic site or epitope which is believed to be of relevance in the protection, neutralization and/or elimination of RSV are not identified in the full immune response of the donor, such antibodies may be raised by immunization/vaccination of a donor with that particular antigen (selected immune response). Generally, neutralization is assessed by in vitro neutralization assays such as plaque reduction, microneutralization or fusion-inhibition assays (e.g. Johnson et al. 1997. J. Infect. Dis. 176:1215-24). Hence, an antibody or antibody composition having a significant effect in one of these assays, when compared to a negative control are considered to be neutralizing. Protection is generally assessed by in vivo challenge experiments in e.g. the cotton rat model (e.g. Johnson et al. 1997. J. Infect. Dis. 176:1215-24) or the murine model (e.g. Taylor et al. 1984. Immunology 52, 137-142 and Mejias, et al. 2005. Antimicrob. Agents Chemother. 49: 4700-4707). The in vivo challenge experiments can either be performed in a prophylactic fashion, where the antibodies are administered prior to the viral challenge or as a treatment, where the antibodies are administered after viral challenge or as a combination of both.

A polyclonal antibody composition of the present invention can be composed of antibodies capable of binding a RSV antigen which is not necessarily known or not necessarily an envelope protein (the antibody binds to infected cells, but not to selected antigens or antigenic sites), but where the antibodies are acquired from a full immune response following a RSV infection, e.g. by obtaining nucleic acid sequences encoding the distinct antibodies from one or more donors with a RSV infection or recovering from a RSV infection. Secondly, antibodies from the same full immune response, which have been selected, based on their ability to bind a particular antigen, antigenic site and/or epitope, can be included in a polyclonal antibody of the present invention. Thirdly, distinct antibodies encoded from V_{H} and V_{L} pairs obtained from one or more donors which have been immunized/vaccinated with a particular RSV related antigen thereby raising a “selected” immune response in these donors, can be included in a polyclonal antibody composition of the present invention. Thus, antibodies derived by any of the mentioned techniques in the present invention may be combined into a single polyclonal antibody. Preferably the nucleic acids encoding the antibodies of the present invention are obtained from human donors and the antibodies produced are fully human antibodies.

The motivation behind the polyclonal antibody compositions of the present invention is: if a donor infected with RSV, raises a humoral immune response against an antigen, these antibodies are likely, at least to some extent, to contribute to viral clearance, and thereby qualify for inclusion in a polyclonal antibody product.

A further aspect of the present invention is to produce an anti-RSV rpAb wherein the composition of distinct antibody members mirrors the humoral immune response with respect to diversity, affinity and specificity against RSV envelope antigens. Preferably, the mirror of the humoral response is established by ensuring that one or more of the following are fulfilled i) the nucleic acid sequences coding for the V_{H} and V_{L} regions of the individual antibody members in such an anti-RSV rpAb are derived from a donor(s) who has raised a humoral immune response against RSV, for example following RSV infection; ii) the V_{H} and V_{L} coding sequences are isolated from the donor(s) such that the original pairing of the V_{H} and V_{L} coding sequences present in the donor(s) is maintained, iii) the V_{H} and V_{L} pairs, coding for the individual members of the rpAb, are selected such that the CDR regions are as diverse as possible; or iv) the specificity of the individual members of the anti-RSV rpAb are selected such that the antibody composition collectively binds antigens that elicit significant antibody responses in mammals. Preferably, the antibody composition collectively binds antigens, antigenic sites and/or epitopes which produce significant antibody titers in a serum sample from said donor(s). Such antigens, antigenic sites and/or epitopes are summarized in Table 1 above, but may also constitute unknown antigens, antigenic sites and/or epitopes as well as non-envelope antigens, as described above. Preferably, the donors are human, and the polyclonal antibody is a fully human antibody.

The present invention has identified a series of V_{H} and V_{L} pairs that can be expressed as full-length antibodies, Fab fragment or other antibody fragments that have binding specificity to a RSV associated antigen. The specific V_{H} and V_{L} pairs are identified by clone number in Table 5 in Example 2. An antibody containing a V_{H} and V_{L} pair as identified in Table 5 is preferably a fully human antibody. However, if desired, chimeric antibodies may also be produced.

A preferred anti-RSV recombinant polyclonal antibody of the present invention is composed of distinct members comprising heavy chain and light chain CDR1, CDR2 and CDR3 regions selected from the group of V_{H} and V_{L} pairs listed in Table 5. Preferably, the CDR regions are maintained in the pairing indicated in Table 5 and inserted into a desired framework. Alternatively, CDR regions from the heavy chain (CDRH) of a first clone are combined with the CDR regions from the light chain (CDRL) of a second clone (scrambling of V_{H} and V_{L} pairs). The CDR regions may also be scrambled within the light chain or heavy chain, for example by combining the CDRL1 region from a first clone with the CDRL2 and CDRL3 region from a second clone. Such scrambling is preferably performed among clones that bind the same antigen. The CDR regions of the present invention may also be subjected to affinity maturation, e.g. by point mutations.

Isolation and selection of variable heavy chain and variable light chain coding pairs

The process of generating an anti-RSV recombinant polyclonal antibody composition involves the isolation of sequences coding for variable heavy chains (V_{H}) and variable light chains (V_{L}) from a suitable source, thereby generating a repertoire of V_{H} and V_{L} coding pairs. Generally, a suitable source for obtaining V_{H} and V_{L} coding sequences are lymphocyte containing cell fractions such as blood, spleen or bone marrow samples from an animal or human which is infected with RSV or recovering from an RSV infection, or from an animal or human immunized/vaccinated with an RSV strain or proteins or DNA derived from such a strain. Preferably, lymphocyte containing fractions are collected from humans or transgenic animals with human immunoglobulin genes. The collected lymphocyte containing cell fraction may be enriched further to obtain a particular lymphocyte population, e.g. cells from the B lymphocyte linage. Preferably, the enrichment is performed using magnetic bead cell sorting (MACS) and/or fluorescence activated cell sorting (FACS), taking advantage of lineage-specific cell surface marker proteins for example for B cells, plasma blast and/or plasma cells. Preferably, the lymphocyte containing cell fraction is enriched with respect to B cells, plasma blasts and/or plasma cells. Even more preferred, cells with high CD38 expression and intermediate CD19 and/or CD45 expression are isolated from blood. These cells are sometimes termed circulating plasma cells, early plasma cells or plasma blasts. For ease, they are just termed plasma cells in the present invention, although the other terms may be used interchangeably.

The isolation of V_{H} and V_{L} coding sequences can either be performed in the classical way where the V_{H} and V_{L} coding sequences are combined randomly in a vector to generate a combinatorial library of V_{H} and V_{L} coding sequences pairs. However, in the present invention it is preferred to mirror the diversity, affinity and specificity of the antibodies produced in a humoral immune response upon RSV infection. This involves the maintenance of the V_{H} and V_{L} pairing originally present in the donor, thereby generating a repertoire of sequence pairs where each pair encodes a variable heavy chain (V_{H}) and a variable light chain (V_{L}) corresponding to a V_{H} and V_{L} pair originally present in an antibody produced by the donor from which the sequences are isolated. This is also termed a cognate pair of V_{H} and V_{L} encoding sequences and the antibody is termed a cognate antibody. Preferably, the V_{H} and V_{L} coding pairs of the present invention, combinatorial or cognate, are obtained from human donors, and therefore the sequences are completely human.

There are several different approaches for the generation of cognate pairs of V_{H} and V_{L} encoding sequences, one approach involves the amplification and isolation of V_{H} and V_{L} encoding sequences from single cells sorted out from a lymphocyte-containing cell fraction. The V_{H} and V_{L} encoding sequences may be amplified separately and paired in a second step or they may be paired during the amplification (Coronella et al. 2000. Nucleic Acids Res. 28: E85; Babcook et al 1996. PNAS 93: 7843-7848 and WO 05/042774). A second approach involves in-cell amplification and pairing of the V_{H} and V_{L} encoding sequences (Embleton et al. 1992. Nucleic Acids Res. 20: 3831-3837; Chapal et al. 1997. BioTechniques 23: 518-524). A third approach is selected lymphocyte antibody method (SLAM) which combines a hemolytic plaque assay with cloning of V_{H} and V_{L} cDNA (Babcook et al. 1996. PNAS 93:7843-7848). In order to obtain a repertoire of V_{H} and V_{L} encoding sequence pairs which resemble the diversity of V_{H} and V_{L} sequence pairs in the donor, a high-throughput method with as little scrambling (random combination) of the V_{H} and V_{L} pairs as possible, is preferred, e.g. as described in WO 05/042774 (hereby incorporated by reference).

In a preferred embodiment of the present invention a repertoire of V_{H} and V_{L} coding pairs, where the member pairs mirror the gene pairs responsible for the humoral immune response resulting from a RSV infection, is generated according to a method comprising the steps i) providing a lymphocyte-containing cell fraction from a donor infected with RSV or recovering from a RSV infection; ii) optionally enriching B cells or plasma cells from said cell fraction; iii) obtaining a population of isolated single cells, comprising distributing cells from said cell fraction individually into a plurality of vessels; iv) amplifying and effecting linkage of the V_{H} and V_{L} coding pairs, in a multiplex overlap extension RT-PCR procedure, using a template derived from said isolated single cells and v) optionally performing a nested PCR of the linked V_{H} and V_{L} coding pairs. Preferably, the isolated cognate V_{H} and V_{L} coding pairs are subjected to a screening procedure as described below.

Once the V_{H} and V_{L} sequence pairs have been generated, a screening procedure to identify sequences encoding V_{H} and V_{L} pairs with binding reactivity towards an RSV associated antigen is performed. Preferably, the RSV associated antigen is a RSV envelope protein, in particular RSV G protein, RSV F protein and RSV SH protein. If the V_{H} and V_{L} sequence pairs are combinatorial a phage display procedure can be applied to enrich for V_{H} and V_{L} pairs coding for antibody fragments binding to RSV prior to screening.

In order to mirror the diversity, affinity and specificity of the antibodies produced in a humoral immune response upon infection with RSV, the present invention has developed a screening procedure for the cognate pairs, in order to obtain the broadest diversity possible. For screening purposes the repertoire of cognate V_{H} and V_{L} coding pairs are expressed individually either as antibody fragments (e.g. scFv or Fab) or as full-length antibodies using either a bacterial or mammalian screening vector transfected into a suitable host cell. The repertoire of Fabs/antibodies is screened for reactivity to virus particles of one or more RSV strains. Preferably, at least two strains, one of subtype A and one of subtype B are used. Subtype A strains are for example Long (ATCC VR-26), A2 (ATCC VR-1540) or more recent Long-like subtype A isolates. Subtype B strains are for example 18537 (ATCC VR-1580), B1 (ATCC VR-1400), 9320 (ATCC VR-955) or more recent 18537-like isolates. In parallel, the repertoire of Fabs/antibodies is screened against selected antigens such as recombinant G protein, recombinant F protein and peptides derived from RSV antigens. The antigenic peptides can for example be selected from the conserved region of the G protein (amino acids 164-176) and the cystein core region (amino acids 171-187 of subtype A as well as subtype B strains) of the G protein and, the extracellular region of the SH-protein (amino acids 42-64 of subtype A and 42-65 of subtype B). Preferably the peptides are biotinylated to facilitate immobilization onto beads or plates during screening. Alternative immobilization means may be used as well. The antigens are selected based on the knowledge of the RSV biology and the expected neutralizing and/or protective effect antibodies capable of binding to these antigens potentially can provide. This screening procedure can likewise be applied to a combinatorial phage display library. The recombinant G and/or F proteins used for screening can be expressed in bacteria, insect cells, mammalian cells or another suitable expression system. The G and/or F protein may either be expressed as a soluble protein (without the transmembrane region) or they may be fused to a third protein, to increase stability. If the G and/or F protein is expressed with a fusion tag, the fusion partner may be cleaved off prior to screening. Preferably, G and/or F proteins representative of both the subtype A and subtype B are expressed and used for screening. Additionally, strain-specific G proteins may be expressed and used for screening. In addition to the primary screening described above, a secondary screening may be performed, in order to ensure that none of the selected sequences encode false positives. In the second screening all the RSV/antigen binding V_{H} and V_{L} pairs identified in the first screening are screened again against both the virus strains and the selected antigens. Generally, immunological assays are suitable for the screening performed in the present invention. Such assays are well know in the art and constitute for example ELISPOTS, ELISA, FLISA, membrane assays (e.g. Western blots), arrays on filters, and FACS. The assays can either be performed without any prior enrichment steps, utilizing polypeptides produced from the sequences encoding the V_{H} and V_{L} pairs. In the event that the repertoire of V_{H} and V_{L} coding pairs are cognate pairs, no enrichment by e.g. phage display is needed prior to the screening. However, in the screening of combinatorial libraries, the immunoassays are preferably performed in combination with or following enrichment methods such as phage display, ribosome display, bacterial surface display, yeast display, eukaryotic virus display, RNA display or covalent display (reviewed in FitzGerald, K., 2000. Drug Discov. Today 5, 253-258).

The V_{H} and V_{L} pair encoding sequences selected in the screening are generally subjected to sequencing, and analyzed with respect to diversity of the variable regions. In particular the diversity in the CDR regions is of interest, but also the V_{H} and V_{L} family representation is of interest. Based on these analyses, sequences encoding V_{H} and V_{L} pairs representing the overall diversity of the RSV binding antibodies isolated from one or more donors are selected. Preferably, sequences with differences in all the CDR regions (CDRH1, CDRH2, CDRH3 and CDRL1, CDRL2 and CDRL3) are selected. If there are sequences with one or more identical or very similar CDR regions which belong to different V_{H} or V_{L} families, these are also selected. Preferably, at least the CDR3 region of the variable heavy chain (CDRH3) differs among the selected sequence pairs. Potentially, the selection of V_{H} and V_{L} sequence pairs can be based solemnly on the variability of the CDRH3 region. During the priming and amplification of the sequences, mutations may occur in the framework regions of the variable region, in particular in the first framework region. Preferably, the errors occurring in the first framework region are corrected in order to ensure that the sequences correspond completely or at least 98% to those of the donor, e.g. such that the sequences are fully human.

When it is ensured that the overall diversity of the collection of selected sequences encoding V_{H} and V_{L} pairs is highly representative of the diversity seen at the genetic level in a humoral response to a RSV infection, it is expected that the overall specificity of antibodies expressed from a collection of selected V_{H} and V_{L} coding pairs also are representative with respect to the specificity of the antibodies produced in the RSV infected donors. An indication of whether the specificity of the antibodies expressed from a collection of selected V_{H} and V_{L} coding pairs are representative of the specificity of the antibodies raised by infected donors can be obtained by comparing the antibody titers towards the virus strains as well as the selected antigens of the donor blood with the specificity of the antibodies expressed from a collection of selected V_{H} and V_{L} coding pairs. Additionally, the specificity of the antibodies expressed from a collection of selected V_{H} and V_{L} coding pairs can be analyzed further. The degree of specificity correlates with the number of different antigens towards which binding reactivity can be detected. In a further embodiment of the present invention the specificity of the individual antibodies expressed from a collection of selected V_{H} and V_{L} coding pairs is analyzed by epitope mapping.

Epitope mapping may be performed by a number of methodologies, which do not necessarily exclude each other. One way to map the epitope-specificity of an antibody clone is to assess the binding to peptides of varying lengths derived from the primary structure of the target antigen. Such peptides may be both linear and conformational and may be used in a number of assay formats, including ELISA, FLISA and surface plasmon resonance (SPR, Biacore). Furthermore, the peptides may be rationally selected using available sequence and structure data to represent e.g. extracellular regions or conserved regions of the target antigen, or the may be designed as a panel of overlapping peptides representing a selected part or all of the antigen (Meloen R H, Puijk W C, Schaaper W M M. Epitope mapping by PEPSCAN. In: Immunology Methods Manual. Ed Iwan Lefkovits 1997, Academic Press, pp 982-988). Specific reactivity of an antibody clone with one or more such peptides will generally be an indication of the epitope specificity. However, peptides are in many cases poor mimics of the epitopes recognized by antibodies raised against proteinaceous antigens, both due to a lack of conformation and due to the generally larger buried surface area of interaction between an antibody and a protein antigen as compared to an antibody and a peptide. A second method for epitope mapping, which allows for the definition of specificities directly on the protein antigen, is by selective epitope masking using existing, well defined antibodies. Reduced binding of a second, probing antibody to the antigen following blocking is generally indicative of shared or overlapping epitopes. Epitope mapping by selective masking may be performed by a number of immunoassays, including, but not restricted to, ELISA and Biacore, which are well known in the art (e.g. Ditzel et al. 1997. J. Mol. Biol. 267:684-695; Aldaz-Carroll et al. 2005. J. Virol. 79: 6260-6271). Yet another potential method for the determination of the epitope specificity of anti-virus antibodies is the selection of viral escape mutants in the presence of antibody. Sequencing of the gene(s) of interest from such escape mutants will generally reveal which amino acids in the antigen(s) that are important for the recognition by the antibody and thus constitute (part of) the epitope.

Preferably, individual members to be comprised in an anti-RSV rpAb of the present invention are selected such that the specificity of the antibody composition collectively covers both RSV subtype A and B, as well as the RSV associated antigens protein F and G, and preferably also SH.

Production of a recombinant polyclonal antibody from selected V_{H} and V_{L} coding pairs

A polyclonal antibody of the present invention is produced from a polyclonal expression cell line in one or a few bioreactors or equivalents thereof. Following this approach the anti-RSV rpAb can be purified from the reactor as a single preparation without having to separate the individual members constituting the anti-RSV rpAb during the process. If the polyclonal antibody is produced in more than one bioreactor, the supernatants from each bioreactor can be pooled prior to the purification, or the purified anti-RSV rpAb can be obtained by pooling the antibodies obtained from individually purified supernatants from each bioreactor.

One way of producing a recombinant polyclonal antibody is described in WO 2004/061104 and WO 2006/007850 (PCT/DK2005/000501) (these references are hereby incorporated by reference). The method described therein, is based on site-specific integration of the antibody coding sequence into the genome of the individual host cells, ensuring that the V_{H} and V_{L} protein chains are maintained in their original pairing during production. Furthermore, the site-specific integration minimizes position effects and therefore the growth and expression properties of the individual cells in the polyclonal cell line are expected to be very similar. Generally, the method involves the following: i) a host cell with one or more recombinase recognition sites; ii) an expression vector with at least one recombinase recognition site compatible with that of the host cell; iii) generation of a collection of expression vectors by transferring the selected V_{H} and V_{L} coding pairs from the screening vector to an expression vector such that a full-length antibody or antibody fragment can be expressed from the vector (such a transfer may not be necessary if the screening vector is identical to the expression vector); iv) transfection of the host cell with the collection of expression vectors and a vector coding for a recombinase capable of combining the recombinase recognition sites in the genome of the host cell with that in the vector; v) obtaining/generating a polyclonal cell line from the transfected host cell and vi) expressing and collecting the polyclonal antibody from the polyclonal cell line.

Preferably mammalian cells such as CHO cells, COS cells, BHK cells, myeloma cells (e.g., Sp2/0 or NS0 cells), fibroblasts such as NIH 3T3, and immortalized human cells, such as HeLa cells, HEK 293 cells, or PER.C6, are used. However, non-mammalian eukaryotic or prokaryotic cells, such as plant cells, insect cells, yeast cells, fungi, E. coli etc., can also be employed. A suitable host cell comprises one or more suitable recombinase recognition sites in its genome. The host cell should also contain a mode of selection which is operably linked to the integration site, in order to be able to select for integrants, (i.e., cells having an integrated copy of an anti-RSV Ab expression vector or expression vector fragment in the integration site). The preparation of cells having an FRT site at a pre-determined location in the genome was described in e.g. U.S. Pat. No. 5,677,177. Preferably, a host cell only has a single integration site, which is located at a site allowing for high expression of the integrant (a so-called hot-spot).

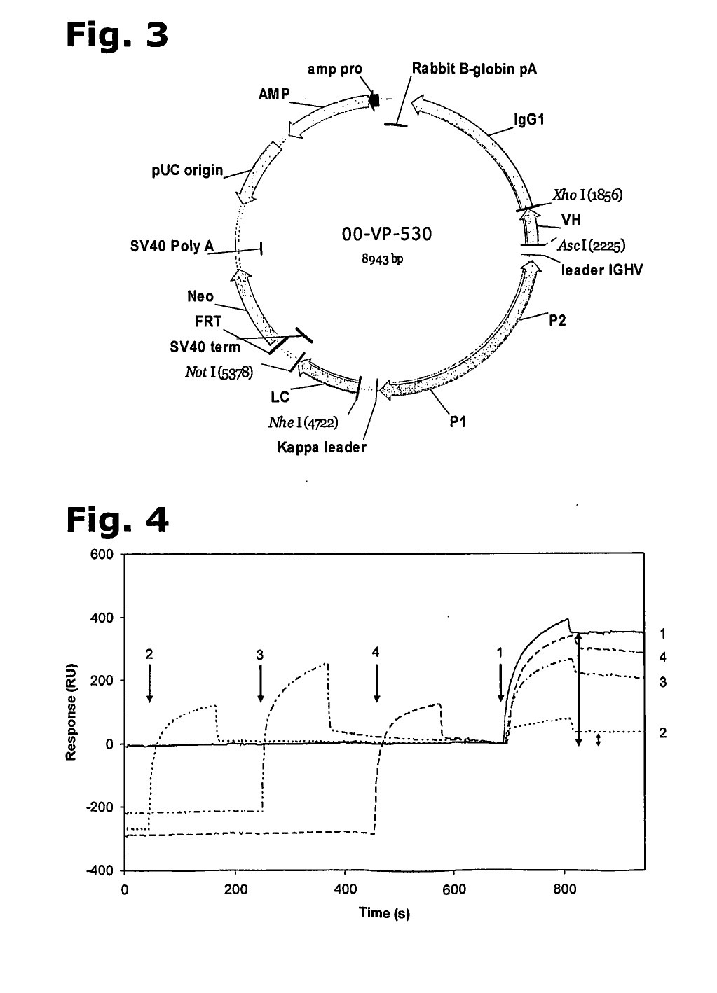

A suitable expression vector comprises a recombination recognition site matching the recombinase recognition site(s) of the host cell. Preferably the recombinase recognition site is linked to a suitable selection gene different from the selection gene used for construction of the host cell. Selection genes are well known in the art, and include glutamine synthetase gene (GS), dihydrofolate reductase gene (DHFR), and neomycin, where GS or DHFR may be used for gene amplification of the inserted V_{H} and V_{L} sequence. The vector may also contain two different recombinase recognition sites to allow for recombinase-mediated cassette exchange (RMCE) of the antibody coding sequence instead of complete integration of the vector. RMCE is described in Langer et al 2002. Nucleic Acids Res. 30, 3067-3077; Schlake and Bode 1994. Biochemistry 33, 12746-12751 and Belteki et al 2003. Nat. biotech. 21, 321-324. Suitable recombinase recognition sites are well known in the art, and include FRT, 10× and attP/attB sites. Preferably the integrating vector is an isotype-encoding vector, where the constant regions (preferably including introns) are present in the vector prior to transfer of the V_{H} and V_{L} coding pair from the screening vector (or the constant regions are already present in the screening vector if screening is performed on full-length antibodies). The constant regions present in the vector can either be the entire heavy chain constant region (CH₁ to CH₃ or to CH₄) or the constant region encoding the Fc part of the antibody (CH₂ to CH₃ or to CH₄). The light chain Kappa or Lambda constant region may also be present prior to transfer. The choice of the number of constant regions present, if any, depends on the screening and transfer system used. The heavy chain constant regions can be selected from the isotypes IgG1, IgG2, IgG3, IgG4, IgA1, IgA2, IgM, IgD and IgE. Preferred isotypes are IgG1 and/or IgG3. Further, the expression vector for site-specific integration of the anti-RSV antibody-encoding nucleic acid contains suitable promoters or equivalent sequences directing high levels of expression of each of the V_{H} and V_{L} chains. FIG. 3 illustrates one possible way to design the expression vector, although numerous other designs are possible.

The transfer of the selected V_{H} and V_{L} coding pairs from the screening vector can be performed by conventional restriction enzyme cleavage and ligation, such that each expression vector molecule contain one V_{H} and V_{L} coding pair. Preferably, the V_{H} and V_{L} coding pairs are transferred individually, they may, however, also be transferred in-mass if desired. When all the selected V_{H} and V_{L} coding pairs are transferred to the expression vector a collection or a library of expression vectors is obtained. Alternative ways of transfer may also be used if desired. If the screening vector is identical to the expression vector, the library of expression vectors is constituted of the V_{H} and V_{L} sequence pairs selected during screening, which are situated in the screening/expression vector.

Methods for transfecting a nucleic acid sequence into a host cell are known in the art. To ensure site-specific integration, a suitable recombinase must be provided to the host cell as well. This is preferably accomplished by co-transfection of a plasmid encoding the recombinase. Suitable recombinases are for example Flp, Cre or phage φC31 integrase, used together with a host cell/vector system with the corresponding recombinase recognition sites. The host cell can either be transfected in bulk, meaning that the library of expression vectors is transfected into the cell line in one single reaction thereby obtaining a polyclonal cell line. Alternatively, the collection of expression vectors can be transfected individually into the host cell, thereby generating a collection of individual cell lines (each cell line produce an antibody with a particular specificity). The cell lines generated upon transfection (individual or polyclonal) are then selected for site specific integrants, and adapted to grow in suspension and serum free media, if they did not already have these properties prior to transfection. If the transfection was performed individually, the individual cell lines are analyzed further with respect to their grow properties and antibody production. Preferably, cell lines with similar proliferation rates and antibody expression levels are selected for the generation of the polyclonal cell line. The polyclonal cell line is then generated by mixing the individual cell lines in a predefined ratio. Generally, a polyclonal master cell bank (pMCB), a polyclonal research cell bank (pRCB) and/or a polyclonal working cell bank (pWCB) is laid down from the polyclonal cell line. The polyclonal cell line is generated by mixing the individual cell lines in a predefined ratio. The polyclonal cell line is distributed into ampoules thereby generating a polyclonal research cell bank (pRCB) or master cell bank (pMCB) from which a polyclonal working cell bank (pWCB) can be generated by expanding cells from the research or master cell bank. The research cell bank is primarily for proof of concept studies, in which the polyclonal cell line may not comprise as many individual antibodies as the polyclonal cell line in the master cell bank. Normally, the pMCB is expanded further to lay down a pWCB for production purposes. Once the pWCB is exhausted a new ampoule from the pMCB can be expanded to lay down a new pWCB.

One embodiment of the present invention is a polyclonal cell line capable of expressing a recombinant polyclonal anti-RSV antibody of the present invention.

A further embodiment of the present invention is a polyclonal cell line wherein each individual cell is capable of expressing a single V_{H} and V_{L} coding pair, and the polyclonal cell line as a whole is capable of expressing a collection of V_{H} and V_{L} encoding pairs, where each V_{H} and V_{L} pair encodes an anti-RSV antibody. Preferably the collection of V_{H} and V_{L} coding pairs are cognate pairs generated according to the methods of the present invention.

A recombinant polyclonal antibody of the present invention is expressed by culturing one ampoule from a pWCB in an appropriate medium for a period of time allowing for sufficient expression of antibody and where the polyclonal cell line remains stable (The window is approximately between 15 days and 50 days). Culturing methods such as fed batch or perfusion may be used. The recombinant polyclonal antibody is obtained from the culture medium and purified by conventional purification techniques. Affinity chromatography combined with subsequent purification steps such as ion-exchange chromatography, hydrophobic interactions and gel filtration has frequently been used for the purification of IgG. Following purification, the presence of all the individual members in the polyclonal antibody composition is assessed, for example by ion-exchange chromatography. The characterization of a polyclonal antibody composition is described in detail in WO 2006/007853 (PCT/DK2005/000504) (hereby incorporated by reference).

An alternatively method of expressing a mixture of antibodies in a recombinant host is described in WO 04/009618. This method produces antibodies with different heavy chains associated with the same light chain from a single cell line. This approach may be applicable if the anti-RSV rpAb is produced from a combinatorial library.

### Therapeutic Compositions

Another aspect of the invention is a pharmaceutical composition comprising as an active ingredient an anti-RSV rpAb or anti-RSV recombinant polyclonal Fab or another anti-RSV recombinant polyclonal antibody fragment. Preferably, the active ingredient of such a composition is an anti-RSV recombinant polyclonal antibody as described in the present invention. Such compositions are intended for prevention and/or treatment of RSV infections. Preferably, the pharmaceutical composition is administered to a human, a domestic animal, or a pet.

The pharmaceutical composition further comprises a pharmaceutically acceptable excipient.

Anti-RSV rpAb or polyclonal fragments thereof may be administered within a pharmaceutically-acceptable diluent, carrier, or excipient, in unit dosage form. Conventional pharmaceutical practice may be employed to provide suitable formulations or compositions to administer to patients infected with RSV, or to patients who may be at high risk if infected with RSV. In a preferred embodiment the administration is prophylactic. In another preferred embodiment the administration is therapeutic, meaning that it is administered after the onset of symptoms relating to RSV infection. Any appropriate route of administration may be employed, for example, administration may be parenteral, intravenous, intra-arterial, subcutaneous, intramuscular, intraperitoneal, intranasal, aerosol, suppository, or oral administration. For example, pharmaceutical formulations may be in the form of, liquid solutions or suspensions; for oral administration, formulations may be in the form of tablets, capsules, chewing gum or pasta, and for intranasal formulations, in the form of powders, nasal drops, or aerosols.

The pharmaceutical compositions of the present invention are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (20th ed.), ed. A. R. Gennaro, 2000, Lippincott Williams & Wilkins, Philadelphia, Pa. and Encyclopedia of Pharmaceutical Technology, eds. J. Swarbrick and J. C. Boylan, 1988-1999, Marcel Dekker, New York, N.Y.).

Preferably solutions or suspensions of the active ingredient, and especially isotonic aqueous solutions or suspensions, are used to prepare pharmaceutical compositions of the present invention. In the case of lyophilized compositions that comprise the active ingredient alone or together with a carrier, for example mannitol, such solutions or suspensions may, if possible, be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

The injection compositions are prepared in customary manner under sterile conditions; the same applies also to introducing the compositions into ampoules or vials and sealing of the containers.

Pharmaceutical compositions for oral administration can be obtained by combining the active ingredient with solid carriers, if desired granulating the resulting mixture, and processing the mixture, if desired or necessary, after the addition of appropriate excipients, into tablets, pills, or capsules, which may be coated with shellac, sugar or both. It is also possible for them to be incorporated into plastics carriers that allow the active ingredients to diffuse or be released in measured amounts.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, tablets, pills, or capsules. The formulations can be administered to human individuals in therapeutically or prophylactically effective amounts (e.g., amounts which prevent, eliminate, or reduce a pathological condition) to provide therapy for a disease or condition. The preferred dosage of therapeutic agent to be administered is likely to depend on such variables as the severity of the RSV infection, the overall health status of the particular patient, the formulation of the compound excipients, and its route of administration.

Therapeutic uses of the compositions according to the invention

The pharmaceutical compositions according to the present invention may be used for the treatment, amelioration or prophylaxis of a disease in a mammal. Conditions that can be treated or prevented with the present pharmaceutical compositions include prevention, and treatment of patients infected with RSV, or at risk of becoming infected with RSV, in particular patients who may be at high risk if infected with RSV. High-risk patients are for example infants and small children. In particular premature infants and children with an underlying problem such as chronic lung disease or congenital heart disease are at the greatest risk for serious illness such as bronchiolitis and pneumonia following RSV infection. Also high-risk adults, such as immunocompromised adults, particularly bone marrow transplant recipients, elderly individuals and individuals with chronic pulmonary disease, can preferably be subjected to prophylactic or therapeutic treatment with a pharmaceutical composition according to the present invention.

One embodiment of the present invention is a method of preventing, treating or ameliorating one or more symptoms associated with a RSV infection in a mammal, comprising administering an effective amount of an anti-RSV recombinant polyclonal antibody of the present invention to said mammal.

A further embodiment of the present invention is the use of an anti-RSV recombinant polyclonal antibody of the present invention for the preparation of a composition for the treatment, amelioration or prevention of one or more symptoms associated with a RSV infection in a mammal.

Preferably, the mammal in the embodiments above is a human, domestic animal or a pet.

In a further embodiment the mammal, subject to the method of preventing treating or ameliorating one or more symptoms associated with a RSV infection, preferably has a body weight above 40 kg.

In embodiments where the subject is a human, it is preferably a premature infant, a child with chronic lung disease or congenital heart disease. In alternative embodiments the human is an immunocompromised adult, in particularly a bone marrow transplant recipient, an elderly individual or an individual with chronic pulmonary disease.

### Diagnostic Use

Another embodiment of the invention is directed to diagnostic kits. Kits according to the present invention comprise an anti-RSV rpAb prepared according to the invention which protein may be labeled with a detectable label or non-labeled for non-label detection. The kit may be used to identify individuals infected with RSV.

### Antibody Molecules of the Present Invention and Aspects Related Thereto

It should be noted that the novel antibody molecules disclosed herein are believed to contribute to the state of the art in their own right. Hence, the present invention also relates to any one of the antibody molecules disclosed herein as well as to fragments and analogues of these antibodies, where said fragments or analogues at least incorporate the CDRs of the antibodies disclosed herein.

For instance it has been found by the present inventors that some of the fully human antibody molecules which have been isolated from human donors include binding sites that exhibit extremely high improved kinetic profiles over known prior art monoclonal antibodies when it comes to antigen binding. Thus, even though much focus is put on polyclonal antibody compositions in the present disclosure, all subject matter relating to utilization of polyclonal antibodies set forth herein is also relevant for any one of the single antibody molecules disclosed herein—i.e. all disclosures relating to formulation, dosage, administration etc. which relate to polyclonal antibody compositions of the present invention apply mutatis mutandis to the individual antibody molecules, antibody fragments and antibody analogues disclosed herein, preferably also the framework sequences.

Hence, the present invention also relates to an isolated human anti RSV-antibody molecule selected from the antibody molecules set forth in Table 5 herein, or a specifically binding fragment of said antibody molecule or a synthetic or semi-synthetic antibody analogue, said binding fragment or analogue comprising at least the complementarity-determining regions (CDRs) of said isolated antibody molecule. Often, framework regions from the variable regions of the native human antibody will be included too in the fragments or analogues, since the antigen specificity of antibodies are known to be dependent on the 3D organisation of CDRs and framework regions.

The expression “isolated antibody molecule” is intended to denote a collection of distinct antibodies which are isolated from natural contaminants, and which exhibit the same amino acid sequence (i.e. identical variable and constant regions).

Typically, the antibody molecule, fragment or analogue is derived from the antibodies listed in Table 8, or includes the heavy chain CDR amino acid sequences included in one of SEQ ID Nos: 1-44 and in the accompanying light chain CDR amino acid sequences having a SEQ ID NO which is 88 higher than the amino acid sequence selected from SEQ ID NOs. 144. This means that the antibody molecule, fragment or analogue will include the cognate pairs of variable regions found in the same out of the 44 clones discussed above.

As mentioned above, a number of the present antibody molecules exihibit very high affinities, so the invention also pertains to an isolated antibody molecule, an antibody fragment or a synthetic or semi-synthetic antibody analogue, which comprises CDRs identical to the CDRs in an Fab derived from a human antibody, said Fab having a dissociation constant, K_{D}, for the RSV G protein of at most 500 nM when measured performing surface plasmon resonance analysis on a Biacore 3000, using recombinant RSV G protein immobilized onto the sensor surface at very low density to avoid limitations in mass transport. The isolated antibody molecule, antibody fragment or synthetic or semi-synthetic antibody typically exhibit a lower K_{D} of at most, 400 nM, such as at most 300 nM, at most 200 nM, at most 100 nM, at most 1 nM, at most 900 pM, at most 800 pM, at most 700, pM, at most 600 pM, at most 500 pM, at most 400 pM, at most 300 pM, at most 200 pM, at most 100 pM, at most 90 pM, and at most 80 μM. Details concerning the Biocore measurements are provided in the examples.

Another embodiment of the invention relates to an isolated antibody molecule, an antibody fragment or a synthetic or semi-synthetic antibody, which comprises an antigen binding site identical to the antigen binding site in an Fab derived from a human antibody, said Fab having a dissociation constant, K_{D}, for the RSV F protein of at most 500 nM when measured performing surface plasmon resonance analysis on a Biacore 3000, using recombinant RSV F protein immobilized onto the sensor surface at very low density to avoid limitations in mass transport. This isolated antibody, antibody fragment or synthetic or semi-synthetic antibody typically exhibits a KD of at most, 400 nM, such as at most 300 nM, at most 200 nM, at most 100 nM, at most 1 nM, at most 900 pM, at most 800 pM, at most 700, pM, at most 600 pM, at most 500 pM, at most 400 pM, at most 300 pM, at most 200 pM, at most 100 pM, at most 90 pM, at most 80 pM, at most 70 pM, at most 60 pM, at most 50 pM, at most 40 pM, at most 30 pM, at most 25 pM at most 20 pM, at most 15 pM, at most 10 pM, at most 9 pM, at most 8 pM, at most 7 pM, at most 6 pM, and at most 5 pM.

A specially useful antibody molecule or specifically binding fragment or synthetic or semi-synthetic antibody analogue comprises the CDRs of a human antibody produced in clone No. 810, 818, 819, 824, 825, 827, 858 or 894.

As mentioned above, these useful antibody molecules of the present invention may be formulated in the same way and for the same applications as the polyclonal formulations of the present invention. Hence, the present invention relates to an antibody composition comprising an antibody molecule, specifically binding fragment or synthetic or semi-synthetic antibody analogue discussed in this section in admixture with a pharmaceutically acceptable carrier, excipient, vehicle or diluent. The composition may comprise more than one binding specificity, and may e.g. include 2 distinct antibody molecules of the invention and/or specifically binding fragments and/or synthetic or semi-synthetic antibody analogues of the invention. The composition may even comprise at least 3 distinct antibody molecules and/or antibody fragments and/or synthetic or semisynthetic antibody analogues, specifically binding fragments or synthetic or semi-synthetic antibody analogues of the invention, and may therefore constitute a composition comprising at 4, 5, 6, 7, 9, 10, 11, 12, 13, 15, 16, 17, 18, 19, 20, 21, 22, 23, 24, 25, 26, 27, 28, 29 or 30 distinct antibody molecules and/or fragments and/or synthetic or semi-synthetic antibody analogues.

Especially interesting compositions include at least one antibody molecule, fragment or analogue of the invention which binds the RSV F protein and at least one antibody, fragment or analogue of the invention which binds the RSV G protein.

Also a part of the present invention is an isolated nucleic acid fragment which encodes the amino acid sequence of at least one CDR defined of an antibody molecule of the present invention, such as a nucleic acid fragment, which at least encodes the CDRs of an antibody produced by one of the clones listed in table 5. The nucleic acid fragment is typically DNA, but can also be RNA.

Another embodiment is an isolated nucleic acid fragment, which encodes the CDR sequences of a heavy chain amino acid sequence set forth in any one of SEQ ID NOs 1-44, or an isolated nucleic acid fragment, which encodes the CDR sequences of a light chain amino acid sequence set forth in any one of SEQ ID NOs 89-132. Preferred nucleic acid fragments of the invention encode the CDR sequences of a heavy chain amino acid sequence set forth in any one of SEQ ID NOs 1-44 and set forth in the accompanying light chain CDR amino acid sequences having a SEQ ID NO which is 88 higher than the amino acid sequence selected from SEQ ID NOs. 144. This of course means that the nucleic acid fragment will encode the cognate pairs of variable regions found in the same out of the 44 clones discussed above. The nucleic acid fragment may therefor include coding sequences comprised in SEQ ID NOs: 45-88 and/or 133-176.

Conveniently the nucleic acid fragments are introduced in a vector, which is also part of the present invention. Such a vector may be capable of autonomous replication, and is typically selected from the group consisting of a plasmid, a phage, a cosmid, a mini-chromosome, and a virus.

In the event the vector of the invention is an expression vector, it will preferably have the following outline (cf. also an exemplary vector in FIG. 3):
in the 5′→3′ direction and in operable linkage at least one promoter for driving expression of a first nucleic acid fragment discussed above, which encodes at least one light chain CDR together with any necessary framework regions, optionally a nucleic acid sequence encoding a leader peptide, said first nucleic acid fragment, optionally a nucleic acid sequence encoding constant regions of an antibody, and optionally a nucleic acid sequence encoding a first terminator, and/or
in the 5′→3′ direction and in operable linkage at least one promoter for driving expression of a second nucleic acid fragment of the invention, which encodes at least one heavy chain CDR together with any necessary framework regions, optionally a nucleic acid sequence encoding a leader peptide, said second nucleic acid fragment, optionally a nucleic acid sequence encoding constant regions, and optionally a nucleic acid sequence encoding a second terminator.

Such a vector is especially useful if it can be used to stably transform a host cell, which can subsequently be cultured in order to obtain the recombinant expression product. So, the preferred vector is one, which, when introduced into a host cell, is integrated in the host cell genome.

Hence, the invention also pertains to a transformed cell carrying the vector of the invention discussed in this section and also to a stable cell line which carries this vector and which expresses the nucleic acid fragment of the invention discussed in this section. Both the transformed cell and the cell line optionally secretes or carries its recombinant expression product (i.e. the inventive antibody molecule, antibody fragment or analogue) on its surface.

### Example 1

This example is a collection of the methods applied to illustrate the present invention.

a. Sorting of Lambda-Negative Plasma Blasts from Donor Blood

The peripheral blood mononuclear cells (PBMC) were isolated from blood drawn from donors using Lymphoprep (Axis Shield) and gradient centrifugation according to the manufacturer's instructions. The isolated PBMC were either cryopreserved in FCS; 10% DMSO at −150° C. or used directly. The B cell fraction was labeled with anti-CD19 antibody and isolated from the PBMC fraction using magnetic cell sorting (MACS). The PBMC (1×10⁶ cells) were incubated with anti-CD19-FITC conjugated antibody (BD Pharmingen) for 20 min at 4° C. Cells were washed twice in, and re-suspended in MACS buffer (Miltenyi Biotec). Anti-FITC MicroBeads (Miltenyi Biotec) were mixed with the labeled cells and incubated for 15 min at 4° C. The washing procedure was repeated before the cell-bead suspension was applied to a LS MACS column (Miltenyi Biotec). The CD19 positive cell fraction was eluted from the column according to the manufactures instructions and either stored in FCS-10% DMSO, or single-cell sorted directly.

Plasma blasts were selected from the CD19⁺ B cell fraction by fluorescence activated cell sorting (FACS) based on the expression profile of CD19, CD38, and CD45 cell surface proteins. CD19 is a B-cell marker that is also expressed on plasma cell precursors, while CD38 is highly expressed on plasma blasts and plasma cells. The plasma blasts apparently have a somewhat lower expression of CD19 and CD45 than the rest of the CD19⁺ cells, which allows for the separation of a discrete population. The cells were washed in FACS buffer (PBS; 1% BSA) and stained for 20 min with anti-CD19-FITC, anti-CD38-APC, anti-Lambda-PE (BD Pharmingen). The Lambda-light chain staining was included in order to allow exclusion of cells that cannot serve as template for the PCR (see Section c). The stained cells were washed and re-suspended in FACS buffer.

The flow rate of the cells during the FACS was set at approximately 200 events/sec and the cell concentration was 5×10⁵/ml to obtain a high plasma cell rescue. The following set of gates was used. Each gate is a daughter of the former.

Gate 1: FSC/SSC gate. The lymphocyte population having the highest FSC was selected, thereby ensuring sorting of living cells.

Gate 2: SSCh/SSCw. This gate ensured sorting of single cells (doublet discrimination).

Gate 3: Events representing the plasma blasts were gated in the CD38/CD19 dot plot as CD38 High/CD19 intermediate.

Gate 4: Since the PCR procedure described in Section c only amplifies Kappa light chains, Lambda-negative events were gated in a Lambda/CD19 dot plot.

As an alternative or in addition to gate 3, the plasma blasts could also be identified as CD38high and CD45intermediate in a CD45/CD38 dot plot. This will require staining of the cells with anti-CD45-PerCP.

The resulting population that fulfilled these four criteria was single-cell sorted into 96-well PCR plates containing a sorting buffer (see Section c). The plates containing the cells were stored at −80° C.

b. ELISpot

ELISpot was used to estimate the percentage of plasma blasts expressing anti-RSV antibodies in obtained cell samples, i.e., PBMC, MACS-purified CD19⁺ cells, or a population of FACS sorted plasma blasts. 96-well plates with a nitrocellulose surface (Millipore) were coated with a solution of 25 μg/ml inactivated RSV Long particles (HyTest). The wells were blocked by incubation with RPMI, 2% milk powder and left at 4° C. for approximately 5 h followed by 1 h incubation at 37° C. Plates were washed and the cell samples were added in RPMI culture medium to each well followed by incubation at standard tissue culture conditions for 24 h. The secreted RSV-specific antibodies will bind to the immobilized virus particles surrounding the antibody producing cell. The cells were removed by washing three times in PBS; 0.01% Tween20 and three times in PBS. HRP-conjugated anti-human IgG (H+L) (CalTag) and HRP-conjugated anti-human IgA (Serotec) were added and allowed to react with the immobilized antibodies for 1 h at 37° C. The washing procedure was repeated and the chromogen substrate (3-amino-9-ethylcarbazole solubilized in N,N-DMF (di-methyl formamide)) was added. The color development was terminated after 4 min by addition of H₂O. Red spots were identified at the sites where antigen-specific antibody-secreting cells had been located.

c. Linkage of Cognate V_{H} and V_{L} Pairs

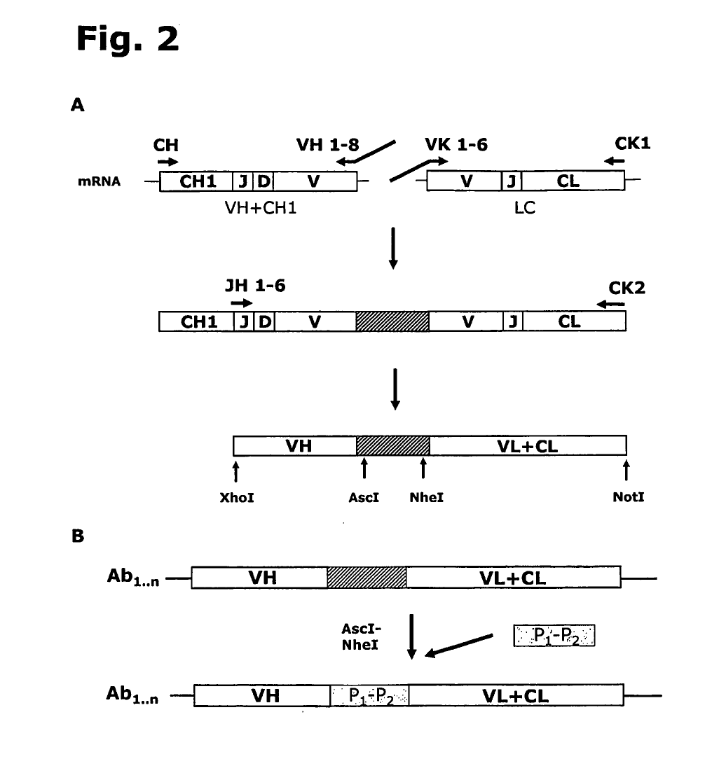

The linkage of V_{H} and V_{L} coding sequences was performed on the single cells obtained as described in Section a, facilitating cognate pairing of the V_{H} and V_{L} coding sequences. The procedure utilized a two step PCR procedure based on a one-step multiplex overlap-extension RT-PCR followed by a nested PCR. The primer mixes used in the present example only amplify Kappa light chains. Primers capable of amplifying Lambda light chains could, however, be added to the multiplex primer mix and nested PCR primer mix if desired. If Lambda primers are added, the sorting procedure in Section a should be adapted such that Lambda positive cells are not excluded. The principle for linkage of cognate V_{H} and V_{L} sequences is illustrated in FIG. 2.

The 96-well PCR plates produced in Section a, were thawed and the sorted cells served as template for the multiplex overlap-extension RT-PCR. The sorting buffer added to each well before the single-cell sorting contained reaction buffer (OneStep RT-PCR Buffer; Qiagen), primers for RT-PCR (see Table 2) and RNase inhibitor (RNasin, Promega). This was supplemented with OneStep RT-PCR Enzyme Mix (25× dilution; Qiagen) and dNTP mix (200 μM each) to obtain the given final concentration in a 20-μl reaction volume.

The plates were incubated for 30 min at 55° C. to allow for reverse transcription of the RNA from each cell. Following the RT, the plates were subjected to the following PCR cycle: 10 min at 94° C., 35×(40 sec at 94° C., 40 sec at 60° C., 5 min at 72° C.), 10 min at 72° C.

The PCR reactions were performed in H20BIT Thermal cycler with a Peel Seal Basket for 24 96-well plates (ABgene) to facilitate a high-throughput. The PCR plates were stored at −20° C. after cycling.

TABLE 2

RT-PCR multiplex overlap-extension primer mix

| | Final | | SEQ |
|---|---|---|---|
| Primer | Conc. | | ID |
| name | nM | Sequence | NO: |
| VH set | | | |
| Ch-IgG | 0.2 | GACSGATGGGCCCTTGGTGG | 179 |
| CH-IgA | 0.2 | GAGTGGCTCCTGGGGGAAGA | 180 |
| VH-1 | 0.04 | TATTCCCATGGCGCGCCCAGRTGCAGCTGGTGCART | 181 |
| VH-2 | 0.04 | TATTCCCATGGCGCGCCSAGGTCCAGCTGGTRCAGT | 182 |
| VH-3 | 0.04 | TATTCCCATGGCGCGCCCAGRTCACCTTGAAGGAGT | 183 |
| VH-4 | 0.04 | TATTCCCATGGCGCGCCSAGGTGCAGCTGGTGGAG | 184 |
| VH-5 | 0.04 | TATTCCCATGGCGCGCCCAGGTGCAGCTACAGCAGT | 185 |
| VH-6 | 0.04 | TATTCCCATGGCGCGCCCAGSTGCAGCTGCAGGAGT | 186 |
| VH-7 | 0.04 | TATTCCCATGGCGCGCCGARGTGCAGCTGGTGCAGT | 187 |
| VH-8 | 0.04 | TATTCCCATGGCGCGCCCAGGTACAGCTGCAGCAGTC | 188 |
| LC set | | | |
| CK1 | 0.2 | ATATATATGCGGCCGCTTATTAACACTCTCCCCTGTTG | 189 |
| VL-1 | 0.04 | GGCGCGCCATGGGAATAGCTAGCCGACATCCAGWTGACCCAGTCT | 190 |
| VL-2 | 0.04 | GGCGCGCCATGGGAATAGCTAGCCGATGTTGTGATGACTCAGTCT | 191 |
| VL-3 | 0.04 | GGCGCGCCATGGGAATAGCTAGCCGAAATTGTGWTGACRCAGTCT | 192 |
| VL-4 | 0.04 | GGCGCGCCATGGGAATAGCTAGCCGATATTGTGATGACCCACACT | 193 |
| VL-5 | 0.04 | GGCGCGCCATGGGAATAGCTAGCCGAAACGACACTCACGCAGT | 194 |
| VL-6 | 0.04 | GGCGCGCCATGGGAATAGCTAGCCGAAATTGTGCTGACTCAGTCT | 195 |

W = A/T, R = A/G, S = G/C

For the nested PCR step, 96-well PCR plates were prepared with the following mixture in each well (20-μl reactions) to obtain the given final concentration: 1× FastStart buffer (Roche), dNTP mix (200 μM each), nested primer mix (see Table 3), Phusion DNA Polymerase (0.08 U; Finnzymes) and FastStart High Fidelity Enzyme Blend (0.8 U; Roche). As template for the nested PCR, 1 μl was transferred from the multiplex overlap-extension PCR reactions. The nested PCR plates were subjected to the following PCR cycle: 35×(30 sec at 95° C., 30 sec at 60° C., 90 sec at 72° C.), 10 min at 72° C.

Randomly selected reactions were analyzed on a 1% agarose gel to verify the presence of an overlap-extension fragment of approximately 1070 bp.

The plates were stored at −20° C. until further processing of the PCR fragments.

TABLE 3

Nested primer set

| | Final | | |
|---|---|---|---|
| Primer | Conc. | | SEQ |
| name | nM | Sequence | ID |
| CK2 | 0.2 | ACCGCCTCCACCGGCGGCCGCTTATTAACACTCTCCCCTGTTGAAGCTCTT | 196 |
| PJ 1-2 | 0.2 | GGAGGCGCTCGAGACGGTGACCAGGGTGCC | 197 |
| PJ 3 | 0.2 | GGAGGCGCTCGAGACGGTGACCATTGTCCC | 198 |
| PJ 4-5 | 0.2 | GGAGGCGCTCGAGACGGTGACCAGGGTTCC | 199 |
| PJ 6 | 0.2 | GGAGGCGCTCGAGACGGTGACCGTGGTCCC | 200 |

d. Insertion of Cognate V_{H} and V_{L} Coding Pairs into a Screening Vector

In order to identify antibodies with binding specificity to RSV particles or antigens, the V_{H} and V_{L} coding sequences obtained as described in Section c were expressed as full-length antibodies. This involved insertion of the repertoire of V_{H} and V_{L} coding pairs into an expression vector and transformation into a host cell.

A two-step cloning procedure was employed for generation of a repertoire of expression vectors containing the linked V_{H} and V_{L} coding pairs. Statistically, if the repertoire of expression vectors contains ten times as many recombinant plasmids as the number of cognate paired V_{H} and V_{L} PCR products used for generation of the screening repertoire, there is 99% likelihood that all unique gene pairs are represented. Thus, if 400 overlap-extension V-gene fragments were obtained in Section c, a repertoire of at least 4000 clones was generated for screening.

Briefly, the repertoires of linked V_{H} and V_{L} coding pairs from the nested PCR in Section c were pooled (without mixing pairs from different donors). The PCR fragments were cleaved with XhoI and NotI DNA endonucleases at the recognition sites introduced into the termini of PCR products. The cleaved and purified fragments were ligated into an XhoI/NotI digested mammalian IgG expression vector (FIG. 3) by standard ligation procedures. The ligation mix was electroporated into E. coli and added to 2×YT plates containing the appropriated antibiotic and incubated at 37° C. over night. The amplified repertoire of vectors was purified from cells recovered from the plates using standard DNA purification methods (Qiagen). The plasmids were prepared for insertion of promoter-leader fragments by cleavage using AscI and NheI endonucleases. The restriction sites for these enzymes were located between the V_{H} and V_{L} coding gene pairs. Following purification of the vector, an AscI-NheI digested bi-directional mammalian promoter-leader fragment was inserted into the AscI and NheI restriction sites by standard ligation procedures. The ligated vector was amplified in E. coli and the plasmid was purified using standard methods. The generated repertoire of screening vectors was transformed into E. coli by conventional procedures. Colonies obtained were consolidated into 384-well master plates and stored. The number of arrayed colonies exceeded the number of input PCR products by at least 3-fold, thus giving 95% percent likelihood for presence of all unique V-gene pairs obtained in Section c.

e. Screening

The bacterial colonies arrayed in Section d were inoculated into culture medium in similar 384-well plates and grown overnight. DNA for transfection was prepared from each well in the cell culture plate. The day prior to transfection 384-well plates were seeded with CHO Flp-In cells (Invitrogen) at 3000 cells/well in 20 μl culture medium. The cells were transfected with the DNA using Fugene6 (Roche) according to the manufactures instructions. After 2-3 days incubation the full-length antibody-containing supernatants were harvested and stored for screening purposes.

Screening was performed using the Applied Biosystems 8200 FMAT™ System, a homogeneous bead-based soluble capture FLISA (fluorescent linked immunosorbent assay) (Swartzman et al. 1999, Anal. Biochem. 271:143-151). A number of antigens, including virus particles, recombinant G protein and biotinylated peptides derived from RSV antigens, were used for the screening. The peptides were derived from the conserved region (amino acids 164-176) and the cystein core region (amino acids 171-187, strain Long and 18537) of the G protein and the extracellular region of the SH-protein (amino acids 42-64 of the A2 strain and 42-65 of the 18537 strain). Inactivated virus particles of RSV strain Long (HyTest) were immobilized on polystyrene beads by incubating 300 μl 5% w/v beads (6.79 μm diameter, Spherotech Inc.) with 300 μl virus stock (protein concentration: 200 μg/ml). Soluble recombinant G protein (amino acids 66-292 of the 18537 strain sequence) was similarly immobilized directly on polystyrene beads, whereas the biotinylated peptides were captured on precoated streptavidin polystyrene beads (6.0-8.0 μm diameter, Gerlinde Kisker) at saturating concentrations. The coating mixture was incubated overnight and washed twice in PBS. Beads were re-suspended in 50 ml PBS containing 1% bovine serum albumin (PBS/BSA) and 5 μl goat-anti-human IgG Alexa 647 conjugate (Molecular probes). Ten μl of re-suspended coating mixture was added to 20 μl antibody-containing supernatant in FMAT-compatible 384-well plates and incubated for approximately 12 h, after which the fluorescence at the bead surface in individual wells was measured. A fluorescence event was recognized as positive if its intensity was at least six standard deviations above the background baseline.

The clones resulting in primary hits were retrieved from the original master plates and collected in new plates. DNA was isolated from these clones and submitted for DNA sequencing of the V-genes. The sequences were aligned and all the unique clones were selected.

The selected clones were further validated. Briefly, 2×10⁶ Freestyle 293 cells (Invitrogen) were transfected with 1.7 μg DNA from the selected clones and 0.3 μg pAdVAntage plasmid (Promega) in 2 ml Freestyle medium (Invitrogen) according to the manufacturers' instructions. After two days, supernatants were tested for IgG expression and reactivity with the different antigens used for the primary screening as well as recombinant purified F protein and an E. coli produced fragment of the G protein (amino acids 127-203 of the 18537 strain sequence) by FLISA and/or ELISA. Antibody supernatants were tested in serial dilutions allowing for a ranking of clones according to antigen reactivity.

f. Clone Repair

When using a multiplex PCR approach as described in Section c, a certain degree of intra- and inter-V-gene family cross-priming is expected due to the high degree of homology. The cross-priming introduces amino acids that are not naturally occurring in the immunoglobulin framework with several potential consequences, e.g. structural changes and increased immunogenicity, all resulting in a decreased therapeutic activity.

In order to eliminate these drawbacks and to ensure that selected clones mirror the natural humoral immune response, such cross-priming mutations were corrected in a process called clone repair.

In the first step of the clone repair procedure, the V_{H} sequence was PCR amplified with a primer set containing the sequence corresponding to the V_{H}-gene the clone of interest originated from, thereby correcting any mutations introduced by cross-priming. The PCR fragment was digested with XhoI and AscI and ligated back into the XhoI/AscI digested mammalian expression vector (FIG. 3) using conventional ligation procedures. The ligated vector was amplified in E. coli and the plasmid was purified by standard methods. The V_{H} sequence was sequenced to verify the correction and the vector was digested with NheI/NotI to prepare it for insertion of the light chain.

In the second step the complete light chain was PCR amplified with a primer set containing the sequence corresponding to the V_{L}-gene the clone of interest originated from, thereby correcting any mutations introduced by cross-priming. The PCR fragment was digested with NheI/NotI and ligated into the V_{H} containing vector prepared above. The ligation product was amplified in E. coli and the plasmid was purified by standard methods. Subsequently, the light chain was sequenced to verify the correction.

In the case where the Kappa constant region of a selected clone contained mutations, introduced during the amplification of the genes as described in Section c, it was replaced by an unmutated constant region. This was done in an overlap PCR where the repaired V_{L}-gene (amplified without the constant region) was fused to a constant region with correct sequence (obtained in a separate PCR). The whole sequence was amplified and cloned into the V_{H} containing vector as described above and the repaired light chain was sequenced to verify the correction.

g. Generation of a Polyclonal Cell Line

The generation of a polyclonal expression cell line producing a recombinant polyclonal antibody is a multi-step procedure involving the generation of individual expression cell lines which each express a unique antibody from a single V_{H} and V_{L} gene sequence. The polyclonal cell line is obtained by mixing the individual cell lines and distributing the mixture into ampoules thereby generating a polyclonal research cell bank (pRCB) or master cell bank (pMCB) from which a polyclonal working cell bank (pWCB) can be generated by expanding cells from the research or master cell bank. Generally, the polyclonal cell lines from the pRCB are used directly without generating a pWCB.

The individual steps in the process of generating a polyclonal cell line are described below.

g-1 Transfection and Selection of Mammalian Cell Lines

The Flp-In CHO cell line (Invitrogen) was used as starting cell line. In order to obtain a more homogenous cell line the parental Flp-In CHO cell line was sub-cloned by limited dilution and several clones were selected and expanded. Based on growth behavior one clone, CHO-Flp-In (019), was selected as starting cell line. The CHO-Flp-In (019) cells were cultured as adherent cells in HAM-F12 with 10% fetal calf serum (FCS).

The individual plasmid preparations each containing a selected and repaired V_{H} and V_{L} coding pair obtained in Section f, were co-transfected with Flp recombinase encoding plasmid into ˜19×10⁶ CHO-Flp-In (019) cells (for further details, see WO 04/061104) in a T175 flask using Fugene6 (Roche). Cells were trypsinated after 24 h and transferred to a 2-layer (1260 cm²) cell factory (Nunc). Recombinant cell lines were selected by culturing in the presence of 500 μg/ml Geneticin, which was added 48 h after transfection. Approximately two weeks later clones appeared. Clones were counted and cells were trypsinated and hereafter cultured as pools of clones expressing one of the RSV-specific antibodies.

g-2 Adaptation to Serum Free Suspension Culture

The individual adherent anti-RSV antibody expressing cell cultures were trypsinated, centrifuged and transferred to separate shaker flasks (250 ml) with 1.15×10⁶ cells/ml in appropriate serum free medium (Excell302, JRH Biosciences; 500 μg/ml Geneticin, anti-clumping agent (1:250) and 4 mM L-glutamin). Growth and cell morphology were followed over several weeks. After 4-6 weeks the cell lines usually showed good and stable growth behavior with doubling times below 30 h and the adapted individual cell lines were then cryopreserved in multiple ampoules.

The individual antibodies expressed during adaptation were purified from the supernatants using the method described in Section i). The purified antibody was used for the characterization of antigen specificity and biochemical properties as described below.

g-3 Characterization of Cell Lines

All the individual cell lines were characterized with respect to antibody production and proliferation. This was performed with the following assays:

Production:

The production of recombinant antibodies of the individual expression cell lines were followed during the adaptation by Kappa specific ELISA. ELISA plates were coated overnight with goat-anti-human Fc purified antibody (Serotec) in carbonate buffer, pH 9.6. Plates were washed 6 times with washing buffer (PBS; 0.05% Tween 20) and blocked by incubation for 1 h in washing buffer containing 2% skim milk. Cell culture media supernatants were added and the incubated extended for 1 h. Plates were washed 6 times in washing buffer and secondary antibodies (goat-anti-human Kappa HRP, Serotec) were added and the incubation repeated. After vigorous washing the ELISA was developed with TMB substrate and reaction stopped by addition of H₂SO₄. Plates were read at 450 nm.

Further, intracellular staining was used to determine the general expression level as well as to determine the homogeneity of the cell population in relation to expression of recombinant antibody. 5×10⁵ cells were washed in cold FACS buffer (PBS; 2% FCS) before fixation by incubation in CellFix (BD-Biosciences) for 20 min. Cells were pelleted and permeabilized in ice cold methanol for 10 min and washed twice in FACS buffer. The suspension was fluorescently tagged antibody (Goat F(ab′)₂ Fragment, Anti-human IgG(H+L)-PE, Beckman Coulter) was added. After 20 min on ice the cells were washed and re-suspended in FACS buffer followed by FACS analysis.

Proliferation:

Aliquots of the cell suspensions were taken two to three times a week and cell number, cell size and viability was determined by Vi-Cell XR (Cell viability analyzer, Beckman Coulter) analysis. The doubling time for the cell cultures was calculated using the cell numbers derived from Vi-Cell measurements.

g-4 Characterization of the Antigen Specificity of the Individual Antibodies

The antigen and epitope specificity of the individually expressed antibodies was assessed in order to allow for the generation of an anti-RSV rpAb with a well-characterized specificity. As already described in Section e, the antibodies identified during screening were validated by assessing their binding specificity to single RSV antigens (recombinant G protein, recombinant or purified F protein) or peptide fragments thereof (conserved region and cystein-core motif of protein G, subtype A and B, and the extracellular domain of SH protein, subtype A and B) by FLISA, ELISA and surface plasmon resonance (SPR; Biacore). The epitope specificities were determined in ELISA by competition with well-characterized commercial antibodies, some of which are shown in Table 4. Not necessarily all the antibodies shown in Table 4 were used in the characterization of each individual antibody of the present invention, and potentially other antibodies or antibody fragments which have been characterized with respect to the antigen, antigenic site and/or epitope they bind may also be used. Briefly, the antibodies or antibody fragments used for epitope blocking were incubated with the immobilized antigen (RSV Long particles, HyTest) in large excess, i.e. concentrations 100 times the ones giving 75% maximum binding, as determined empirically (Ditzel et al., J. Mol. Biol. 1997, 267:684-695). Following washing, the individual antibody clones were incubated with the blocked antigen at various concentrations and any bound human IgG was detected using a Goat-anti-Human HRP conjugate (Serotec) according to standard ELISA protocols. Epitope specificities were further characterized by pair-wise competition between different antibody clones in Biacore using saturating concentrations (empirically determined) of both blocking and probing antibodies. Purified F or G protein immobilized by direct amine coupling (Biacore) was used as antigen. In both the ELISA- and Biacore-based epitope mapping, the reduced binding following epitope blocking was compared to the uncompeted binding.

TABLE 4
Monoclonal antibodies for epitope mapping
of anti-F and anti-G antibodies

| | | Antigenic | | |
|---|---|---|---|---|
| MAb/Fab | Antigen | Site | Epitope (aa) | Ref. |
| 131-2a | F | F1 | F1a | 1, 2 |
| 9C5 | F | F1 | F1a | 5 |
| 92-11c | F | F1 | F1b | 1, 2 |
| 102-10b | F | F1 | F1c | 1, 2 |
| 133-1h | F | C | F2 | 1, 2, 3 |
| 130-8f | F | C | F2 (241/421) | 1, 2, 3, 4 |
| 143-6C | F | A/II | F3 | 1, 2, 3 |
| Palivizumab | F | A/II | (272) | 8 |
| 1153 | F | A/II | (262) | 3, 4 |
| 1142 | F | A/II | | 3 |
| 1200 | F | A/II | (272) | 2, 4 |
| 1214 | F | A/II | (276) | 3, 4 |
| 1237 | F | A/II | (276) | 3, 4 |
| 1129 | F | A/II | (275) | 3, 4 |
| 1121 | F | A/II | | 3 |
| 1112 | F | B/I | (389) | 3, 6 |
| 1269 | F | B/I | (389) | 3, 6 |
| 1243 | F | C | (241/421) | 3, 6 |
| Fab 19 | F | A/II | (266) | 7 |
| RSVF2-5 | F | IV | (429) | 4 |
| Mab19 | F | IV | (429) | 12 |
| 7.936 | F | V | (432-447) | 13 |
| 9.432 | F | VI | (436) | 13 |
| 63-10f | G (A) | G11 | GCRR (A171-187) | 1, 2 |
| 130-6d | G (A) | G12 | (A174-214) | 1, 2, 9 |
| 131-2g | G (A + B) | G13 | (150-173) | 1, 2, 9 |
| 143-5a | G (A + B) | G5a | | 2 |
| L9 | G (A + B) | A1/B1 | Conserved (164-176) | 14, 15 |
| 8C5 | G | ND | | 5 |
| 1C2 | G (A) | ND | GCRR (A172-188) | 10, 11 |
| 3F4 | G (A) | ND | | 10, 11 |
| 4G4 | G (A) | ND | GCRR (A172-188) | 10, 11 |

The column “Antigen” indicates the RSV associated antigen bound by the Mab/Fab, and if a subtype specificity is known this is indicated in ( ). The column “Epitope (aa)” indicates the name of the epitope recognized by the MAb/Fab, further in (amino acid positions resulting in RSV escape mutants, or peptides/protein fragments towards which binding has been show, are indicated. The numbered references (Ref.) given in Table 4 correspond to:
1. Anderson et al., J. Clin. Microbiol. 1986, 23:475-480.
2. Anderson et al., J. Virol. 1988, 62:1232-4238.
3. Beeler & van Wyke Coelingh, J. Virol. 1989, 63:2941-2950.
4. Crowe et al., JID 1998, 177:1073-1076.
5. Sominina et al., Vestn Ross Akad Med Nauk 1995, 9:49-54.
6. Collins et al., Fields Virology, p. 1313-1351.
7. Crowe et al., Virology 1998, 252:373-375.
8. Zhao & Sullender, J. Virol. 2004, 79:3962-3968.
9. Sullender, Virology 1995, 209:70-79.
10. Morgan et al., J. Gen. Virol. 1987, 68:2781-2788.
11. McGill et al., J. Immunol. Methods 2005, 297:143-152.
12. Arbiza et al., J. Gen. Virol. 1992, 73:2225-2234.
13. Lopez et al. J. Virol. 1998, 72:6922-6928.
14. Walsh et al., J. Gen. Virol. 1989, 70:2953-2961.
15. Walsh et al., J. Gen. Virol. 1998, 79:479-487.

Furthermore, the antibody clones were also characterized in terms of binding to human laryngeal epithelial HEp-2 cells (ATCC CLL-23) infected with different RSV strains (Long and B1) by FACS. Briefly, HEp-2 cells were infected with either the RSV Long (ATCC number VR-26) strain or the RSV B1 (ATCC number VR-1400) strain in serum-free medium at a ratio of 0.1 pfu/cell for 24 (Long strain) or 48 h (B1 strain). Following detachment and wash the cells were dispensed in 96-well plates and incubated with dilutions (4 pM-200 μM) of the individual anti-RSV antibodies for 1 h at 37° C. The cells were fixed in 1% formaldehyde and cell surface-bound antibody was detected by incubation with goat F(ab)₂ anti-human IgG-PE conjugate (Beckman Coulter) for 30 min at 4° C. Binding to mock-infected HEp-2 cells was similarly analyzed. Selected clones identified as protein G-specific were also tested for cross-reactivity with recombinant human fractalkine (CX3CL1; R&D systems) by ELISA. Anti-human CX3CL1/Fractalkine monoclonal antibody (R&D systems) was used as a positive control.

g-5 Characterization of Binding Kinetics of the Individual Antibodies

Kinetic analysis of the antibodies of the invention was performed using surface plasmon resonance analysis on a Biacore 3000 (Biacore AB, Uppsala, Sweden), using recombinant antigens immobilized onto the sensor surface at very low density to avoid limitations in mass transport. The analysis was performed with Fab fragments prepared from individual antibody clones using the ImmunoPure Fab preparation Kit (Pierce). Briefly, a total of 200 resonance units (RU) recombinant protein F or a total of 50 RU recombinant protein G was conjugated to a CM5 chip surface using the Amine Coupling Kit (Biacore) according to the manufacturer's instructions. The Fab fragments were injected over the chip surface in serial dilutions, starting at an optimized concentration that did not result in RUmax values above 25 when tested on the chip with immobilized protein. The association rate constant (ka) and dissociation constant (kd) were evaluated globally using the predefined 1:1 (Langmuir) association and dissociation models in the BIAevaluation 4.1 software (BIAcore).

By performing the kinetic analyses on Fab fragments, it is ensured that the data obtained truly reflects the binding affinities towards RSV protein. If one used complete antibodies, the data would reflect binding avidities, which cannot readily be translated into a meaningful measure of the exact nature of the antibodies' binding characteristics vs. the antigen.

g-6 Characterization of the Biochemical Properties of Individual Antibodies

Heterogeneity is a common phenomenon in antibodies and recombinant proteins. Antibody modifications typically occur during expression, e.g. a post-translational modifications like N-glycosylation, proteolytic fragmentation, and N- and C-terminal heterogeneity resulting in size or charge heterogeneity. In addition, modifications like methionine oxidation and deamidation can occur during subsequent short or long term storage. Since these parameters need to be well-defined for therapeutic antibodies, they were analyzed prior to the generation of the polyclonal cell line.

The methods used for characterization of purified individual antibodies (see Section i) included SDS-PAGE (reducing and non-reducing conditions), weak cation exchange chromatography (IEX), size exclusion chromatography (SEC), and RP-HPLC (reducing and non-reducing conditions). The SDS-PAGE analysis under reducing and non-reducing conditions and SEC indicated that the purified antibodies were indeed intact with minute amounts of fragmented and aggregated forms. IEX profile analysis of the purified antibodies resulted in profiles with single peaks or chromatograms with multiple peaks, indicating charge heterogeneity in these particular antibodies. Antibody preparations resulting in multiple peaks in the IEX analysis and/or aberrant migration of either the light or heavy chain in SDS gels, or unusual RP-HPLC profiles were analyzed in detail for intact N-termini by N-terminal sequencing and for heterogeneity caused by differences in the oligosaccharide profiles. In addition, selected antibodies were analyzed for the presence of additional N-glycosylation sites in the variable chains using enzymatic treatment and subsequent SDS-PAGE analysis.

g-7 Establishment of a Polyclonal Cell Line for Anti-RSV Recombinant Polyclonal Antibody Production

From the collection of established expression cell lines, a subset is selected to be mixed for the generation of a polyclonal cell line and the polyclonal research/master cell bank (pRCB/pMCB). The selection parameters can be defined according to the use of the polyclonal antibody to be produced from the polyclonal cell line and the performance of the individual cell lines. Generally the following parameters are considered:
Cell line characteristics; to optimize the stability of the polyclonal cell line, individual cell lines with doubling times between 21 and 30 hours and antibody productivity above 1 pg/cell/day are preferred.
Reactivity; the antigens/antigenic sites and epitopes which the anti-RSV rpAb shall exert reactivity against are carefully considered.
Protein chemistry; preferably antibodies with well-defined biochemical characteristics are included in the final anti-RSV rpAb.

The selected individual cell lines each expressing a recombinant anti-RSV antibody are thawed and expanded at 37° C. in serum free medium in shaker flasks to reach at least 4×10⁸ cells of each clone having a population doubling time of 21-34 hours. The viabilities are preferably in the range of 93% to 96%. The polyclonal cell line is prepared by mixing 2×10⁶ cells from each cell line. The polyclonal cell line is distributed into freeze ampoules containing 5.6×10⁷ cells and cryopreserved. This collection of vials with a polyclonal cell line is termed the polyclonal research/master cell bank (pRCB/pMCB) from which the polyclonal working cell bank (pWCB) can be generated by expanding one ampoule from the pRCB/pMCB to reach a sufficient number of cells to lay down a polyclonal working cell bank (pWCB) of approximately 200 ampoules with the same cell density as the ampoules of the pRCB/pMCB. Samples from the cell banks are tested for mycoplasma and sterility.

h. Expression of a Recombinant Polyclonal Anti-RSV Antibody

Recombinant polyclonal anti-RSV antibody batches are produced in 5 liter bioreactors (B. Braun Biotech International, Melsungen, Germany). Briefly, vials from the pRCB or pWCB are thawed and expanded in shaker flasks (Corning). Cells in seed train are cultured in ExCell 302 medium with G418 and with anti-clumping agent at 37° C., 5% CO₂. The bioreactors are inoculated with 0.6×10⁶ cells/ml suspended in 3 l ExCell 302 medium without G418 and without anti-clumping agent. The cell numbers/viable cells are monitored daily by CASY or ViCell counting. At 50 h, 2000 ml ExCell 302 medium is supplemented and after 92 h a temperature downshift from 37° C. to 32° C. is performed. The cell culture supernatant is harvested after 164 h and subjected to purification as described in Section i).

i. Purification of Individual Anti-RSV Antibodies and Polyclonal Anti-RSV Antibodies

The antibodies expressed as described in Section g.g-2 and h, all of the IgG1 isotype, were affinity purified using a MabSelect SuRe column (Protein-A). The individual antibodies interacted with immobilized Protein A at pH 7.4, whereas contaminating proteins were washed from the column. The bound antibodies were subsequently eluted from the column by lowering of the pH to 2.7. The fractions containing antibodies, determined from absorbance measurements at 280 nm, were pooled and buffer changed using a G-25 column into 5 mM sodium acetate, 150 mM NaCl, pH 5 and stored at −20° C.

j. In Vitro Neutralization Assays

j-1 Preparation of Live RSV for In Vitro Use

Human laryngeal epithelial HEp-2 cells (ATCC CLL-23) were seeded in 175 cm² flasks at 1×10⁷ cells/flask. The cells were infected with either the RSV Long (ATCC number VR-26), the RSV B1 (ATCC number VR-1400) or the RSV B Wash/18537 (Advanced Biotechnologies Inc.) strain in 3 ml serum-free medium at a ratio of 0.1 pfu/cell. Cells were infected for 2 h at 37° C.; 5% CO₂ followed by addition of 37 ml of complete MEM medium. Cells were incubated until cytopathic effects were visible. The cells were detached by scraping and the media and cells were sonicated for 20 sec and aliquoted, snap frozen in liquid nitrogen and stored at −80° C.

j-2 Plaque Reduction Neutralization Test (PRNT)

HEp-2 cells were seeded in 96-well culture plates at 2×10⁴ cells/well, and incubated overnight at 37° C.; 5% CO₂. The test substances were diluted in serum-free MEM and allowed to pre-incubate with RSV in the absence or presence of complement (Complement sera from rabbit, Sigma) for 30 min at 37° C. This mixture was applied to the monolayer of HEp-2 cells and incubated for 24 h at 37° C.; 5% CO₂. The cells were fixed with 80% acetone; 20% PBS for 20 min. After washing, biotinylated goat anti-RSV antibody (AbD Serotec) was added (1:200) in PBS with 1% BSA and incubated for 1 h at room temperature. After washing, HRP-avidin was added and allowed to incubate for 30 min. Plaques were developed by incubation with 3-amino-9-ethylcarbazole (AEC) substrate for 25 min (RSV Long) or 45 min (RSV B1). Plaques were counted in a Bioreader (Bio-Sys GmbH). EC₅₀ values (effective concentrations required to induce a 50% reduction in the number of plaques) were calculated where applicable to allow for a comparison of the potencies.

j-3 Fusion Inhibition Assay

The fusion inhibition assay was essentially performed as the plaque reduction neutralization assay except that RSV was allowed to infect before addition of test substances. In practice, virus was added in serum-free medium to the mono-layer of HEp-2 cells for 1.5 h. Supernatants were removed and test substances were added in complete MEM medium with or without complement (Complement sera from rabbit, Sigma). The plates were incubated overnight and processed as described above for the plaque reduction neutralization assay.

j-4 Microneutralization Assay

In addition to the PRNT and fusion inhibition assay described in Sections j-2 and j-3, a microneutralization assay based on the detection of RSV proteins was employed for the determination of RSV neutralization and fusion inhibition.

For the neutralization test, the test substances were diluted in serum-free MEM and allowed to pre-incubate with RSV in the absence or presence of complement (Complement sera from rabbit, Sigma) in 96-well culture plates for 30 min at room temperature. Trypsinated HEp-2 cells were added at 1.5×10⁴ cells/well, and incubated for 2-3 days at 37° C.; 5% CO₂. The cells were washed and fixed with 80% acetone; 20% PBS for 15 minutes at 4° C. and dried. The plates were then blocked with PBS with 0.5% gelatin for 30 min at room temperature and stained with a pool of murine monoclonal antibodies against RSV proteins (NCL-RSV3, Novocastra), diluted 1:200 in PBS with 0.5% gelatin and 0.5% Tween-20, for 2 h at room temperature. After washing, Polyclonal Rabbit anti-mouse Immunoglobulin HRP-conjugate (P0260; DakoCytomation), diluted 1:1000 in PBS with 0.5% gelatin and 0.5% Tween-20 was added and allowed to incubate for 2 h at room temperature. The plates were washed and developed by addition of ortho-phenylendiamine. The reaction was stopped by addition of H₂SO₄ and the plates were read in an ELISA plate reader at 490 nm.

The fusion inhibition assay was essentially performed as the microneutralization test with the exception that virus was added to cells and incubated for 1.5 h at 37° C.; 5% CO₂ before the test substances, diluted in complete MEM, were added. The plates were incubated for 2-3 days at 37° C.; 5% CO₂ and developed as described above.

k. In Vivo Protection Assays

k-1 Mouse Challenge Model

7-8-weeks old female BALB/c mice were inoculated intraperitoneally with 0.2 ml antibody preparation on day −1 of study. Placebo treated mice were similarly inoculated i.p. with 0.1 ml PBS buffer. On day 0 of study, the mice were anesthetized using inhaled isofluorane and inoculated intranasally with 10⁻⁶-10⁻⁷ pfu of RSV strain A2 in 50 μl or with cell lysate (mock inoculum). Animals were allowed 30 seconds to aspirate the inoculum whilst held upright until fully recovered from the anaesthesia.

Five days after challenge, the mice were killed with an overdose of sodium pentobarbitone. At post-mortem, blood was obtained by exsanguination from the axillary vessels for preparation of sera. Lungs were removed and homogenized in 2.5 ml buffer with sterile sand. Lung homogenates were centrifuged to sediment sand and cell debris and supernatants were aliquoted and stored at −70° C.

The virus load was determined by quantification of the number of RSV RNA copies in the lung samples using reverse transcriptase (RT-) PCR. RNA was extracted from the lung homogenate samples using the MagNA Pure LC Total Nucleic Acid kit (Roche Diagnostics) automated extraction system according to the manufacturer's instructions. Detection of RSV RNA was performed by single-tube real-time RT-PCR using the LightCycler instrument and reagents (Roche Diagnostics) with primers and fluorophore-labeled probes specific for the N gene of RSV subtype A as described by Whiley et al. (J. Clinical Microbiol. 2002, 40: 4418-22). Samples with known RSV RNA copy numbers were similarly analyzed to derive a standard curve.

The levels of different cytokines and chemokines in lung tissue samples were determined by a commercial multiplexed immunoassay at Rules-Based Medicine (Austin, Tex.) using their rodent multi-analyte profile (MAP).

k-2 Cotton Rat Challenge Model

6-8-weeks old female cotton rats (Sigmodon hispidus) are inoculated intraperitoneally with 0.5 ml antibody preparation or placebo (PBS) on day −1 of study. 24 hours later, the animals are lightly anaesthetised with isofluorane and given an intranasal challenge of 10⁻⁶-10⁻⁷ pfu RSV strain A2 or control medium (mock inoculum). A total volume of 100 μl inoculum is administered and distributed evenly to both nares. After completion of the intranasal challenge each animal is held in the upright position for a minimum of 30 seconds to allow full inspiration of the inoculum. Five days after challenge, the animals are killed by lethal intraperitoneal injection of pentobarbitone and exsanguinated by cardiac puncture. Serum samples are obtained and frozen at −80° C. and each animal is dissected under aseptic conditions for removal of lungs and nasal tissue. The tissue samples are homogenized and the supernatants stored in aliquots at −80° C.

The virus load in the tissue samples is determined by quantification of the number of RSV RNA copies by a Taq-Man real-time assay based on the method of Van Elden et al. (J Clin Microbiol. 2003, 41(9):4378-4381). Briefly, RNA is extracted from the lung homogenate samples using the RNeasy (Qiagen) method according to the manufacturer's instructions. The extracted RNA is reverse transcribed into cDNA and subsequently amplified by PCR using the Superscript III Platinum One Step Quantitative RT-PCR System (Invitrogen) with primers and labelled probes specific for the N gene of RSV subtype A. Samples with known RSV concentrations are similarly analyzed to derive a standard curve.

### Example 2

In the present Example the isolation, screening, selection and banking of clones containing cognate V_{H} and V_{L} pairs expressed as full-length antibodies with anti-RSV specificity was illustrated.

### Donors

A total of 89 donors were recruited among the employees and parents of the children who were hospitalized at the Department of Paediatrics at Hvidovre Hospital (Denmark) during the RSV season. A initial blood sample of 18 ml was drawn, CD19⁺ B cells were purified (Example 1, Section a) and screened for the presence of anti-RSV antibodies using ELISpot (Example 1, Section b) and the frequency of plasma cells was determined by FACS analysis.

Eleven donors were found positive in the screening of the initial blood samples and a second blood sample of 450 ml was collected from ten of these. The plasma blasts were single-cell sorted according to Example 1, Section a. ELISpot was performed on a fraction of the CD19 positive B cells.

Four donors with ELISpot frequencies in the second blood donation between 0.2 and 0.6% RSV specific plasma cells (IgG⁺ and IgA⁺) of the total plasma cell population were identified. These frequencies were considered high enough to proceed to linkage of repertoires of cognate V_{H} and V_{L} pairs.

### Isolation of Cognate V_{H} and V_{L} Coding Pairs

The nucleic acids encoding the antibody repertoires were isolated from the single cell-sorted plasma cells from the five donors, by multiplex overlap-extension RT-PCR (Example 1, section c). The multiplex overlap-extension RT-PCR creates a physical link between the heavy chain variable region gene fragment (V_{H}) and the full-length light chain (LC). The protocol was designed to amplify antibody genes of all V_{H}-gene families and the kappa light chain, by using two primer sets, one for V_{H} amplification and one for the LC amplification. Following the reverse transcription and multiplex overlap-extension PCR, the linked sequences were subjected to a second PCR amplification with a nested primer set.

Each donor was processed individually, and 1480 to 2450 overlap products were generated by the multiplex overlap-extension RT-PCR. The generated collection of cognate linked V_{H} and V_{L} coding pairs from each donor were pooled and inserted into a mammalian IgG expression vector (FIG. 3) as described in Example 1 section d). The generated repertoires were transformed into E. coli, and consolidated into twenty 384-well master plates and stored. The repertoires constituted between 1×10⁶ and 3.6×10⁶ clones per donor.

### Screening

IgG antibody-containing supernatants were obtained from CHO cells transiently transfected with DNA prepared from bacterial clones from the master plates. The supernatants were screened as described in Example 1, section e. Approximately 600 primary hits were sequenced and aligned. The majority fell in clusters of two or more members, but there were also clones that only were isolated once, so-called singletons. Representative clones from each cluster and the singletons were subjected to validation studies as described in Example 1, section e). A number of the primary hits were excluded from further characterization due to unwanted sequence features such as unpaired cysteins, non-conservative mutations, which are potential PCR errors, insertions and/or deletion of multiple codons, and truncations.

A total of 85 unique clones passed the validation. These are summarized in Table 5. Each clone number specifies a particular V_{H} and V_{L} pair. The IGHV and IGKV gene family is indicated for each clone and specifies the frame work regions (FR) of the selected clones. The amino acid sequence of the complementarity determining regions (CDR) of an antibody expressed from each clone are shown, where CDRH1, CDRH2, CDRH3 indicate the CDR regions 1, 2 and 3 of the heavy chain and CDRL1, CDRL2 and CDRL3 indicate the CDR regions 1, 2 and 3 of the light chain.

The complete variable heavy and light chain sequence can be established from the information in Table 5.

Further details to the individual columns of Table 5 are given below.

The IGHV and IGKV gene family names, were assigned according to the official HUGO/IMGT nomenclature (IMGT; Lefranc & Lefranc, 2001, The Immunoglobulin FactsBook, Academic Press). Numbering and alignments are according to Chothia (Al-Lazikani et al. 1997 J. Mol. Biol. 273:927-48). Clone 809 has a 2 codon insertion 5′ to CDRH1, which likely translates into an extended CDR loop. Clone 831 has a 1 codon deletion at position 31 in CDRH1.

The column “Ag” indicates the RSV associated antigen recognized by the antibody produced from the named clone, as determined by ELISA, FLISA and/or Biacore. “+” indicates that the clone binds to RSV particles and/or RSV-infected cells, but that the antigen has not been identified.

The column “Epitope” indicates the antigenic site or epitope recognized by the antibody produced from the named clone (see Table 4 and below). “U” indicates that the epitope is unknown. UCI and UCII refer to unknown cluster I and II. Antibodies belonging to these clusters have similar reactivity profiles but have currently not been assigned to a particular epitope. Some antibodies recognize complex epitopes, such as A&C. Epitopes indicated in ( ) have only been identified in ELISA.

TABLE 5

Summary of sequence and specificity of each unique validated
clone.

| | | CDRH1 | CDRH2 | CDRH3 |
|---|---|---|---|---|
| | IGHV | 3 3 | 5 6 | 9 0 0 |
| Clone | gene | 1ab2345 | 012abc3456789012345 | 234567890abcdefghijklmn123 |
| 735 | 4-59 | D--YDWS | NIN---YRGNTNYNPSLKS | CARDVGYGGGQYFAM--------DVW |
| 736 | 3-30 | T--YGMH | FIRY--DGSTQDYVDSVKG | CAKDMDYYGSRSYSVTYYYGM--DVW |
| 743 | 1-69 | T--YALT | RITP--MFDITNYAQKFQG | CARRGAVALVPAAEDPYYYGM--DVW |
| 744 | 1-2 | G--YYMH | WINT--SSGGTNYAQKFQG | CAREDGTMGTNSWYGWF------DPW |
| 793 | 3-11 | D--YYMS | YINR--GGTTIYYADSVKG | CARGLILALPTATVELGAF----DIW |
| 794 | 1-18 | N--YGLN | WINA--YNDNTYYSPSLQG | CARSYRSQTDILTGRYKGPGDVFDNW |
| 795 | 4-30-4 | SGDYYWS | YIF---HSGTTYYNPSLKS | CARDVDDFPVWGMNRYL------ALW |
| 796 | 3-30 | H--FGMH | IISY--DGNNVHYADSVKG | CAKDDVATDLAAYYYF-------DVW |
| 797 | 1-18 | R--FGIS | WISA--DNGNTYYAQNFQD | CVRGGVVTNRVYYYYGM------DVW |
| 798 | 7-4-1 | S--YVMN | WINT--NIGDPAYAQDFTG | CAWFGEFGLF-------------DYW |
| 799 | 3-30 | N--YGMH | VISY--DGRNKYFADSVKG | CARGSVQVWLHLGLF--------DNW |
| 800 | 3-33 | D--YGMN | VIWH--DGSNKNYLDSVKG | CARTPYEFWSGYYF---------DFW |
| 801 | 3-33 | S--YAMH | VIYY--EGSNEYYADSVKG | CARKWLGM---------------DFW |
| 802 | 3-48 | S--YEMN | YIGT--GGSDIYYGDSVKG | CARARPGYKV-------------DFW |
| 803 | 4-30-4 | SGDYFWS | YIY---SSGSTFYNASLKS | CARGGTLYTTGGEM---------HIW |
| 804 | 3-64 | N--YAMH | ATST--DGGSTYYADSLKG | CARRFWGFGNFF-----------DYW |
| 805 | 4-59 | G--DFWS | YIY---YRGSTYYNPSLKS | CAREGHHSGSGDYYSFF------DYW |
| 806 | 5-51 | S--YWIG | IVYP--GDSDTTYSPSFQG | CVRRGGFCTATGCYAGHWF----DPW |
| 808 | 2-70 | TTRMSVS | RID---WDDDKYYSTSLKT | CARIVFHTSGGYYNPYM------DVW |
| 809 | 5-51 | FVSTWIG | IINP--ADSDTRYSPSFQG | CARRAYDSGWHF-----------EHW |
| 810 | 1-69 | N--YAIN | RIIP--VFDTTNYAQKFQG | CLRGSTRGWDTDGF---------DIW |
| 811 | 1-46 | N--YYIH | VINP--NGGSTTSAQKFQD | CARQRSVTGGFDAWLLIPDAS--NTW |
| 812 | 1-69 | S--YSIS | MILP--ISGTTNYAQTFQG | CARVFREFSTSTLDPYYF-----DYW |
| 813 | 5-51 | S--YWIG | IIYP--GDSDTRNSPSFQG | CVRQGGYYDRNGYHEKYAF----DIW |
| 814 | 3-30-3 | D--YAMH | VISY--DGANEYYAESVKG | CARAGRSSMNEEVIMYF------DNW |
| 816 | 3-23 | T--YAMT | VIRA--SGDSEIYADSVRG | CANIGQRRYCSGDHCYGHF----DYW |
| 817 | 3-30 | T--HGMH | IISL--DGIKTHYADSVKG | CAKDHIGGTNAYFEWTVPF----DGW |
| 818 | 2-70 | AGRVGVS | RID---WDDDKAFRTSLKT | CARTQVFASGGYYLYYL------DHW |
| 819 | 4-30-4 | GADYYWS | FIY---DSGSTYYNPSLRS | CARDLGYGGNSYSHSYYYGL---DVW |
| 822 | 5-51 | N--SWIG | IIYP--GDSTTTYTPSFQG | CARQGRGF---------------GLW |
| 823 | 4-b | SG-HFWG | SIF---HSGTTFHNPSLKS | CARVHGGGF--------------DHW |
| 824 | 4-59 | N--YYWG | HIY---FGGNTNYNPSLQS | CARDSSNWPAGY-----------EDW |
| 825 | 1-18 | S--NGLS | WISA--SSGNKKYAPKFQG | CAKDGGTYVPYSDAF--------DFW |
| 827 | 1-24 | A--LSKH | FFDP--EDGDTGYAQKFQG | CATVAAAGNF-------------DNW |
| 828 | 1-3 | T--NGLH | LINA--GNGDTRFSQKFQG | CARIAITMVRNPF----------DIW |
| 829 | 2-70 | RNRMSVS | RID---WDDDKFYNTSLQT | CARTGIYDSSGYYLYYF------DYW |
| 830 | 1-18 | T--YGVS | WISA--YNGNTYYLQKLQG | CARDRVGGSSSEVLSRAKNYGL-DVW |
| 831 | 1-3 | ---YAMH | WINV--GNGQTKYSQRFQG | CARRASQYGEVYGNYF-------DYW |
| 833 | 3-30 | Y--IGMH | AISY--DGSNKQYADSVKG | CAKDDFGNSNGVFFMSRV-----AFW |
| 834 | 1-18 | T--YGLN | WVSA--HNGNTYYAEKFHD | CVRGFNEQQLVPGLSFWF-----DYW |
| 835 | 1-18 | S--YGFS | WSSV--YNGDTNYAQKFHG | CARDRNVVLLPAAPFGGM-----DVW |
| 836 | 4-b | SG-HYWG | SIY---DSGNTYYTPSLKS | CARGSPGDAF-------------DIW |
| 838 | 3-30 | T--FGMH | VISY--EGNKKYYADSVKG | CAAQTPYFNESSGLV--------PDW |
| 839 | 3-30 | S--YGLH | EISY--DGGSKFYTDSVKG | CARDLGDGYTAWGWF--------DPW |
| 841 | 1-18 | S--FGIS | WISA--YNGNTDYAQRLQD | CTRDESMLRGVTEGFGPI-----DYW |
| 842 | 1-18 | R--YGIS | WISA--YNGNTYYAQNLQG | CVISFDSTIAAAEYF--------DYW |
| 843 | 1-18 | N--SGVS | WISA--YNGNTYYRQSLQD | CAREGHYSGSSSYQRDDAF----DIW |
| 845 | 1-18 | S--YGIS | WIGT--DNGNTYYAQKFQG | CARGGTIEATPEREYYYYGM---DVW |
| 846 | 4-30-2 | SGGYSWS | YIY---HSGSTYYNPSLKS | CASRSFYGDY-------------VYW |
| 848 | 4-61 | SDKNYWS | RLY---PSGNTDYHPSLKS | CAKEGSGWYF-------------ESW |
| 849 | 3-73 | G--STMH | RIRSKANSYATEYAASVKG | CTRHVGEMSTIWWYF--------DLW |
| 850 | 1-3 | T--YTLH | LINA--ANGHTKYSQRFQG | CAKSGSHYGEVYGAYF-------DYW |
| 851 | 1-18 | S--LGFS | WTSA--HNGNTYYAEEFQD | CARDRGPGYSDSSFYVF------DYW |
| 852 | 1-69 | G--YTIH | RLVP--SLNIPNYAQKFQG | CTRAPRGSTASHLLF--------DYW |
| 853 | 5-51 | N--YWIG | VIFP--ADSDARYSPSFQG | CARPKYYFDSSGQFSEMYYF---DFW |
| 855 | 1-18 | N--YAFS | WISG--SNGNTYYAEKFQG | CARDLLRSTYF------------DYW |
| 856 | 1-18 | N--YGFS | WISA--YNGNTYYAQNLQG | CARDGNTAGVDMWSRDGF-----DIW |
| 857 | 3-23 | S--YAMN | GISG--SGGSTYYGDSVKG | CAKEPWIDIVVASVISPYYYDGMDVW |
| 858 | 1-69 | G--YTIS | RVVP--TLGFPNYAQKFQG | CARMNLGSHSGRPGF--------DMW |
| 859 | 3-33 | K--YGIH | VISY--DGSKKYFTDSVKG | CATGGGVNVTSWSDVEHSSSL--GYW |
| 861 | 3-30 | S--YGMH | FIWN--DGSNKYYADSVKG | CVKDEVYDSSGYYLYYF------DSW |
| 863 | 3-23 | S--YTMS | SISA--STVLTYYADSVKG | CAKDYDFWSGYPGGQYWFF----DLW |
| 866 | 1-18 | T--YGIS | WISA--DNGNTYYAQKFQG | CVRGGTYSSDVEYYYYGM-----DVW |
| 867 | 1-69 | R--YTIH | RVVP--SLGIPNYAPKFQG | CARLTLGSYIGRPGF--------DSW |
| 868 | 4-b | NA-YYWG | SIH---HSGSAYYNSSLKS | CARDTILTFGEPHWF--------DPW |
| 869 | 3-30 | Y--YAMH | VISY--GETNKLYADSVKG | CARDLRYLTYYSGSGD-------DSW |
| 870 | 4-59 | N--YYWS | EIS---NTWSTNYNPSLKS | CARGLFYDSGGYYLFYF------QHW |
| 871 | 3-33 | N--YGMH | VIWY--DDSNKQYGDSVKG | CARASEYSISWRHRGVL------DYW |
| 874 | 3-30 | H--YGMH | VISH--DGNIKYSADSVKG | CHGEGYSTSWLGTAAL-------DYW |
| 879 | 3-23 | A--YAMS | AISG--GGGTTYYADSVKG | CAKTRGYSYTWGDAF--------DLW |
| 880 | 2-5 | TSKLGVG | LVD---WDDDRRYRPSLKS | CAHSAYYTSSGYYLQYF------HHW |
| 881 | 3-48 | S--YEMT | HIGN--SGSMIYYADSVKG | CARSDYYDSSGYYLLYL------DSW |
| 884 | 1-3 | N--FAMH | YINA--VNGNTQYSQKFQG | CARNNGGSAIIF-----------YYW |
| 885 | 4-b | SN-YYWG | SMH---HSGSSYYKPSLKS | CARDLVVVTDISIKNYF------DPW |
| 886 | 3-30 | S--YGMH | VISN--DGSNKYYADSVKG | CAKTTDQRLLVDWF---------DPW |
| 887 | 2-70 | TSRMSVS | RID---WDDDKYYSTSLKT | CARTLVYAPDSYYLYYF------DYW |
| 888 | 4-39 | SSNFYWG | SIF---YSGTTYYNPSLKS | CARHGFRYCNNGVCSINLDAF--DIW |
| 889 | 1-18 | T--YGIS | WISA--YNGNTFYAQRLQG | CARDLRMLPGGLPTRRGM-----DVW |
| 890 | 1-46 | K--FYIH | IINP--SGGSTTYAQTFQD | CARGIREGGVSVEDWMLVYSWF-DPW |
| 891 | 3-30 | S--YTMH | VVSY--DGNHNDYADSVKG | CVRAPGSMGL-------------DVW |
| 892 | 3-15 | N--AWMS | LIKSHFEGGATDYAAPVKG | CAPLGGPTPF-------------DYW |
| 893 | 3-30 | I--YGMH | VISY--DGAKKFYANSVKG | CATASTYFYDSR-----------DYW |
| 894 | 3-33 | D--YGMH | VIWH--DGSNIRYADSVRG | CARVPFQIWSGLYF---------DHW |
| 924 | 4-b | SE-YYWG | SVH---HSGSTYYNPSLKS | CARDRVALGVHYWYF--------DIW |
| 955 | 1-46 | D--YCMH | ILNP--DGGTTFYAEKFQD | CAILIARAYCGLADGQEGDF---DTW |

| | | CDRL1 | CDRL2 | CDRL3 | | |
|---|---|---|---|---|---|---|
| | IGKV | 2 3 3 | 5 | 8 9 9 | | |
| Clone | gene | 45678901abcdef234 | 0123456 | 89012345ab678 | Ag | Epitope |
| 735 | 3-11 | RASQSVNS------HLA | NTFNRVT | CQQRSNWPPALTF | F | UCI |
| 736 | 1-39 | RASQRISN------HLN | GASTLQS | CQQSYRTPP-INF | F | A/II |
| 743 | 2-28 | RSSQSLLHS-NGNNYLD | LASNRAS | CMQSLQT---PTF | G | Centr. dom |
| 744 | 3-20 | RASQSVSSS-----YLA | GASSRAT | CQQYDSSLSTWTF | F | A/II |
| 793 | 1-39 | RASQSITG------YLN | ATSTLQS | CQQSYNT---LTF | G | conserved |
| 794 | 1-12 | RASEGISS------WLA | AASTLQS | CQQTNSFP--YTF | G | GCRRA |
| 795 | 3-20 | RASQSVSSS-----YLA | GASTGAT | CQQYGRTP--YTF | F | UCI |
| 796 | 2-29 | RSSQSLLRS-DGKTFLY | EVSSRFS | CMQGLKIR--RTF | G | Conserved |
| 797 | 1-9 | RASQGISS------YLA | AASTLQS | CQQVDTYP--LTF | G | GCRRA |
| 798 | 1-16 | RASQDINN------YLA | AASSLQS | CQQYKSLP--FTF | G | GCRRA |
| 799 | 1-5 | RASQSVSS------WVA | EASNLES | CQQYHSYSG-YTF | F | U |
| 800 | 1D-13 | RASQGITD------SLA | AASRLES | CQQYSKSP--ATF | F | F1 |
| 801 | 2-28 | RSSQSLLNS-NGFNYVD | LGSNRAS | CMQALETP--LTF | F | F1 |
| 802 | 1-9 | RASQGISS------YLA | VASILES | CQQSKSFP--PTF | F | U |
| 803 | 3-20 | RASQTVSSS-----YLV | GASTRAT | CQQYGGSG--LTF | F | U |
| 804 | 3-20 | RASQSVSSG-----YLA | GASGRAT | CQQYFGSP--YTF | F | F1 |
| 805 | 1-39 | RASQGINT------YLN | AASSLQS | CQQSANSP--HTF | F | (F1) |
| 806 | 3-20 | RASQSISSG-----YLA | GASHRAT | CQQYGSSL--WTF | + | U |
| 808 | 1-39 | RASQTIAS------YLS | TASSLQS | CQHSYNTP--YTF | F | (F1) |
| 809 | 3D-15 | RASQSVGS------KLA | GASTRAT | CQQYNNWPP-YTF | F | (F1) |
| 810 | 1D-17 | RASQGISN------YLV | AASSLQS | CLQHNISP--YTF | F | A/II |
| 811 | 4-1 | RSSETVLYTSKNQSYLA | WASTRES | CQQFFRSP--FTF | G | Conserved |
| 812 | 3-20 | RASQSVSSS-----YIA | AASRRAT | CQHYGNSL--FTF | F | F1 |
| 813 | 1-5 | RASQSISS------WLA | KSSILES | CQHYNSYS--GTF | F | (F1) |
| 814 | 1-5 | RASQSIGS------RLA | DASSLES | CQQYNRDSP-WTF | G | Conserved |
| 816 | 2-28 | RSSQSLLHS-DGRYYVD | LASNRAS | CMQGLHTP--WTF | G | Conserved |
| 817 | 3-15 | WASQTIGG------NLA | GASTRAT | CQQYKNW---YTF | F | A/II |
| 818 | 1-39 | RASQTIAS------YVN | AASNLQS | CQQSYSYRA-LTF | F | B/I/F1 |
| 819 | 3-11 | RASQSVSS------SLA | DASYRVT | CQQRSNWPPGLTF | F | A/II |
| 822 | 1D-33 | QASQDITY------YLS | DVSNLER | CQQYDFLP--YTF | F | U |
| 823 | 1D-33 | QASQDIGD------SLN | DASNLET | CQHYVNLPPSFTF | + | U |
| 824 | 1D-13 | RPSQDISS------ALA | GASTLDY | CQQFNTYP--FTF | F | F1 & C |
| 825 | 4-1 | KSSQSVLYNSNNKNYLA | LASTREY | CQQYYQTP--LTF | F | UCI |
| 827 | 1-39 | RASQFISS------YLH | AASTLQS | CQQSYTNP--YTF | F | A & C/IV |
| 828 | 1-5 | RASQSIGS------WLA | KESNLES | CQQYKND---WTF | + | A & C |
| 829 | 1-39 | RASQSIAS------YLN | AASSLHS | CQHSYSTR--FTF | F | U (F1) |
| 830 | 1-5 | RASQSVTS------ELA | KASSLES | CQQYNSFP--YTF | G | GCRRA |
| 831 | 1-5 | RASQNIYN------WLA | DASTLES | CQQYNSLS--PTF | F | A/II |
| 833 | 1-12 | RANQDIDN------YLA | GASKLQT | CQQAKSFP--FTF | G | Centr. dom |
| 834 | 1-12 | RASQGISK------RLA | GASSLQH | CQQADSFP--FTF | G | GCRRA |
| 835 | 1-9 | RASQGISS------YLA | AASTLQS | CQQLNSYP--RTF | G | GCRR |
| 836 | 1-12 | RASQGIGT------WLA | AASRLQS | CQQAYSFP--RTF | F | (A/II) |
| 838 | 1-27 | RASQGISN------YLA | AASTLQS | CQKYNSAP--QTF | G | Conserved |
| 839 | 3-20 | RASQSVGGR-----SLA | DASNRAT | CQQYGSPP--WTF | G | GCRRA |
| 841 | 4-1 | RSSQSVLYSSNNKNYLA | WASTRAS | CQQFHSTP--RTF | G | GCRRA |
| 842 | 1-5 | RASQTISN------SLA | KASTLES | CQQYNSFS--FTF | G | GCRRA |
| 843 | 1-16 | RASQGISN------YLA | TTSTLRS | CQQYHSFP--YTF | G | GCRRA |
| 845 | 1-9 | RASQGISS------YLA | AASTLQS | CQQLNTYP--LTF | G | GCRRA |
| 846 | 3-20 | RASQSVSSS-----YLA | GASSRAT | CQQYGSSP--FTF | F | U |
| 848 | 1-5 | RASQGISA------WLA | DASTLAS | CQQYRSYS--YTF | F | U |
| 849 | 1-39 | RASQSISS------YLN | AASSLQS | CQQSYSTP--YTF | F | U |
| 850 | 1-5 | RASQNIYN------WLA | DASSLES | CQQYNIYS--PTF | F | (A/II) |
| 851 | 2-24 | RSSQSLVNS-DGNTYLS | QISKRFS | CMQATQFP--FTF | G | GCRRA |
| 852 | 1D-33 | QASQDVSY------YLN | DTSNLVT | CLQYHYLP--YTF | F | U |
| 853 | 3-20 | RASQSVSSN-----YLA | GASSRAA | CQQYGNSP--LTF | G | Centr. dom |
| 855 | 1D-12 | RASQAISN------WLA | AASSLQS | CQQADTFP--FTF | G | GCRRA |
| 856 | 2-40 | RSSQSLLDSNDGNTYLD | TFSYRAS | CMQRIEFP--YTF | G | GCRRA |
| 857 | 2-28 | RSSQSLLHR-NEYNYLD | WGSNRAS | CMQTLQTP--RTF | F | F1 |
| 858 | 1D-33 | QASQDISN------YLN | DATKLET | CQHFANLP--YTF | F | B/I/F1 |
| 859 | 1-27 | RASQGIRN------YLA | AASTLQS | CQRYNSAP--LTF | G | Conserved |
| 861 | 1-39 | RASQIIAS------YLN | AASSLQS | CQQSYSTPI-FTF | F | F1 |
| 863 | 3-11 | RTSQSVSS------YLA | DASNRAT | CQQRSDW---LTF | F | A/II |
| 866 | 1-9 | RASQGISI------YLA | AASTLQT | CQQLNIYP--LTF | G | GCRRA |
| 867 | 1D-33 | QASQDINN------YLN | DATDLET | CQHFANLP--YTF | F | (F1) |
| 868 | 3-15 | RASQSIKN------NLA | GASARAT | CQEYNNWPL-LTF | G | Conserved |
| 869 | 3-20 | RASQSLSDN-----YLA | GASSRPT | CQQYGTTP--ITF | G | Conserved |
| 870 | 1-39 | RASQRIAS------YLN | AASSLQS | CQQSYSTPI-YTF | F | (F1) |
| 871 | 1D-33 | QASQSISN------YLN | DASNLES | CQQYDNFP--YTF | F | UCI |
| 874 | 1-27 | RASQSIRN------FLA | AASTLQS | CQKYNSAP--WTF | G | Conserved |
| 879 | 3-15 | RASQSVTS------NLA | GASTRAT | CQQYNNWP--QTF | F | U |
| 880 | 1-39 | RASQTIAS------YVN | AASSLQS | CQQSYSFP--YTF | F | UCII |
| 881 | 1-39 | RASQTIAS------YVN | AASNLQS | CQQSYSVPR-LTF | F | UCII |
| 884 | 1-39 | RSSQTISV------FLN | AASSLHS | CQESFSS---STF | F | U |
| 885 | 3-11 | RASQSVTK------YLA | DASNRAT | CQHRRSW---PTF | + | U |
| 886 | 3-15 | RASQSVSS------NLA | SASTRAT | CQQYNMWPP-WTF | F | A/II |
| 887 | 1-39 | RASQTIAS------YVN | AASRLQS | CQQSYSIP--WTF | F | U (F1) |
| 888 | 2-28 | RSSQSLLRT-NGYNYLD | LGSIRAS | CMQSLQTS--ITF | G | GCRR |
| 889 | 1-5 | RASQSISS------WLA | KASSLES | CQQYNSYP--YTF | G | GCRRA |
| 890 | 1-39 | RASQNIRT------FIN | AASKLES | CQQGHSTP--YTF | G | Conserved |
| 891 | 2-28 | RSSQSLLHR-NGYNHLD | LGSNRAS | CMQALQTP--RTF | G | Centr. dom |
| 892 | 1-17 | RAGQGIRN------DLG | GASTLQS | CLQHNSYP--WTF | + | U |
| 893 | 2-24 | RSSRSLVHS-DGNTYLS | KISNRFS | CLQATQF---LTF | G | Conserved |
| 894 | 3-15 | RASQSVGN------NLA | GASTRAT | CQQYDKWP--ETF | F | UCI |
| 924 | 3-15 | RASQSVSS------HLA | GASTRAT | CQQYDNWL--PTF | G | Centr. dom |
| 955 | 1-5 | RASRSITS------WLA | KASSLQS | CQQYNSYP--LTF | SH | A2 aa42-64 |

The amino acid sequences from top to bottom in the column termed CDRH1 are set forth in the same order in SEQ ID NOs: 201-285.
The amino acid sequences from top to bottom in the column termed CDRH2 are set forth in the same order in SEQ ID NOs. 286-370.
The amino acid sequences from top to bottom in the column termed CDRH3 are set forth in the same order in SEQ ID NOs: 371-455.
The amino acid sequences from top to bottom in the column termed CDRL1 are set forth in the same order in SEQ ID NOs. 456-540.
The amino acid sequences from top to bottom in the column termed CDRL2 are set forth in the same order in SEQ ID NOs: 541-625.
The amino acid sequences from top to bottom in the column termed CDRL3 are set forth in the same order in SEQ ID NOs. 626-710.

### Characterization of Antigen Specificity

During validation the antigen specificity of the clones was determined to some degree by the binding to viral particles, soluble G and F protein as well as fragments of the G protein.

For clones with anti-F reactivity the specificity of the individual antibodies expressed from the clones was assessed further in order to determine the antigenic site and, if possible, the epitope bound by the individual clones (see Example 1, Section g-4). FIG. 4, illustrates characterization of the epitope specificity of antibody obtained from clone 801 using Biacore analysis. The analysis show that when protein F is blocked by 133-1 h or Palivizumab (antigenic site C and II, respectively) prior to injection of antibody 801 into the Biacore cell, a high degree of antibody 801 binding can be detected. The binding of competed 801 antibody is reduced a little when compared to binding of uncompeted 801 antibody. The reduction is however so low that it is more likely to be due to steric hindrance than direct competition for the binding site. Blockage of protein F with the 9c5 antibody (antigenic site F1) prior to injection of antibody 801 into the Biacore cell shows an almost complete inhibition of antibody 801 binding to the F protein. It is therefore concluded that antibody 801 binds protein F at the F1 site, or very close to it.

For clones with anti-G reactivity the specificity of the individual antibodies expressed from the clones was assessed further to determine whether the individual antibody binds to the central domain of the G protein, to the conserved region, or to the GCRR, and also whether the epitope is conserved or subtype specific. This was done by ELISA and/or FLISA using the following G protein fragments:

G(B): residue 66-292 from RSV strain 18537 (expressed in DG44 CHO cells)
G(B) Fragment: Residue 127-203 from RSV strain 18537 (expressed in E. coli)
GCRR A: Residues 171-187 from RSV strain Long (synthesized with selectively formed cystein bridges)
GCRR B: Residues 171-187 from RSV strain 18537 (synthesized with selectively formed cystein bridges)
G conserved: Residues 164-176

Additional epitope analyses were also performed on the anti-G reactive clones by competition assays as described in Example 1, Section g-4.

Further, one of the clones identified in a screening procedure as described in Example 1, Section e, produces an SH specific antibody. Additionally, a number of clones bind one or more of the tested RSV strains, but the antigen has not been determined.

Data relating to antigen specificity for all the validated clones are summarized in Table 5. None of the validated clones bind to human laryngeal epithelial cells, nor does any of the tested G-specific clones (793, 816, 835, 841, 853, 855, 856, and 888) bind to human fractalkine (CX3CL1).

### Characterization of Binding Kinetics

The binding affinity for recombinant RSV antigens was determined by surface plasmon resonance for a number antibody clones. The analysis was performed with Fab fragments prepared by enzymatic cleavage of the full-length antibodies. Data for a number of high-affinity antibody clones with K_{D} values in the picomolar to nanomolar range is presented in Table 6. Fab fragments derived from commercially available Palivizumab (Synagis) were similarly analyzed for reference.

TABLE 6
Kinetic binding constants and affinities of selected clones.

| Fab clone | kₒₙ | k_{off} | t_{1/2} | K_{D} |
|---|---|---|---|---|
| (antigen) | (10⁵ M⁻¹ s⁻¹) | (10⁻⁵ 1/s) | (min) | (pM) |
| 735 (F) | 4.07 | 9.18 | 130 | 226 |
| 810 (F) | 17.40 | 34.80 | 33 | 200 |
| 818 (F) | 1.92 | 2.20 | 530 | 115 |
| 817 (F) | 0.92 | 7.54 | 150 | 820 |
| 819 (F) | 3.56 | 4.99 | 230 | 140 |
| 825 (F) | 7.72 | 15.00 | 77 | 195 |
| 858 (F) | 4.97 | 0.34 | 3400 | 7 |
| 831 (F) | 3.72 | 42 | 28 | 1130 |
| 796 (G) | 8.33 | 40.3 | 28.67 | 480 |
| 811 (G) | 4.98 | 17.1 | 68 | 340 |
| 816 (G) | 20.20 | 17.80 | 65 | 90 |
| 838 (G) | 2.64 | 5.06 | 230 | 190 |
| 853 (G) | 17.7 | 140 | 8.25 | 790 |
| 859 (G) | 3.8 | 4.63 | 250 | 120 |
| Synagis (F) | 2.00 | 75.70 | 15 | 3780 |

### Generation of a Cell Bank of Clones Expressing an Individual Antibody

A subset of 47 unique cognate V_{H} and V_{L} coding pairs corresponding to clone nr 735, 736, 744, 793, 795, 796, 799, 800, 801, 804, 810, 811, 812, 814, 816, 817, 818, 819, 824, 825, 827, 828, 829, 830, 831, 835, 838, 841, 853, 855, 856, 857, 858, 859, 861, 863, 868, 870, 871, 880, 881, 884, 885, 886, 888, 894 and 955 in Table 5 were selected for the generation of stable individual expression cell lines which each express a unique antibody from a single V_{H} and V_{L} gene sequence. The full sequences (DNA and deduced amino acid) of 44 selected clones (the above-identified except 828, 885, and 955) are shown in SEQ ID NOs 1-176.

The 44 clones are characterized by producing the following V_{H} sequences, which are set forth in SEQ ID NOs. 1-44:

Clone No. 735:
QVQLQESGPGLVKPSETLSLTCTVSNGAIGDYDWSWIRQSPGKGLEWIGNINYRGNTNYNPSLKSRVTM
SLRTSTMQFSLKLSSATAADTAVYYCARDVGYGGGQYFAMDVWSPGTTVTVSS
Clone No. 736:
QVQLVESGGGVVQPGGSLRLSCTASGFTFSTYGMHWVRQAPGKGLEWVAFIRYDGSTQDYVDSVKGRF
TISRDNSKNMVYVQMNSLRVEDTAVYYCAKDMDYYGSRSYSVTYYYGMDVWGQGTTVTVSS
Clone No. 744:
QVQLVQSGAEVKKPGASVKVSCKASGYTFSGYYMHWVRQAPGQGLEWMGWINTSSGGTNYAQKFQG
RVTMTRDTSISTAHMELRRLRSDDTAVYYCAREDGTMGTNSWYGWFDPWGQGTLVTVSS
Clone No. 793:
QVQLVESGGGLVKPGGSLRLSCAASGFPFGDYYMSWIRQAPGKGLEWVAYINRGGTTIYYADSVKGRFT
ISRDNAKNSLFLQMNSLRAGDTALYYCARGLILALPTATVELGAFDIWGQGTMVTVSS
Clone No. 795:
QVQLQESGPGLVKPSQTLSLTCTVSGASISSGDYYWSWIRQSPRKGLEWIGYIFHSGTTYYNPSLKSRAV
ISLDTSKNQFSLRLTSVTAADTAVYYCARDVDDFPVWGMNRYLALWGRGTLVTVSS
Clone No. 796:
QVQLVESGGGVVQPGRSLRLSCAASGFSFSHFGMHWVRQVPGKGLEWVAIISYDGNNVHYADSVKGRF
TISRDNSKNTLFLQMNSLRDDDTGVYYCAKDDVATDLAAYYYFDVWGRGTLVTVSS
Clone No. 799:
QVQLVESGGGVVQPGRSLKLSCEASGFNFNNYGMHWVRQAPGKGLEWVAVISYDGRNKYFADSVKGR
FIISRDDSRNTVFLQMNSLRVEDTAVYYCARGSVQVWLHLGLFDNWGQGTLVTVSS
Clone No. 800:
QVQLVESGGAVVQPGRSLRLSCEVSGFSFSDYGMNWVRQGPGKGLEWVAVIWHDGSNKNYLDSVKGR
FTVSRDNSKNTLFLQMNSLRAEDTAVYYCARTPYEFWSGYYFDFWGQGTLVTVSS
Clone No. 801:
QVQLVESGGGVVQPGRSLRLSCAASGFPFNSYAMHWVRQAPGKGLEWVAVIYYEGSNEYYADSVKGRF
TISRDNSKNTLYLQMDSLRAEDTAVYYCARKWLGMDFWGQGTLVTVSS
Clone No. 804:
EVQLVESGGGLVRPGGSLRLSCSASGFTFSNYAMHWVRQAPGKRLEYVSATSTDGGSTYYADSLKGTFT
ISRDNSKNTLYLQMSSLSTEDTAIYYCARRFWGFGNFFDYWGRGTLVTVSS
Clone No. 810:
QVQLVQSGAEVKKSGSSVKVSCRASGGTFGNYAINWVRQAPGQGLEWVGRIIPVFDTTNYAQKFQGRV
TITADRSTNTAIMQLSSLRPQDTAMYYCLRGSTRGWDTDGFDIWGQGTMVTVSS
Clone No. 811:
QVQLVQSGAVVETPGASVKVSCKASGYIFGNYYIHWVRQAPGQGLEWMAVINPNGGSTTSAQKFQDRI
TVTRDTSTTTVYLEVDNLRSEDTATYYCARQRSVTGGFDAWLLIPDASNTWGQGTMVTVSS
Clone No. 812:
QVQLVQSGAEMKKPGSSVKVSCKASGGSFSSYSISWVRQAPGRGLEWVGMILPISGTTNYAQTFQGRVI
ISADTSTSTAYMELTSLTSEDTAVYFCARVFREFSTSTLDPYYFDYWGQGTLVTVSS
Clone No. 814:
QVQLVESGGGVVQPGKSVRLSCVGSGFRLMDYAMHWVRQAPGKGLDWVAVISYDGANEYYAESVKGR
FTVSRDNSDNTLYLQMKSLRAEDTAVYFCARAGRSSMNEEVIMYFDNWGLGTLVTVSS
Clone No. 816:
EVQLLESGGGLVQPGGSLRLSCVASGFTFSTYAMTWVRQAPGKGLEWVSVIRASGDSEIYADSVRGRFT
ISRDNSKNTVFLQMDSLRVEDTAVYFCANIGQRRYCSGDHCYGHFDYWGQGTLVTVSS
Clone No. 817:
QVQLVESGGGVVQPGRSLRLSCAASGFGFNTHGMHWVRQAPGKGLEWLSIISLDGIKTHYADSVKGRF
TISRDNSKNTVFLQLSGLRPEDTAVYYCAKDHIGGTNAYFEWTVPFDGWGQGTLVTVSS
Clone No. 818:
QVTLRESGPAVVKPTETLTLTCAFSGFSLNAGRVGVSWIRQPPGQAPEWLARIDWDDDKAFRTSLKTRLS
ISKDSSKNQVVLTLSNMDPADTATYYCARTQVFASGGYYLYYLDHWGQGTLVTVSS
Clone No. 819:
QVQLQESGPGLVKPSQTLSLTCTVSSGAISGADYYWSWIRQPPGKGLEWVGFIYDSGSTYYNPSLRSRV
TISIDTSKKQFSLKLTSVTAADTAVYYCARDLGYGGNSYSHSYYYGLDVWGRGTTVTVSS
Clone No. 824:
QVQLQESGPGLVKPSETLSLTCTVSGGSIGNYYWGWIRQPPGKGLEWIGHIYFGGNTNYNPSLQSRVTIS
VDTSRNQFSLKLNSVTAADTAVYYCARDSSNWPAGYEDWGQGTLVTVSS
Clone No. 825:
QVQLVQSGAEVKKPGASVKVSCKVSGYTFTSNGLSWVRQAPGQGFEWLGWISASSGNKKYAPKFQGR
VTLTTDISTSTAYMELRSLRSDDTAVYYCAKDGGTYVPYSDAFDFWGQGTMVTVSS
Clone No. 827:
QVQLVQSGAEVKKPGASVKVSCRVSGHTFTALSKHWMRQGPGGGLEWMGFFDPEDGDTGYAQKFQGR
VTMTEDTATGTAYMELSSLTSDDTAVYYCATVAAAGNFDNWGQGTLVTVSS
Clone No. 829:
QVTLKESGPALVKATQTLTLTCTFSGFSLSRNRMSVSWIRQPPGKALEWLARIDWDDDKFYNTSLQTRLT
ISKDTSKNQVVLTMTNMDPVDTATYYCARTGIYDSSGYYLYYFDYWGQGTLVTVSS
Clone No. 830:
QVQLVQSGAEVKVPGASVKVSCKASGYTFTTYGVSWVRQAPGQGLEWMGWISAYNGNTYYLQKLQGR
VTMTTDTSTSTAYMELRGLRSDDTAMYYCARDRVGGSSSEVLSRAKNYGLDVWGQGTTVTVSS
Clone No. 831:
QVQLVQSGAEVKKPGASVKVSCKASANIFTYAMHWVRQAPGQRLEWMGWINVGNGQTKYSQRFQGRV
TITRDTSATTAYMELSTLRSEDTAVYYCARRASQYGEVYGNYFDYWGQGTLVTVSS
Clone No. 835:
QVQLVQSGAEVKRPGASVKVSCKASGYTFISYGFSWVRQAPGQGLEWMGWSSVYNGDTNYAQKFHGR
VNMTTDTSTNTAYMELRGLRSDDTAVYFCARDRNVVLLPAAPFGGMDVWGQGTMVTVSS
Clone No. 838:
QVQLVESGGGVVQPGTSLRLSCAASGFTFSTFGMHWVRQAPGKGLEWVAVISYDGNKKYYADSVKGRF
TISRDNSKNTLYLQVNSLRVEDTAVYYCAAQTPYFNESSGLVPDWGQGTLVTVSS
Clone No. 841:
QVQLVQSGAEVKKPGASVKVSCKASGYTFISFGISWVRQAPGQGLEWMGWISAYNGNTDYAQRLQDRV
TMTRDTATSTAYLELRSLKSDDTAVYYCTRDESMLRGVTEGFGPIDYWGQGTLVTVSS
Clone No. 853:
EVQLVQSGAEVKKPGQSLKISCKTSGYIFTNYWIGWVRQRPGKGLEWMGVIFPADSDARYSPSFQGQVT
ISADKSIGTAYLQWSSLKASDTAIYYCARPKYYFDSSGQFSEMYYFDFWGQGTLVTVSS
Clone No. 855:
QVQLVQSGPEVKKPGASVKVSCKASGYVLTNYAFSWVRQAPGQGLEWLGWISGSNGNTYYAEKFQGRV
TMTTDTSTSTAYMELRSLRSDDTAVYFCARDLLRSTYFDYWGQGTLVTVSS
Clone No. 856:
QVQLVQSGAEVKKPGASVKVSCKASGYTFSNYGFSWVRQAPGRGLEWMGWISAYNGNTYYAQNLQGR
VTMTTDTSTTTAYMVLRSLRSDDTAMYYCARDGNTAGVDMWSRDGFDIWGQGTMVTVSS
Clone No. 857:
EVQLLESGGGLVQPGGPLRLSCVASGFSFSSYAMNWIRLAPGKGLEWVSGISGSGGSTYYGDSVKGRFT
ISRDNSKNTLYLQMNSLRAEDTAVYYCAKEPWIDIWASVISPYYYDGMDVWGQGTTVTVSS
Clone No. 858:
QVQLVQSGAEVKKPGSSVKVSCKASGGSFDGYTISWLRQAPGQGLEWMGRVVPTLGFPNYAQKFQGRV
TVTADRSTNTAYLELSRLTSEDTAVYYCARMNLGSHSGRPGFDMWGQGTLVTVSS
Clone No. 859:
QVQLVESGGGVVQPGRSLRLSCAVSGSSFSKYGIHWVRQAPGKGLEWVAVISYDGSKKYFTDSVKGRF
TIARDNSQNTVFLQMNSLRAEDTAVYYCATGGGVNVTSWSDVEHSSSLGYWGLGTLVTVSS
Clone No. 861:
QVQLVESGGGVVQPGGSLRLSCAASGFTFSSYGMHWVRQAPGKGLEWVAFIWNDGSNKYYADSVKGR
FTISRDNSKNTLYLQMNSLRAEDTAVYYCVKDEVYDSSGYYLYYFDSWGQGTLVTVSS
Clone No. 863:
EVQLLESGGGLVQPGGSLRLSCAASGFTFSSYTMSWVRQAPGKGLEWVSSISASTVLTYYADSVKGRFTI
SRDNSKNTLYLQMSSLRAEDTAVYYCAKDYDFWSGYPGGQYWFFDLWGRGTLVTVSS
Clone No. 868:
QVQLQESGPGLVTPSETLSVTCTVSNYSIDNAYYWGWIRQPPGKGLEWIGSIHHSGSAYYNSSLKSRATI
SIDTSKNQFSLNLRSVTAADTAVYYCARDTILTFGEPHWFDPWGQGTLVTVSS
Clone No. 870:
QVQLQESGPGLVKPSETLSLTCTVSGDSISNYYWSWIRQPPGKGLEWIGEISNTWSTNYNPSLKSRVTIS
LDMPKNQLSLKLSSVTAADTAVYYCARGLFYDSGGYYLFYFQHWGQGTLVTVSS
Clone No. 871:
QVQLVESGGGVVQPGRSLRVSCAASGFTFSNYGMHWVRQAPGKGLEWVAVIWYDDSNKQYGDSVKG
RFTISRDNSKSTLYLQMDRLRVEDTAVYYCARASEYSISWRHRGVLDYWGQGTLVTVSS
Clone No. 880:
QITLKESGPTLVRPTQTLTLTCTFSGESLSTSKLGVGWIRQPPGKALEWLALVDWDDDRRYRPSLKSRLTV
TKDTSKNQVVLTMTNMDPVDTATYYCAHSAYYTSSGYYLQYFHHWGPGTLVTVSS
Clone No. 881:
EVQLVESGGGVVQPGGSLRLSCEVSGFTFNSYEMTWVRQAPGKGLEWVSHIGNSGSMIYYADSVKGRF
TISRDNAKNSLYLQMNSLRVEDTAVYYCARSDYYDSSGYYLLYLDSWGHGTLVTVSS
Clone No. 884:
QVQLVQSGAEVRKPGASVKVSCKASGHTFINFAMHWVRQAPGQGLEWMGYINAVNGNTQYSQKFQGR
VTFTRDTSANTAYMELSSLRSEDTAVYYCARNNGGSAIIFYYWGQGTLVTVSS
Clone No. 886:
QVQLVESGGGVVQPGRSLRLSCAASGFSFSSYGMHWVRQAPGKGLEWVAVISNDGSNKYYADSVKGR
FTISRDNSKKTMYLQMNSLRAEDTAVYFCAKTTDQRLLVDWFDPWGQGTLVTVSS
Clone No. 888:
QLQLQESGPGLVKPSETLSLTCTASGGSINSSNFYWGWIRQPPGKGLEWIGSIFYSGTTYYNPSLKSRVTI
SVDTSKNQFSLKLSPVTAADTAVYHCARHGFRYCNNGVCSINLDAFDIWGQGTMVTVSS
Clone No. 894:
QVQLVESGGGVVQPGKSLRLSCAASGFRFSDYGMHWVRQAPSKGLEWVAVIWHDGSNIRYADSVRGR
FSISRDNSKNTLYLQMNSMRADDTAFYYCARVPFQIWSGLYFDHWGQGTLVTVSS

These V_{H} amino acid sequences are in the clones encoded by the following nucleic acid sequences, which are also set forth as SEQ ID NOs. 45-88:

Clone No. 735:
caggtgcagctgcaggagtcgggcccaggactggtgaagccttcggagaccctgtccctcacgtgcactgtgtctaatggcgccatc
ggcgactacgactggagctggattcgtcagtccccagggaagggactggagtggattgggaacataaattacagagggaacacc
aactacaacccctccctcaagagtcgagtcaccatgtccctacgcacgtccacgatgcagttctccctgaagctgagctctgcgaccg
ctgcggacacggccgtctattactgtgcgagagatgtaggctacggtggcgggcagtatttcgcgatggacgtctggagcccaggg
accacggtcaccgtctcgagt
Clone No. 736:
caggtgcagctggtggagtctgggggaggcgtggtccagcctggggggtccctgagactctcctgtacagcgtctggattcaccttc
agtacctatggcatgcactgggtccgccaggctcccggcaaggggctggaatgggtggcatttatacggtatgatggaagtactca
agactatgtagactccgtgaagggccgattcaccatctccagagacaattccaagaatatggtgtatgtgcagatgaacagcctgag
agttgaggacacggctgtctattactgtgcgaaagacatggattactatggttcgcggagttattctgtcacctactactacggaatgg
acgtctggggccaagggaccacggtcaccgtctcgagt
Clone No. 744:
caggtgcagctggtgcagtctggggctgaggtgaagaagcctggggcctcagtgaaggtctcctgcaaggcttctggatacaccttc
agcggctattatatgcactgggtgcgacaggcccctggacaagggcttgagtggatgggatggatcaacactagcagtggtggcac
aaactatgcgcagaagtttcagggcagggtcaccatgaccagggacacgtccatcagcacagcccacatggaactgaggaggctg
agatctgacgacacggccgtgtattattgtgcgagagaggacggcaccatgggtactaatagttggtatggctggttcgacccctgg
ggccagggaaccctggtcaccgtctcgagt
Clone No. 793:
caggtgcagctggtggagtctgggggaggcttggtcaagcctggggggtccctgagactctcctgtgcggcctctggattccccttcg
gtgactactacatgagctggatccgccaggctccagggaagggactggagtgggttgcatacattaatagaggtggcactaccata
tactacgcagactctgtgaagggccgattcaccatctccagggacaacgccaagaactccctgtttctgcaaatgaacagcctgaga
gccggggacacggccctctattactgtgcgagagggctaattctagcactaccgactgctacggttgagttaggagcttttgatatctg
gggccaagggacaatggtcaccgtctcgagt
Clone No. 795:
caggtgcagctgcaggagtcgggcccaggactggtgaagccttcacagaccctgtccctcacctgcactgtctctggtgcctccatca
gcagtggtgattattactggagttggatccgtcagtctccaaggaagggcctggagtggattgggtacatcttccacagtgggacca
cgtactacaacccgtccctcaagagtcgagctgtcatctcactggacacgtccaagaaccaattctccctgaggctgacgtctgtgact
gccgcagacacggccgtctattattgtgccagagatgtcgacgattttcccgtttggggtatgaatcgatatcttgccctctggggccg
gggaaccctggtcaccgtctcgagt
Clone No. 796:
caggtgcagctggtggagtctgggggaggcgtggtccagcctgggaggtccctgagactctcctgtgcagcctctggattcagcttc
agtcactttggcatgcactgggtccgccaggttccaggcaaggggctggagtgggtggcaattatatcatatgatgggaataatgta
cactatgccgactccgtaaagggccgattcaccatctccagagacaattccaagaacacgctgtttctgcaaatgaacagcctgaga
gatgacgacacgggtgtgtattactgtgcgaaggacgacgtggcgacagatttggctgcctactactacttcgatgtctggggccgt
ggcaccctggtcaccgtctcgagt
Clone No. 799:
caggtgcagctggtggagtctgggggcggcgtggtccagcctgggaggtccctgaaactctcttgtgaagcctctggattcaacttc
aataattatggcatgcactgggtccgccaggcaccaggcaaggggctggagtgggtggcagttatttcatatgacggaagaaataa
gtattttgctgactccgtgaagggccgattcatcatctccagagacgattccaggaacacagtgtttctgcaaatgaacagcctgcga
gttgaagatacggccgtctattactgtgcgagaggcagcgtacaagtctggctacatttgggactttttgacaactggggccaggga
accctggtcaccgtctcgagt
Clone No. 800:
caggtgcagctggtggagtctgggggagccgtggtccagcctgggaggtccctgagactctcctgtgaagtgtctggattcagtttc
agtgactatggcatgaactgggtccgccagggtccaggcaaggggctggagtgggtggcagttatatggcatgacggaagtaata
aaaattatctagactccgtgaagggccgattcaccgtctccagagacaattccaagaacacattgtttctgcaaatgaacagcctgag
agccgaagacacggctgtatattactgtgcgaggacgccttacgagttttggagtggctattactttgacttctggggccagggaacc
ctggtcaccgtctcgagt
Clone No. 801:
caggtgcagctggtggagtctgggggaggcgtggtccagcctgggaggtccctgagactctcctgtgcagcgtctggattccccttc
aatagctatgccatgcactgggtccgccaggctccaggcaaggggctggagtgggtggcagtgatatattatgaagggagtaatga
atattatgcagactccgtgaagggccgattcaccatctccagagacaattccaagaacactctgtatttgcaaatggatagcctgaga
gccgaggacacggctgtctattactgtgcgaggaagtggctggggatggacttctggggccagggaaccctggtcaccgtctcgag
t
Clone No. 804:
gaggtgcagctggtggagtctgggggaggcttggtccggcctggggggtccctgagactctcctgttcagcctctggattcaccttca
gtaactatgctatgcactgggtccgccaggctccagggaagagactggaatatgtttcagctactagtactgatggggggagcacat
actacgcagactccctaaagggcacattcaccatctccagagacaattccaagaacacactgtatcttcaaatgagcagtctcagtac
tgaggacacggctatttattactgcgcccgccgattctggggatttggaaacttttttgactactggggccggggaaccctggtcaccg
tctcgagt
Clone No. 810:
caggtgcagctggtgcagtctggggctgaggtgaagaagtccgggtcctcggtgaaggtctcctgcagggcttctggaggcaccttc
ggcaattatgctatcaactgggtgcgacaggcccctggacaagggcttgagtgggtgggaaggatcatccctgtctttgatacaaca
aactacgcacagaagttccagggcagagtcacgattaccgcggacagatccacaaacacagccatcatgcaactgagcagtctgc
gacctcaggacacggccatgtattattgtttgagaggttccacccgtggctgggatactgatggttttgatatctggggccaagggac
aatggtcaccgtctcgagt
Clone No. 811:
caggttcagctggtgcagtctggggctgtcgtggagacgcctggggcctcagtgaaggtctcctgcaaggcatctggatacatcttc
ggcaactactatatccactgggtgcggcaggcccctggacaagggcttgagtggatggcagttatcaatcccaatggtggtagcac
aacttccgcacagaagttccaagacagaatcaccgtgaccagggacacgtccacgaccactgtctatttggaggttgacaacctgag
atctgaggacacggccacatattattgtgcgagacagagatctgtaacagggggctttgacgcgtggcttttaatcccagatgcttct
aatacctggggccaggggacaatggtcaccgtctcgagt
Clone No. 812:
caggtgcagctggtgcagtctggggctgagatgaagaagcctgggtcctcggtgaaggtctcctgcaaggcttctggaggctccttc
agcagctattctatcagctgggtgcgacaggcccctggacgagggcttgagtgggtgggaatgatcctgcctatctctggtacaaca
aactacgcacagacatttcagggcagagtcatcattagcgcggacacatccacgagcacagcctacatggagctgaccagcctcac
atctgaagacacggccgtgtatttctgtgcgagagtctttagagaatttagcacctcgacccttgacccctactactttgactactgggg
ccagggaaccctggtcaccgtctcgagt
Clone No. 814:
caggtgcagctggtggagtctgggggaggcgtggtccagcctgggaagtccgtgagactctcctgtgtaggctctggcttcaggctc
atggactatgctatgcactgggtccgccaggctccaggcaagggactggattgggtggcagttatttcatatgatggagccaatgaa
tactacgcagagtccgtgaagggccgattcaccgtctccagagacaattcagacaacactctgtatctacaaatgaagagcctgaga
gctgaggacacggctgtgtatttctgtgcgagagcgggccgttcctctatgaatgaagaagttattatgtactttgacaactggggcct
gggaaccctggtcaccgtctcgagt
Clone No. 816:
gaggtgcagctgttggagtctgggggaggcttggtccagcctggggggtccctgagactctcctgtgtagcctccggattcaccttta
gtacctacgccatgacctgggtccgccaggctccagggaaggggctggagtgggtctcagtcattcgtgctagtggtgatagtgaaa
tctacgcagactccgtgaggggccggttcaccatctccagagacaattccaagaacacggtgtttctgcaaatggacagcctgagag
tcgaggacacggccgtatatttctgtgcgaatataggccagcgtcggtattgtagtggtgatcactgctacggacactttgactactgg
ggccagggaaccctggtcaccgtctcgagt
Clone No. 817:
caggtgcagctggtggagtctgggggaggcgtggtccaacctgggaggtccctgagactctcctgtgcagcctctggattcggcttc
aacacccatggcatgcactgggtccgccaggctccaggcaaggggctggagtggctgtcaattatctcacttgatgggattaagacc
cactatgcagactccgtgaagggccgattcaccatctccagagacaattccaagaacacggtgtttctacaattgagtggcctgaga
cctgaagacacggctgtatattactgtgcgaaagatcatattggggggacgaacgcatattttgaatggacagtcccgtttgacggct
ggggccagggaaccctggtcaccgtctcgagt
Clone No. 818:
caggtcaccttgagggagtctggtccagcggtggtgaagcccacagaaacgctcactctgacctgcgccttctctgggttctcactca
acgccggtagagtgggtgtgagttggatccgtcagcccccagggcaggccccggaatggcttgcacgcattgattgggatgatgat
aaagcgttccgcacatctctgaagaccagactcagcatctccaaggactcctccaaaaaccaggtggtccttacactgagcaacatg
gaccctgcggacacagccacatattactgtgcccggacacaggtcttcgcaagtggaggctactacttgtactaccttgaccactggg
gccagggaaccctggtcaccgtctcgagt
Clone No. 819:
caggtgcagctgcaggagtcgggcccaggactggtgaagccttcacagaccctgtccctcacctgcactgtctctagtggcgccatc
agtggtgctgattactactggagttggatccgccagcccccagggaagggcctggagtgggttgggttcatctatgacagtgggagc
acctactacaacccgtccctcaggagtcgagtgaccatatcaatagacacgtccaagaagcagttctccctgaagctgacctctgtga
ctgccgcagacacggccgtgtattactgtgccagagatctaggctacggtggtaactcttactcccactcctactactacggtttggac
gtctggggccgagggaccacggtcaccgtctcgagt
Clone No. 824:
caggtgcagctgcaggagtcgggcccaggactggtgaagccttcggagaccctgtccctcacctgcactgtctctggtggctccatc
ggaaattactactggggctggatccggcagcccccagggaagggacttgagtggattgggcatatctacttcggtggcaacaccaa
ctacaacccttccctccagagtcgagtcaccatttcagtcgacacgtccaggaaccagttctccctgaagttgaactctgtgaccgccg
cggacacggccgtgtattactgtgcgagggatagcagcaactggcccgcaggctatgaggactggggccagggaaccctggtcac
cgtctcgagt
Clone No. 825:
caggttcagctggtgcagtctggagctgaggtgaagaagcctggggcctcagtgaaggtctcctgcaaggtttctggttacaccttta
ccagtaatggtctcagctgggtgcgacaggcccctggacaagggtttgagtggctgggatggatcagcgctagtagtggaaacaa
aaagtatgccccgaaattccagggaagagtcaccttgaccacagacatttccacgagcacagcctacatggaactgaggagtctga
gatctgacgatacggccgtatattactgtgcgaaagatgggggcacctacgtgccctattctgatgcctttgatttctggggccaggg
gacaatggtcaccgtctcgagt
Clone No. 827:
caggtccagctggtacagtctggggctgaggtgaagaagcctggggcctcagtgaaggtctcctgcagggtttccggacacactttc
actgcattatccaaacactggatgcgacagggtcctggaggagggcttgagtggatgggattttttgatcctgaagatggtgacaca
ggctacgcacagaagttccagggcagagtcaccatgaccgaggacacagccacaggcacagcctacatggagctgagcagcctg
acatctgacgacacggccgtatattattgtgcaacagtagcggcagctggaaactttgacaactggggccagggaaccctggtcac
cgtctcgagt
Clone No. 829:
caggtcaccttgaaggagtctggtcctgcgctggtgaaagccacacagaccctgacactgacctgcaccttctctgggttttcactcag
taggaatagaatgagtgtgagctggatccgtcagcccccagggaaggccctggagtggcttgcacgcattgattgggatgatgata
aattctacaacacatctctgcagaccaggctcaccatctccaaggacacctccaaaaaccaggtggtccttacaatgaccaacatgg
accctgtggacacagccacctattactgcgcacggactgggatatatgatagtagtggttattacctctactactttgactactggggc
cagggaaccctggtcaccgtctcgagt
Clone No. 830:
caggtgcagctggtgcagtctggagctgaggtgaaggtgcctggggcctcagtgaaggtctcctgcaaggcttctggttacaccttta
ccacttacggtgtcagctgggtgcggcaggcccctggacaagggcttgagtggatgggttggatcagcgcttacaatggtaacacat
actatctacagaagctccagggcagagtcaccatgaccacagacacatccacgagcacagcctacatggagctgcggggcctgag
gtctgacgacacggccatgtattactgtgcgagagatcgtgttgggggcagctcgtccgaggttctatcgcgggccaaaaactacgg
tttggacgtctggggccaagggaccacggtcaccgtctcgagt
Clone No. 831:
caggttcagctggtgcagtctggggctgaggtgaagaagcctggggcctcagttaaggtttcctgcaaggcttctgcaaacatcttca
cttatgcaatgcattgggtgcgccaggcccccggacaaaggcttgagtggatgggatggatcaacgttggcaatggtcagacaaaa
tattcacagaggttccagggcagagtcaccattaccagggacacgtccgcgactacagcctacatggagctgagcaccctgagatct
gaggacacggctgtgtattactgtgcgaggcgtgcgagccaatatggggaggtctatggcaactactttgactactggggccaggg
aaccctggtcaccgtctcgagt
Clone No. 835:
caggtgcagctggtgcagtctggagctgaggtgaagaggcctggggcctcagtgaaggtctcctgcaaggcttcaggttacaccttt
atcagctatggtttcagctgggtgcgacaggcccctggacaagggcttgagtggatgggatggagcagcgtttacaatggtgacac
aaactatgcacagaagttccacggcagagtcaacatgacgactgacacatcgacgaacacggcctacatggaactcaggggcctg
agatctgacgacacggccgtgtatttctgtgcgagggatcgcaatgttgttctacttccagctgctccttttggaggtatggacgtctgg
ggccaagggacaatggtcaccgtctcgagt
Clone No. 838:
caggtgcagctggtggagtctgggggaggcgtggtccagccggggacttccctgagactctcctgtgcagcctctggattcaccttca
gtacgtttggcatgcactgggtccgccaggctccaggcaaggggctggagtgggtggcagttatatcatatgatggaaataagaaa
tactatgcagactccgtgaagggccgattcaccatctccagagacaattccaagaacacgctgtatctgcaagtgaacagcctgaga
gtcgaggacacggctgtgtattactgtgcggcccaaactccatatttcaatgagagcagtgggttagtgccggactggggccagggc
accctggtcaccgtctcgagt
Clone No. 841:
caggtgcagctggtgcagtctggagctgaggtgaagaagcctggggcctcagtgaaggtctcctgcaaggcttctggttacaccttt
atcagttttggcatcagctgggtgcgacaggcccctggacaaggacttgagtggatgggatggatcagcgcttacaatggtaacac
agactatgcacagaggctccaggacagagtcaccatgactagagacacagccacgagcacagcctacttggagctgaggagcctg
aaatctgacgacacggccgtgtactattgcactagagacgagtcgatgcttcggggagttactgaaggattcggacccattgactac
tggggccagggaaccctggtcaccgtctcgagt
Clone No. 853:
gaagtgcagctggtgcagtctggagcagaggtgaaaaagccggggcagtctctgaagatctcctgtaagacttctggatacatcttt
accaactactggatcggctgggtgcgccagaggcccgggaaaggcctggagtggatgggggtcatctttcctgctgactctgatgcc
agatacagcccgtcgttccaaggccaggtcaccatctcagccgacaagtccatcggtactgcctacctgcagtggagtagcctgaag
gcctcggacaccgccatatattactgtgcgagaccgaaatattactttgatagtagtgggcaattctccgagatgtactactttgacttc
tggggccagggaaccctggtcaccgtctcgagt
Clone No. 855:
caggttcagctggtgcagtctggacctgaggtgaagaagcctggggcctcagtgaaggtctcctgcaaggcttctggttatgtgttga
ccaactatgccttcagctgggtgcggcaggcccctggacaagggcttgagtggctgggatggatcagcggctccaatggtaacaca
tactatgcagagaagttccagggccgagtcaccatgaccacagacacatccacgagcacagcctacatggagctgaggagtctga
gatctgacgacacggccgtttatttctgtgcgagagatcttctgcggtccacttactttgactactggggccagggaaccctggtcacc
gtctcgagt
Clone No. 856:
caggtgcagctggtgcagtctggagctgaggtgaagaagcctggggcctcagtgaaggtctcctgcaaggcttctggttacacctttt
ccaactacggtttcagctgggtgcgacaggcccctggacgagggcttgagtggatgggatggatcagcgcttacaatggtaacaca
tactatgcacagaacctccagggcagagtcaccatgaccacagacacatccacgaccacagcctacatggtactgaggagcctgag
atctgacgacacggccatgtattactgtgcgagagatggaaatacagcaggggttgatatgtggtcgcgtgatggttttgatatctgg
ggccaggggacaatggtcaccgtctcgagt
Clone No. 857:
gaggtgcagctgttggagtctgggggaggcttggtacagcctggggggcccctgaggctctcctgtgtagcctctggattcagcttta
gcagctatgccatgaactggatccgcctggctccagggaaggggctggagtgggtctcaggtattagtggtagcggtggtagcactt
actacggagactccgtgaagggccggttcaccatctccagagacaattccaagaacacgctgtatctgcaaatgaacagcctgaga
gccgaggacacggccgtatattactgtgcgaaagagccgtggatcgatatagtagtggcatctgttatatccccctactactacgacg
gaatggacgtctggggccaagggaccacggtcaccgtctcgagt
Clone No. 858:
caggttcagctggtgcagtctggggctgaggtgaagaagcctgggtcctcggtgaaggtctcctgcaaggcctctggaggatccttc
gacggctacactatcagctggctgcgacaggcccctggacaggggcttgagtggatgggaagggtcgtccctacacttggttttcca
aactacgcacagaagttccaaggcagagtcaccgttaccgcggacagatccaccaacacagcctacttggaattgagcagactgac
atctgaagacacggccgtatattactgtgcgaggatgaatctcggatcgcatagcgggcgccccgggttcgacatgtggggccaag
gaaccctggtcaccgtctcgagt
Clone No. 859:
caggtgcagctggtggagtctgggggaggcgtggtccagcctgggaggtccttgagactctcctgtgcagtgtctggatccagcttc
agtaaatatggcatacactgggtccgccaggctccaggcaaggggctggagtgggtggcagttatatcgtatgatggaagtaaaa
agtatttcacagactccgtgaagggccgattcaccatcgccagagacaattcccagaacacggtttttctgcaaatgaacagcctga
gagccgaggacacggctgtctattactgtgcgacaggagggggtgttaatgtcacctcgtggtccgacgtagagcactcgtcgtcctt
aggctactggggcctgggaaccctggtcaccgtctcgagt
Clone No. 861:
caggtgcagctggtggagtctgggggaggcgtggtccagcctggggggtccctgagactctcctgtgcagcgtctggattcaccttc
agtagctatggcatgcactgggtccgccaggctccaggcaaggggctggagtgggtggcatttatatggaatgatggaagtaataa
atactatgcagactccgtgaagggccgattcaccatctccagagacaattccaagaacacgctgtatctgcaaatgaacagcctgag
agctgaggacacggctgtgtattactgtgtgaaagatgaggtctatgatagtagtggttattacctgtactactttgactcttggggcc
agggaaccctggtcaccgtctcgagt
Clone No. 863:
gaggtgcagctgttggagtctgggggaggcttggtacagcctggggggtccctgagactctcctgtgcagcctctggattcacgttta
gctcctataccatgagctgggtccgccaggctccagggaaggggctggagtgggtctcaagtattagtgctagtactgttctcacata
ctacgcagactccgtgaagggccgcttcaccatctccagagacaattccaagaacacgctgtatctgcaaatgagtagcctgagagc
cgaggacacggccgtatattactgtgcgaaagattacgatttttggagtggctatcccgggggacagtactggttcttcgatctctgg
ggccgtggcaccctggtcaccgtctcgagt
Clone No. 868:
caggtgcagctgcaggagtcgggcccaggactggtgacgccttcggagaccctgtccgtcacttgcactgtctctaattattccatcg
acaatgcttactactggggctggatccggcagcccccagggaagggtctggagtggataggcagtatccatcatagtgggagcgcc
tactacaattcgtccctcaagagtcgagccaccatatctatagacacgtccaagaaccaattctcgttgaacctgaggtctgtgaccgc
cgcagacacggccgtatattactgtgcgcgcgataccatcctcacgttcggggagccccactggttcgacccctggggccagggaac
cctggtcaccgtctcgagt
Clone No. 870:
caggtgcagctgcaggagtcgggcccaggactggtgaagccttcggagaccttgtccctcacctgcactgtctcaggtgactccatc
agtaattactactggagttggatccggcagcccccagggaagggactggagtggattggagaaatatctaacacttggagcaccaa
ttacaacccctccctcaagagtcgagtcaccatatctctagacatgcccaagaaccagttgtccctgaagctgagctctgtgaccgctg
cggacacggccgtatattactgtgcgagagggcttttctatgacagtggtggttactacttgttttacttccaacactggggccagggc
accctggtcaccgtctcgagt
Clone No. 871:
caggtgcagctggtggagtctgggggaggcgtggtccagcctgggaggtccctgagagtctcctgtgcagcgtctggattcaccttc
agtaactatggcatgcactgggtccgccaggctccaggcaaggggctggagtgggtggcagttatatggtatgatgacagtaataa
acagtatggagactccgtgaagggccgattcaccatctccagagacaattccaagagtacgctgtatctgcaaatggacagactga
gagtcgaggacacggctgtgtattattgtgcgagagcctccgagtatagtatcagctggcgacacaggggggtccttgactactggg
gccagggaaccctggtcaccgtctcgagt
Clone No. 880:
cagatcaccttgaaggagtctggtcctacgctggtgagacccacacagaccctcacactgacctgcaccttctctgggttctcactcag
cactagtaaactgggtgtgggctggatccgtcagcccccaggaaaggccctggagtggcttgcactcgttgattgggatgatgatag
gcgctacaggccatctttgaagagcaggctcaccgtcaccaaggacacctccaaaaaccaggtggtccttacaatgaccaacatgg
accctgtggacacagccacatattactgtgcacacagtgcctactatactagtagtggttattaccttcaatacttccatcactggggcc
cgggcaccctggtcaccgtctcgagt
Clone No. 881:
gaggtgcagctggtggagtctgggggaggcgtggtacagcctggaggctccctgagactctcctgtgaagtctccggattcaccttc
aatagttatgaaatgacctgggtccgccaggccccagggaaggggctggagtgggtttcacacattggtaatagtggttctatgata
tactacgctgactctgtgaagggccgattcaccatctccagagacaacgccaagaactcactatatctgcaaatgaacagcctgaga
gtcgaggacacggctgtttattactgtgcgaggtcagattactatgatagtagtggttattatctcctctacttagactcctggggccat
ggaaccctggtcaccgtctcgagt
Clone No. 884:
caggtgcagctggtgcagtctggggctgaggtgaggaagcctggggcctcagtgaaggtttcctgcaaggcttctggacatactttc
attaactttgctatgcattgggtgcgccaggcccccggacaggggcttgagtggatgggatacatcaacgctgtcaatggtaacaca
cagtattcacagaagttccagggcagagtcacctttacgagggacacatccgcgaacacagcctacatggagctgagcagcctgag
atctgaagacacggctgtgtattactgtgcgagaaacaatgggggctctgctatcattttttactactggggccagggaaccctggtc
accgtctcgagt
Clone No. 886:
caggtgcagctggtggagtctgggggaggcgtggtccagcctgggaggtccctgagactctcctgtgcagcctctggattcagcttc
agtagctatggcatgcactgggtccgccaggctccaggcaaggggctggagtgggtggcagttatatcaaatgatggaagtaataa
atactatgcagactccgtgaagggccgattcaccatctccagagacaattccaagaaaacgatgtatctgcaaatgaacagcctgag
agctgaggacacggctgtgtatttctgtgcgaagacaacagaccagcggctattagtggactggttcgacccctggggccagggaa
ccctggtcaccgtctcgagt
Clone No. 888:
cagctgcagctgcaggagtcgggcccaggactggtgaagccatcggagaccctgtccctcacctgcactgcctctggtggctccatc
aacagtagtaatttctactggggctggatccgccagcccccagggaaggggctggagtggattgggagtatcttttatagtgggacc
acctactacaacccgtccctcaagagtcgagtcaccatatccgtagacacgtccaagaaccagttctccctgaagctgagccctgtga
ccgccgcagacacggctgtctatcactgtgcgagacatggcttccggtattgtaataatggtgtatgctctataaatctcgatgcttttg
atatctggggccaagggacaatggtcaccgtctcgagt
Clone No. 894:
caggtgcagctggtggagtctgggggaggcgtcgtccagcctggaaagtccctgagactctcctgtgcagcgtctggattcagattc
agtgactacggcatgcactgggtccggcaggctccaagcaaggggctggagtgggtggcagttatctggcatgacggaagtaata
taaggtatgcagactccgtgaggggccgattttccatctccagagacaattccaagaacacgctgtatttgcaaatgaacagcatga
gagccgacgacacggctttttattattgtgcgagagtcccgttccagatttggagtggtctttattttgaccactggggccagggaacc
ctggtcaccgtctcgagt

In the same clones, the complete amino acid sequences of the light chains (i.e. light chains including constant and variable regions) have the following amino acid sequences, which are also set forth as SEQ ID NOs: 89-132:

Clone No. 735:
EIVLTQSPATLSLSPGERATLSCRASQSVNSHLAWYQQKPGQAPRLLIYN
TFNRVTGIPARFSGSGSGTDFTLTISSLATEDFGVYYCQQRSNWPPALTF
GGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC
Clone No. 736:
DIQMTQSPSSLSASVGDRVTFTCRASQRISNHLNWYQQKPGKAPKLLIFG
ASTLQSGAPSRFSGSGSGTDFTLTITNVQPDDFATYYCQQSYRTPPINFG
QGTRLDIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 744:
EIVLTQSPGTLSLSPGERATLSCRASQSVSSSYLAWYQQKPGQAPRLLIY
GASSRATGIPDRFSGSGSGTDFTLTISRLEPEDFAVYYCQQYDSSLSTWT
FGQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
Clone No. 793:
DIQMTQSPSSLSASVGDRVTITCRASQSITGYLNWYQQKPGKAPKLLIYA
TSTLQSEVPSRFSGSGSGTDFTLTISSLQPEDFATYYCQQSYNTLTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Clone No. 795:
EIVLTQSPGTLSLSPGERATLSCRASQSVSSSYLAWYQQKPGQAPRLLIH
GASTGATGTPDRFSGSGSGTDFTLTISTLEPEDFAVYYCQQYGRTPYTFG
QGTKLENKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 796:
DIVMTQTPLSLSVTPGQPASISCRSSQSLLRSDGKTFLYWYLQKPGQSPQ
PLMYEVSSRFSGVPDRFSGSGSGADFTLNISRVETEDVGIYYCMQGLKIR
RTFGPGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Clone No. 799:
DIQMTQSPSTLSASVGDRVTFSCRASQSVSSWVAWYQQKPGKAPKLLISE
ASNLESGVPSRFSGSGSGTEFTLTISSLQPEDFATYYCQQYHSYSGYTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 800:
AIQLTQSPSSLSASVGDRVTLTCRASQGITDSLAWYQQKPGKAPKVLLYA
ASRLESGVPSRFSGRGSGTDFTLTISSLQPEDFATYYCQQYSKSPATFGP
GTKVEIRRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 801:
DIVMTQSPLSLPVTPGEPASISCRSSQSLLNSNGFNYVDWYLQKPGQSPQ
LLIYLGSNRASGVPDRFSGSGSGTDFTLKISRVEAEDVGVYYCMQALETP
LTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Clone No. 804:
EIVLTQSPGTLSLSPGGRATLSCRASQSVSSGYLAWYQQKPGQAPRLLIY
GASGRATGIPDRFSGSGSGTDFTLTISRLEPEDFAVYYCQQYFGSPYTFG
QGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 810:
NIQMTQSPSAMSASVGDRVTITCRASQGISNYLVWFQQKPGKVPKRLIYA
ASSLQSGVPSRFSGSGSGTEFTLTISSLQPEDFATYYCLQHNISPYTFGQ
GTKLETKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 811:
DIVMTQSPDSLAVSLGERATINCRSSETVLYTSKNQSYLAWYQQKARQPP
KLLLYWASTRESGVPARFSGSGSGTDFTLAISSLQAEDVAVYYCQQFFRS
PFTFGPGTRLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
Clone No. 812:
EIVLTQSPGTLSLSPGERVTLSCRASQSVSSSYIAWYQQKPGQAPRLVIY
AASRRATGVPDRFSGSGSATDFTLTISRLEPEDLAVYYCQHYGNSLFTFG
PGTKVDVKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 814:
DIQMTQSPSTLSASVGDRVTITCRASQSIGSRLAWYQQQPGKAPKFLIYD
ASSLESGVPSRFSGSGSGTEFTLTISSLQPEDLATYYCQQYNRDSPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 816:
DIVMTQSPLSLPVTPGEPASISCRSSQSLLHSDGRYYVDWYLQKPGQSPH
LLIYLASNRASGVPDRFTGSGSGTDFTLKISRVEAEDVGVYYCMQGLHTP
WTFGQGTKVDIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Clone No. 817:
EIVMTQSPATLSASPGERATLSCWASQTIGGNLAWYQQKPGQAPRLLIYG
ASTRATGVPARFSGSGSGTEFTLAISSLQSEDFAVYYCQQYKNWYTFGQG
TKLELKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Clone No. 818:
DIQMTQSPSSLSASVGDRVTITCRASQTIASYVNWYQQKPGRAPSLLIYA
ASNLQSGVPPRFSGSGSGTDFTLTISGLQPDDFATYYCQQSYSYRALTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 819:
EIVLTQSPATLSLSPGERATLSCRASQSVSSSLAWYQQTPGQAPRLLIYD
ASYRVTGIPARFSGSGSGIDFTLTISSLEPEDFAVYYCQQRSNWPPGLTF
GGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC
Clone No. 824:
AIQLTQSPSSLSASVGDTVTVTCRPSQDISSALAWYQQKPGKPPKLLIYG
ASTLDYGVPLRFSGTASGTHFTLTISSLQPEDFATYYCQQFNTYPFTFGP
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 825:
DIVMTQSPDSLAVSLGERATINCKSSQSVLYNSNNKNYLAWYQQKPGQPP
KLLIHLASTREYGVPDRFSGSGSGTDFALIISSLQAEDVAVYYCQQYYQT
PLTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
Clone No. 827:
DIQMTQSPSSLAASVGDRVTITCRASQFISSYLHWYQQRPGKAPKLLMYA
ASTLQSGVPSRFSGSGSGTDFTLTISSLQPEDFATYYCQQSYTNPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC
Clone No. 829:
DIQMTQSPSSLSASVGDRVTITCRASQSIASYLNWYQQKPGKAPKLLMYA
ASSLHSGVPSRFSGSGSGTDFTLTISSLQPEDFATYYCQHSYSTRFTFGP
GTKVDVKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 830:
DIQMTQSPSTLSASVGDRVTITCRASQSVTSELAWYQQKPGKAPNFLIYK
ASSLESGVPSRFSGSGSGTEFTLTISSLQPDDFATYYCQQYNSFPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 831:
DIQMTQSPSTLSASVGDRLTITCRASQNIYNWLAWYQQKPGKAPKLLIYD
ASTLESGVPSRFSGSGSGTEFTLTISSLQPDDFATYYCQQYNSLSPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 835:
DIQLTQSPSFLSASLEDRVTITCRASQGISSYLAWYQQKPGKAPKLLLDA
ASTLQSGVPSRFSGSGSGTEFTLTISSLQPEDFATYYCQQLNSYPRTFGQ
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 838:
DIQMTQSPSSLSASVGDRVSITCRASQGISNYLAWYQQKPGKVPKLLIYA
ASTLQSGVPSRFSGSGSGTDFTLTISSLQPEDVATYYCQKYNSAPQTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 841:
DIVMTQSPDSLAVSLGERATINCRSSQSVLYSSNNKNYLAWYQQKPGQPP
KLLVYWASTRASGVPDRFSGSGSGTDFTLTLSSLQAEDVAVYYCQQFHST
PRTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
Clone No. 853:
EIVLTQSPGTLSLSPGERATLSCRASQSVSSNYLAWYQQKPGQAPRLLIY
GASSRAAGMPDRFSGSGSGTDFTLTISRLEPEDFAVYYCQQYGNSPLTFG
GGTEVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 855:
DIQMTQSPSSVSASVGDRVTITCRASQAISNWLAWYQQKPGKAPKLLIYA
ASSLQSGVPSRFSGSGSGTDFTLTISGLQPEDFATYYCQQADTFPFTFGP
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 856:
DIVMTQTPLSLPVTPGEPASISCRSSQSLLDSNDGNTYLDWYLQKPGQSP
QLLIYTFSYRASGVPDRFSGSGSGTDFTLKISRVEAEDVGVYYCMQRIEF
PYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
Clone No. 857:
DIVMTQSPLSLPVTPGEPASISCRSSQSLLHRNEYNYLDWYLQKPGQSPQ
LLIYWGSNRASGVPDRFSGSGSGTDFTLKISRVEAEDVGVYYCMQTLQTP
RTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Clone No. 858:
DIQMTQSPSSVSASVGDRVTITCQASQDISNYLNWYQQKPGKAPKLLIFD
ATKLETGVPTRFIGSGSGTDFTVTITSLQPEDVATYYCQHFANLPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 859:
DIQMTQSPSSLSASVGDRVTITCRASQGIRNYLAWYQQKPGKVPKLLVFA
ASTLQSGVPSRFSGSGSGTDFTLTISSLQPEDVATYYCQRYNSAPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC
Clone No. 861:
DIQMTQSPSSLSASVGDRVTITCRASQIIASYLNWYQQKPGRAPKLLIYA
ASSLQSGVPSRFSGSGSGTDFTLTISSLQPEDFATYYCQQSYSTPIFTFG
PGTKVNIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 863:
EIVLTQSPATLSLSPGERATLSCRTSQSVSSYLAWYQQKPGQAPRLLIYD
ASNRATGIPARFSGSGSGTDFTLTISSLEPEDFAVYYCQQRSDWLTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Clone No. 868:
EIVMTQSPATLSVSPGERATLSCRASQSIKNNLAWYQVKPGQAPRLLTSG
ASARATGIPGRFSGSGSGTDFTLTISSLQSEDIAVYYCQEYNNWPLLTFG
GGTKVEIQRTVMPSVFIFPPSDEQLKSGTASVVCLLNNFYPREAKVQWKV
DNALQSGNSQEVTEQDSKDSTYSLSSTLTLSKADYEKHKVYACEVTHQGL
SSPVTKSFNRGEC
Clone No. 870:
DIQMTQSPPSLSASVGDRVTITCRASQRIASYLNWYQQKPGRAPKLLIFA
ASSLQSGVPSRFSGSGSGTDFTLTISSLQPEDYATYYCQQSYSTPIYTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 871:
DIQMTQSPSSLSASVGDRVTITCQASQGISNYLNWYQQKPGKAPKLLIFD
ASNLESEVPSRFSGRGSGTDFTFSISSLQPEDIATYFCQQYDNFPYTFGQ
GTKLEIKRTVAAPSVFIFPPSDEQLKSGTASWCLLNNFYPREAKVQWKVD
NALQSGNSQESVTEQDSKDSTYSLSSTLTLSKADYEKHKVYACEVTHQGL
SSPVTKSFNRGEC
Clone No. 880:
DIQMTQSPSSLAASVGDRVTITCRASQTIASYVNWYQQKPGKAPNLLIYA
ASSLQSGVPSRFSGSGSGTDFTLTISSLQPEDFASYFCQQSYSFPYTFGQ
GTKLDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Clone No. 881:
DIQMTQSPSSLSASVGDRVTITCRASQTIASYVNWYQQKPGKAPKLLIYA
ASNLQSGVPSRFSGSGSGTDFTLTISSLQPEDFATYYCQQSYSVPRLTFG
GGTKVDITRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 884:
DIQMTQSPSSLSASVGDRVTITCRSSQTISVFLNWYQQKPGKAPKLLIYA
ASSLHSAVPSRFSGSGSGTDFTLTISSLQPEDSATYYCQESFSSSTFGGG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Clone No. 886:
EIVMTQSPATLSVSPGETATLSCRASQSVSSNLAWYQHKPGQAPRLLIHS
ASTRATGIPARFSGSGSGTEFTLTISSLQSEDFAVYYCQQYNMWPPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Clone No. 888:
DIVMTQSPLSLPVTPGAPASISCRSSQSLLRTNGYNYLDWYLQKPGQSPQ
LLIYLGSIRASGVPDRFSGSGSGTDFTLKISRVEAEDVGVYYCMQSLQTS
ITFGQGTRLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Clone No. 894:
EIVMTQSPATLSVSPGERATLSCRASQSVGNNLAWYQQRPGQAPRLLIYG
ASTRATGIPARFSGSGSGTEFTLTISSLQSEDFAVYYCQQYDKWPETFGQ
GTKVDIKR1VAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The light chain encoding nucleic acid fragments in these clones have the following nucleic acid sequences, which are also provided as SEQ ID NOs: 133-176:

Clone No 735:
gaaattgtgttgacacagtctccagccaccctgtccttgtctccaggaga
aagagccaccctctcctgcagggccagtcagagtgttaacagccacttag
cctggtaccaacagaaacctggccaggctcccaggctcctcatctataat
acattcaatagggtcactggcatcccagccaggttcagtggcagtgggtc
tgggacagacttcactctcaccatcagcagccttgcgactgaagattttg
gcgtttattactgtcagcagcgtagcaactggcctcccgccctcactttc
ggcggagggaccaaagtggagatcaaacgaactgtggctgcaccatctgt
cttcatcttcccgccatctgatgagcagttgaaatctggaactgcctctg
ttgtgtgcctgctgaataacttctatcccagagaggccaaagtacagtgg
aaggtggataacgccctccaatcgggtaactcccaggagagtgtcacaga
gcaggacagcaaggacagcacctacagcctcagcagcaccctgacgctga
gcaaagcagactacgagaaacacaaagtctacgcctgcgaagtcacccat
cagggcctgagctcgcccgtcacaaagagcttcaacaggggagagtgt
Clone No 736:
gacatccagatgacccagtctccatcctccctgtctgcatctgtgggaga
cagagtcaccttcacttgccgggccagtcagaggattagcaaccatttaa
attggtatcaacaaaagccagggaaagcccctaaactcctgatctttggt
gcatccactcttcaaagtggggccccatcaaggttcagtggcagtggatc
tgggacagatttcactctcaccatcactaatgtacaacctgacgattttg
caacttactactgtcaacagagttacagaactcccccgatcaacttcggc
caagggacacgcctggacattaagcgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 744:
gaaattgtgttgacgcagtctccaggcaccctgtctttgtctccagggga
aagagccaccctctcctgcagggccagtcagagtgttagcagcagctact
tagcctggtatcagcagaaacctggccaggctcccaggctcctcatctat
ggtgcatccagcagggccactggcatcccagacaggttcagtggcagtgg
gtctgggacagacttcactctcaccatcagcagactggagcctgaagatt
ttgcagtgtattactgtcagcagtatgatagctcactttctacgtggacg
ttcggccaagggaccaaggtggaaatcaaacgaactgtggctgcaccatc
tgtcttcatcttcccgccatctgatgagcagttgaaatctggaactgcct
ctgttgtgtgcctgctgaataacttctatcccagagaggccaaagtacag
tggaaggtggataacgccctccaatcgggtaactcccaggagagtgtcac
agagcaggacagcaaggacagcacctacagcctcagcagcaccctgacgc
tgagcaaagcagactacgagaaacacaaagtctacgcctgcgaagtcacc
catcagggcctgagctcgcccgtcacaaagagcttcaacaggggagagtg
t
Clone No 793:
gacatccagatgacccagtctccatcctccctgtctgcatctgtaggaga
cagagtcaccatcattgccgggcaagtcagagcattaccggctatttaaa
ttggtatcagcagaaaccagggaaagcccctaaactcctgatctatgcta
catccactttgcaaagtgaggtcccatcaaggttcagtggcagtggatct
gggacagatttcactctcaccatcagcagtcttcaacctgaagattttgc
aacttactactgtcaacagagttataataccctcactttcggcggaggga
ccaaggtggagatcaaacgaactgtggctgcaccatctgtcttcatcttc
ccgccatctgatgagcagttgaaatctggaactgcctctgttgtgtgcct
gctgaataacttctatcccagagaggccaaagtacagtggaaggtggata
acgccctccaatcgggtaactcccaggagagtgtcacagagcaggacagc
aaggacagcacctacagcctcagcagcaccctgacgctgagcaaagcaga
ctacgagaaacacaaagtctacgcctgcgaagtcacccatcagggcctga
gctcgcccgtcacaaagagcttcaacaggggagagtgt
Clone No 795:
gaaattgtgttgacgcagtctccaggcaccctgtctttgtctccagggga
aagagccaccctctcctgcagggccagtcagagtgttagcagcagctact
tagcctggtatcagcagaaacctggccaggctcccaggctcctcatacat
ggcgcatccaccggggccactggcaccccagacaggttcagtggcagtgg
gtctgggacagacttcactctcaccatcagtacactggagcctgaagatt
ttgcagtgtattactgtcagcaatatggtaggacaccgtacacttttggc
caggggaccaagctggagaacaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 796:
gatattgtgatgacccagactccactctctctgtccgtcacccctggaca
gccggcctccatctcctgcaggtctagtcagagcctcctgcgaagtgatg
gaaagacgtttttgtattggtatctgcagaagccaggccagtctccccaa
cccctaatgtatgaggtgtccagccggttctctggagtgccagataggtt
cagtggcagcgggtcaggggcagatttcacactgaacatcagccgggtgg
agactgaggatgttgggatctattactgcatgcaaggtttgaaaattcgt
cggacgtttggcccagggaccaaggtcgaaatcaagcgaactgtggctgc
accatctgtcttcatcttcccgccatctgatgagcagttgaaatctggaa
ctgcctctgttgtgtgcctgctgaataacttctatcccagagaggccaaa
gtacagtggaaggtggataacgccctccaatcgggtaactcccaggagag
tgtcacagagcaggacagcaaggacagcacctacagcctcagcagcaccc
tgacgctgagcaaagcagactacgagaaacacaaagtctacgcctgcgaa
gtcacccatcagggcctgagctcgcccgtcacaaagagcttcaacagggg
agagtgt
Clone No 799:
gacatccagatgacccagtctccttccaccctgtctgcatctgtaggaga
cagagtcaccttctcttgccgggccagtcagagtgttagtagttgggtgg
cctggtatcagcagaaaccaggaaaagcccctaagctcctgatctctgag
gcctccaatttggaaagtggggtcccatcccggttcagcggcagtggatc
cgggacagaattcactctcaccatcagcagcctgcagcctgaagattttg
caacttattactgccaacagtatcatagttactctgggtacacttttggc
caggggaccaagttggaaatcaagcgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 800:
gccatccagttgacccagtctccatcgtccctgtctgcatctgtaggcga
cagagtcaccctcacttgccgggcgagtcagggcattaccgattctttag
cctggtatcagcagaaaccagggaaagcccctaaggtcctgctctatgct
gcttccagattggaaagtggggtcccatccaggttcagtggccgtggatc
tgggacggatttcactctcaccatcagcagcctgcagcctgaagactttg
caacttattactgtcaacagtattctaagtcccctgcgacgttcggccca
gggaccaaggtggaaatcagacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 801:
gatattgtgatgacccagtctccactctccctgcccgtcacccctggaga
gccggcctccatctcctgcaggtctagtcagagcctcctaaatagtaatg
gattcaactatgtggattggtacctgcagaagccagggcagtctccacaa
ctcctgatctatttgggttctaatcgggcctccggggtccctgacaggtt
cagtggcagtggatcaggcacagattttacactgaaaatcagcagagtgg
aggctgaggatgttggggtttattactgcatgcaagctctagaaactccg
ctcactttcggcggagggaccaaggtggagatcaaacgaactgtggctgc
accatctgtcttcatcttcccgccatctgatgagcagttgaaatctggaa
ctgcctctgttgtgtgcctgctgaataacttctatcccagagaggccaaa
gtacagtggaaggtggataacgccctccaatcgggtaactcccaggagag
tgtcacagagcaggacagcaaggacagcacctacagcctcagcagcaccc
tgacgctgagcaaagcagactacgagaaacacaaagtctacgcctgcgaa
gtcacccatcagggcctgagctcgcccgtcacaaagagcttcaacagggg
agagtgt
Clone No 804:
gaaattgtgttgacgcagtctccaggcaccctgtctttgtctccaggggg
aagagccaccctctcctgcagggccagtcagagtgttagcagcggctact
tagcctggtaccagcagaaacctggccaggctcccaggctcctcatctat
ggtgcatccggcagggccactggcatcccagacaggttcagtggcagtgg
gtctgggacagacttcactctcaccatcagcagactggagcctgaagatt
ttgcagtgtattactgtcagcagtattttggctcaccgtacacttttggc
caggggaccaagctggagctcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 810:
aacatccagatgacccagtctccatctgccatgtctgcatctgtaggaga
cagagtcaccatcacttgtcgggcgagtcagggcattagtaattatttag
tctggtttcagcagaaaccagggaaagtccctaagcgcctgatctatgct
gcatccagtttgcaaagtggggtcccatcaaggttcagcggcagtggatc
tgggacagaattcactctcacaatcagcagcctgcagcctgaagattttg
caacttattactgtctacagcataatatttccccttacacttttggccag
gggaccaagctggagaccaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 811:
gacatcgtgatgacccagtctccagactccctggctgtgtctctgggcga
gagggccaccatcaactgcaggtccagtgagactgttttatacacctcta
aaaatcagagctacttagcttggtaccagcagaaagcacgacagcctcct
aaactactcctttactgggcatctacccgggaatccggggtccctgcccg
attcagtggcagcggatctgggacagatttcactctcgccatcagcagcc
tgcaggctgaagatgtggcagtttattactgtcagcaattttttaggagt
cctttcactttcggccccgggaccagactggagattaaacgaactgtggc
tgcaccatctgtcttcatcttcccgccatctgatgagcagttgaaatctg
gaactgcctctgttgtgtgcctgctgaataacttctatcccagagaggcc
aaagtacagtggaaggtggataacgccctccaatcgggtaactcccagga
gagtgtcacagagcaggacagcaaggacagcacctacagcctcagcagca
ccctgacgctgagcaaagcagactacgagaaacacaaagtctacgcctgc
gaagtcacccatcagggcctgagctcgcccgtcacaaagagcttcaacag
gggagagtgt
Clone No 812:
gaaattgtgttgacgcagtctccaggcaccctgtctttgtctccagggga
aagagttaccctctcttgcagggccagtcagagtgttagcagcagttaca
tagcctggtaccagcagaagcctggccaggctcccaggctcgtcatctat
gctgcatcccgcagggccactggcgtcccagacaggttcagtggcagtgg
gtctgcgacagacttcactctcaccatcagtagactggagcctgaagatc
ttgcagtgtattactgtcagcactatggtaactcactattcactttcggc
cctgggaccaaggtggatgtcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 814:
gacatccagatgacccagtctccctccaccctgtctgcatctgtcggaga
cagagtcaccatcacttgccgggccagtcagagtattggtagccggttgg
cctggtatcagcagcaaccagggaaagcccctaaattcctgatctatgat
gcctccagtttggaaagtggggtcccatcaaggttcagcggcagtggatc
agggacagaattcactctcaccatcagcagcctgcagccggaggatcttg
caacttattactgccaacagtacaatagagattctccgtggacgttcggc
caagggaccaaggtggaaatcaagcgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 816:
gatattgtgatgacccagtctccactctccctgcccgtcaccccaggaga
gccggcctccatctcctgcaggtctagtcagagcctcctgcatagtgatg
gacgctactatgtggattggtacctgcagaagccagggcagtctccacac
ctcctgatctatttggcttctaatcgggcctccggggtccctgacaggtt
cactggcagtggatcaggcacagattttacactgaaaatcagcagagtgg
aggctgaggatgttggcgtttattactgcatgcaaggtctacacactcct
tggacgttcggccaggggaccaaggtggacatcaagcgaactgtggctgc
accatctgtcttcatcttcccgccatctgatgagcagttgaaatctggaa
ctgcctctgttgtgtgcctgctgaataacttctatcccagagaggccaaa
gtacagtggaaggtggataacgccctccaatcgggtaactcccaggagag
tgtcacagagcaggacagcaaggacagcacctacagcctcagcagcaccc
tgacgctgagcaaagcagactacgagaaacacaaagtctacgcctgcgaa
gtcacccatcagggcctgagctcgcccgtcacaaagagcttcaacagggg
agagtgt
Clone No 817:
gaaattgtaatgacacagtctccagccaccctgtctgcgtccccagggga
aagagccaccctctcctgttgggccagtcagactattggaggcaacttag
cctggtaccagcagaaacctggccaggctcccaggctcctcatctatggt
gcatccaccagggccactggtgtcccagccaggttcagtggcagtgggtc
tgggacagagttcactctcgccatcagcagcctgcagtctgaagattttg
cagtttattactgtcagcagtataaaaactggtacacttttggccagggg
accaagctggagctcaaacgaactgtggctgcaccatctgtcttcatctt
cccgccatctgatgagcagttgaaatctggaactgcctctgttgtgtgcc
tgctgaataacttctatcccagagaggccaaagtacagtggaaggtggat
aacgccctccaatcgggtaactcccaggagagtgtcacagagcaggacag
caaggacagcacctacagcctcagcagcaccctgacgctgagcaaagcag
actacgagaaacacaaagtctacgcctgcgaagtcacccatcagggcctg
agctcgcccgtcacaaagagcttcaacaggggagagtgt
Clone No 818:
gacatccagatgacccagtctccatcctccctgtctgcatctgtaggaga
cagagtcaccatcacttgccgggcaagtcagaccattgccagttacgtaa
attggtaccaacaaaaaccagggagagcccctagtctcctgatctatgct
gcatctaacttgcagagtggggtcccaccaaggttcagtggcagtggatc
tgggacagacttcactctcaccatcagcggtctgcaacctgacgattttg
caacttattactgtcaacagagttacagttatcgagcgctcactttcggc
ggagggaccaaggtggagatcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 819:
gaaattgtgttgacacagtctccagccaccctgtcgttgtccccagggga
aagagccaccctctcctgcagggccagtcagagtgttagcagctccttag
cctggtaccaacagacacctggccaggctcccaggcttctcatctatgat
gcgtcctacagggtcactggcatcccagccaggttcagtggcagtgggtc
tgggatagacttcactctcaccatcagcagcctagagcctgaagattttg
cagtttactattgtcagcagcgtagcaactggcctccggggctcactttc
ggcggggggaccaaggtggagatcaaacgaactgtggctgcaccatctgt
cttcatcttcccgccatctgatgagcagttgaaatctggaactgcctctg
ttgtgtgcctgctgaataacttctatcccagagaggccaaagtacagtgg
aaggtggataacgccctccaatcgggtaactcccaggagagtgtcacaga
gcaggacagcaaggacagcacctacagcctcagcagcaccctgacgctga
gcaaagcagactacgagaaacacaaagtctacgcctgcgaagtcacccat
cagggcctgagctcgcccgtcacaaagagcttcaacaggggagagtgt
Clone No 824:
gccatccagttgacccagtctccatcctccctgtctgcatctgttggaga
cacagtcaccgtcacttgccggccaagtcaggacattagcagtgctttag
cctggtatcagcagaaaccagggaaacctcctaagctcctgatctatggt
gcctccactttggattatggggtcccattaaggttcagcggcactgcatc
tgggacacatttcactctcaccatcagcagcctgcaacctgaagattttg
caacttattactgtcaacagtttaatacttacccattcactttcggccct
gggaccaaagtggatatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 825:
gacatcgtgatgacccagtctccagactccctggctgtgtctctgggcga
gagggccaccatcaactgcaagtccagccagagtgttttatacaactcca
acaataagaactacttagcctggtatcagcagaaaccaggacagcctcct
aagctcctcattcacttggcatctacccgggaatacggggtccctgaccg
attcagtggcagcgggtctgggacagatttcgctctcatcatcagcagcc
tgcaggctgaagatgtggcagtttattactgtcaacaatattatcaaact
cctctaacttttggccaggggaccaaggtggagatcaaacgaactgtggc
tgcaccatctgtcttcatcttcccgccatctgatgagcagttgaaatctg
gaactgcctctgttgtgtgcctgctgaataacttctatcccagagaggcc
aaagtacagtggaaggtggataacgccctccaatcgggtaactcccagga
gagtgtcacagagcaggacagcaaggacagcacctacagcctcagcagca
ccctgacgctgagcaaagcagactacgagaaacacaaagtctacgcctgc
gaagtcacccatcagggcctgagctcgcccgtcacaaagagcttcaacag
gggagagtgt
Clone No 827:
gacatccagatgacccagtctccatcctccctggctgcatctgtaggaga
cagagtcaccatcacttgccgggcaagtcagttcattagcagctatttac
attggtatcagcaaagaccaggcaaggcccctaaactcctgatgtatgct
gcctccactttgcaaagtggggtcccatcaaggttcagtggcagtggatc
tgggacagatttcactctcaccatcagcagtctgcaacctgaagattttg
caacttactactgtcaacagagttacactaacccatacacttttggccag
gggaccaagctggagatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 829:
gacatccagatgacccagtctccatcctccctatctgcatctgtaggaga
cagagtcaccatcacttgccgggcaagtcagagcattgccagctatttaa
attggtatcagcagaaaccagggaaagcccccaaactcctgatctatgct
gcatccagtttgcatagtggggtcccatcaagattcagtggcagtggatc
tgggacagatttcactctcaccatcagcagtctgcaacctgaagattttg
caacttactactgtcaacacagttacagtactcgattcactttcggccct
gggaccaaagtggatgtcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 830:
gacatccagatgacccagtctccttcgaccctgtctgcatctgtaggaga
cagagtcaccatcacttgccgggccagtcagagtgttactagtgagttgg
cctggtatcagcagaaaccagggaaagcccctaacttcctgatctataag
gcgtctagtttagaaagtggggtcccatcaaggttcagcggcagtggatc
tgggacagaattcactctcaccatcagcagcctgcagcctgatgattttg
caacttattactgccaacagtataatagttttccgtacacttttggccag
gggaccaagctggagatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 831:
gacatccagatgacccagtctccttccaccctgtctgcatctgtaggcga
cagactcaccatcacttgccgggccagtcagaatatttataactggttgg
cctggtatcagcagaaaccagggaaagcccctaaactcctgatctatgac
gcctccactttggaaagtggggtcccatcaaggttcagcggcagtggatc
tgggacagagttcactctcaccatcagcagcctgcagcctgatgattttg
cgacttattactgccaacaatataatagtttgtctccgacgttcggccaa
gggaccaaggtggaaatcaagcgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 835:
gacatccagttgacccagtctccatccttcctgtctgcatctttagaaga
cagagtcactatcacttgccgggccagtcagggcattagcagttatttag
cctggtatcagcaaaaaccagggaaagcccctaagctcctgctcgatgct
gcatccactttgcaaagtggggtcccatcaaggttcagcggcagtggatc
tgggacagagttcactctcacaatcagcagcctgcagcctgaagattttg
caacttattactgtcaacagcttaatagttaccctcggacgttcggccaa
gggaccaaggtggacatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 838:
gacatccagatgacccagtctccatcctccctgtctgcatctgtaggaga
cagagtcagcatcacttgccgggcgagtcagggcattagcaattatttag
cctggtatcagcagaaaccagggaaggttcctaagctcctgatctatgct
gcatccactttgcaatcaggggtcccatctcggttcagtggcagtggatc
tgggacagatttcactctcaccatcagcagcctgcagcctgaggatgttg
caacttattactgtcaaaagtataacagtgcccctcaaacgttcggccaa
gggaccaaggtggaaatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 841:
gacatcgtgatgacccagtctccagactccctggctgtgtctctgggcga
gagggccaccatcaactgcaggtccagccagagtgttttatacagctcca
acaataagaactacttagcttggtaccagcagaaaccaggacagcctcct
aagctgctcgtttactgggcatcaacccgggcatccggggtccctgaccg
attcagtggcagcgggtctgggacagatttcactctcaccctcagcagcc
tgcaggctgaagatgtggcagtttattactgtcagcagtttcatagtact
cctcggacgttcggccaagggaccaaggtggagatcaaacgaactgtggc
tgcaccatctgtcttcatcttcccgccatctgatgagcagttgaaatctg
gaactgcctctgttgtgtgcctgctgaataacttctatcccagagaggcc
aaagtacagtggaaggtggataacgccctccaatcgggtaactcccagga
gagtgtcacagagcaggacagcaaggacagcacctacagcctcagcagca
ccctgacgctgagcaaagcagactacgagaaacacaaagtctacgcctgc
gaagtcacccatcagggcctgagctcgcccgtcacaaagagcttcaacag
gggagagtgt
Clone No 853:
gaaattgtgttgacgcagtctccaggcaccctgtctttgtctccagggga
aagagccaccctctcctgcagggccagtcagagtgttagcagcaactact
tagcctggtaccagcagaaacctggccaggctcccaggctcctcatctat
ggtgcatccagcagggccgctggcatgccagacaggttcagtggcagtgg
gtctgggacagacttcactctcaccatcagcagactggagcctgaagatt
ttgcagtgtattactgtcagcagtatggtaactcaccgctcactttcggc
ggagggaccgaggtggagatcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 855:
gacatccagatgacccagtctccatcttctgtgtctgcatctgtaggaga
cagagtcaccatcacttgtcgggcgagtcaggctattagtaactggttag
cctggtatcagcagaaaccaggaaaagcccctaagctcctgatctatgct
gcatccagtttgcaaagtggggtcccatcaagattcagcggcagtggatc
tgggacagatttcactctcactatcagcggcctgcagcctgaggattttg
caacttactattgtcaacaggctgacactttccctttcactttcggccct
gggaccaaagtggatatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 856:
gatattgtgatgacccagactccactctccctgcccgtcacccctggaga
gccggcctccatctcctgcaggtctagtcagagcctcttggatagtaatg
atggaaacacctatttggactggtacctgcagaagccagggcagtctcca
cagctcctgatttatacattttcctatcgggcctctggagtcccagacag
gttcagtggcagtgggtctggcactgatttcacactgaaaatcagcaggg
tggaggccgaggatgttggagtttattactgcatgcaacgtatcgagttt
ccgtacacttttggccaggggaccaagctggagatcaaacgaactgtggc
tgcaccatctgtcttcatcttcccgccatctgatgagcagttgaaatctg
gaactgcctctgttgtgtgcctgctgaataacttctatcccagagaggcc
aaagtacagtggaaggtggataacgccctccaatcgggtaactcccagga
gagtgtcacagagcaggacagcaaggacagcacctacagcctcagcagca
ccctgacgctgagcaaagcagactacgagaaacacaaagtctacgcctgc
gaagtcacccatcagggcctgagctcgcccgtcacaaagagcttcaacag
gggagagtgt
Clone No 857:
gatattgtgatgacccagtctccactctccctgcccgtcacccctggaga
gccggcctccatctcctgcaggtctagtcagagcctcctgcatagaaatg
agtacaactatttggattggtacttgcagaagccagggcagtctccacag
ctcctgatctattggggttctaatcgggcctccggggtccctgacaggtt
cagtggcagtggatcaggcacagattttacactgaaaatcagcagagtgg
aggctgaggatgttggggtttattactgcatgcaaactctacaaactcct
cggacgttcggccaagggaccaaggtggaaatcaaacgaactgtggctgc
accatctgtcttcatcttcccgccatctgatgagcagttgaaatctggaa
ctgcctctgttgtgtgcctgctgaataacttctatcccagagaggccaaa
gtacagtggaaggtggataacgccctccaatcgggtaactcccaggagag
tgtcacagagcaggacagcaaggacagcacctacagcctcagcagcaccc
tgacgctgagcaaagcagactacgagaaacacaaagtctacgcctgcgaa
gtcacccatcagggcctgagctcgcccgtcacaaagagcttcaacagggg
agagtgt
Clone No 858:
gacatccagatgacccagtctccatcctccgtgtctgcatctgtgggaga
cagagtcaccatcacttgccaggcgagtcaagacattagcaactatttaa
attggtatcagcagaaaccagggaaagcccctaagctcctgatcttcgat
gcaaccaaattggagacaggggtcccaacaaggttcattggaagtggatc
tgggacagattttactgtcaccatcaccagcctgcagcctgaagatgttg
caacatattactgtcaacactttgctaatctcccatacacttttggccag
gggaccaagctggagatcaagcgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 859:
gacatccagatgacccagtctccatcttccctgtctgcatctgtaggaga
cagagtcaccatcacttgccgggcgagtcagggcattaggaattatttag
cctggtatcagcagaaaccagggaaagttcctaagctcctggtctttgct
gcatccactttgcaatcaggggtcccatctcggttcagtggcagtggatc
tgggacagatttcactctcaccatcagcagcctgcagcctgaggatgttg
caacttattactgtcaaaggtataacagtgccccgctcactttcggcgga
gggacgaaggtggagatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 861:
gacatccagatgacccagtctccatcctccctgtctgcatctgtaggaga
cagagtcaccatcacttgccgggcaagtcagatcattgccagctatttaa
attggtatcagcagaaaccaggcagagcccctaagctcctgatctatgct
gcatccagtttgcaaagtggggtcccatcaaggttcagtggcagtggatc
tgggacagatttcactctcaccatcagcagtctgcaacctgaagattttg
caacttactactgtcaacagagttacagtacccccatattcactttcggc
cctgggaccaaggtgaatatcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 863:
gaaattgtgttgacacagtctccagccaccctgtctttgtctccagggga
aagagccaccctctcctgcaggaccagtcagagtgttagcagctacttag
cctggtaccaacagaaacctggccaggctcccaggctcctcatctatgat
gcttccaatagggccactggcatcccagccaggttcagtggcagtgggtc
tgggacagacttcactctcaccatcagcagcctagagcctgaagattttg
cagtttattactgtcagcagcgtagtgactggctcactttcggcggaggg
accaaggtggagatcaaacgaactgtggctgcaccatctgtcttcatctt
cccgccatctgatgagcagttgaaatctggaactgcctctgttgtgtgcc
tgctgaataacttctatcccagagaggccaaagtacagtggaaggtggat
aacgccctccaatcgggtaactcccaggagagtgtcacagagcaggacag
caaggacagcacctacagcctcagcagcaccctgacgctgagcaaagcag
actacgagaaacacaaagtctacgcctgcgaagtcacccatcagggcctg
agctcgcccgtcacaaagagcttcaacaggggagagtgt
Clone No 868:
gaaattgtaatgacacagtctccagccaccctgtctgtgtctccagggga
aagagccaccctctcctgcagggccagtcagagtattaaaaacaacttgg
cctggtaccaggtgaaacctggccaggctcccaggctcctcacctctggt
gcatccgccagggccactggaattccaggcaggttcagtggcagtgggtc
tgggactgacttcactctcaccatcagcagcctccagtctgaagatattg
cagtttattactgtcaggagtataataattggcccctgctcactttcggc
ggagggaccaaggtggagatccaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 870:
gacatccagatgacccagtctcctccctccctgtctgcatctgtgggaga
cagagtcaccatcacttgccgggcaagtcagaggattgccagctatttaa
attggtatcagcagaaaccagggagagcccctaagctcctgatctttgct
gcatccagtttacaaagtggggtcccatcaaggttcagtggcagtggatc
tgggacagacttcactctcaccatcagtagtctgcaacctgaagattatg
cgacttactactgtcaacagagttacagtactcccatctacacttttggc
caggggaccaagctggagatcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 871:
gacatccagatgacccagtctccatcctccctgtctgcatctgtaggaga
cagagtcaccatcacttgccaggcgagtcagggcattagcaactatttaa
attggtatcaacagaaaccagggaaagcccctaagctcctgatcttcgat
gcatccaatttggaatcagaggtcccatcaaggttcagtggacgtggatc
tgggacagattttactttctccatcagcagcctgcagcctgaagatattg
caacatatttctgtcaacagtatgataatttcccgtacacttttggccag
gggaccaagctggagatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 880:
gacatccagatgacccagtctccatcctccctggctgcatctgtaggaga
cagagtcaccatcacctgccgggcaagtcagacgattgccagttatgtaa
attggtatcaacagaaaccagggaaagcccctaatctcctgatctatgct
gcatccagtttgcaaagtggggtcccatcaaggttcagtggcagtggatc
tgggacagatttcactctcaccatcagcagtctgcaacctgaagattttg
catcttacttctgtcaacagagttacagtttcccgtacacttttggccag
gggaccaagctggatatcaaacgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt
Clone No 881:
gacatccagatgacccagtctccatcctccctgtctgcatctgtaggaga
cagagtcaccatcacttgccgggcaagtcagaccattgccagctatgtaa
attggtatcagcagaaaccagggaaagcccctaagctcctgatctatgct
gcatccaatttgcaaagtggggtcccttcaaggttcagtggcagtggatc
tgggacagatttcactctcaccatcagcagtctgcaacctgaagattttg
caacttactactgtcaacagagttacagtgtccctcggctcactttcggc
ggagggaccaaggtggacatcacacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 884:
gacatccagatgacccagtctccatcctccctgtctgcatctgtaggaga
cagagtcaccatcacttgccggtcaagtcagaccattagcgtctttttaa
attggtatcagcagaaaccagggaaagcccctaagctcctgatctatgcc
gcatccagtttgcacagtgcggtcccatcaaggttcagtggcagtggatc
tgggacagatttcactctcaccatcagcagtctgcaacctgaagattctg
caacttactactgtcaagagagtttcagtagctcaactttcggcggaggg
accaaggtggagatcaaacgaactgtggctgcaccatctgtcttcatctt
cccgccatctgatgagcagttgaaatctggaactgcctctgttgtgtgcc
tgctgaataacttctatcccagagaggccaaagtacagtggaaggtggat
aacgccctccaatcgggtaactcccaggagagtgtcacagagcaggacag
caaggacagcacctacagcctcagcagcaccctgacgctgagcaaagcag
actacgagaaacacaaagtctacgcctgcgaagtcacccatcagggcctg
agctcgcccgtcacaaagagcttcaacaggggagagtgt
Clone No 886:
gaaattgtaatgacacagtctccagccaccctgtctgtgtctccagggga
aacagccaccctctcctgcagggccagtcagagtgttagcagcaacttag
cctggtaccaacataaacctggccaggctcccaggctcctcatccatagt
gcatccaccagggccactgggatcccagccaggttcagtggcagtgggtc
tgggacagagttcactctcaccataagcagcctgcagtctgaagattttg
cagtttattactgtcagcagtataatatgtggcctccctggacgttcggc
caagggaccaaggtggaaatcaaacgaactgtggctgcaccatctgtctt
catcttcccgccatctgatgagcagttgaaatctggaactgcctctgttg
tgtgcctgctgaataacttctatcccagagaggccaaagtacagtggaag
gtggataacgccctccaatcgggtaactcccaggagagtgtcacagagca
ggacagcaaggacagcacctacagcctcagcagcaccctgacgctgagca
aagcagactacgagaaacacaaagtctacgcctgcgaagtcacccatcag
ggcctgagctcgcccgtcacaaagagcttcaacaggggagagtgt
Clone No 888:
gatattgtgatgacccagtctccactctccctgcccgtcacccctggagc
gccggcctccatctcctgcaggtctagtcagagcctcctgcgtactaatg
gatacaactatttggattggtacctgcagaagccagggcagtctccacag
ctcctgatctatttgggttctattcgggcctccggggtccctgacaggtt
cagtggcagtggctcaggcacagattttacactgaaaatcagcagagtgg
aggctgaggatgttggggtttattactgcatgcaatctctacaaacttcg
atcaccttcggccaagggacacgactggagattaaacgaactgtggctgc
accatctgtcttcatcttcccgccatctgatgagcagttgaaatctggaa
ctgcctctgttgtgtgcctgctgaataacttctatcccagagaggccaaa
gtacagtggaaggtggataacgccctccaatcgggtaactcccaggagag
tgtcacagagcaggacagcaaggacagcacctacagcctcagcagcaccc
tgacgctgagcaaagcagactacgagaaacacaaagtctacgcctgcgaa
gtcacccatcagggcctgagctcgcccgtcacaaagagcttcaacagggg
agagtgt
Clone No 894:
gaaattgtaatgacacagtctccagccaccctgtctgtgtctccggggga
aagagccaccctctcctgcagggctagtcagagtgttggcaacaacttag
cctggtaccagcagagacctggccaggctcccagactcctcatctatggt
gcgtccaccagggccactggtatcccagccaggttcagtggcagtgggtc
tgggacagagttcactctcaccatcagcagcctgcagtctgaggattttg
cagtttattactgtcagcagtatgataagtggcctgagacgttcggccag
gggaccaaggtggacatcaagcgaactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgt

In all of the above-discussed 44 clones, the encoded antibodies include the same constant IgG heavy chain, which has the following amino acid sequence (SEQ ID NO: 178):

SASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVE
PKSCDKTHTCPPCPAPELLGGPSVFLFPPKPKDTLMISRTPEVTCVVVDV
SHEDPEVKFNWYVDGVEVHNAKTKPREEQYNSTYRWSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The genomic sequence encoding this heavy chain has the following nucleic acid sequence (SEQ ID NO: 177):

agtgcctccaccaagggcccatcggtcttccccctggcaccctcctccaa
gagcacctctgggggcacagcggccctgggctgcctggtcaaggactact
tccccgaaccggtgacggtgtcgtggaactcaggcgccctgaccagcggc
gtgcacaccttcccggctgtcctacagtcctcaggactctactccctcag
cagcgtggtgaccgtgccctccagcagcttgggcacccagacctacatct
gcaacgtgaatcacaagcccagcaacaccaaggtggacaagagagttggt
gagaggccagcacagggagggagggtgtctgctggaagccaggctcagcg
ctcctgcctggacgcatcccggctatgcagtcccagtccagggcagcaag
gcaggccccgtctgcctcttcacccggaggcctctgcccgccccactcat
gctcagggagagggtcttctggctttttccccaggctctgggcaggcaca
ggctaggtgcccctaacccaggccctgcacacaaaggggcaggtgctggg
ctcagacctgccaagagccatatccgggaggaccctgcccctgacctaag
cccaccccaaaggccaaactctccactccctcagctcggacaccttctct
cctcccagattccagtaactcccaatcttctctctgcagagcccaaatct
tgtgacaaaadcacacatgcccaccgtgcccaggtaagccagcccaggcc
tcgccctccagctcaaggcgggacaggtgccctagagtagcctgcatcca
gggacaggccccagccgggtgctgacacgtccacctccatctcttcctca
gcacctgaactcctggggggaccgtcagtcttcctcttccccccaaaacc
caaggacaccctcatgatctcccggacccctgaggtcacatgcgtggtgg
tggacgtgagccacgaagaccctgaggtcaagttcaactggtacgtggac
ggcgtggaggtgcataatgccaagacaaagccgcgggaggagcagtacaa
cagcacgtaccgtgtggtcagcgtcctcaccgtcctgcaccaggactggc
tgaatggcaaggagtacaagtgcaaggtctccaacaaagccctcccagcc
cccatcgagaaaaccatctccaaagccaaaggtgggacccgtggggtgcg
agggccacatggacagaggccggctcggcccaccctctgccctgagagtg
accgctgtaccaacctctgtccctacagggcagccccgagaaccacaggt
gtacaccctgcccccatcccgggaggagatgaccaagaaccaggtcagcc
tgacctgcctggtcaaaggcttctatcccagcgacatcgccgtggagtgg
gagagcaatgggcagccggagaacaactacaagaccacgcctcccgtgct
ggactccgacggctccttcttcctctatagcaagctcaccgtggacaaga
gcaggtggcagcaggggaacgtcttctcatgctccgtgatgcatgaggct
ctgcacaaccactacacgcagaagagcctctccctgtccccgggtaaatg
a

In this sequence exons are indicated by double underlining. Further, the initial Ser-encoding nucleotides agt (bold underline) are created as a consequence of the introduction into the XhoI digested expression vector of an XhoI digested PCR product encoding the variable heavy chain site in the IgG expression vector.

The above-discussed V_{H} and V_{L} coding pairs were selected according to the binding specificity to various antigens and peptides in ELISA and/or FLISA, epitope mapping, antigen diversity, and sequence diversity. The selected cognate V-gene pairs were subjected to clone repair (Example 1, Section f) if errors were identified. The individual expression constructs were co-transfected with a Flp-recombinase expressing plasmid into the CHO-FlpIn recipient cell line (Invitrogen), followed by antibiotic selection of integrants. The transfections, selection, and adaptation to serum free culture was performed as described in Example 1, section g-1 and g-2.

The stably transfected, serum free suspension culture adapted individual expression cell lines were cryopreserved in multiple ampoules, to generate a cell bank of individual antibody producing cell lines.

### Example 3

In vitro neutralization experiments have been performed both with single antibody clones and with combinations of purified antibodies. All the antibody mixtures described below are constituted of a number of individual anti-RSV antibodies of the present invention, which were combined into a mixture using equal amounts of the different antibodies.

### Testing of Single Antibodies

Initially, the neutralizing activity of each antibody was determined in the PRNT in the presence of complement against RSV subtype A and B strains as described above in Example 1, section j-2. The EC₅₀ values of a number of the purified antibodies are shown in Table 7. Interestingly, while most anti-F antibodies individually exhibited virus neutralizing activity, no EC₅₀ values could be determined for the majority of the anti-RSV protein G antibodies, indicating that these antibodies are not capable of neutralizing the vireo individually. Blank fields indicate that the analysis has not been performed yet. ND indicates that an EC₅₀ value could not be determined in the PRNT due to a very low or lacking neutralizing activity.

TABLE 7
EC₅₀ values of purified anti-RSV protein F and protein G antibodies
against RSV subtype A and B.

EC₅₀ value (μg/ml)

| Clone | Antigen-specificity | Subtype A | Subtype B |
|---|---|---|---|
| 793 | G | 2.52 | |
| 800 | F | 0.15 | 0.16 |
| 810 | F | 0.06 | 0.14 |
| 816 | G | ND | |
| 818 | F | 1.86 | 0.21 |
| 819 | F | 0.18 | |
| 824 | F | 0.03 | 0.02 |
| 825 | F | 0.12 | 0.04 |
| 827 | F | 0.16 | 0.10 |
| 831 | F | 0.08 | 1.66 |
| 853 | G | 1.49 | |
| 855 | G | 6.35 | ND |
| 856 | G | ND | |
| 858 | F | ND | |
| 868 | G | ND | |
| 880 | F | 0.38 | 0.40 |
| 888 | G | 0.14 | |
| 894 | F | 0.08 | 0.07 |

### Mixtures of Anti-F Antibodies

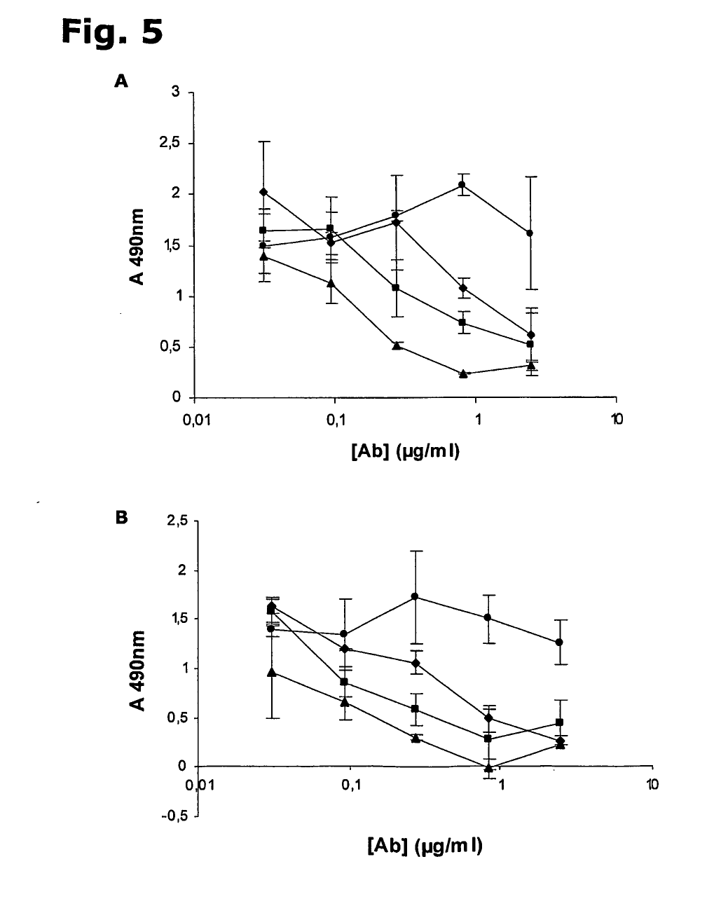

The ability of mixtures of anti-RSV protein F antibodies to neutralize RSV strains of subtype A and B was compared with the neutralizing effect obtained using Palivizumab (also an anti-F antibody). The neutralization capability was assessed using the microneutralization test or the PRNT as described in Example 1, Section j. In an initial experiment two antibody mixtures, anti-F(I) and anti-F(II), containing five and eleven distinct anti-F antibodies, respectively were compared against Palivizumab using the microneutralizating test. Anti-F(I) is composed of antibodies obtained from clones 810, 818, 819, 825 and 827. Antibodies 810 and 819 bind to antigenic site A/II, antibody 818 to site B/I or F1, antibody 825 binds to a complex epitope overlapping with sites A and C and antibody 827 binds to another complex epitope (see Table 5). Anti-F(II) is composed of antibodies obtained from clones 735, 800, 810, 818, 819, 825, 827, 863, 880, 884 and 894. Anti-F(II) contains multiple binders to some of the defined antigenic sites: antibodies 810, 819 and 863 binds A/II, antibodies 800 and 818 binds F1 (or B/I), antibodies 827 and 825 to the complex epitopes described above, antibodies 735 and 894 belong to unknown cluster (UC)I, antibody 880 to UCII, and 884 binds to another currently unknown epitope (see Table 5). As shown in FIG. 5, both composition Anti-F(I) and F(II) are more potent than Palivizumab with respect to neutralization of RSV strains of both subtypes.

FIG. 5 also shows that the combination of five antibodies (anti-F(I)) appears to be more potent than the combination of eleven antibodies (Anti-F(II)). The anti-F(I) mixture contains some of the most potent individually neutralizing antibodies of the different epitope specificities that have been defined so far. The anti-F(II) mixture contains the same five highly potent antibodies, but it also contains additional binders to some of the defined epitopes and the included antibodies also display a wider range of neutralizing activity on their own. It is thus possible that the activity of the highly potent antibodies becomes diluted in the anti-F(II) combination due to competition for binding to the neutralizing epitopes on the F protein. However, since there potentially are other effects than the neutralizing effect associated with each individual antibody, e.g. increased phagocytosis, increased antibody-dependent cellular cytotoxicity (ADCC), anti-inflammatory effects, complement activation, and a decreased likelihood of generating escape mutants, when considered in vivo, this result should not be taken as an indication that a mixture of five is better than a mixture of eleven antibodies when used in vivo.

Both the in vitro assays and the combinations of clones have been refined since this initial experiment and a number of combinations of F-specific antibody clones that are highly potent in the presence of complement have been identified. The neutralizing potencies, expressed as EC₅₀ values (effective concentrations required to induce a 50% reduction in the number of plaques), of additional anti-F antibody compositions are listed in Table 8. Irrespective of the exact number of clones in the compositions, the majority of the tested combinations of F-specific antibodies are more potent than Palivizumab with respect to neutralization of RSV strain subtype A.

### Mixtures of Anti-G Antibodies

The ability of mixtures of anti-G antibodies to neutralize RSV strains of subtype A was tested using the PRNT as described in Example 1, section j-2. The EC₅₀ values from the tested anti-G antibody compositions are listed in Table 8. Most of the compositions of two anti-G antibodies did not exhibit a markedly increased ability to neutralize virus compared to the individual anti-G antibodies. Some combinations of two or three anti-G antibodies never reached 100% neutralization of the virus, irrespective of the concentration. However, when additional anti-G antibodies were added to the composition the potency increased, possibly indicating a synergistic neutralizing effect between the anti-G antibodies. FIG. 7 shows an example of the increase in potency when combining multiple G-specific clones.

### Mixtures of Anti-F and Anti-G Antibodies

The ability of mixtures of anti-RSV protein F and protein G antibodies to neutralize RSV subtype B strain was compared with the neutralizing effect obtained using Palivizumab. The neutralization capability was assessed using either the microneutralization fusion inhibition assay as described in Example 1, Section j-4 or the plaque reduction neutralization assay (Example 1, section j-2).

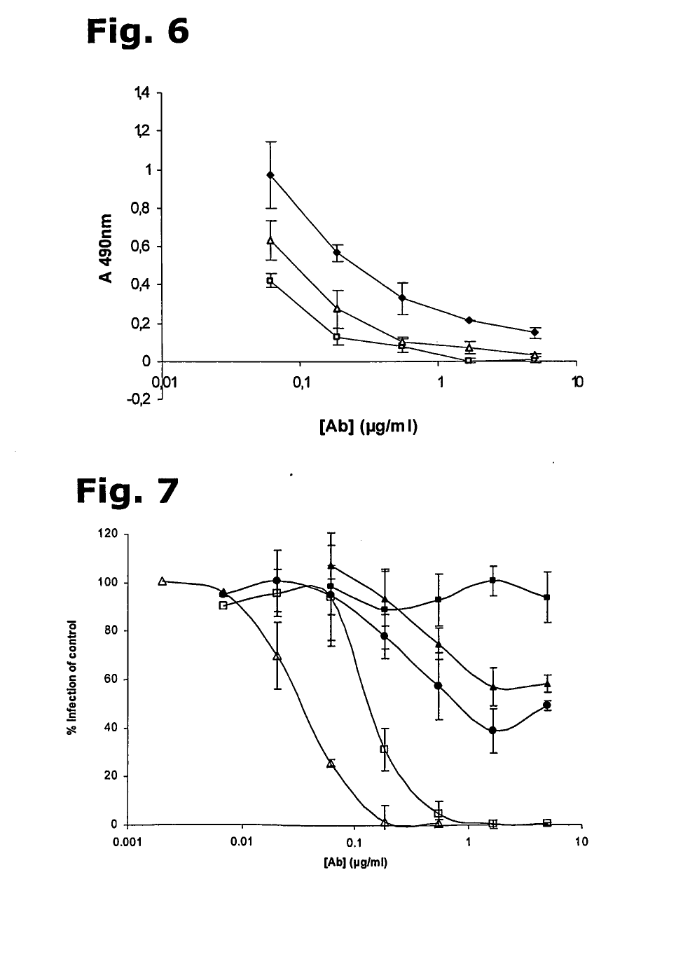

Initially, the neutralizing activity of two antibody mixtures, anti-F(I) G and anti-F(II)G, was measured in the microneutralization fusion inhibition assay. Each of these mixtures contains the anti-F antibodies of composition anti-F(I) and anti-F(II) described above as well as anti-G antibodies obtained from clones 793, 796, 838, 841, 856 and 888, where antibodies 793, 796, 838 bind to the conserved region of the G protein, 841, 856 binds to the GCRR of RSV subtype A and 888 binds to the GCRR of both subtypes (see Table 5). As shown in FIG. 6, both composition Anti-F(I) G and F(II)G are more potent than Palivizumab with respect to neutralization of the RSV B1 strain. Further, the neutralizing activity of the two mixtures is more or less equal. Thus, it seems that when the anti-F antibodies are combined with a number of protein G-specific clones, the potency difference previously observed between the two anti-F antibody mixtures is diminished. This may indicate a general increase in the neutralizing activity when antibodies that recognize a wide range of antigens and epitopes on RSV are combined.

A large number of different combinations of both anti-F and anti-G antibodies have since then been tested in the PRNT in the presence or absence of complement. EC₅₀ values obtained by this assay in the presence of active complement are presented in Table 8. All of the tested compositions including both anti-F and anti-G antibodies do neutralize RSV subtype A and the majority of these are more potent than Palivizumab.

The results also show that antibodies with naturally high affinities could repeatedly be obtained from human donors using the antibody cloning technique of the present invention.

TABLE 8
EC₅₀ values of combinations of anti-RSV
antibodies against RSV subtype A and B.

| | | EC50 value | |
|---|---|---|---|
| Compo- | | (μg/ml) | |
| sition | | Subtype | Subtype |
| number | Antibodies in composition | A | B |
| 1 | 810, 818, 819, 825, 827 | 0.19 | 0.38 |
| 2 | 810, 818, 819, 825, 827, 831, 858, 863, 884, | 0.34 | |
| | 894, 793, 796, 816, 838, 853, 856, 859, 888 | | |
| 3 | 810, 818, 825, 827, 884, 886, 793, 853, 868, | 0.30 | |
| | 888 | | |
| 4 | 810, 818, 825, 827, 831, 858, 884, 886, 793, | 0.19 | |
| | 796, 816, 853, 856, 868, 888 | | |
| 5 | 810, 818, 825, 827, 831, 858, 884, 886, 793, | 0.21 | |
| | 853, 868, 888 | | |
| 6 | 810, 819, 825, 827, 831, 793, 853, 856, 858, | 0.20 | |
| | 868 | | |
| 7 | 810, 811, 817, 819, 825, 827, 831, 838, 853, | 0.18 | |
| | 856, 858, 859, 863, 868 | | |
| 8 | 800, 801, 811, 838, 853, 855, 859, 861, 880, | ND | |
| | 894, 736, 795, 796, 799 | | |
| 9 | 810, 818, 825 | 0.14 | 0.29 |
| 10 | 810, 818, 819, 825, 827, 884 | 0.21 | 0.42 |
| 11 | 810, 818, 819, 825, 827, 884, 886 | 0.15 | 0.29 |
| 12 | 793, 816, 853, 856 | 0.06 | |
| 13 | 793, 816, 853, 855, 856 | 0.03 | |
| 14 | 793, 868, 888, 853, 856 | 0.34 | |
| 15 | 793, 796, 818, 816, 838, 853, 855, 856, 859, | 0.11 | |
| | 868, 888 | | |
| 16 | 810, 818, 827 | 0.11 | 0.21 |
| 17 | 810, 818, 825, 827, 858, 886 | 0.10 | 0.16 |
| 18 | 810, 818, 825, 827, 858, 886, 793, 816, 853, | 0.04 | 0.15 |
| | 855, 856 | | |
| 19 | 818, 825, 827, 858, 886, 793, 816, 853, 855, | 0.06 | |
| | 856 | | |
| 20 | 810, 818, 819, 825, 827, 858, 793, 816, 853, | 0.10 | |
| | 855, 856 | | |
| 21 | 810, 793, 816, 853, 855, 856 | 0.04 | |
| 22 | 818, 825, 827, 831, 858, 886, 793, 816, 853, | 0.06 | |
| | 855, 856 | | |
| 23 | 818, 825, 827, 831, 858, 819, 793, 816, 853, | 0.06 | |
| | 855, 856 | | |
| 24 | 818, 827, 831, 858, 819, 793, 816, 853, 855, | 0.06 | |
| | 856 | | |
| 25 | 810, 818, 819, 824, 825, 827, 858, 793, 816, | 0.07 | |
| | 853, 855, 856 | | |
| 26 | 831, 818, 819, 824, 825, 827, 858, 793, 816, | 0.08 | |
| | 853, 855, 856 | | |
| 27 | 831, 818, 819, 824, 827, 858, 793, 816, 853, | 0.05 | |
| | 855, 856 | | |
| 28 | 810, 818, 824 | 0.06 | 0.04 |
| 29 | 810, 824 | 0.05 | |
| 30 | 818, 824 | 0.04 | |
| 31 | 810, 818 | 0.11 | |
| 32 | 824, 793, 816, 853, 855, 856 | 0.05 | |
| 33 | 810, 818, 819, 824, 825, 827, 858, 894, 793, | 0.07 | 0.03 |
| | 816, 853, 855, 856 | | |
| 34 | 810, 818, 819, 824, 825, 827, 894, 793, 816, | 0.07 | |
| | 853, 855, 856 | | |
| 35 | 793, 816 | 5.94 | |
| 36 | 855, 856 | ND | |
| 37 | 793, 856 | ND | |
| 38 | 793, 853 | 2.35 | |
| 39 | 853, 856 | 0.21 | |
| 40 | 793, 853, 856 | 2.84 | |
| 41 | 793, 816, 853 | 1.97 | |
| 42 | 853, 855, 856 | 0.25 | |
| 43 | 793, 816, 853, 856 | 0.45 | |
| 44 | 793, 853, 855 | 0.26 | |
| 45 | 793, 853, 855, 856 | 0.16 | |
| 46 | 816, 853, 855, 856 | 0.07 | |
| 47 | 816, 856 | 0.06 | |
| 48 | 816, 853 | 0.75 | |
| 49 | 816, 853, 856 | 0.07 | |
| 50 | 810, 818, 824, 816 | 0.09 | |
| 51 | 810, 818, 824, 853 | 0.11 | |
| 52 | 810, 818, 824, 856 | 0.10 | |
| 53 | 810, 818, 824, 816, 853 | 0.09 | |
| 54 | 810, 818, 824, 816, 856 | 0.05 | |
| 55 | 810, 818, 824, 853, 856 | 0.08 | |
| 56 | 810, 818, 824, 816, 853, 856 | 0.05 | 0.03 |
| | Palivizumab | 0.14 | |

Blank fields indicate that the analysis has not been performed yet. ND indicates that an EC₅₀ value could not be determined in the PRNT due to a very low or lacking neutralizing activity.

### Example 4

### Reduction of Viral Loads in the Lungs of RSV-Infected Mice

The in vivo protective capacity of combinations of purified antibodies of the invention against RSV infection has been demonstrated in the BALB/c mouse model (Taylor et al. 1984. Immunology 52, 137-142; Mejias, et al. 2005. Antimicrob. Agents Chemother. 49: 4700-4707) as described in Example 1, Section k-1. In Table 9, data from an experiment with three different anti-RSV rpAb consisting of equal amounts of different antibody clones of the invention (described in table 8) are presented in comparison with data from uninfected control animals and placebo (PBS) treated animals of the same experiment. Each treatment group contained 5 mice and the samples were obtained on day five post-infection, which is approximately at the peak of virus replication in this model. As shown in Table 9, the rpAb combinations effectively reduce the virus load by at least an order of magnitude when given prophylactically. Copy numbers are presented as means±standard deviations. The copy number was at or below the limit of detection of this assay, i.e., 3.8 log 10 RNA copies/ml, for two of the treatment groups.

TABLE 9
Virus loads in the lungs of mice following
prophylaxis and RSV challenge.

| | Virus load by | |
|---|---|---|
| | RT-PCR (log10 RNA | New |
| Treatment group | copies/ml) | data |
| Uninfected | Negative | |
| PBS | 5.14 ± 0.09 | 4.25 |
| Anti-RSV rpAb 18 (50 mg/kg) | ND | |
| Anti-RSV rpAb 18 (5 mg/kg) | 4.61 ± 0.22 | 3.64 |
| Anti-RSV rpAb 9 (50 mg/kg) Small F Hi | ND | |
| Anti-RSV rpAb 9 (5 mg/kg) Small F Lo | 4.74 ± 0.38 | 3.82 |
| Anti-RSV rpAb 17 (50 mg/kg) Large F Hi | 4.41 ± 0.14 | 3.04 |
| Anti-RSV rpAb 17 (5 mg/kg) Large F Lo | 4.69 ± 0.05 | 3.90 |

Cytokine and Chemokine Levels in Lung Samples from RSV Infected Mice

Lung samples from a pilot mouse prophylaxis study were analyzed by a commercial multiplexed immunoassay to determine the levels of different cytokines and chemokines following RSV infection and antibody prophylaxis with rpAb 18 (Table 8) as described in Example 1, Section k-1. Samples from uninfected and untreated animals were also analyzed to obtain normative data for the BALB/c mouse. All samples were obtained on day five post-infection. Data are presented as means±standard deviations.

The analysis showed that the levels of a number of cytokines and chemokines that have been indicated as important markers of RSV infection and the subsequent inflammatory response, both in humans and mice, including interferon (IFN)-γ, interleukin (IL)-1β, IL-4, IL-6, IL-8 (KC/GROα), IL-10, macrophage inflammatory protein (MIP)-1α, Regulated upon activation of normal T cell expressed and secreted (RANTES, CCL5) and tumor necrosis factor (TNF)-α were increased in the lungs of the placebo-treated animals, whereas the lungs of the animals treated with approx. 50 mg/kg of rpAb had levels more or less on par with the uninfected control animals. Similar results were also obtained with other anti-RSV rpAb combinations. It should be noted that mice do not have a clear-cut homologue for IL-8, but they have a functional homologue for human GROα (similar function to IL-8) named KC.

The kinetics of the inflammatory response and the dose-response effects of antibody prophylaxis remain to be investigated.

TABLE 10
Levels of cytokines and chemokines in
lung tissue from RSV infected mice

| Level in tissue | Uninfected | | anti-RSV rpAb |
|---|---|---|---|
| sample (pg/ml) | control mice | Placebo treated mice | treated mice |
| IL-1β | 270 ± 71 | 570 ± 100 | 310 ± 140 |
| IL-4 | 7.7 ± 4.7 | 26 ± 4.6 | 14 ± 8.5 |
| IL-6 | 6.4 ± 2.6 | 22 ± 12 | 8.2 ± 3.8 |
| IL-10 | 120 ± 17 | 320 ± 58 | 170 ± 41 |
| IFN-γ | 20 ± 7.6 | 420 ± 88 | 81 ± 72 |
| KC/GROα (IL-8) | 51 ± 38 | 290 ± 83 | 94 ± 99 |
| MIP-1α (CCL3) | 39 ± 16 | 940 ± 170 | 160 ± 110 |
| RANTES (CCL5) | 60 ± 28 | 380 ± 32 | 140 ± 66 |
| TNF-α | 18 ± 6.1 | 95 ± 10 | 38 ± 25 |

1. An anti-RSV recombinant polyclonal antibody capable of neutralizing RSV subtype A and B, wherein said polyclonal antibody comprises distinct antibody members which in union specifically bind at least three different epitopes on at least one RSV envelope protein.

2. The anti-RSV recombinant polyclonal antibody according to claim 1, wherein said polyclonal antibody comprises distinct antibody members which together provide specific reactivity against at least two RSV envelope proteins.

3. The anti-RSV recombinant polyclonal antibody according to claim 1, wherein the at least one RSV envelope protein is selected from the group consisting of RSV G protein, RSV F protein and RSV SH protein.

4. The anti-RSV recombinant polyclonal antibody according to claim 1, wherein said antibody comprises at least two distinct anti-G antibody members and at least one distinct anti-F antibody member.

5. The anti-RSV recombinant polyclonal antibody according to claim 4, wherein the first anti-G antibody member is capable of specifically binding a conserved epitope on the G-protein, and the second anti-G antibody member is capable of specifically binding the G protein cysteine-rich region (GCRR), and the anti-F antibody member is directed against at least one of the antigenic sites I, II, IV, V, VI, C, or F1 on the F protein.

6. The anti-RSV recombinant polyclonal antibody according to claim 4, wherein at least a part of the anti-G reactivity is directed against the CX3C motif.

7. The anti-RSV recombinant polyclonal antibody according to claim 5, wherein the anti-G reactivity additionally is directed against at least one strain specific epitope.

8. The anti-RSV recombinant polyclonal antibody according to claim 4, wherein the anti-F reactivity is directed against antigenic site II and antigenic site IV.

9. The anti-RSV recombinant polyclonal antibody according to claim 3, wherein the anti-envelope protein reactivity is directed against the SH protein.

10. The anti-RSV recombinant polyclonal antibody according to claim 1, wherein the distinct antibody members mirror the humoral immune response in a donor with respect to diversity, affinity and specificity against RSV envelope antigens.

11. The anti-RSV recombinant polyclonal antibody according to claim 1, wherein the antibody members are encoded by nucleic acid sequences obtained from one or more human donors who have raised a humoral immune response against RSV, and the polyclonal antibody is a fully human antibody.

12. The anti-RSV recombinant polyclonal antibody according to claim 10, wherein the distinct antibody members are constituted of V_{H} and V_{L} pairs originally present in the donor.

13. The anti-RSV recombinant polyclonal antibody according to claim 1, wherein the antibody members comprise
(i) a V_{H} domain comprising one or more CDR regions encoded by SEQ ID NOs: 201-455 and;
(ii) a V_{L} domain comprising one or more CDR regions encoded by SEQ ID NOs: 456-710.

14. A pharmaceutical composition comprising an anti-RSV recombinant polyclonal antibody according to claim 1 and a pharmaceutically acceptable excipient.

15. A method of preventing, treating or ameliorating one or more symptoms associated with a RSV infection in a mammal, comprising administering an effective amount of the pharmaceutical composition according to claim 14 to said mammal.

16. The method according to claim 15, wherein the effective amount is at most 100 mg of the antibody per kg of body weight.

17. The method according to claim 15, wherein the effective amount is at least 0.01 mg of the antibody per kg of body weight.

18. The method according to claim 15, wherein the effective amount is between 0.1-20 mg antibody per kg of body weight.

19. The method according to claim 15, wherein the composition is administered at least 1 time per year.

20. The method according to claim 19, wherein the composition is administered at regular intervals during the period of the year where there is an increased risk of attracting an RSV infection.

21. The method according to claim 20, wherein the regular intervals are weekly, bi-weekly, monthly, or bimonthly.

22. (canceled)

23. A method for generating a repertoire of V_{H} and V_{L} coding pairs, wherein the V_{H} and V_{L} coding pairs mirror the gene pairs responsible for the humoral immune response resulting from a RSV infection, said method comprising:
a. providing a lymphocyte-containing cell fraction from an RSV infected donor or from a donor recovering from an RSV infection;
b. optionally enriching B cells or plasma cells from said cell fraction;
c. obtaining a population of isolated single cells, comprising distributing cells from said cell fraction individually into a plurality of vessels;
d. amplifying and effecting linkage of the V_{H} and V_{L} coding pairs, in a multiplex overlap extension RT-PCR procedure, using a template derived from said isolated single cells; and
e. optionally performing a nested PCR of the linked V_{H} and V_{L} coding pairs.

24. A polyclonal cell line capable of expressing a recombinant polyclonal anti-RSV antibody according to claim 1.

25. A polyclonal cell line wherein each individual cell is capable of expressing a single V_{H} and V_{L} coding pair and the polyclonal cell line as a whole is capable of expressing a collection of V_{H} and V_{L} coding pairs, wherein each V_{H} and V_{L} coding pair encodes an anti-RSV antibody.

26. The polyclonal cell line generated according to the method of claim 23.

27. An isolated human anti RSV-antibody molecule or a specifically binding fragment of said antibody molecule or a synthetic or semi-synthetic antibody analogue thereof, said antibody molecule, binding fragment or analogue comprising:
(i) a V_{H} domain comprising one or more CDR regions encoded by SEQ ID NOs: 201-455 and;
(ii) a V_{L} domain comprising one or more CDR regions encoded by SEQ ID NOs: 456-710.

28. The antibody molecule, fragment or analogue according to claim 27, which includes a heavy chain amino acid sequence selected from SEQ ID Nos: 1-44 and a light chain amino acid sequence selected from SEQ ID NOs: 89-132.

29. An isolated antibody molecule, an antibody fragment or a synthetic or semi-synthetic antibody analogue, which comprises CDRs identical to the CDRs in an Fab derived from a human antibody, said Fab having a dissociation constant, K_{D}, for the RSV G protein of at most 500 mM.

30. The isolated antibody molecule, antibody fragment or synthetic or semi-synthetic antibody according to claim 29, wherein the K_{D} is at most 400 nM.

31. An isolated antibody molecule, an antibody fragment or a synthetic or semi-synthetic antibody, which comprises an antigen binding site identical to the antigen binding site in an Fab derived from a human antibody, said Fab having a dissociation constant, K_{D}, for the RSV F protein of at most 500 nM.

32. The isolated antibody, antibody fragment or synthetic or semi-synthetic antibody according to claim 31, wherein the K_{D} is at most 400 nM.

33. The antibody molecule or specifically binding fragment or synthetic or semi-synthetic antibody analogue according to claim 27, which comprises an amino acid sequence selected from the group consisting of:
(i) SEQ ID NOs: 11 and 99;
(ii) SEQ ID NOs: 17 and 105;
(iii) SEQ ID) NOs: 18 and 106;
(iv) SEQ ID NOs: 19 and 107;
(v) SEQ ID NOs: 20 and 108;
(vi) SEQ ID NOs: 21 and 109;
(vii) SEQ ID NOs: 32 and 120; and
(viii) SEQ ID NOs: 44 and 132.

34. An antibody composition comprising an antibody molecule, specifically binding fragment or synthetic or semi-synthetic antibody analogue according to claim 27 in admixture with a pharmaceutically acceptable carrier, excipient, vehicle or diluent.

35. The composition according to claim 34, which comprises 2 distinct antibody molecules specifically binding fragments or synthetic or semi-synthetic antibody analogues.

36. The composition according to claim 34, which comprises at least 3 distinct antibody molecules antibody fragments or synthetic or semisynthetic antibody analogues.

37. The composition according to claim 34 which includes at least one antibody molecule, fragment or analogue which binds the RSV F protein and which includes at least one antibody, fragment or analogue which binds the RSV G protein.

38. An isolated nucleic acid fragment which encodes an amino acid sequence selected from the group consisting of SEQ ID NOs: 11, 17-21, 32, 44, 105-109, 120, 132 and 201-710.

39. The isolated nucleic acid fragment of claim 38, which encodes an amino acid sequence selected from the group consisting of SEQ ID NOs: 201-710.

40. An isolated nucleic acid fragment, which encodes a heavy chain amino acid sequence set forth in any one of SEQ ID NOs: 1-44.

41. An isolated nucleic acid fragment, which encodes a light chain amino acid sequence set forth in any one of SEQ ID NOs: 89-132.

42. An isolated nucleic acid fragment, which encodes a heavy chain amino acid sequence set forth in any one of SEQ ID NOs: 1-44 and a light chain amino acid sequence set forth in any one of SEQ ID NOs: 89-132.

43. The nucleic acid fragment according to claim 38, which includes a coding sequence set forth in any one of SEQ ID NOs: 45-88 or 133-176.

44. A vector, comprising the nucleic acid fragment according to claim 38.

45. The vector according to claim 44 being capable of autonomous replication.

46. The vector according to claim 44 being selected from the group consisting of a plasmid, a phage, a cosmid, a mini-chromosome, and a virus.

47. The vector according to claim 44, comprising,
(i) in the 5′→3′ direction and in operable linkage at least one promoter for driving expression of a first nucleic acid fragment according to claim 38, which encodes at least one light chain CDR together with necessary framework regions, optionally a nucleic acid sequence encoding a leader peptide, said first nucleic acid fragment, optionally a nucleic acid sequence encoding constant regions, and optionally a nucleic acid sequence encoding a first terminator, or
(ii) in the 5′→3′ direction and in operable linkage at least one promoter for driving expression of a second nucleic acid fragment according to claim 38 which encodes at least one heavy chain CDR together with necessary framework regions, optionally a nucleic acid sequence encoding a leader peptide, said second nucleic acid fragment, optionally a nucleic acid sequence encoding constant regions, and optionally a nucleic acid sequence encoding a second terminator.

48. The vector according to claim 44 which, when introduced into a host cell, is integrated in the host cell genome.

49. A transformed cell carrying the vector of claim 44.

50. A stable cell line which carries the vector according to claim 44 which optionally secretes or carries its recombinant expression product on its surface.


SEQUENCE LISTING (714 sequences)

SEQ ID NO: 1 - protein, 122 aa, homo sapiens
QVQLQESGPGLVKPSETLSLTCTVSNGAIGDYDWSWIRQSPGKGLEWIGNINYRGNTNYNPSLKSRVTMSLRTSTMQFSLKLSSATAADTAVYYCARDVGYGGGQYFAMDVWSPGTTVTVSS

SEQ ID NO: 2 - protein, 129 aa, homo sapiens
QVQLVESGGGVVQPGGSLRLSCTASGFTFSTYGMHWVRQAPGKGLEWVAFIRYDGSTQDYVDSVKGRFTISRDNSKNMVYVQMNSLRVEDTAVYYCAKDMDYYGSRSYSVTYYYGMDVWGQGTTVTVSS

SEQ ID NO: 3 - protein, 125 aa, homo sapiens
QVQLVQSGAEVKKPGASVKVSCKASGYTFSGYYMHWVRQAPGQGLEWMGWINTSSGGTNYAQKFQGRVTMTRDTSISTAHMELRRLRSDDTAVYYCAREDGTMGTNSWYGWFDPWGQGTLVTVSS

SEQ ID NO: 4 - protein, 127 aa, homo sapiens
QVQLVESGGGLVKPGGSLRLSCAASGFPFGDYYMSWIRQAPGKGLEWVAYINRGGTTIYYADSVKGRFTISRDNAKNSLFLQMNSLRAGDTALYYCARGLILALPTATVELGAFDIWGQGTMVTVSS

SEQ ID NO: 5 - protein, 126 aa, homo sapiens
QVQLQESGPGLVKPSQTLSLTCTVSGASISSGDYYWSWIRQSPRKGLEWIGYIFHSGTTYYNPSLKSRAVISLDTSKNQFSLRLTSVTAADTAVYYCARDVDDFPVWGMNRYLALWGRGTLVTVSS

SEQ ID NO: 6 - protein, 124 aa, homo sapiens
QVQLVESGGGVVQPGRSLRLSCAASGFSFSHFGMHWVRQVPGKGLEWVAIISYDGNNVHYADSVKGRFTISRDNSKNTLFLQMNSLRDDDTGVYYCAKDDVATDLAAYYYFDVWGRGTLVTVSS

SEQ ID NO: 7 - protein, 123 aa, homo sapiens
QVQLVESGGGVVQPGRSLKLSCEASGFNFNNYGMHWVRQAPGKGLEWVAVISYDGRNKYFADSVKGRFIISRDDSRNTVFLQMNSLRVEDTAVYYCARGSVQVWLHLGLFDNWGQGTLVTVSS

SEQ ID NO: 8 - protein, 122 aa, homo sapiens
QVQLVESGGAVVQPGRSLRLSCEVSGFSFSDYGMNWVRQGPGKGLEWVAVIWHDGSNKNYLDSVKGRFTVSRDNSKNTLFLQMNSLRAEDTAVYYCARTPYEFWSGYYFDFWGQGTLVTVSS

SEQ ID NO: 9 - protein, 116 aa, homo sapiens
QVQLVESGGGVVQPGRSLRLSCAASGFPFNSYAMHWVRQAPGKGLEWVAVIYYEGSNEYYADSVKGRFTISRDNSKNTLYLQMDSLRAEDTAVYYCARKWLGMDFWGQGTLVTVSS

SEQ ID NO: 10 - protein, 120 aa, homo sapiens
EVQLVESGGGLVRPGGSLRLSCSASGFTFSNYAMHWVRQAPGKRLEYVSATSTDGGSTYYADSLKGTFTISRDNSKNTLYLQMSSLSTEDTAIYYCARRFWGFGNFFDYWGRGTLVTVSS

SEQ ID NO: 11 - protein, 122 aa, homo sapiens
QVQLVQSGAEVKKSGSSVKVSCRASGGTFGNYAINWVRQAPGQGLEWVGRIIPVFDTTNYAQKFQGRVTITADRSTNTAIMQLSSLRPQDTAMYYCLRGSTRGWDTDGFDIWGQGTMVTVSS

SEQ ID NO: 12 - protein, 129 aa, homo sapiens
QVQLVQSGAVVETPGASVKVSCKASGYIFGNYYIHWVRQAPGQGLEWMAVINPNGGSTTSAQKFQDRITVTRDTSTTTVYLEVDNLRSEDTATYYCARQRSVTGGFDAWLLIPDASNTWGQGTMVTVSS

SEQ ID NO: 13 - protein, 126 aa, homo sapiens
QVQLVQSGAEMKKPGSSVKVSCKASGGSFSSYSISWVRQAPGRGLEWVGMILPISGTTNYAQTFQGRVIISADTSTSTAYMELTSLTSEDTAVYFCARVFREFSTSTLDPYYFDYWGQGTLVTVSS

SEQ ID NO: 14 - protein, 125 aa, homo sapiens
QVQLVESGGGVVQPGKSVRLSCVGSGFRLMDYAMHWVRQAPGKGLDWVAVISYDGANEYYAESVKGRFTVSRDNSDNTLYLQMKSLRAEDTAVYFCARAGRSSMNEEVIMYFDNWGLGTLVTVSS

SEQ ID NO: 15 - protein, 127 aa, homo sapiens
EVQLLESGGGLVQPGGSLRLSCVASGFTFSTYAMTWVRQAPGKGLEWVSVIRASGDSEIYADSVRGRFTISRDNSKNTVFLQMDSLRVEDTAVYFCANIGQRRYCSGDHCYGHFDYWGQGTLVTVSS

SEQ ID NO: 16 - protein, 127 aa, homo sapiens
QVQLVESGGGVVQPGRSLRLSCAASGFGFNTHGMHWVRQAPGKGLEWLSIISLDGIKTHYADSVKGRFTISRDNSKNTVFLQLSGLRPEDTAVYYCAKDHIGGTNAYFEWTVPFDGWGQGTLVTVSS

SEQ ID NO: 17 - protein, 126 aa, homo sapiens
QVTLRESGPAVVKPTETLTLTCAFSGFSLNAGRVGVSWIRQPPGQAPEWLARIDWDDDKAFRTSLKTRLSISKDSSKNQVVLTLSNMDPADTATYYCARTQVFASGGYYLYYLDHWGQGTLVTVSS

SEQ ID NO: 18 - protein, 129 aa, homo sapiens
QVQLQESGPGLVKPSQTLSLTCTVSSGAISGADYYWSWIRQPPGKGLEWVGFIYDSGSTYYNPSLRSRVTISIDTSKKQFSLKLTSVTAADTAVYYCARDLGYGGNSYSHSYYYGLDVWGRGTTVTVSS

SEQ ID NO: 19 - protein, 119 aa, homo sapiens
QVQLQESGPGLVKPSETLSLTCTVSGGSIGNYYWGWIRQPPGKGLEWIGHIYFGGNTNYNPSLQSRVTISVDTSRNQFSLKLNSVTAADTAVYYCARDSSNWPAGYEDWGQGTLVTVSS

SEQ ID NO: 20 - protein, 123 aa, homo sapiens
QVQLVQSGAEVKKPGASVKVSCKVSGYTFTSNGLSWVRQAPGQGFEWLGWISASSGNKKYAPKFQGRVTLTTDISTSTAYMELRSLRSDDTAVYYCAKDGGTYVPYSDAFDFWGQGTMVTVSS

SEQ ID NO: 21 - protein, 118 aa, homo sapiens
QVQLVQSGAEVKKPGASVKVSCRVSGHTFTALSKHWMRQGPGGGLEWMGFFDPEDGDTGYAQKFQGRVTMTEDTATGTAYMELSSLTSDDTAVYYCATVAAAGNFDNWGQGTLVTVSS

SEQ ID NO: 22 - protein, 126 aa, homo sapiens
QVTLKESGPALVKATQTLTLTCTFSGFSLSRNRMSVSWIRQPPGKALEWLARIDWDDDKFYNTSLQTRLTISKDTSKNQVVLTMTNMDPVDTATYYCARTGIYDSSGYYLYYFDYWGQGTLVTVSS

SEQ ID NO: 23 - protein, 130 aa, homo sapiens
QVQLVQSGAEVKVPGASVKVSCKASGYTFTTYGVSWVRQAPGQGLEWMGWISAYNGNTYYLQKLQGRVTMTTDTSTSTAYMELRGLRSDDTAMYYCARDRVGGSSSEVLSRAKNYGLDVWGQGTTVTVSS

SEQ ID NO: 24 - protein, 123 aa, homo sapiens
QVQLVQSGAEVKKPGASVKVSCKASANIFTYAMHWVRQAPGQRLEWMGWINVGNGQTKYSQRFQGRVTITRDTSATTAYMELSTLRSEDTAVYYCARRASQYGEVYGNYFDYWGQGTLVTVSS

SEQ ID NO: 25 - protein, 126 aa, homo sapiens
QVQLVQSGAEVKRPGASVKVSCKASGYTFISYGFSWVRQAPGQGLEWMGWSSVYNGDTNYAQKFHGRVNMTTDTSTNTAYMELRGLRSDDTAVYFCARDRNVVLLPAAPFGGMDVWGQGTMVTVSS

SEQ ID NO: 26 - protein, 123 aa, homo sapiens
QVQLVESGGGVVQPGTSLRLSCAASGFTFSTFGMHWVRQAPGKGLEWVAVISYDGNKKYYADSVKGRFTISRDNSKNTLYLQVNSLRVEDTAVYYCAAQTPYFNESSGLVPDWGQGTLVTVSS

SEQ ID NO: 27 - protein, 126 aa, homo sapiens
QVQLVQSGAEVKKPGASVKVSCKASGYTFISFGISWVRQAPGQGLEWMGWISAYNGNTDYAQRLQDRVTMTRDTATSTAYLELRSLKSDDTAVYYCTRDESMLRGVTEGFGPIDYWGQGTLVTVSS

SEQ ID NO: 28 - protein, 128 aa, homo sapiens
EVQLVQSGAEVKKPGQSLKISCKTSGYIFTNYWIGWVRQRPGKGLEWMGVIFPADSDARYSPSFQGQVTISADKSIGTAYLQWSSLKASDTAIYYCARPKYYFDSSGQFSEMYYFDFWGQGTLVTVSS

SEQ ID NO: 29 - protein, 119 aa, homo sapiens
QVQLVQSGPEVKKPGASVKVSCKASGYVLTNYAFSWVRQAPGQGLEWLGWISGSNGNTYYAEKFQGRVTMTTDTSTSTAYMELRSLRSDDTAVYFCARDLLRSTYFDYWGQGTLVTVSS

SEQ ID NO: 30 - protein, 126 aa, homo sapiens
QVQLVQSGAEVKKPGASVKVSCKASGYTFSNYGFSWVRQAPGRGLEWMGWISAYNGNTYYAQNLQGRVTMTTDTSTTTAYMVLRSLRSDDTAMYYCARDGNTAGVDMWSRDGFDIWGQGTMVTVSS

SEQ ID NO: 31 - protein, 131 aa, homo sapiens
EVQLLESGGGLVQPGGPLRLSCVASGFSFSSYAMNWIRLAPGKGLEWVSGISGSGGSTYYGDSVKGRFTISRDNSKNTLYLQMNSLRAEDTAVYYCAKEPWIDIVVASVISPYYYDGMDVWGQGTTVTVSS

SEQ ID NO: 32 - protein, 123 aa, homo sapiens
QVQLVQSGAEVKKPGSSVKVSCKASGGSFDGYTISWLRQAPGQGLEWMGRVVPTLGFPNYAQKFQGRVTVTADRSTNTAYLELSRLTSEDTAVYYCARMNLGSHSGRPGFDMWGQGTLVTVSS

SEQ ID NO: 33 - protein, 129 aa, homo sapiens
QVQLVESGGGVVQPGRSLRLSCAVSGSSFSKYGIHWVRQAPGKGLEWVAVISYDGSKKYFTDSVKGRFTIARDNSQNTVFLQMNSLRAEDTAVYYCATGGGVNVTSWSDVEHSSSLGYWGLGTLVTVSS

SEQ ID NO: 34 - protein, 125 aa, homo sapiens
QVQLVESGGGVVQPGGSLRLSCAASGFTFSSYGMHWVRQAPGKGLEWVAFIWNDGSNKYYADSVKGRFTISRDNSKNTLYLQMNSLRAEDTAVYYCVKDEVYDSSGYYLYYFDSWGQGTLVTVSS

SEQ ID NO: 35 - protein, 127 aa, homo sapiens
EVQLLESGGGLVQPGGSLRLSCAASGFTFSSYTMSWVRQAPGKGLEWVSSISASTVLTYYADSVKGRFTISRDNSKNTLYLQMSSLRAEDTAVYYCAKDYDFWSGYPGGQYWFFDLWGRGTLVTVSS

SEQ ID NO: 36 - protein, 123 aa, homo sapiens
QVQLQESGPGLVTPSETLSVTCTVSNYSIDNAYYWGWIRQPPGKGLEWIGSIHHSGSAYYNSSLKSRATISIDTSKNQFSLNLRSVTAADTAVYYCARDTILTFGEPHWFDPWGQGTLVTVSS

SEQ ID NO: 37 - protein, 124 aa, homo sapiens
QVQLQESGPGLVKPSETLSLTCTVSGDSISNYYWSWIRQPPGKGLEWIGEISNTWSTNYNPSLKSRVTISLDMPKNQLSLKLSSVTAADTAVYYCARGLFYDSGGYYLFYFQHWGQGTLVTVSS

SEQ ID NO: 38 - protein, 125 aa, homo sapiens
QVQLVESGGGVVQPGRSLRVSCAASGFTFSNYGMHWVRQAPGKGLEWVAVIWYDDSNKQYGDSVKGRFTISRDNSKSTLYLQMDRLRVEDTAVYYCARASEYSISWRHRGVLDYWGQGTLVTVSS

SEQ ID NO: 39 - protein, 126 aa, homo sapiens
QITLKESGPTLVRPTQTLTLTCTFSGFSLSTSKLGVGWIRQPPGKALEWLALVDWDDDRRYRPSLKSRLTVTKDTSKNQVVLTMTNMDPVDTATYYCAHSAYYTSSGYYLQYFHHWGPGTLVTVSS

SEQ ID NO: 40 - protein, 125 aa, homo sapiens
EVQLVESGGGVVQPGGSLRLSCEVSGFTFNSYEMTWVRQAPGKGLEWVSHIGNSGSMIYYADSVKGRFTISRDNAKNSLYLQMNSLRVEDTAVYYCARSDYYDSSGYYLLYLDSWGHGTLVTVSS

SEQ ID NO: 41 - protein, 120 aa, homo sapiens
QVQLVQSGAEVRKPGASVKVSCKASGHTFINFAMHWVRQAPGQGLEWMGYINAVNGNTQYSQKFQGRVTFTRDTSANTAYMELSSLRSEDTAVYYCARNNGGSAIIFYYWGQGTLVTVSS

SEQ ID NO: 42 - protein, 122 aa, homo sapiens
QVQLVESGGGVVQPGRSLRLSCAASGFSFSSYGMHWVRQAPGKGLEWVAVISNDGSNKYYADSVKGRFTISRDNSKKTMYLQMNSLRAEDTAVYFCAKTTDQRLLVDWFDPWGQGTLVTVSS

SEQ ID NO: 43 - protein, 130 aa, homo sapiens
QLQLQESGPGLVKPSETLSLTCTASGGSINSSNFYWGWIRQPPGKGLEWIGSIFYSGTTYYNPSLKSRVTISVDTSKNQFSLKLSPVTAADTAVYHCARHGFRYCNNGVCSINLDAFDIWGQGTMVTVSS

SEQ ID NO: 44 - protein, 122 aa, homo sapiens
QVQLVESGGGVVQPGKSLRLSCAASGFRFSDYGMHWVRQAPSKGLEWVAVIWHDGSNIRYADSVRGRFSISRDNSKNTLYLQMNSMRADDTAFYYCARVPFQIWSGLYFDHWGQGTLVTVSS

SEQ ID NO: 45 - DNA, 366 bp, homo sapiens
caggtgcagctgcaggagtcgggcccaggactggtgaagccttcggagaccctgtccctcacgtgcactgtgtctaatggcgccatcggcgactacgactggagctggattcgtcagtccccagggaagggactggagtggattgggaacataaattacagagggaacaccaactacaacccctccctcaagagtcgagtcaccatgtccctacgcacgtccacgatgcagttctccctgaagctgagctctgcgaccgctgcggacacggccgtctattactgtgcgagagatgtaggctacggtggcgggcagtatttcgcgatggacgtctggagcccagggaccacggtcaccgtctcgagt

SEQ ID NO: 46 - DNA, 387 bp, homo sapiens
caggtgcagctggtggagtctgggggaggcgtggtccagcctggggggtccctgagactctcctgtacagcgtctggattcaccttcagtacctatggcatgcactgggtccgccaggctcccggcaaggggctggaatgggtggcatttatacggtatgatggaagtactcaagactatgtagactccgtgaagggccgattcaccatctccagagacaattccaagaatatggtgtatgtgcagatgaacagcctgagagttgaggacacggctgtctattactgtgcgaaagacatggattactatggttcgcggagttattctgtcacctactactacggaatggacgtctggggccaagggaccacggtcaccgtctcgagt

SEQ ID NO: 47 - DNA, 375 bp, homo sapiens
caggtgcagctggtgcagtctggggctgaggtgaagaagcctggggcctcagtgaaggtctcctgcaaggcttctggatacaccttcagcggctattatatgcactgggtgcgacaggcccctggacaagggcttgagtggatgggatggatcaacactagcagtggtggcacaaactatgcgcagaagtttcagggcagggtcaccatgaccagggacacgtccatcagcacagcccacatggaactgaggaggctgagatctgacgacacggccgtgtattattgtgcgagagaggacggcaccatgggtactaatagttggtatggctggttcgacccctggggccagggaaccctggtcaccgtctcgagt

SEQ ID NO: 48 - DNA, 381 bp, homo sapiens
caggtgcagctggtggagtctgggggaggcttggtcaagcctggggggtccctgagactctcctgtgcggcctctggattccccttcggtgactactacatgagctggatccgccaggctccagggaagggactggagtgggttgcatacattaatagaggtggcactaccatatactacgcagactctgtgaagggccgattcaccatctccagggacaacgccaagaactccctgtttctgcaaatgaacagcctgagagccggggacacggccctctattactgtgcgagagggctaattctagcactaccgactgctacggttgagttaggagcttttgatatctggggccaagggacaatggtcaccgtctcgagt

SEQ ID NO: 49 - DNA, 378 bp, homo sapiens
caggtgcagctgcaggagtcgggcccaggactggtgaagccttcacagaccctgtccctcacctgcactgtctctggtgcctccatcagcagtggtgattattactggagttggatccgtcagtctccaaggaagggcctggagtggattgggtacatcttccacagtgggaccacgtactacaacccgtccctcaagagtcgagctgtcatctcactggacacgtccaagaaccaattctccctgaggctgacgtctgtgactgccgcagacacggccgtctattattgtgccagagatgtcgacgattttcccgtttggggtatgaatcgatatcttgccctctggggccggggaaccctggtcaccgtctcgagt

SEQ ID NO: 50 - DNA, 372 bp, homo sapiens
caggtgcagctggtggagtctgggggaggcgtggtccagcctgggaggtccctgagactctcctgtgcagcctctggattcagcttcagtcactttggcatgcactgggtccgccaggttccaggcaaggggctggagtgggtggcaattatatcatatgatgggaataatgtacactatgccgactccgtaaagggccgattcaccatctccagagacaattccaagaacacgctgtttctgcaaatgaacagcctgagagatgacgacacgggtgtgtattactgtgcgaaggacgacgtggcgacagatttggctgcctactactacttcgatgtctggggccgtggcaccctggtcaccgtctcgagt

SEQ ID NO: 51 - DNA, 369 bp, homo sapiens
caggtgcagctggtggagtctgggggcggcgtggtccagcctgggaggtccctgaaactctcttgtgaagcctctggattcaacttcaataattatggcatgcactgggtccgccaggcaccaggcaaggggctggagtgggtggcagttatttcatatgacggaagaaataagtattttgctgactccgtgaagggccgattcatcatctccagagacgattccaggaacacagtgtttctgcaaatgaacagcctgcgagttgaagatacggccgtctattactgtgcgagaggcagcgtacaagtctggctacatttgggactttttgacaactggggccagggaaccctggtcaccgtctcgagt

SEQ ID NO: 52 - DNA, 366 bp, homo sapiens
caggtgcagctggtggagtctgggggagccgtggtccagcctgggaggtccctgagactctcctgtgaagtgtctggattcagtttcagtgactatggcatgaactgggtccgccagggtccaggcaaggggctggagtgggtggcagttatatggcatgacggaagtaataaaaattatctagactccgtgaagggccgattcaccgtctccagagacaattccaagaacacattgtttctgcaaatgaacagcctgagagccgaagacacggctgtatattactgtgcgaggacgccttacgagttttggagtggctattactttgacttctggggccagggaaccctggtcaccgtctcgagt

SEQ ID NO: 53 - DNA, 348 bp, homo sapiens
caggtgcagctggtggagtctgggggaggcgtggtccagcctgggaggtccctgagactctcctgtgcagcgtctggattccccttcaatagctatgccatgcactgggtccgccaggctccaggcaaggggctggagtgggtggcagtgatatattatgaagggagtaatgaatattatgcagactccgtgaagggccgattcaccatctccagagacaattccaagaacactctgtatttgcaaatggatagcctgagagccgaggacacggctgtctattactgtgcgaggaagtggctggggatggacttctggggccagggaaccctggtcaccgtctcgagt

SEQ ID NO: 54 - DNA, 360 bp, homo sapiens
gaggtgcagctggtggagtctgggggaggcttggtccggcctggggggtccctgagactctcctgttcagcctctggattcaccttcagtaactatgctatgcactgggtccgccaggctccagggaagagactggaatatgtttcagctactagtactgatggggggagcacatactacgcagactccctaaagggcacattcaccatctccagagacaattccaagaacacactgtatcttcaaatgagcagtctcagtactgaggacacggctatttattactgcgcccgccgattctggggatttggaaacttttttgactactggggccggggaaccctggtcaccgtctcgagt

SEQ ID NO: 55 - DNA, 366 bp, homo sapiens
caggtgcagctggtgcagtctggggctgaggtgaagaagtccgggtcctcggtgaaggtctcctgcagggcttctggaggcaccttcggcaattatgctatcaactgggtgcgacaggcccctggacaagggcttgagtgggtgggaaggatcatccctgtctttgatacaacaaactacgcacagaagttccagggcagagtcacgattaccgcggacagatccacaaacacagccatcatgcaactgagcagtctgcgacctcaggacacggccatgtattattgtttgagaggttccacccgtggctgggatactgatggttttgatatctggggccaagggacaatggtcaccgtctcgagt

SEQ ID NO: 56 - DNA, 387 bp, homo sapiens
caggttcagctggtgcagtctggggctgtcgtggagacgcctggggcctcagtgaaggtctcctgcaaggcatctggatacatcttcggcaactactatatccactgggtgcggcaggcccctggacaagggcttgagtggatggcagttatcaatcccaatggtggtagcacaacttccgcacagaagttccaagacagaatcaccgtgaccagggacacgtccacgaccactgtctatttggaggttgacaacctgagatctgaggacacggccacatattattgtgcgagacagagatctgtaacagggggctttgacgcgtggcttttaatcccagatgcttctaatacctggggccaggggacaatggtcaccgtctcgagt

SEQ ID NO: 57 - DNA, 378 bp, homo sapiens
caggtgcagctggtgcagtctggggctgagatgaagaagcctgggtcctcggtgaaggtctcctgcaaggcttctggaggctccttcagcagctattctatcagctgggtgcgacaggcccctggacgagggcttgagtgggtgggaatgatcctgcctatctctggtacaacaaactacgcacagacatttcagggcagagtcatcattagcgcggacacatccacgagcacagcctacatggagctgaccagcctcacatctgaagacacggccgtgtatttctgtgcgagagtctttagagaatttagcacctcgacccttgacccctactactttgactactggggccagggaaccctggtcaccgtctcgagt

SEQ ID NO: 58 - DNA, 375 bp, homo sapiens
caggtgcagctggtggagtctgggggaggcgtggtccagcctgggaagtccgtgagactctcctgtgtaggctctggcttcaggctcatggactatgctatgcactgggtccgccaggctccaggcaagggactggattgggtggcagttatttcatatgatggagccaatgaatactacgcagagtccgtgaagggccgattcaccgtctccagagacaattcagacaacactctgtatctacaaatgaagagcctgagagctgaggacacggctgtgtatttctgtgcgagagcgggccgttcctctatgaatgaagaagttattatgtactttgacaactggggcctgggaaccctggtcaccgtctcgagt

SEQ ID NO: 59 - DNA, 381 bp, homo sapiens
gaggtgcagctgttggagtctgggggaggcttggtccagcctggggggtccctgagactctcctgtgtagcctccggattcacctttagtacctacgccatgacctgggtccgccaggctccagggaaggggctggagtgggtctcagtcattcgtgctagtggtgatagtgaaatctacgcagactccgtgaggggccggttcaccatctccagagacaattccaagaacacggtgtttctgcaaatggacagcctgagagtcgaggacacggccgtatatttctgtgcgaatataggccagcgtcggtattgtagtggtgatcactgctacggacactttgactactggggccagggaaccctggtcaccgtctcgagt

SEQ ID NO: 60 - DNA, 381 bp, homo sapiens
caggtgcagctggtggagtctgggggaggcgtggtccaacctgggaggtccctgagactctcctgtgcagcctctggattcggcttcaacacccatggcatgcactgggtccgccaggctccaggcaaggggctggagtggctgtcaattatctcacttgatgggattaagacccactatgcagactccgtgaagggccgattcaccatctccagagacaattccaagaacacggtgtttctacaattgagtggcctgagacctgaagacacggctgtatattactgtgcgaaagatcatattggggggacgaacgcatattttgaatggacagtcccgtttgacggctggggccagggaaccctggtcaccgtctcgagt

SEQ ID NO: 61 - DNA, 378 bp, homo sapiens
caggtcaccttgagggagtctggtccagcggtggtgaagcccacagaaacgctcactctgacctgcgccttctctgggttctcactcaacgccggtagagtgggtgtgagttggatccgtcagcccccagggcaggccccggaatggcttgcacgcattgattgggatgatgataaagcgttccgcacatctctgaagaccagactcagcatctccaaggactcctccaaaaaccaggtggtccttacactgagcaacatggaccctgcggacacagccacatattactgtgcccggacacaggtcttcgcaagtggaggctactacttgtactaccttgaccactggggccagggaaccctggtcaccgtctcgagt

SEQ ID NO: 62 - DNA, 387 bp, homo sapiens
caggtgcagctgcaggagtcgggcccaggactggtgaagccttcacagaccctgtccctcacctgcactgtctctagtggcgccatcagtggtgctgattactactggagttggatccgccagcccccagggaagggcctggagtgggttgggttcatctatgacagtgggagcacctactacaacccgtccctcaggagtcgagtgaccatatcaatagacacgtccaagaagcagttctccctgaagctgacctctgtgactgccgcagacacggccgtgtattactgtgccagagatctaggctacggtggtaactcttactcccactcctactactacggtttggacgtctggggccgagggaccacggtcaccgtctcgagt

SEQ ID NO: 63 - DNA, 357 bp, homo sapiens
caggtgcagctgcaggagtcgggcccaggactggtgaagccttcggagaccctgtccctcacctgcactgtctctggtggctccatcggaaattactactggggctggatccggcagcccccagggaagggacttgagtggattgggcatatctacttcggtggcaacaccaactacaacccttccctccagagtcgagtcaccatttcagtcgacacgtccaggaaccagttctccctgaagttgaactctgtgaccgccgcggacacggccgtgtattactgtgcgagggatagcagcaactggcccgcaggctatgaggactggggccagggaaccctggtcaccgtctcgagt

SEQ ID NO: 64 - DNA, 369 bp, homo sapiens
caggttcagctggtgcagtctggagctgaggtgaagaagcctggggcctcagtgaaggtctcctgcaaggtttctggttacacctttaccagtaatggtctcagctgggtgcgacaggcccctggacaagggtttgagtggctgggatggatcagcgctagtagtggaaacaaaaagtatgccccgaaattccagggaagagtcaccttgaccacagacatttccacgagcacagcctacatggaactgaggagtctgagatctgacgatacggccgtatattactgtgcgaaagatgggggcacctacgtgccctattctgatgcctttgatttctggggccaggggacaatggtcaccgtctcgagt

SEQ ID NO: 65 - DNA, 354 bp, homo sapiens
caggtccagctggtacagtctggggctgaggtgaagaagcctggggcctcagtgaaggtctcctgcagggtttccggacacactttcactgcattatccaaacactggatgcgacagggtcctggaggagggcttgagtggatgggattttttgatcctgaagatggtgacacaggctacgcacagaagttccagggcagagtcaccatgaccgaggacacagccacaggcacagcctacatggagctgagcagcctgacatctgacgacacggccgtatattattgtgcaacagtagcggcagctggaaactttgacaactggggccagggaaccctggtcaccgtctcgagt

SEQ ID NO: 66 - DNA, 378 bp, homo sapiens
caggtcaccttgaaggagtctggtcctgcgctggtgaaagccacacagaccctgacactgacctgcaccttctctgggttttcactcagtaggaatagaatgagtgtgagctggatccgtcagcccccagggaaggccctggagtggcttgcacgcattgattgggatgatgataaattctacaacacatctctgcagaccaggctcaccatctccaaggacacctccaaaaaccaggtggtccttacaatgaccaacatggaccctgtggacacagccacctattactgcgcacggactgggatatatgatagtagtggttattacctctactactttgactactggggccagggaaccctggtcaccgtctcgagt

SEQ ID NO: 67 - DNA, 390 bp, homo sapiens
caggtgcagctggtgcagtctggagctgaggtgaaggtgcctggggcctcagtgaaggtctcctgcaaggcttctggttacacctttaccacttacggtgtcagctgggtgcggcaggcccctggacaagggcttgagtggatgggttggatcagcgcttacaatggtaacacatactatctacagaagctccagggcagagtcaccatgaccacagacacatccacgagcacagcctacatggagctgcggggcctgaggtctgacgacacggccatgtattactgtgcgagagatcgtgttgggggcagctcgtccgaggttctatcgcgggccaaaaactacggtttggacgtctggggccaagggaccacggtcaccgtctcgagt

SEQ ID NO: 68 - DNA, 369 bp, homo sapiens
caggttcagctggtgcagtctggggctgaggtgaagaagcctggggcctcagttaaggtttcctgcaaggcttctgcaaacatcttcacttatgcaatgcattgggtgcgccaggcccccggacaaaggcttgagtggatgggatggatcaacgttggcaatggtcagacaaaatattcacagaggttccagggcagagtcaccattaccagggacacgtccgcgactacagcctacatggagctgagcaccctgagatctgaggacacggctgtgtattactgtgcgaggcgtgcgagccaatatggggaggtctatggcaactactttgactactggggccagggaaccctggtcaccgtctcgagt

SEQ ID NO: 69 - DNA, 378 bp, homo sapiens
caggtgcagctggtgcagtctggagctgaggtgaagaggcctggggcctcagtgaaggtctcctgcaaggcttcaggttacacctttatcagctatggtttcagctgggtgcgacaggcccctggacaagggcttgagtggatgggatggagcagcgtttacaatggtgacacaaactatgcacagaagttccacggcagagtcaacatgacgactgacacatcgacgaacacggcctacatggaactcaggggcctgagatctgacgacacggccgtgtatttctgtgcgagggatcgcaatgttgttctacttccagctgctccttttggaggtatggacgtctggggccaagggacaatggtcaccgtctcgagt

SEQ ID NO: 70 - DNA, 369 bp, homo sapiens
caggtgcagctggtggagtctgggggaggcgtggtccagccggggacttccctgagactctcctgtgcagcctctggattcaccttcagtacgtttggcatgcactgggtccgccaggctccaggcaaggggctggagtgggtggcagttatatcatatgatggaaataagaaatactatgcagactccgtgaagggccgattcaccatctccagagacaattccaagaacacgctgtatctgcaagtgaacagcctgagagtcgaggacacggctgtgtattactgtgcggcccaaactccatatttcaatgagagcagtgggttagtgccggactggggccagggcaccctggtcaccgtctcgagt

SEQ ID NO: 71 - DNA, 378 bp, homo sapiens
caggtgcagctggtgcagtctggagctgaggtgaagaagcctggggcctcagtgaaggtctcctgcaaggcttctggttacacctttatcagttttggcatcagctgggtgcgacaggcccctggacaaggacttgagtggatgggatggatcagcgcttacaatggtaacacagactatgcacagaggctccaggacagagtcaccatgactagagacacagccacgagcacagcctacttggagctgaggagcctgaaatctgacgacacggccgtgtactattgcactagagacgagtcgatgcttcggggagttactgaaggattcggacccattgactactggggccagggaaccctggtcaccgtctcgagt

SEQ ID NO: 72 - DNA, 384 bp, homo sapiens
gaagtgcagctggtgcagtctggagcagaggtgaaaaagccggggcagtctctgaagatctcctgtaagacttctggatacatctttaccaactactggatcggctgggtgcgccagaggcccgggaaaggcctggagtggatgggggtcatctttcctgctgactctgatgccagatacagcccgtcgttccaaggccaggtcaccatctcagccgacaagtccatcggtactgcctacctgcagtggagtagcctgaaggcctcggacaccgccatatattactgtgcgagaccgaaatattactttgatagtagtgggcaattctccgagatgtactactttgacttctggggccagggaaccctggtcaccgtctcgagt

SEQ ID NO: 73 - DNA, 357 bp, homo sapiens
caggttcagctggtgcagtctggacctgaggtgaagaagcctggggcctcagtgaaggtctcctgcaaggcttctggttatgtgttgaccaactatgccttcagctgggtgcggcaggcccctggacaagggcttgagtggctgggatggatcagcggctccaatggtaacacatactatgcagagaagttccagggccgagtcaccatgaccacagacacatccacgagcacagcctacatggagctgaggagtctgagatctgacgacacggccgtttatttctgtgcgagagatcttctgcggtccacttactttgactactggggccagggaaccctggtcaccgtctcgagt

SEQ ID NO: 74 - DNA, 378 bp, homo sapiens
caggtgcagctggtgcagtctggagctgaggtgaagaagcctggggcctcagtgaaggtctcctgcaaggcttctggttacaccttttccaactacggtttcagctgggtgcgacaggcccctggacgagggcttgagtggatgggatggatcagcgcttacaatggtaacacatactatgcacagaacctccagggcagagtcaccatgaccacagacacatccacgaccacagcctacatggtactgaggagcctgagatctgacgacacggccatgtattactgtgcgagagatggaaatacagcaggggttgatatgtggtcgcgtgatggttttgatatctggggccaggggacaatggtcaccgtctcgagt

SEQ ID NO: 75 - DNA, 393 bp, homo sapiens
gaggtgcagctgttggagtctgggggaggcttggtacagcctggggggcccctgaggctctcctgtgtagcctctggattcagctttagcagctatgccatgaactggatccgcctggctccagggaaggggctggagtgggtctcaggtattagtggtagcggtggtagcacttactacggagactccgtgaagggccggttcaccatctccagagacaattccaagaacacgctgtatctgcaaatgaacagcctgagagccgaggacacggccgtatattactgtgcgaaagagccgtggatcgatatagtagtggcatctgttatatccccctactactacgacggaatggacgtctggggccaagggaccacggtcaccgtctcgagt

SEQ ID NO: 76 - DNA, 369 bp, homo sapiens
caggttcagctggtgcagtctggggctgaggtgaagaagcctgggtcctcggtgaaggtctcctgcaaggcctctggaggatccttcgacggctacactatcagctggctgcgacaggcccctggacaggggcttgagtggatgggaagggtcgtccctacacttggttttccaaactacgcacagaagttccaaggcagagtcaccgttaccgcggacagatccaccaacacagcctacttggaattgagcagactgacatctgaagacacggccgtatattactgtgcgaggatgaatctcggatcgcatagcgggcgccccgggttcgacatgtggggccaaggaaccctggtcaccgtctcgagt

SEQ ID NO: 77 - DNA, 387 bp, homo sapiens
caggtgcagctggtggagtctgggggaggcgtggtccagcctgggaggtccttgagactctcctgtgcagtgtctggatccagcttcagtaaatatggcatacactgggtccgccaggctccaggcaaggggctggagtgggtggcagttatatcgtatgatggaagtaaaaagtatttcacagactccgtgaagggccgattcaccatcgccagagacaattcccagaacacggtttttctgcaaatgaacagcctgagagccgaggacacggctgtctattactgtgcgacaggagggggtgttaatgtcacctcgtggtccgacgtagagcactcgtcgtccttaggctactggggcctgggaaccctggtcaccgtctcgagt

SEQ ID NO: 78 - DNA, 375 bp, homo sapiens
caggtgcagctggtggagtctgggggaggcgtggtccagcctggggggtccctgagactctcctgtgcagcgtctggattcaccttcagtagctatggcatgcactgggtccgccaggctccaggcaaggggctggagtgggtggcatttatatggaatgatggaagtaataaatactatgcagactccgtgaagggccgattcaccatctccagagacaattccaagaacacgctgtatctgcaaatgaacagcctgagagctgaggacacggctgtgtattactgtgtgaaagatgaggtctatgatagtagtggttattacctgtactactttgactcttggggccagggaaccctggtcaccgtctcgagt

SEQ ID NO: 79 - DNA, 381 bp, homo sapiens
gaggtgcagctgttggagtctgggggaggcttggtacagcctggggggtccctgagactctcctgtgcagcctctggattcacgtttagctcctataccatgagctgggtccgccaggctccagggaaggggctggagtgggtctcaagtattagtgctagtactgttctcacatactacgcagactccgtgaagggccgcttcaccatctccagagacaattccaagaacacgctgtatctgcaaatgagtagcctgagagccgaggacacggccgtatattactgtgcgaaagattacgatttttggagtggctatcccgggggacagtactggttcttcgatctctggggccgtggcaccctggtcaccgtctcgagt

SEQ ID NO: 80 - DNA, 369 bp, homo sapiens
caggtgcagctgcaggagtcgggcccaggactggtgacgccttcggagaccctgtccgtcacttgcactgtctctaattattccatcgacaatgcttactactggggctggatccggcagcccccagggaagggtctggagtggataggcagtatccatcatagtgggagcgcctactacaattcgtccctcaagagtcgagccaccatatctatagacacgtccaagaaccaattctcgttgaacctgaggtctgtgaccgccgcagacacggccgtatattactgtgcgcgcgataccatcctcacgttcggggagccccactggttcgacccctggggccagggaaccctggtcaccgtctcgagt

SEQ ID NO: 81 - DNA, 372 bp, homo sapiens
caggtgcagctgcaggagtcgggcccaggactggtgaagccttcggagaccttgtccctcacctgcactgtctcaggtgactccatcagtaattactactggagttggatccggcagcccccagggaagggactggagtggattggagaaatatctaacacttggagcaccaattacaacccctccctcaagagtcgagtcaccatatctctagacatgcccaagaaccagttgtccctgaagctgagctctgtgaccgctgcggacacggccgtatattactgtgcgagagggcttttctatgacagtggtggttactacttgttttacttccaacactggggccagggcaccctggtcaccgtctcgagt

SEQ ID NO: 82 - DNA, 375 bp, homo sapiens
caggtgcagctggtggagtctgggggaggcgtggtccagcctgggaggtccctgagagtctcctgtgcagcgtctggattcaccttcagtaactatggcatgcactgggtccgccaggctccaggcaaggggctggagtgggtggcagttatatggtatgatgacagtaataaacagtatggagactccgtgaagggccgattcaccatctccagagacaattccaagagtacgctgtatctgcaaatggacagactgagagtcgaggacacggctgtgtattattgtgcgagagcctccgagtatagtatcagctggcgacacaggggggtccttgactactggggccagggaaccctggtcaccgtctcgagt

SEQ ID NO: 83 - DNA, 378 bp, homo sapiens
cagatcaccttgaaggagtctggtcctacgctggtgagacccacacagaccctcacactgacctgcaccttctctgggttctcactcagcactagtaaactgggtgtgggctggatccgtcagcccccaggaaaggccctggagtggcttgcactcgttgattgggatgatgataggcgctacaggccatctttgaagagcaggctcaccgtcaccaaggacacctccaaaaaccaggtggtccttacaatgaccaacatggaccctgtggacacagccacatattactgtgcacacagtgcctactatactagtagtggttattaccttcaatacttccatcactggggcccgggcaccctggtcaccgtctcgagt

SEQ ID NO: 84 - DNA, 375 bp, homo sapiens
gaggtgcagctggtggagtctgggggaggcgtggtacagcctggaggctccctgagactctcctgtgaagtctccggattcaccttcaatagttatgaaatgacctgggtccgccaggccccagggaaggggctggagtgggtttcacacattggtaatagtggttctatgatatactacgctgactctgtgaagggccgattcaccatctccagagacaacgccaagaactcactatatctgcaaatgaacagcctgagagtcgaggacacggctgtttattactgtgcgaggtcagattactatgatagtagtggttattatctcctctacttagactcctggggccatggaaccctggtcaccgtctcgagt

SEQ ID NO: 85 - DNA, 360 bp, homo sapiens
caggtgcagctggtgcagtctggggctgaggtgaggaagcctggggcctcagtgaaggtttcctgcaaggcttctggacatactttcattaactttgctatgcattgggtgcgccaggcccccggacaggggcttgagtggatgggatacatcaacgctgtcaatggtaacacacagtattcacagaagttccagggcagagtcacctttacgagggacacatccgcgaacacagcctacatggagctgagcagcctgagatctgaagacacggctgtgtattactgtgcgagaaacaatgggggctctgctatcattttttactactggggccagggaaccctggtcaccgtctcgagt

SEQ ID NO: 86 - DNA, 366 bp, homo sapiens
caggtgcagctggtggagtctgggggaggcgtggtccagcctgggaggtccctgagactctcctgtgcagcctctggattcagcttcagtagctatggcatgcactgggtccgccaggctccaggcaaggggctggagtgggtggcagttatatcaaatgatggaagtaataaatactatgcagactccgtgaagggccgattcaccatctccagagacaattccaagaaaacgatgtatctgcaaatgaacagcctgagagctgaggacacggctgtgtatttctgtgcgaagacaacagaccagcggctattagtggactggttcgacccctggggccagggaaccctggtcaccgtctcgagt

SEQ ID NO: 87 - DNA, 390 bp, homo sapiens
cagctgcagctgcaggagtcgggcccaggactggtgaagccatcggagaccctgtccctcacctgcactgcctctggtggctccatcaacagtagtaatttctactggggctggatccgccagcccccagggaaggggctggagtggattgggagtatcttttatagtgggaccacctactacaacccgtccctcaagagtcgagtcaccatatccgtagacacgtccaagaaccagttctccctgaagctgagccctgtgaccgccgcagacacggctgtctatcactgtgcgagacatggcttccggtattgtaataatggtgtatgctctataaatctcgatgcttttgatatctggggccaagggacaatggtcaccgtctcgagt

SEQ ID NO: 88 - DNA, 366 bp, homo sapiens
caggtgcagctggtggagtctgggggaggcgtcgtccagcctggaaagtccctgagactctcctgtgcagcgtctggattcagattcagtgactacggcatgcactgggtccggcaggctccaagcaaggggctggagtgggtggcagttatctggcatgacggaagtaatataaggtatgcagactccgtgaggggccgattttccatctccagagacaattccaagaacacgctgtatttgcaaatgaacagcatgagagccgacgacacggctttttattattgtgcgagagtcccgttccagatttggagtggtctttattttgaccactggggccagggaaccctggtcaccgtctcgagt

SEQ ID NO: 89 - protein, 216 aa, homo sapiens
EIVLTQSPATLSLSPGERATLSCRASQSVNSHLAWYQQKPGQAPRLLIYNTFNRVTGIPARFSGSGSGTDFTLTISSLATEDFGVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 215 aa, homo sapiens
DIQMTQSPSSLSASVGDRVTFTCRASQRISNHLNWYQQKPGKAPKLLIFGASTLQSGAPSRFSGSGSGTDFTLTITNVQPDDFATYYCQQSYRTPPINFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 217 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSLSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 213 aa, homo sapiens
DIQMTQSPSSLSASVGDRVTITCRASQSITGYLNWYQQKPGKAPKLLIYATSTLQSEVPSRFSGSGSGTDFTLTISSLQPEDFATYYCQQSYNTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 215 aa, homo sapiens
EIVLTQSPGTLSLSPGERATLSCRASQSVSSSYLAWYQQKPGQAPRLLIHGASTGATGTPDRFSGSGSGTDFTLTISTLEPEDFAVYYCQQYGRTPYTFGQGTKLENKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 219 aa, homo sapiens
DIVMTQTPLSLSVTPGQPASISCRSSQSLLRSDGKTFLYWYLQKPGQSPQPLMYEVSSRFSGVPDRFSGSGSGADFTLNISRVETEDVGIYYCMQGLKIRRTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 215 aa, homo sapiens
DIQMTQSPSTLSASVGDRVTFSCRASQSVSSWVAWYQQKPGKAPKLLISEASNLESGVPSRFSGSGSGTEFTLTISSLQPEDFATYYCQQYHSYSGYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 214 aa, homo sapiens
AIQLTQSPSSLSASVGDRVTLTCRASQGITDSLAWYQQKPGKAPKVLLYAASRLESGVPSRFSGRGSGTDFTLTISSLQPEDFATYYCQQYSKSPATFGPGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 219 aa, homo sapiens
DIVMTQSPLSLPVTPGEPASISCRSSQSLLNSNGFNYVDWYLQKPGQSPQLLIYLGSNRASGVPDRFSGSGSGTDFTLKISRVEAEDVGVYYCMQALETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 215 aa, homo sapiens
EIVLTQSPGTLSLSPGGRATLSCRASQSVSSGYLAWYQQKPGQAPRLLIYGASGRATGIPDRFSGSGSGTDFTLTISRLEPEDFAVYYCQQYFGSPYTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 214 aa, homo sapiens
NIQMTQSPSAMSASVGDRVTITCRASQGISNYLVWFQQKPGKVPKRLIYAASSLQSGVPSRFSGSGSGTEFTLTISSLQPEDFATYYCLQHNISPYTFGQGTKLETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 220 aa, homo sapiens
DIVMTQSPDSLAVSLGERATINCRSSETVLYTSKNQSYLAWYQQKARQPPKLLLYWASTRESGVPARFSGSGSGTDFTLAISSLQAEDVAVYYCQQFFRSPFTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 215 aa, homo sapiens
EIVLTQSPGTLSLSPGERVTLSCRASQSVSSSYIAWYQQKPGQAPRLVIYAASRRATGVPDRFSGSGSATDFTLTISRLEPEDLAVYYCQHYGNSLFTFGPGTKVDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 215 aa, homo sapiens
DIQMTQSPSTLSASVGDRVTITCRASQSIGSRLAWYQQQPGKAPKFLIYDASSLESGVPSRFSGSGSGTEFTLTISSLQPEDLATYYCQQYNRDSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 219 aa, homo sapiens
DIVMTQSPLSLPVTPGEPASISCRSSQSLLHSDGRYYVDWYLQKPGQSPHLLIYLASNRASGVPDRFTGSGSGTDFTLKISRVEAEDVGVYYCMQGLHTPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 213 aa, homo sapiens
EIVMTQSPATLSASPGERATLSCWASQTIGGNLAWYQQKPGQAPRLLIYGASTRATGVPARFSGSGSGTEFTLAISSLQSEDFAVYYCQQYKNWYTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 215 aa, homo sapiens
DIQMTQSPSSLSASVGDRVTITCRASQTIASYVNWYQQKPGRAPSLLIYAASNLQSGVPPRFSGSGSGTDFTLTISGLQPDDFATYYCQQSYSYRALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 216 aa, homo sapiens
EIVLTQSPATLSLSPGERATLSCRASQSVSSSLAWYQQTPGQAPRLLIYDASYRVTGIPARFSGSGSGIDFTLTISSLEPEDFAVYYCQQRSNWPPGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 214 aa, homo sapiens
AIQLTQSPSSLSASVGDTVTVTCRPSQDISSALAWYQQKPGKPPKLLIYGASTLDYGVPLRFSGTASGTHFTLTISSLQPEDFATYYCQQFNTYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 220 aa, homo sapiens
DIVMTQSPDSLAVSLGERATINCKSSQSVLYNSNNKNYLAWYQQKPGQPPKLLIHLASTREYGVPDRFSGSGSGTDFALIISSLQAEDVAVYYCQQYYQTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 214 aa, homo sapiens
DIQMTQSPSSLAASVGDRVTITCRASQFISSYLHWYQQRPGKAPKLLMYAASTLQSGVPSRFSGSGSGTDFTLTISSLQPEDFATYYCQQSYTNPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 214 aa, homo sapiens
DIQMTQSPSSLSASVGDRVTITCRASQSIASYLNWYQQKPGKAPKLLIYAASSLHSGVPSRFSGSGSGTDFTLTISSLQPEDFATYYCQHSYSTRFTFGPGTKVDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 214 aa, homo sapiens
DIQMTQSPSTLSASVGDRVTITCRASQSVTSELAWYQQKPGKAPNFLIYKASSLESGVPSRFSGSGSGTEFTLTISSLQPDDFATYYCQQYNSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 214 aa, homo sapiens
DIQMTQSPSTLSASVGDRLTITCRASQNIYNWLAWYQQKPGKAPKLLIYDASTLESGVPSRFSGSGSGTEFTLTISSLQPDDFATYYCQQYNSLSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 214 aa, homo sapiens
DIQLTQSPSFLSASLEDRVTITCRASQGISSYLAWYQQKPGKAPKLLLDAASTLQSGVPSRFSGSGSGTEFTLTISSLQPEDFATYYCQQLNSYPRTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 214 aa, homo sapiens
DIQMTQSPSSLSASVGDRVSITCRASQGISNYLAWYQQKPGKVPKLLIYAASTLQSGVPSRFSGSGSGTDFTLTISSLQPEDVATYYCQKYNSAPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 220 aa, homo sapiens
DIVMTQSPDSLAVSLGERATINCRSSQSVLYSSNNKNYLAWYQQKPGQPPKLLVYWASTRASGVPDRFSGSGSGTDFTLTLSSLQAEDVAVYYCQQFHSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, homo sapiens
EIVLTQSPGTLSLSPGERATLSCRASQSVSSNYLAWYQQKPGQAPRLLIYGASSRAAGMPDRFSGSGSGTDFTLTISRLEPEDFAVYYCQQYGNSPLTFGGGTEVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 214 aa, homo sapiens
DIQMTQSPSSVSASVGDRVTITCRASQAISNWLAWYQQKPGKAPKLLIYAASSLQSGVPSRFSGSGSGTDFTLTISGLQPEDFATYYCQQADTFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 220 aa, homo sapiens
DIVMTQTPLSLPVTPGEPASISCRSSQSLLDSNDGNTYLDWYLQKPGQSPQLLIYTFSYRASGVPDRFSGSGSGTDFTLKISRVEAEDVGVYYCMQRIEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 219 aa, homo sapiens
DIVMTQSPLSLPVTPGEPASISCRSSQSLLHRNEYNYLDWYLQKPGQSPQLLIYWGSNRASGVPDRFSGSGSGTDFTLKISRVEAEDVGVYYCMQTLQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 214 aa, homo sapiens
DIQMTQSPSSVSASVGDRVTITCQASQDISNYLNWYQQKPGKAPKLLIFDATKLETGVPTRFIGSGSGTDFTVTITSLQPEDVATYYCQHFANLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 214 aa, homo sapiens
DIQMTQSPSSLSASVGDRVTITCRASQGIRNYLAWYQQKPGKVPKLLVFAASTLQSGVPSRFSGSGSGTDFTLTISSLQPEDVATYYCQRYNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 215 aa, homo sapiens
DIQMTQSPSSLSASVGDRVTITCRASQIIASYLNWYQQKPGRAPKLLIYAASSLQSGVPSRFSGSGSGTDFTLTISSLQPEDFATYYCQQSYSTPIFTFGPGTKVNIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 213 aa, homo sapiens
EIVLTQSPATLSLSPGERATLSCRTSQSVSSYLAWYQQKPGQAPRLLIYDASNRATGIPARFSGSGSGTDFTLTISSLEPEDFAVYYCQQRSDWLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 215 aa, homo sapiens
EIVMTQSPATLSVSPGERATLSCRASQSIKNNLAWYQVKPGQAPRLLTSGASARATGIPGRFSGSGSGTDFTLTISSLQSEDIAVYYCQEYNNWPLLTFGGGTKVEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 215 aa, homo sapiens
DIQMTQSPPSLSASVGDRVTITCRASQRIASYLNWYQQKPGRAPKLLIFAASSLQSGVPSRFSGSGSGTDFTLTISSLQPEDYATYYCQQSYSTPIYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 214 aa, homo sapiens
DIQMTQSPSSLSASVGDRVTITCQASQGISNYLNWYQQKPGKAPKLLIFDASNLESEVPSRFSGRGSGTDFTFSISSLQPEDIATYFCQQYDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 214 aa, homo sapiens
DIQMTQSPSSLAASVGDRVTITCRASQTIASYVNWYQQKPGKAPNLLIYAASSLQSGVPSRFSGSGSGTDFTLTISSLQPEDFASYFCQQSYSFPYTFGQGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 215 aa, homo sapiens
DIQMTQSPSSLSASVGDRVTITCRASQTIASYVNWYQQKPGKAPKLLIYAASNLQSGVPSRFSGSGSGTDFTLTISSLQPEDFATYYCQQSYSVPRLTFGGGTKVDITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 213 aa, homo sapiens
DIQMTQSPSSLSASVGDRVTITCRSSQTISVFLNWYQQKPGKAPKLLIYAASSLHSAVPSRFSGSGSGTDFTLTISSLQPEDSATYYCQESFSSSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 215 aa, homo sapiens
EIVMTQSPATLSVSPGETATLSCRASQSVSSNLAWYQHKPGQAPRLLIHSASTRATGIPARFSGSGSGTEFTLTISSLQSEDFAVYYCQQYNMWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 219 aa, homo sapiens
DIVMTQSPLSLPVTPGAPASISCRSSQSLLRTNGYNYLDWYLQKPGQSPQLLIYLGSIRASGVPDRFSGSGSGTDFTLKISRVEAEDVGVYYCMQSLQTSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 214 aa, homo sapiens
EIVMTQSPATLSVSPGERATLSCRASQSVGNNLAWYQQRPGQAPRLLIYGASTRATGIPARFSGSGSGTEFTLTISSLQSEDFAVYYCQQYDKWPETFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 648 bp, homo sapiens
gaaattgtgttgacacagtctccagccaccctgtccttgtctccaggagaaagagccaccctctcctgcagggccagtcagagtgttaacagccacttagcctggtaccaacagaaacctggccaggctcccaggctcctcatctataatacattcaatagggtcactggcatcccagccaggttcagtggcagtgggtctgggacagacttcactctcaccatcagcagccttgcgactgaagattttggcgtttattactgtcagcagcgtagcaactggcctcccgccctcactttcggcggagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 134 - DNA, 645 bp, homo sapiens
gacatccagatgacccagtctccatcctccctgtctgcatctgtgggagacagagtcaccttcacttgccgggccagtcagaggattagcaaccatttaaattggtatcaacaaaagccagggaaagcccctaaactcctgatctttggtgcatccactcttcaaagtggggccccatcaaggttcagtggcagtggatctgggacagatttcactctcaccatcactaatgtacaacctgacgattttgcaacttactactgtcaacagagttacagaactcccccgatcaacttcggccaagggacacgcctggacattaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 135 - DNA, 651 bp, homo sapiens
gaaattgtgttgacgcagtctccaggcaccctgtctttgtctccaggggaaagagccaccctctcctgcagggccagtcagagtgttagcagcagctacttagcctggtatcagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatgatagctcactttctacgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 136 - DNA, 639 bp, homo sapiens
gacatccagatgacccagtctccatcctccctgtctgcatctgtaggagacagagtcaccatcacttgccgggcaagtcagagcattaccggctatttaaattggtatcagcagaaaccagggaaagcccctaaactcctgatctatgctacatccactttgcaaagtgaggtcccatcaaggttcagtggcagtggatctgggacagatttcactctcaccatcagcagtcttcaacctgaagattttgcaacttactactgtcaacagagttataataccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 137 - DNA, 645 bp, homo sapiens
gaaattgtgttgacgcagtctccaggcaccctgtctttgtctccaggggaaagagccaccctctcctgcagggccagtcagagtgttagcagcagctacttagcctggtatcagcagaaacctggccaggctcccaggctcctcatacatggcgcatccaccggggccactggcaccccagacaggttcagtggcagtgggtctgggacagacttcactctcaccatcagtacactggagcctgaagattttgcagtgtattactgtcagcaatatggtaggacaccgtacacttttggccaggggaccaagctggagaacaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 138 - DNA, 657 bp, homo sapiens
gatattgtgatgacccagactccactctctctgtccgtcacccctggacagccggcctccatctcctgcaggtctagtcagagcctcctgcgaagtgatggaaagacgtttttgtattggtatctgcagaagccaggccagtctccccaacccctaatgtatgaggtgtccagccggttctctggagtgccagataggttcagtggcagcgggtcaggggcagatttcacactgaacatcagccgggtggagactgaggatgttgggatctattactgcatgcaaggtttgaaaattcgtcggacgtttggcccagggaccaaggtcgaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 139 - DNA, 645 bp, homo sapiens
gacatccagatgacccagtctccttccaccctgtctgcatctgtaggagacagagtcaccttctcttgccgggccagtcagagtgttagtagttgggtggcctggtatcagcagaaaccaggaaaagcccctaagctcctgatctctgaggcctccaatttggaaagtggggtcccatcccggttcagcggcagtggatccgggacagaattcactctcaccatcagcagcctgcagcctgaagattttgcaacttattactgccaacagtatcatagttactctgggtacacttttggccaggggaccaagttggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 140 - DNA, 642 bp, homo sapiens
gccatccagttgacccagtctccatcgtccctgtctgcatctgtaggcgacagagtcaccctcacttgccgggcgagtcagggcattaccgattctttagcctggtatcagcagaaaccagggaaagcccctaaggtcctgctctatgctgcttccagattggaaagtggggtcccatccaggttcagtggccgtggatctgggacggatttcactctcaccatcagcagcctgcagcctgaagactttgcaacttattactgtcaacagtattctaagtcccctgcgacgttcggcccagggaccaaggtggaaatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 141 - DNA, 657 bp, homo sapiens
gatattgtgatgacccagtctccactctccctgcccgtcacccctggagagccggcctccatctcctgcaggtctagtcagagcctcctaaatagtaatggattcaactatgtggattggtacctgcagaagccagggcagtctccacaactcctgatctatttgggttctaatcgggcctccggggtccctgacaggttcagtggcagtggatcaggcacagattttacactgaaaatcagcagagtggaggctgaggatgttggggtttattactgcatgcaagctctagaaactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 142 - DNA, 645 bp, homo sapiens
gaaattgtgttgacgcagtctccaggcaccctgtctttgtctccagggggaagagccaccctctcctgcagggccagtcagagtgttagcagcggctacttagcctggtaccagcagaaacctggccaggctcccaggctcctcatctatggtgcatccggcagggccactggcatcccagacaggttcagtggcagtgggtctgggacagacttcactctcaccatcagcagactggagcctgaagattttgcagtgtattactgtcagcagtattttggctcaccgtacacttttggccaggggaccaagctggagctcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 143 - DNA, 642 bp, homo sapiens
aacatccagatgacccagtctccatctgccatgtctgcatctgtaggagacagagtcaccatcacttgtcgggcgagtcagggcattagtaattatttagtctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatatttccccttacacttttggccaggggaccaagctggagaccaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 144 - DNA, 660 bp, homo sapiens
gacatcgtgatgacccagtctccagactccctggctgtgtctctgggcgagagggccaccatcaactgcaggtccagtgagactgttttatacacctctaaaaatcagagctacttagcttggtaccagcagaaagcacgacagcctcctaaactactcctttactgggcatctacccgggaatccggggtccctgcccgattcagtggcagcggatctgggacagatttcactctcgccatcagcagcctgcaggctgaagatgtggcagtttattactgtcagcaattttttaggagtcctttcactttcggccccgggaccagactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 145 - DNA, 645 bp, homo sapiens
gaaattgtgttgacgcagtctccaggcaccctgtctttgtctccaggggaaagagttaccctctcttgcagggccagtcagagtgttagcagcagttacatagcctggtaccagcagaagcctggccaggctcccaggctcgtcatctatgctgcatcccgcagggccactggcgtcccagacaggttcagtggcagtgggtctgcgacagacttcactctcaccatcagtagactggagcctgaagatcttgcagtgtattactgtcagcactatggtaactcactattcactttcggccctgggaccaaggtggatgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 146 - DNA, 645 bp, homo sapiens
gacatccagatgacccagtctccctccaccctgtctgcatctgtcggagacagagtcaccatcacttgccgggccagtcagagtattggtagccggttggcctggtatcagcagcaaccagggaaagcccctaaattcctgatctatgatgcctccagtttggaaagtggggtcccatcaaggttcagcggcagtggatcagggacagaattcactctcaccatcagcagcctgcagccggaggatcttgcaacttattactgccaacagtacaatagagattctccgtggacgttcggccaagggaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 147 - DNA, 657 bp, homo sapiens
gatattgtgatgacccagtctccactctccctgcccgtcaccccaggagagccggcctccatctcctgcaggtctagtcagagcctcctgcatagtgatggacgctactatgtggattggtacctgcagaagccagggcagtctccacacctcctgatctatttggcttctaatcgggcctccggggtccctgacaggttcactggcagtggatcaggcacagattttacactgaaaatcagcagagtggaggctgaggatgttggcgtttattactgcatgcaaggtctacacactccttggacgttcggccaggggaccaaggtggacatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 148 - DNA, 639 bp, homo sapiens
gaaattgtaatgacacagtctccagccaccctgtctgcgtccccaggggaaagagccaccctctcctgttgggccagtcagactattggaggcaacttagcctggtaccagcagaaacctggccaggctcccaggctcctcatctatggtgcatccaccagggccactggtgtcccagccaggttcagtggcagtgggtctgggacagagttcactctcgccatcagcagcctgcagtctgaagattttgcagtttattactgtcagcagtataaaaactggtacacttttggccaggggaccaagctggagctcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 149 - DNA, 645 bp, homo sapiens
gacatccagatgacccagtctccatcctccctgtctgcatctgtaggagacagagtcaccatcacttgccgggcaagtcagaccattgccagttacgtaaattggtaccaacaaaaaccagggagagcccctagtctcctgatctatgctgcatctaacttgcagagtggggtcccaccaaggttcagtggcagtggatctgggacagacttcactctcaccatcagcggtctgcaacctgacgattttgcaacttattactgtcaacagagttacagttatcgagcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 150 - DNA, 648 bp, homo sapiens
gaaattgtgttgacacagtctccagccaccctgtcgttgtccccaggggaaagagccaccctctcctgcagggccagtcagagtgttagcagctccttagcctggtaccaacagacacctggccaggctcccaggcttctcatctatgatgcgtcctacagggtcactggcatcccagccaggttcagtggcagtgggtctgggatagacttcactctcaccatcagcagcctagagcctgaagattttgcagtttactattgtcagcagcgtagcaactggcctccggggctcactttcggcggggggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 151 - DNA, 642 bp, homo sapiens
gccatccagttgacccagtctccatcctccctgtctgcatctgttggagacacagtcaccgtcacttgccggccaagtcaggacattagcagtgctttagcctggtatcagcagaaaccagggaaacctcctaagctcctgatctatggtgcctccactttggattatggggtcccattaaggttcagcggcactgcatctgggacacatttcactctcaccatcagcagcctgcaacctgaagattttgcaacttattactgtcaacagtttaatacttacccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 152 - DNA, 660 bp, homo sapiens
gacatcgtgatgacccagtctccagactccctggctgtgtctctgggcgagagggccaccatcaactgcaagtccagccagagtgttttatacaactccaacaataagaactacttagcctggtatcagcagaaaccaggacagcctcctaagctcctcattcacttggcatctacccgggaatacggggtccctgaccgattcagtggcagcgggtctgggacagatttcgctctcatcatcagcagcctgcaggctgaagatgtggcagtttattactgtcaacaatattatcaaactcctctaacttttggccaggggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 153 - DNA, 642 bp, homo sapiens
gacatccagatgacccagtctccatcctccctggctgcatctgtaggagacagagtcaccatcacttgccgggcaagtcagttcattagcagctatttacattggtatcagcaaagaccaggcaaggcccctaaactcctgatgtatgctgcctccactttgcaaagtggggtcccatcaaggttcagtggcagtggatctgggacagatttcactctcaccatcagcagtctgcaacctgaagattttgcaacttactactgtcaacagagttacactaacccatacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 154 - DNA, 642 bp, homo sapiens
gacatccagatgacccagtctccatcctccctatctgcatctgtaggagacagagtcaccatcacttgccgggcaagtcagagcattgccagctatttaaattggtatcagcagaaaccagggaaagcccccaaactcctgatctatgctgcatccagtttgcatagtggggtcccatcaagattcagtggcagtggatctgggacagatttcactctcaccatcagcagtctgcaacctgaagattttgcaacttactactgtcaacacagttacagtactcgattcactttcggccctgggaccaaagtggatgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 155 - DNA, 642 bp, homo sapiens
gacatccagatgacccagtctccttcgaccctgtctgcatctgtaggagacagagtcaccatcacttgccgggccagtcagagtgttactagtgagttggcctggtatcagcagaaaccagggaaagcccctaacttcctgatctataaggcgtctagtttagaaagtggggtcccatcaaggttcagcggcagtggatctgggacagaattcactctcaccatcagcagcctgcagcctgatgattttgcaacttattactgccaacagtataatagttttccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 156 - DNA, 642 bp, homo sapiens
gacatccagatgacccagtctccttccaccctgtctgcatctgtaggcgacagactcaccatcacttgccgggccagtcagaatatttataactggttggcctggtatcagcagaaaccagggaaagcccctaaactcctgatctatgacgcctccactttggaaagtggggtcccatcaaggttcagcggcagtggatctgggacagagttcactctcaccatcagcagcctgcagcctgatgattttgcgacttattactgccaacaatataatagtttgtctccgacgttcggccaagggaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 157 - DNA, 642 bp, homo sapiens
gacatccagttgacccagtctccatccttcctgtctgcatctttagaagacagagtcactatcacttgccgggccagtcagggcattagcagttatttagcctggtatcagcaaaaaccagggaaagcccctaagctcctgctcgatgctgcatccactttgcaaagtggggtcccatcaaggttcagcggcagtggatctgggacagagttcactctcacaatcagcagcctgcagcctgaagattttgcaacttattactgtcaacagcttaatagttaccctcggacgttcggccaagggaccaaggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 158 - DNA, 642 bp, homo sapiens
gacatccagatgacccagtctccatcctccctgtctgcatctgtaggagacagagtcagcatcacttgccgggcgagtcagggcattagcaattatttagcctggtatcagcagaaaccagggaaggttcctaagctcctgatctatgctgcatccactttgcaatcaggggtcccatctcggttcagtggcagtggatctgggacagatttcactctcaccatcagcagcctgcagcctgaggatgttgcaacttattactgtcaaaagtataacagtgcccctcaaacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 159 - DNA, 660 bp, homo sapiens
gacatcgtgatgacccagtctccagactccctggctgtgtctctgggcgagagggccaccatcaactgcaggtccagccagagtgttttatacagctccaacaataagaactacttagcttggtaccagcagaaaccaggacagcctcctaagctgctcgtttactgggcatcaacccgggcatccggggtccctgaccgattcagtggcagcgggtctgggacagatttcactctcaccctcagcagcctgcaggctgaagatgtggcagtttattactgtcagcagtttcatagtactcctcggacgttcggccaagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 160 - DNA, 645 bp, homo sapiens
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccagcagggccgctggcatgccagacaggttcagtggcagtgggtctgggacagacttcactctcaccatcagcagactggagcctgaagattttgcagtgtattactgtcagcagtatggtaactcaccgctcactttcggcggagggaccgaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 161 - DNA, 642 bp, homo sapiens
gacatccagatgacccagtctccatcttctgtgtctgcatctgtaggagacagagtcaccatcacttgtcgggcgagtcaggctattagtaactggttagcctggtatcagcagaaaccaggaaaagcccctaagctcctgatctatgctgcatccagtttgcaaagtggggtcccatcaagattcagcggcagtggatctgggacagatttcactctcactatcagcggcctgcagcctgaggattttgcaacttactattgtcaacaggctgacactttccctttcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 162 - DNA, 660 bp, homo sapiens
gatattgtgatgacccagactccactctccctgcccgtcacccctggagagccggcctccatctcctgcaggtctagtcagagcctcttggatagtaatgatggaaacacctatttggactggtacctgcagaagccagggcagtctccacagctcctgatttatacattttcctatcgggcctctggagtcccagacaggttcagtggcagtgggtctggcactgatttcacactgaaaatcagcagggtggaggccgaggatgttggagtttattactgcatgcaacgtatcgagtttccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 163 - DNA, 657 bp, homo sapiens
gatattgtgatgacccagtctccactctccctgcccgtcacccctggagagccggcctccatctcctgcaggtctagtcagagcctcctgcatagaaatgagtacaactatttggattggtacttgcagaagccagggcagtctccacagctcctgatctattggggttctaatcgggcctccggggtccctgacaggttcagtggcagtggatcaggcacagattttacactgaaaatcagcagagtggaggctgaggatgttggggtttattactgcatgcaaactctacaaactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 164 - DNA, 642 bp, homo sapiens
gacatccagatgacccagtctccatcctccgtgtctgcatctgtgggagacagagtcaccatcacttgccaggcgagtcaagacattagcaactatttaaattggtatcagcagaaaccagggaaagcccctaagctcctgatcttcgatgcaaccaaattggagacaggggtcccaacaaggttcattggaagtggatctgggacagattttactgtcaccatcaccagcctgcagcctgaagatgttgcaacatattactgtcaacactttgctaatctcccatacacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 165 - DNA, 642 bp, homo sapiens
gacatccagatgacccagtctccatcttccctgtctgcatctgtaggagacagagtcaccatcacttgccgggcgagtcagggcattaggaattatttagcctggtatcagcagaaaccagggaaagttcctaagctcctggtctttgctgcatccactttgcaatcaggggtcccatctcggttcagtggcagtggatctgggacagatttcactctcaccatcagcagcctgcagcctgaggatgttgcaacttattactgtcaaaggtataacagtgccccgctcactttcggcggagggacgaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 166 - DNA, 645 bp, homo sapiens
gacatccagatgacccagtctccatcctccctgtctgcatctgtaggagacagagtcaccatcacttgccgggcaagtcagatcattgccagctatttaaattggtatcagcagaaaccaggcagagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccatattcactttcggccctgggaccaaggtgaatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 167 - DNA, 639 bp, homo sapiens
gaaattgtgttgacacagtctccagccaccctgtctttgtctccaggggaaagagccaccctctcctgcaggaccagtcagagtgttagcagctacttagcctggtaccaacagaaacctggccaggctcccaggctcctcatctatgatgcttccaatagggccactggcatcccagccaggttcagtggcagtgggtctgggacagacttcactctcaccatcagcagcctagagcctgaagattttgcagtttattactgtcagcagcgtagtgactggctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 168 - DNA, 645 bp, homo sapiens
gaaattgtaatgacacagtctccagccaccctgtctgtgtctccaggggaaagagccaccctctcctgcagggccagtcagagtattaaaaacaacttggcctggtaccaggtgaaacctggccaggctcccaggctcctcacctctggtgcatccgccagggccactggaattccaggcaggttcagtggcagtgggtctgggactgacttcactctcaccatcagcagcctccagtctgaagatattgcagtttattactgtcaggagtataataattggcccctgctcactttcggcggagggaccaaggtggagatccaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 169 - DNA, 645 bp, homo sapiens
gacatccagatgacccagtctcctccctccctgtctgcatctgtgggagacagagtcaccatcacttgccgggcaagtcagaggattgccagctatttaaattggtatcagcagaaaccagggagagcccctaagctcctgatctttgctgcatccagtttacaaagtggggtcccatcaaggttcagtggcagtggatctgggacagacttcactctcaccatcagtagtctgcaacctgaagattatgcgacttactactgtcaacagagttacagtactcccatctacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 170 - DNA, 642 bp, homo sapiens
gacatccagatgacccagtctccatcctccctgtctgcatctgtaggagacagagtcaccatcacttgccaggcgagtcagggcattagcaactatttaaattggtatcaacagaaaccagggaaagcccctaagctcctgatcttcgatgcatccaatttggaatcagaggtcccatcaaggttcagtggacgtggatctgggacagattttactttctccatcagcagcctgcagcctgaagatattgcaacatatttctgtcaacagtatgataatttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 171 - DNA, 642 bp, homo sapiens
gacatccagatgacccagtctccatcctccctggctgcatctgtaggagacagagtcaccatcacctgccgggcaagtcagacgattgccagttatgtaaattggtatcaacagaaaccagggaaagcccctaatctcctgatctatgctgcatccagtttgcaaagtggggtcccatcaaggttcagtggcagtggatctgggacagatttcactctcaccatcagcagtctgcaacctgaagattttgcatcttacttctgtcaacagagttacagtttcccgtacacttttggccaggggaccaagctggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 172 - DNA, 645 bp, homo sapiens
gacatccagatgacccagtctccatcctccctgtctgcatctgtaggagacagagtcaccatcacttgccgggcaagtcagaccattgccagctatgtaaattggtatcagcagaaaccagggaaagcccctaagctcctgatctatgctgcatccaatttgcaaagtggggtcccttcaaggttcagtggcagtggatctgggacagatttcactctcaccatcagcagtctgcaacctgaagattttgcaacttactactgtcaacagagttacagtgtccctcggctcactttcggcggagggaccaaggtggacatcacacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 173 - DNA, 639 bp, homo sapiens
gacatccagatgacccagtctccatcctccctgtctgcatctgtaggagacagagtcaccatcacttgccggtcaagtcagaccattagcgtctttttaaattggtatcagcagaaaccagggaaagcccctaagctcctgatctatgccgcatccagtttgcacagtgcggtcccatcaaggttcagtggcagtggatctgggacagatttcactctcaccatcagcagtctgcaacctgaagattctgcaacttactactgtcaagagagtttcagtagctcaactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 174 - DNA, 645 bp, homo sapiens
gaaattgtaatgacacagtctccagccaccctgtctgtgtctccaggggaaacagccaccctctcctgcagggccagtcagagtgttagcagcaacttagcctggtaccaacataaacctggccaggctcccaggctcctcatccatagtgcatccaccagggccactgggatcccagccaggttcagtggcagtgggtctgggacagagttcactctcaccataagcagcctgcagtctgaagattttgcagtttattactgtcagcagtataatatgtggcctccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 175 - DNA, 657 bp, homo sapiens
gatattgtgatgacccagtctccactctccctgcccgtcacccctggagcgccggcctccatctcctgcaggtctagtcagagcctcctgcgtactaatggatacaactatttggattggtacctgcagaagccagggcagtctccacagctcctgatctatttgggttctattcgggcctccggggtccctgacaggttcagtggcagtggctcaggcacagattttacactgaaaatcagcagagtggaggctgaggatgttggggtttattactgcatgcaatctctacaaacttcgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 176 - DNA, 642 bp, homo sapiens
gaaattgtaatgacacagtctccagccaccctgtctgtgtctccgggggaaagagccaccctctcctgcagggctagtcagagtgttggcaacaacttagcctggtaccagcagagacctggccaggctcccagactcctcatctatggtgcgtccaccagggccactggtatcccagccaggttcagtggcagtgggtctgggacagagttcactctcaccatcagcagcctgcagtctgaggattttgcagtttattactgtcagcagtatgataagtggcctgagacgttcggccaggggaccaaggtggacatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 177 - DNA, 1602 bp, Homo sapiens
  CDS (1)..(298)
  CDS (690)..(734)
  CDS (853)..(1182)
  CDS (1280)..(1599)
agtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 178 - protein, 331 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - DNA, 20 bp, artificial sequence
  Synthetic DNA primer
gacngatgggcccttggtgg

SEQ ID NO: 180 - DNA, 20 bp, artificial sequence
  Synthetic DNA primer
gagtggctcctgggggaaga

SEQ ID NO: 181 - DNA, 36 bp, artificial sequence
  Synthetic DNA primer
tattcccatggcgcgcccagntgcagctggtgcant

SEQ ID NO: 182 - DNA, 36 bp, artificial sequence
  Synthetic DNA primer
tattcccatggcgcgccnaggtccagctggtncagt

SEQ ID NO: 183 - DNA, 36 bp, artificial sequence
  Synthetic DNA primer
tattcccatggcgcgcccagntcaccttgaaggagt

SEQ ID NO: 184 - DNA, 35 bp, artificial sequence
  Synthetic DNA primer
tattcccatggcgcgccnaggtgcagctggtggag

SEQ ID NO: 185 - DNA, 36 bp, artificial sequence
  Synthetic DNA primer
tattcccatggcgcgcccaggtgcagctacagcagt

SEQ ID NO: 186 - DNA, 36 bp, artificial sequence
  Synthetic DNA primer
tattcccatggcgcgcccagntgcagctgcaggagt

SEQ ID NO: 187 - DNA, 36 bp, artificial sequence
  Synthetic DNA primer
tattcccatggcgcgccgangtgcagctggtgcagt

SEQ ID NO: 188 - DNA, 37 bp, artificial sequence
  Synthetic DNA primer
tattcccatggcgcgcccaggtacagctgcagcagtc

SEQ ID NO: 189 - DNA, 38 bp, artificial sequence
  Synthetic DNA primer
atatatatgcggccgcttattaacactctcccctgttg

SEQ ID NO: 190 - DNA, 45 bp, artificial sequence
  Synthetic DNA primer
ggcgcgccatgggaatagctagccgacatccagntgacccagtct

SEQ ID NO: 191 - DNA, 45 bp, artificial sequence
  Synthetic DNA primer
ggcgcgccatgggaatagctagccgatgttgtgatgactcagtct

SEQ ID NO: 192 - DNA, 45 bp, artificial sequence
  Synthetic DNA primer
ggcgcgccatgggaatagctagccgaaattgtgntgacncagtct

SEQ ID NO: 193 - DNA, 45 bp, artificial sequence
  Synthetic DNA primer
ggcgcgccatgggaatagctagccgatattgtgatgacccacact

SEQ ID NO: 194 - DNA, 43 bp, artificial sequence
  Synthetic DNA primer
ggcgcgccatgggaatagctagccgaaacgacactcacgcagt

SEQ ID NO: 195 - DNA, 45 bp, artificial sequence
  Synthetic DNA primer
ggcgcgccatgggaatagctagccgaaattgtgctgactcagtct

SEQ ID NO: 196 - DNA, 51 bp, artificial sequence
  Synthetic DNA primer
accgcctccaccggcggccgcttattaacactctcccctgttgaagctctt

SEQ ID NO: 197 - DNA, 30 bp, artificial sequence
  Synthetic DNA primer
ggaggcgctcgagacggtgaccagggtgcc

SEQ ID NO: 198 - DNA, 30 bp, artificial sequence
  Synthetic DNA primer
ggaggcgctcgagacggtgaccattgtccc

SEQ ID NO: 199 - DNA, 30 bp, artificial sequence
  Synthetic DNA primer
ggaggcgctcgagacggtgaccagggttcc

SEQ ID NO: 200 - DNA, 30 bp, artificial sequence
  Synthetic DNA primer
ggaggcgctcgagacggtgaccgtggtccc

SEQ ID NO: 201 - protein, 5 aa, Homo sapiens
DYDWS

SEQ ID NO: 202 - protein, 5 aa, Homo sapiens
TYGMH

SEQ ID NO: 203 - protein, 5 aa, Homo sapiens
TYALT

SEQ ID NO: 204 - protein, 5 aa, Homo sapiens
GYYMH

SEQ ID NO: 205 - protein, 5 aa, Homo sapiens
DYYMS

SEQ ID NO: 206 - protein, 5 aa, Homo sapiens
NYGLN

SEQ ID NO: 207 - protein, 7 aa, Homo sapiens
SGDYYWS

SEQ ID NO: 208 - protein, 5 aa, Homo sapiens
HFGMH

SEQ ID NO: 209 - protein, 5 aa, Homo sapiens
RFGIS

SEQ ID NO: 210 - protein, 5 aa, Homo sapiens
SYVMN

SEQ ID NO: 211 - protein, 5 aa, Homo sapiens
NYGMH

SEQ ID NO: 212 - protein, 5 aa, Homo sapiens
DYGMN

SEQ ID NO: 213 - protein, 5 aa, Homo sapiens
SYAMH

SEQ ID NO: 214 - protein, 5 aa, Homo sapiens
SYEMN

SEQ ID NO: 215 - protein, 7 aa, Homo sapiens
SGDYFWS

SEQ ID NO: 216 - protein, 5 aa, Homo sapiens
NYAMH

SEQ ID NO: 217 - protein, 5 aa, Homo sapiens
GDFWS

SEQ ID NO: 218 - protein, 5 aa, Homo sapiens
SYWIG

SEQ ID NO: 219 - protein, 7 aa, Homo sapiens
TTRMSVS

SEQ ID NO: 220 - protein, 7 aa, Homo sapiens
FVSTWIG

SEQ ID NO: 221 - protein, 5 aa, Homo sapiens
NYAIN

SEQ ID NO: 222 - protein, 5 aa, Homo sapiens
NYYIH

SEQ ID NO: 223 - protein, 5 aa, Homo sapiens
SYSIS

SEQ ID NO: 224 - protein, 5 aa, Homo sapiens
SYWIG

SEQ ID NO: 225 - protein, 5 aa, Homo sapiens
DYAMH

SEQ ID NO: 226 - protein, 5 aa, Homo sapiens
TYAMT

SEQ ID NO: 227 - protein, 5 aa, Homo sapiens
THGMH

SEQ ID NO: 228 - protein, 7 aa, Homo sapiens
AGRVGVS

SEQ ID NO: 229 - protein, 7 aa, Homo sapiens
GADYYWS

SEQ ID NO: 230 - protein, 5 aa, Homo sapiens
NSWIG

SEQ ID NO: 231 - protein, 6 aa, Homo sapiens
SGHFWG

SEQ ID NO: 232 - protein, 5 aa, Homo sapiens
NYYWG

SEQ ID NO: 233 - protein, 5 aa, Homo sapiens
SNGLS

SEQ ID NO: 234 - protein, 5 aa, Homo sapiens
ALSKH

SEQ ID NO: 235 - protein, 5 aa, Homo sapiens
TNGLH

SEQ ID NO: 236 - protein, 7 aa, Homo sapiens
RNRMSVS

SEQ ID NO: 237 - protein, 5 aa, Homo sapiens
TYGVS

SEQ ID NO: 238 - protein, 4 aa, Homo sapiens
YAMH

SEQ ID NO: 239 - protein, 5 aa, Homo sapiens
YIGMH

SEQ ID NO: 240 - protein, 5 aa, Homo sapiens
TYGLN

SEQ ID NO: 241 - protein, 5 aa, Homo sapiens
SYGFS

SEQ ID NO: 242 - protein, 6 aa, Homo sapiens
SGHYWG

SEQ ID NO: 243 - protein, 5 aa, Homo sapiens
TFGMH

SEQ ID NO: 244 - protein, 5 aa, Homo sapiens
SYGLH

SEQ ID NO: 245 - protein, 5 aa, Homo sapiens
SFGIS

SEQ ID NO: 246 - protein, 5 aa, Homo sapiens
RYGIS

SEQ ID NO: 247 - protein, 5 aa, Homo sapiens
NSGVS

SEQ ID NO: 248 - protein, 5 aa, Homo sapiens
SYGIS

SEQ ID NO: 249 - protein, 7 aa, Homo sapiens
SGGYSWS

SEQ ID NO: 250 - protein, 7 aa, Homo sapiens
SDKNYWS

SEQ ID NO: 251 - protein, 5 aa, Homo sapiens
GSTMH

SEQ ID NO: 252 - protein, 5 aa, Homo sapiens
TYTLH

SEQ ID NO: 253 - protein, 5 aa, Homo sapiens
SLGFS

SEQ ID NO: 254 - protein, 5 aa, Homo sapiens
GYTIH

SEQ ID NO: 255 - protein, 5 aa, Homo sapiens
NYWIG

SEQ ID NO: 256 - protein, 5 aa, Homo sapiens
NYAFS

SEQ ID NO: 257 - protein, 5 aa, Homo sapiens
NYGFS

SEQ ID NO: 258 - protein, 5 aa, Homo sapiens
SYAMN

SEQ ID NO: 259 - protein, 5 aa, Homo sapiens
GYTIS

SEQ ID NO: 260 - protein, 5 aa, Homo sapiens
KYGIH

SEQ ID NO: 261 - protein, 5 aa, Homo sapiens
SYGMH

SEQ ID NO: 262 - protein, 5 aa, Homo sapiens
SYTMS

SEQ ID NO: 263 - protein, 5 aa, Homo sapiens
TYGIS

SEQ ID NO: 264 - protein, 5 aa, Homo sapiens
RYTIH

SEQ ID NO: 265 - protein, 6 aa, Homo sapiens
NAYYWG

SEQ ID NO: 266 - protein, 5 aa, Homo sapiens
YYAMH

SEQ ID NO: 267 - protein, 5 aa, Homo sapiens
NYYWS

SEQ ID NO: 268 - protein, 5 aa, Homo sapiens
NYGMH

SEQ ID NO: 269 - protein, 5 aa, Homo sapiens
HYGMH

SEQ ID NO: 270 - protein, 5 aa, Homo sapiens
AYAMS

SEQ ID NO: 271 - protein, 7 aa, Homo sapiens
TSKLGVG

SEQ ID NO: 272 - protein, 5 aa, Homo sapiens
SYEMT

SEQ ID NO: 273 - protein, 5 aa, Homo sapiens
NFAMH

SEQ ID NO: 274 - protein, 6 aa, Homo sapiens
SNYYWG

SEQ ID NO: 275 - protein, 5 aa, Homo sapiens
SYGMH

SEQ ID NO: 276 - protein, 7 aa, Homo sapiens
TSRMSVS

SEQ ID NO: 277 - protein, 7 aa, Homo sapiens
SSNFYWG

SEQ ID NO: 278 - protein, 5 aa, Homo sapiens
TYGIS

SEQ ID NO: 279 - protein, 5 aa, Homo sapiens
KFYIH

SEQ ID NO: 280 - protein, 5 aa, Homo sapiens
SYTMH

SEQ ID NO: 281 - protein, 5 aa, Homo sapiens
NAWMS

SEQ ID NO: 282 - protein, 5 aa, Homo sapiens
IYGMH

SEQ ID NO: 283 - protein, 5 aa, Homo sapiens
DYGMH

SEQ ID NO: 284 - protein, 6 aa, Homo sapiens
SEYYWG

SEQ ID NO: 285 - protein, 5 aa, Homo sapiens
DYCMH

SEQ ID NO: 286 - protein, 16 aa, Homo sapiens
NINYRGNTNYNPSLKS

SEQ ID NO: 287 - protein, 17 aa, Homo sapiens
FIRYDGSTQDYVDSVKG

SEQ ID NO: 288 - protein, 17 aa, Homo sapiens
RITPMFDITNYAQKFQG

SEQ ID NO: 289 - protein, 17 aa, Homo sapiens
WINTSSGGTNYAQKFQG

SEQ ID NO: 290 - protein, 17 aa, Homo sapiens
YINRGGTTIYYADSVKG

SEQ ID NO: 291 - protein, 17 aa, Homo sapiens
WINAYNDNTYYSPSLQG

SEQ ID NO: 292 - protein, 16 aa, Homo sapiens
YIFHSGTTYYNPSLKS

SEQ ID NO: 293 - protein, 17 aa, Homo sapiens
IISYDGNNVHYADSVKG

SEQ ID NO: 294 - protein, 17 aa, Homo sapiens
WISADNGNTYYAQNFQD

SEQ ID NO: 295 - protein, 17 aa, Homo sapiens
WINTNTGDPAYAQDFTG

SEQ ID NO: 296 - protein, 17 aa, Homo sapiens
VISYDGRNKYFADSVKG

SEQ ID NO: 297 - protein, 17 aa, Homo sapiens
VIWHDGSNKNYLDSVKG

SEQ ID NO: 298 - protein, 17 aa, Homo sapiens
VIYYEGSNEYYADSVKG

SEQ ID NO: 299 - protein, 17 aa, Homo sapiens
YIGTGGSDIYYGDSVKG

SEQ ID NO: 300 - protein, 16 aa, Homo sapiens
YIYSSGSTFYNASLKS

SEQ ID NO: 301 - protein, 17 aa, Homo sapiens
ATSTDGGSTYYADSLKG

SEQ ID NO: 302 - protein, 16 aa, Homo sapiens
YIYYRGSTYYNPSLKS

SEQ ID NO: 303 - protein, 17 aa, Homo sapiens
IVYPGDSDTTYSPSFQG

SEQ ID NO: 304 - protein, 16 aa, Homo sapiens
RIDWDDDKYYSTSLKT

SEQ ID NO: 305 - protein, 17 aa, Homo sapiens
IINPADSDTRYSPSFQG

SEQ ID NO: 306 - protein, 17 aa, Homo sapiens
RIIPVFDTTNYAQKFQG

SEQ ID NO: 307 - protein, 17 aa, Homo sapiens
VINPNGGSTTSAQKFQD

SEQ ID NO: 308 - protein, 17 aa, Homo sapiens
MILPISGTTNYAQTFQG

SEQ ID NO: 309 - protein, 17 aa, Homo sapiens
IIYPGDSDTRNSPSFQG

SEQ ID NO: 310 - protein, 17 aa, Homo sapiens
VISYDGANEYYAESVKG

SEQ ID NO: 311 - protein, 17 aa, Homo sapiens
VIRASGDSEIYADSVRG

SEQ ID NO: 312 - protein, 17 aa, Homo sapiens
IISLDGIKTHYADSVKG

SEQ ID NO: 313 - protein, 16 aa, Homo sapiens
RIDWDDDKAFRTSLKT

SEQ ID NO: 314 - protein, 16 aa, Homo sapiens
FIYDSGSTYYNPSLRS

SEQ ID NO: 315 - protein, 17 aa, Homo sapiens
IIYPGDSTTTYTPSFQG

SEQ ID NO: 316 - protein, 16 aa, Homo sapiens
SIFHSGTTFHNPSLKS

SEQ ID NO: 317 - protein, 16 aa, Homo sapiens
HIYFGGNTNYNPSLQS

SEQ ID NO: 318 - protein, 17 aa, Homo sapiens
WISASSGNKKYAPKFQG

SEQ ID NO: 319 - protein, 17 aa, Homo sapiens
FFDPEDGDTGYAQKFQG

SEQ ID NO: 320 - protein, 17 aa, Homo sapiens
LINAGNGDTRFSQKFQG

SEQ ID NO: 321 - protein, 16 aa, Homo sapiens
RIDWDDDKFYNTSLQT

SEQ ID NO: 322 - protein, 17 aa, Homo sapiens
WISAYNGNTYYLQKLQG

SEQ ID NO: 323 - protein, 17 aa, Homo sapiens
WINVGNGQTKYSQRFQG

SEQ ID NO: 324 - protein, 17 aa, Homo sapiens
AISYDGSNKQYADSVKG

SEQ ID NO: 325 - protein, 17 aa, Homo sapiens
WVSAHNGNTYYAEKFHD

SEQ ID NO: 326 - protein, 17 aa, Homo sapiens
WSSVYNGDTNYAQKFHG

SEQ ID NO: 327 - protein, 16 aa, Homo sapiens
SIYDSGNTYYTPSLKS

SEQ ID NO: 328 - protein, 17 aa, Homo sapiens
VISYDGNKKYYADSVKG

SEQ ID NO: 329 - protein, 17 aa, Homo sapiens
EISYDGGSKFYTDSVKG

SEQ ID NO: 330 - protein, 17 aa, Homo sapiens
WISAYNGNTDYAQRLQD

SEQ ID NO: 331 - protein, 17 aa, Homo sapiens
WISAYNGNTYYAQNLQG

SEQ ID NO: 332 - protein, 17 aa, Homo sapiens
WISAYNGNTYYRQSLQD

SEQ ID NO: 333 - protein, 17 aa, Homo sapiens
WIGTDNGNTYYAQKFQG

SEQ ID NO: 334 - protein, 16 aa, Homo sapiens
YIYHSGSTYYNPSLKS

SEQ ID NO: 335 - protein, 16 aa, Homo sapiens
RLYPSGNTDYHPSLKS

SEQ ID NO: 336 - protein, 19 aa, Homo sapiens
RIRSKANSYATEYAASVKG

SEQ ID NO: 337 - protein, 17 aa, Homo sapiens
LINAANGHTKYSQRFQG

SEQ ID NO: 338 - protein, 17 aa, Homo sapiens
WTSAHNGNTYYAEEFQD

SEQ ID NO: 339 - protein, 17 aa, Homo sapiens
RLVPSLNIPNYAQKFQG

SEQ ID NO: 340 - protein, 17 aa, Homo sapiens
VIFPADSDARYSPSFQG

SEQ ID NO: 341 - protein, 17 aa, Homo sapiens
WISGSNGNTYYAEKFQG

SEQ ID NO: 342 - protein, 17 aa, Homo sapiens
WISAYNGNTYYAQNLQG

SEQ ID NO: 343 - protein, 17 aa, Homo sapiens
GISGSGGSTYYGDSVKG

SEQ ID NO: 344 - protein, 17 aa, Homo sapiens
RVVPTLGFPNYAQKFQG

SEQ ID NO: 345 - protein, 17 aa, Homo sapiens
VISYDGSKKYFTDSVKG

SEQ ID NO: 346 - protein, 17 aa, Homo sapiens
FIWNDGSNKYYADSVKG

SEQ ID NO: 347 - protein, 17 aa, Homo sapiens
SISASTVLTYYADSVKG

SEQ ID NO: 348 - protein, 17 aa, Homo sapiens
WISADNGNTYYAQKFQG

SEQ ID NO: 349 - protein, 17 aa, Homo sapiens
RVVPSLGIPNYAPKFQG

SEQ ID NO: 350 - protein, 16 aa, Homo sapiens
SIHHSGSAYYNSSLKS

SEQ ID NO: 351 - protein, 17 aa, Homo sapiens
VISYGETNKLYADSVKG

SEQ ID NO: 352 - protein, 16 aa, Homo sapiens
EISNTWSTNYNPSLKS

SEQ ID NO: 353 - protein, 17 aa, Homo sapiens
VIWYDDSNKQYGDSVKG

SEQ ID NO: 354 - protein, 17 aa, Homo sapiens
VISHDGNIKYSADSVKG

SEQ ID NO: 355 - protein, 17 aa, Homo sapiens
AISGGGGTTYYADSVKG

SEQ ID NO: 356 - protein, 16 aa, Homo sapiens
LVDWDDDRRYRPSLKS

SEQ ID NO: 357 - protein, 17 aa, Homo sapiens
HIGNSGSMIYYADSVKG

SEQ ID NO: 358 - protein, 17 aa, Homo sapiens
YINAVNGNTQYSQKFQG

SEQ ID NO: 359 - protein, 16 aa, Homo sapiens
SMHHSGSSYYKPSLKS

SEQ ID NO: 360 - protein, 17 aa, Homo sapiens
VISNDGSNKYYADSVKG

SEQ ID NO: 361 - protein, 16 aa, Homo sapiens
RIDWDDDKYYSTSLKT

SEQ ID NO: 362 - protein, 16 aa, Homo sapiens
SIFYSGTTYYNPSLKS

SEQ ID NO: 363 - protein, 17 aa, Homo sapiens
WISAYNGNTFYAQRLQG

SEQ ID NO: 364 - protein, 17 aa, Homo sapiens
IINPSGGSTTYAQTFQD

SEQ ID NO: 365 - protein, 17 aa, Homo sapiens
VVSYDGNHNDYADSVKG

SEQ ID NO: 366 - protein, 19 aa, Homo sapiens
LIKSHFEGGATDYAAPVKG

SEQ ID NO: 367 - protein, 17 aa, Homo sapiens
VISYDGAKKFYANSVKG

SEQ ID NO: 368 - protein, 17 aa, Homo sapiens
VIWHDGSNIRYADSVRG

SEQ ID NO: 369 - protein, 16 aa, Homo sapiens
SVHHSGSTYYNPSLKS

SEQ ID NO: 370 - protein, 17 aa, Homo sapiens
ILNPDGGTTFYAEKFQD

SEQ ID NO: 371 - protein, 18 aa, Homo sapiens
CARDVGYGGGQYFAMDVW

SEQ ID NO: 372 - protein, 24 aa, Homo sapiens
CAKDMDYYGSRSYSVTYYYGMDVW

SEQ ID NO: 373 - protein, 24 aa, Homo sapiens
CARRGAVALVPAAEDPYYYGMDVW

SEQ ID NO: 374 - protein, 20 aa, Homo sapiens
CAREDGTMGTNSWYGWFDPW

SEQ ID NO: 375 - protein, 22 aa, Homo sapiens
CARGLILALPTATVELGAFDIW

SEQ ID NO: 376 - protein, 26 aa, Homo sapiens
CARSYRSQTDILTGRYKGPGDVFDNW

SEQ ID NO: 377 - protein, 20 aa, Homo sapiens
CARDVDDFPVWGMNRYLALW

SEQ ID NO: 378 - protein, 19 aa, Homo sapiens
CAKDDVATDLAAYYYFDVW

SEQ ID NO: 379 - protein, 20 aa, Homo sapiens
CVRGGVVTNRVYYYYGMDVW

SEQ ID NO: 380 - protein, 13 aa, Homo sapiens
CAWFGEFGLFDYW

SEQ ID NO: 381 - protein, 18 aa, Homo sapiens
CARGSVQVWLHLGLFDNW

SEQ ID NO: 382 - protein, 17 aa, Homo sapiens
CARTPYEFWSGYYFDFW

SEQ ID NO: 383 - protein, 11 aa, Homo sapiens
CARKWLGMDFW

SEQ ID NO: 384 - protein, 13 aa, Homo sapiens
CARARPGYKVDFW

SEQ ID NO: 385 - protein, 17 aa, Homo sapiens
CARGGTLYTTGGEMHIW

SEQ ID NO: 386 - protein, 15 aa, Homo sapiens
CARRFWGFGNFFDYW

SEQ ID NO: 387 - protein, 20 aa, Homo sapiens
CAREGHHSGSGDYYSFFDYW

SEQ ID NO: 388 - protein, 22 aa, Homo sapiens
CVRRGGFCTATGCYAGHWFDPW

SEQ ID NO: 389 - protein, 20 aa, Homo sapiens
CARIVFHTSGGYYNPYMDVW

SEQ ID NO: 390 - protein, 15 aa, Homo sapiens
CARRAYDSGWHFEHW

SEQ ID NO: 391 - protein, 17 aa, Homo sapiens
CLRGSTRGWDTDGFDIW

SEQ ID NO: 392 - protein, 24 aa, Homo sapiens
CARQRSVTGGFDAWLLIPDASNTW

SEQ ID NO: 393 - protein, 21 aa, Homo sapiens
CARVFREFSTSTLDPYYFDYW

SEQ ID NO: 394 - protein, 22 aa, Homo sapiens
CVRQGGYYDRNGYHEKYAFDIW

SEQ ID NO: 395 - protein, 20 aa, Homo sapiens
CARAGRSSMNEEVIMYFDNW

SEQ ID NO: 396 - protein, 22 aa, Homo sapiens
CANIGQRRYCSGDHCYGHFDYW

SEQ ID NO: 397 - protein, 22 aa, Homo sapiens
CAKDHIGGTNAYFEWTVPFDGW

SEQ ID NO: 398 - protein, 20 aa, Homo sapiens
CARTQVFASGGYYLYYLDHW

SEQ ID NO: 399 - protein, 23 aa, Homo sapiens
CARDLGYGGNSYSHSYYYGLDVW

SEQ ID NO: 400 - protein, 11 aa, Homo sapiens
CARQGRGFGLW

SEQ ID NO: 401 - protein, 12 aa, Homo sapiens
CARVHGGGFDHW

SEQ ID NO: 402 - protein, 15 aa, Homo sapiens
CARDSSNWPAGYEDW

SEQ ID NO: 403 - protein, 18 aa, Homo sapiens
CAKDGGTYVPYSDAFDFW

SEQ ID NO: 404 - protein, 13 aa, Homo sapiens
CATVAAAGNFDNW

SEQ ID NO: 405 - protein, 16 aa, Homo sapiens
CARIAITMVRNPFDIW

SEQ ID NO: 406 - protein, 20 aa, Homo sapiens
CARTGIYDSSGYYLYYFDYW

SEQ ID NO: 407 - protein, 25 aa, Homo sapiens
CARDRVGGSSSEVLSRAKNYGLDVW

SEQ ID NO: 408 - protein, 19 aa, Homo sapiens
CARRASQYGEVYGNYFDYW

SEQ ID NO: 409 - protein, 21 aa, Homo sapiens
CAKDDFGNSNGVFFMSRVAFW

SEQ ID NO: 410 - protein, 21 aa, Homo sapiens
CVRGFNEQQLVPGLSFWFDYW

SEQ ID NO: 411 - protein, 21 aa, Homo sapiens
CARDRNVVLLPAAPFGGMDVW

SEQ ID NO: 412 - protein, 13 aa, Homo sapiens
CARGSPGDAFDIW

SEQ ID NO: 413 - protein, 18 aa, Homo sapiens
CAAQTPYFNESSGLVPDW

SEQ ID NO: 414 - protein, 18 aa, Homo sapiens
CARDLGDGYTAWGWFDPW

SEQ ID NO: 415 - protein, 21 aa, Homo sapiens
CTRDESMLRGVTEGFGPIDYW

SEQ ID NO: 416 - protein, 18 aa, Homo sapiens
CVISFDSTIAAAEYFDYW

SEQ ID NO: 417 - protein, 22 aa, Homo sapiens
CAREGHYSGSSSYQRDDAFDIW

SEQ ID NO: 418 - protein, 23 aa, Homo sapiens
CARGGTIEATPEREYYYYGMDVW

SEQ ID NO: 419 - protein, 13 aa, Homo sapiens
CASRSFYGDYVYW

SEQ ID NO: 420 - protein, 13 aa, Homo sapiens
CAKEGSGWYFESW

SEQ ID NO: 421 - protein, 18 aa, Homo sapiens
CTRHVGEMSTIWWYFDLW

SEQ ID NO: 422 - protein, 19 aa, Homo sapiens
CAKSGSHYGEVYGAYFDYW

SEQ ID NO: 423 - protein, 20 aa, Homo sapiens
CARDRGPGYSDSSFYVFDYW

SEQ ID NO: 424 - protein, 18 aa, Homo sapiens
CTRAPRGSTASHLLFDYW

SEQ ID NO: 425 - protein, 23 aa, Homo sapiens
CARPKYYFDSSGQFSEMYYFDFW

SEQ ID NO: 426 - protein, 14 aa, Homo sapiens
CARDLLRSTYFDYW

SEQ ID NO: 427 - protein, 21 aa, Homo sapiens
CARDGNTAGVDMWSRDGFDIW

SEQ ID NO: 428 - protein, 26 aa, Homo sapiens
CAKEPWIDIVVASVISPYYYDGMDVW

SEQ ID NO: 429 - protein, 18 aa, Homo sapiens
CARMNLGSHSGRPGFDMW

SEQ ID NO: 430 - protein, 24 aa, Homo sapiens
CATGGGVNVTSWSDVEHSSSLGYW

SEQ ID NO: 431 - protein, 20 aa, Homo sapiens
CVKDEVYDSSGYYLYYFDSW

SEQ ID NO: 432 - protein, 22 aa, Homo sapiens
CAKDYDFWSGYPGGQYWFFDLW

SEQ ID NO: 433 - protein, 21 aa, Homo sapiens
CVRGGTYSSDVEYYYYGMDVW

SEQ ID NO: 434 - protein, 18 aa, Homo sapiens
CARLTLGSYTGRPGFDSW

SEQ ID NO: 435 - protein, 18 aa, Homo sapiens
CARDTILTFGEPHWFDPW

SEQ ID NO: 436 - protein, 19 aa, Homo sapiens
CARDLRYLTYYSGSGDDSW

SEQ ID NO: 437 - protein, 20 aa, Homo sapiens
CARGLFYDSGGYYLFYFQHW

SEQ ID NO: 438 - protein, 20 aa, Homo sapiens
CARASEYSISWRHRGVLDYW

SEQ ID NO: 439 - protein, 19 aa, Homo sapiens
CHGEGYSTSWLGTAALDYW

SEQ ID NO: 440 - protein, 18 aa, Homo sapiens
CAKTRGYSYTWGDAFDLW

SEQ ID NO: 441 - protein, 20 aa, Homo sapiens
CAHSAYYTSSGYYLQYFHHW

SEQ ID NO: 442 - protein, 20 aa, Homo sapiens
CARSDYYDSSGYYLLYLDSW

SEQ ID NO: 443 - protein, 15 aa, Homo sapiens
CARNNGGSAIIFYYW

SEQ ID NO: 444 - protein, 20 aa, Homo sapiens
CARDLVVVTDISIKNYFDPW

SEQ ID NO: 445 - protein, 17 aa, Homo sapiens
CAKTTDQRLLVDWFDPW

SEQ ID NO: 446 - protein, 20 aa, Homo sapiens
CARTLVYAPDSYYLYYFDYW

SEQ ID NO: 447 - protein, 24 aa, Homo sapiens
CARHGFRYCNNGVCSINLDAFDIW

SEQ ID NO: 448 - protein, 21 aa, Homo sapiens
CARDLRMLPGGLPTRRGMDVW

SEQ ID NO: 449 - protein, 25 aa, Homo sapiens
CARGIREGGVSVEDWMLVYSWFDPW

SEQ ID NO: 450 - protein, 13 aa, Homo sapiens
CVRAPGSMGLDVW

SEQ ID NO: 451 - protein, 13 aa, Homo sapiens
CAPLGGPTPFDYW

SEQ ID NO: 452 - protein, 15 aa, Homo sapiens
CATASTYFYDSRDYW

SEQ ID NO: 453 - protein, 17 aa, Homo sapiens
CARVPFQIWSGLYFDHW

SEQ ID NO: 454 - protein, 18 aa, Homo sapiens
CARDRVALGVHYWYFDIW

SEQ ID NO: 455 - protein, 23 aa, Homo sapiens
CAILIARAYCGLADGQEGDFDTW

SEQ ID NO: 456 - protein, 11 aa, Homo sapiens
RASQSVNSHLA

SEQ ID NO: 457 - protein, 11 aa, Homo sapiens
RASQRISNHLN

SEQ ID NO: 458 - protein, 16 aa, Homo sapiens
RSSQSLLHSNGNNYLD

SEQ ID NO: 459 - protein, 12 aa, Homo sapiens
RASQSVSSSYLA

SEQ ID NO: 460 - protein, 11 aa, Homo sapiens
RASQSITGYLN

SEQ ID NO: 461 - protein, 11 aa, Homo sapiens
RASEGISSWLA

SEQ ID NO: 462 - protein, 12 aa, Homo sapiens
RASQSVSSSYLA

SEQ ID NO: 463 - protein, 16 aa, Homo sapiens
RSSQSLLRSDGKTFLY

SEQ ID NO: 464 - protein, 11 aa, Homo sapiens
RASQGISSYLA

SEQ ID NO: 465 - protein, 11 aa, Homo sapiens
RASQDINNYLA

SEQ ID NO: 466 - protein, 11 aa, Homo sapiens
RASQSVSSWVA

SEQ ID NO: 467 - protein, 11 aa, Homo sapiens
RASQGITDSLA

SEQ ID NO: 468 - protein, 16 aa, Homo sapiens
RSSQSLLNSNGFNYVD

SEQ ID NO: 469 - protein, 11 aa, Homo sapiens
RASQGISSYLA

SEQ ID NO: 470 - protein, 12 aa, Homo sapiens
RASQTVSSSYLV

SEQ ID NO: 471 - protein, 12 aa, Homo sapiens
RASQSVSSGYLA

SEQ ID NO: 472 - protein, 11 aa, Homo sapiens
RASQGINTYLN

SEQ ID NO: 473 - protein, 12 aa, Homo sapiens
RASQSISSGYLA

SEQ ID NO: 474 - protein, 11 aa, Homo sapiens
RASQTIASYLS

SEQ ID NO: 475 - protein, 11 aa, Homo sapiens
RASQSVGSKLA

SEQ ID NO: 476 - protein, 11 aa, Homo sapiens
RASQGISNYLV

SEQ ID NO: 477 - protein, 17 aa, Homo sapiens
RSSETVLYTSKNQSYLA

SEQ ID NO: 478 - protein, 12 aa, Homo sapiens
RASQSVSSSYIA

SEQ ID NO: 479 - protein, 11 aa, Homo sapiens
RASQSISSWLA

SEQ ID NO: 480 - protein, 11 aa, Homo sapiens
RASQSIGSRLA

SEQ ID NO: 481 - protein, 16 aa, Homo sapiens
RSSQSLLHSDGRYYVD

SEQ ID NO: 482 - protein, 11 aa, Homo sapiens
WASQTIGGNLA

SEQ ID NO: 483 - protein, 11 aa, Homo sapiens
RASQTIASYVN

SEQ ID NO: 484 - protein, 11 aa, Homo sapiens
RASQSVSSSLA

SEQ ID NO: 485 - protein, 11 aa, Homo sapiens
QASQDITYYLS

SEQ ID NO: 486 - protein, 11 aa, Homo sapiens
QASQDIGDSLN

SEQ ID NO: 487 - protein, 11 aa, Homo sapiens
RPSQDISSALA

SEQ ID NO: 488 - protein, 17 aa, Homo sapiens
KSSQSVLYNSNNKNYLA

SEQ ID NO: 489 - protein, 11 aa, Homo sapiens
RASQFISSYLH

SEQ ID NO: 490 - protein, 11 aa, Homo sapiens
RASQSIGSWLA

SEQ ID NO: 491 - protein, 11 aa, Homo sapiens
RASQSIASYLN

SEQ ID NO: 492 - protein, 11 aa, Homo sapiens
RASQSVTSELA

SEQ ID NO: 493 - protein, 11 aa, Homo sapiens
RASQNIYNWLA

SEQ ID NO: 494 - protein, 11 aa, Homo sapiens
RANQDIDNYLA

SEQ ID NO: 495 - protein, 11 aa, Homo sapiens
RASQGISKRLA

SEQ ID NO: 496 - protein, 11 aa, Homo sapiens
RASQGISSYLA

SEQ ID NO: 497 - protein, 11 aa, Homo sapiens
RASQGIGTWLA

SEQ ID NO: 498 - protein, 11 aa, Homo sapiens
RASQGISNYLA

SEQ ID NO: 499 - protein, 12 aa, Homo sapiens
RASQSVGGRSLA

SEQ ID NO: 500 - protein, 17 aa, Homo sapiens
RSSQSVLYSSNNKNYLA

SEQ ID NO: 501 - protein, 11 aa, Homo sapiens
RASQTISNSLA

SEQ ID NO: 502 - protein, 11 aa, Homo sapiens
RASQGISNYLA

SEQ ID NO: 503 - protein, 11 aa, Homo sapiens
RASQGISSYLA

SEQ ID NO: 504 - protein, 12 aa, Homo sapiens
RASQSVSSSYLA

SEQ ID NO: 505 - protein, 11 aa, Homo sapiens
RASQGISAWLA

SEQ ID NO: 506 - protein, 11 aa, Homo sapiens
RASQSISSYLN

SEQ ID NO: 507 - protein, 11 aa, Homo sapiens
RASQNIYNWLA

SEQ ID NO: 508 - protein, 16 aa, Homo sapiens
RSSQSLVNSDGNTYLS

SEQ ID NO: 509 - protein, 11 aa, Homo sapiens
QASQDVSYYLN

SEQ ID NO: 510 - protein, 12 aa, Homo sapiens
RASQSVSSNYLA

SEQ ID NO: 511 - protein, 11 aa, Homo sapiens
RASQAISNWLA

SEQ ID NO: 512 - protein, 17 aa, Homo sapiens
RSSQSLLDSNDGNTYLD

SEQ ID NO: 513 - protein, 16 aa, Homo sapiens
RSSQSLLHRNEYNYLD

SEQ ID NO: 514 - protein, 11 aa, Homo sapiens
QASQDISNYLN

SEQ ID NO: 515 - protein, 11 aa, Homo sapiens
RASQGIRNYLA

SEQ ID NO: 516 - protein, 11 aa, Homo sapiens
RASQIIASYLN

SEQ ID NO: 517 - protein, 11 aa, Homo sapiens
RTSQSVSSYLA

SEQ ID NO: 518 - protein, 11 aa, Homo sapiens
RASQGISIYLA

SEQ ID NO: 519 - protein, 11 aa, Homo sapiens
QASQDINNYLN

SEQ ID NO: 520 - protein, 11 aa, Homo sapiens
RASQSIKNNLA

SEQ ID NO: 521 - protein, 12 aa, Homo sapiens
RASQSLSDNYLA

SEQ ID NO: 522 - protein, 11 aa, Homo sapiens
RASQRIASYLN

SEQ ID NO: 523 - protein, 11 aa, Homo sapiens
QASQGISNYLN

SEQ ID NO: 524 - protein, 11 aa, Homo sapiens
RASQGIRNFLA

SEQ ID NO: 525 - protein, 11 aa, Homo sapiens
RASQSVTSNLA

SEQ ID NO: 526 - protein, 11 aa, Homo sapiens
RASQTIASYVN

SEQ ID NO: 527 - protein, 11 aa, Homo sapiens
RASQTIASYVN

SEQ ID NO: 528 - protein, 11 aa, Homo sapiens
RSSQTISVFLN

SEQ ID NO: 529 - protein, 11 aa, Homo sapiens
RASQSVTKYLA

SEQ ID NO: 530 - protein, 11 aa, Homo sapiens
RASQSVSSNLA

SEQ ID NO: 531 - protein, 11 aa, Homo sapiens
RASQTIASYVN

SEQ ID NO: 532 - protein, 16 aa, Homo sapiens
RSSQSLLRTNGYNYLD

SEQ ID NO: 533 - protein, 11 aa, Homo sapiens
RASQSISSWLA

SEQ ID NO: 534 - protein, 11 aa, Homo sapiens
RASQNIRTFIN

SEQ ID NO: 535 - protein, 16 aa, Homo sapiens
RSSQSLLHRNGYNHLD

SEQ ID NO: 536 - protein, 11 aa, Homo sapiens
RAGQGIRNDLG

SEQ ID NO: 537 - protein, 16 aa, Homo sapiens
RSSRSLVHSDGNTYLS

SEQ ID NO: 538 - protein, 11 aa, Homo sapiens
RASQSVGNNLA

SEQ ID NO: 539 - protein, 11 aa, Homo sapiens
RASQSVSSHLA

SEQ ID NO: 540 - protein, 11 aa, Homo sapiens
RASRSITSWLA

SEQ ID NO: 541 - protein, 7 aa, Homo sapiens
NTFNRVT

SEQ ID NO: 542 - protein, 7 aa, Homo sapiens
GASTLQS

SEQ ID NO: 543 - protein, 7 aa, Homo sapiens
LASNRAS

SEQ ID NO: 544 - protein, 7 aa, Homo sapiens
GASSRAT

SEQ ID NO: 545 - protein, 7 aa, Homo sapiens
ATSTLQS

SEQ ID NO: 546 - protein, 7 aa, Homo sapiens
AASTLQS

SEQ ID NO: 547 - protein, 7 aa, Homo sapiens
GASTGAT

SEQ ID NO: 548 - protein, 7 aa, Homo sapiens
EVSSRFS

SEQ ID NO: 549 - protein, 7 aa, Homo sapiens
AASTLQS

SEQ ID NO: 550 - protein, 7 aa, Homo sapiens
AASSLQS

SEQ ID NO: 551 - protein, 7 aa, Homo sapiens
EASNLES

SEQ ID NO: 552 - protein, 7 aa, Homo sapiens
AASRLES

SEQ ID NO: 553 - protein, 7 aa, Homo sapiens
LGSNRAS

SEQ ID NO: 554 - protein, 7 aa, Homo sapiens
VASILES

SEQ ID NO: 555 - protein, 7 aa, Homo sapiens
GASTRAT

SEQ ID NO: 556 - protein, 7 aa, Homo sapiens
GASGRAT

SEQ ID NO: 557 - protein, 7 aa, Homo sapiens
AASSLQS

SEQ ID NO: 558 - protein, 7 aa, Homo sapiens
GASHRAT

SEQ ID NO: 559 - protein, 7 aa, Homo sapiens
TASSLQS

SEQ ID NO: 560 - protein, 7 aa, Homo sapiens
GASTRAT

SEQ ID NO: 561 - protein, 7 aa, Homo sapiens
AASSLQS

SEQ ID NO: 562 - protein, 7 aa, Homo sapiens
WASTRES

SEQ ID NO: 563 - protein, 7 aa, Homo sapiens
AASRRAT

SEQ ID NO: 564 - protein, 7 aa, Homo sapiens
KSSILES

SEQ ID NO: 565 - protein, 7 aa, Homo sapiens
DASSLES

SEQ ID NO: 566 - protein, 7 aa, Homo sapiens
LASNRAS

SEQ ID NO: 567 - protein, 7 aa, Homo sapiens
GASTRAT

SEQ ID NO: 568 - protein, 7 aa, Homo sapiens
AASNLQS

SEQ ID NO: 569 - protein, 7 aa, Homo sapiens
DASYRVT

SEQ ID NO: 570 - protein, 7 aa, Homo sapiens
DVSNLER

SEQ ID NO: 571 - protein, 7 aa, Homo sapiens
DASNLET

SEQ ID NO: 572 - protein, 7 aa, Homo sapiens
GASTLDY

SEQ ID NO: 573 - protein, 7 aa, Homo sapiens
LASTREY

SEQ ID NO: 574 - protein, 7 aa, Homo sapiens
AASTLQS

SEQ ID NO: 575 - protein, 7 aa, Homo sapiens
KESNLES

SEQ ID NO: 576 - protein, 7 aa, Homo sapiens
AASSLHS

SEQ ID NO: 577 - protein, 7 aa, Homo sapiens
KASSLES

SEQ ID NO: 578 - protein, 7 aa, Homo sapiens
DASTLES

SEQ ID NO: 579 - protein, 7 aa, Homo sapiens
GASKLQT

SEQ ID NO: 580 - protein, 7 aa, Homo sapiens
GASSLQH

SEQ ID NO: 581 - protein, 7 aa, Homo sapiens
AASTLQS

SEQ ID NO: 582 - protein, 7 aa, Homo sapiens
AASRLQS

SEQ ID NO: 583 - protein, 7 aa, Homo sapiens
AASTLQS

SEQ ID NO: 584 - protein, 7 aa, Homo sapiens
DASNRAT

SEQ ID NO: 585 - protein, 7 aa, Homo sapiens
WASTRAS

SEQ ID NO: 586 - protein, 7 aa, Homo sapiens
KASTLES

SEQ ID NO: 587 - protein, 7 aa, Homo sapiens
TTSTLRS

SEQ ID NO: 588 - protein, 7 aa, Homo sapiens
AASTLQS

SEQ ID NO: 589 - protein, 7 aa, Homo sapiens
GASSRAT

SEQ ID NO: 590 - protein, 7 aa, Homo sapiens
DASTLAS

SEQ ID NO: 591 - protein, 7 aa, Homo sapiens
AASSLQS

SEQ ID NO: 592 - protein, 7 aa, Homo sapiens
DASSLES

SEQ ID NO: 593 - protein, 7 aa, Homo sapiens
QISKRFS

SEQ ID NO: 594 - protein, 7 aa, Homo sapiens
DTSNLVT

SEQ ID NO: 595 - protein, 7 aa, Homo sapiens
GASSRAA

SEQ ID NO: 596 - protein, 7 aa, Homo sapiens
AASSLQS

SEQ ID NO: 597 - protein, 7 aa, Homo sapiens
TFSYRAS

SEQ ID NO: 598 - protein, 7 aa, Homo sapiens
WGSNRAS

SEQ ID NO: 599 - protein, 7 aa, Homo sapiens
DATKLET

SEQ ID NO: 600 - protein, 7 aa, Homo sapiens
AASTLQS

SEQ ID NO: 601 - protein, 7 aa, Homo sapiens
AASSLQS

SEQ ID NO: 602 - protein, 7 aa, Homo sapiens
DASNRAT

SEQ ID NO: 603 - protein, 7 aa, Homo sapiens
AASTLQT

SEQ ID NO: 604 - protein, 7 aa, Homo sapiens
DATDLET

SEQ ID NO: 605 - protein, 7 aa, Homo sapiens
GASARAT

SEQ ID NO: 606 - protein, 7 aa, Homo sapiens
GASSRPT

SEQ ID NO: 607 - protein, 7 aa, Homo sapiens
AASSLQS

SEQ ID NO: 608 - protein, 7 aa, Homo sapiens
DASNLES

SEQ ID NO: 609 - protein, 7 aa, Homo sapiens
AASTLQS

SEQ ID NO: 610 - protein, 7 aa, Homo sapiens
GASTRAT

SEQ ID NO: 611 - protein, 7 aa, Homo sapiens
AASSLQS

SEQ ID NO: 612 - protein, 7 aa, Homo sapiens
AASNLQS

SEQ ID NO: 613 - protein, 7 aa, Homo sapiens
AASSLHS

SEQ ID NO: 614 - protein, 7 aa, Homo sapiens
DASNRAT

SEQ ID NO: 615 - protein, 7 aa, Homo sapiens
SASTRAT

SEQ ID NO: 616 - protein, 7 aa, Homo sapiens
AASRLQS

SEQ ID NO: 617 - protein, 7 aa, Homo sapiens
LGSIRAS

SEQ ID NO: 618 - protein, 7 aa, Homo sapiens
KASSLES

SEQ ID NO: 619 - protein, 7 aa, Homo sapiens
AASKLES

SEQ ID NO: 620 - protein, 7 aa, Homo sapiens
LGSNRAS

SEQ ID NO: 621 - protein, 7 aa, Homo sapiens
GASTLQS

SEQ ID NO: 622 - protein, 7 aa, Homo sapiens
KISNRFS

SEQ ID NO: 623 - protein, 7 aa, Homo sapiens
GASTRAT

SEQ ID NO: 624 - protein, 7 aa, Homo sapiens
GASTRAT

SEQ ID NO: 625 - protein, 7 aa, Homo sapiens
KASSLQS

SEQ ID NO: 626 - protein, 13 aa, Homo sapiens [sequence omitted]

SEQ ID NO: 627 - protein, 12 aa, Homo sapiens
CQQSYRTPPINF

SEQ ID NO: 628 - protein, 10 aa, Homo sapiens [sequence omitted]

SEQ ID NO: 629 - protein, 13 aa, Homo sapiens
CQQYDSSLSTWTF

SEQ ID NO: 630 - protein, 10 aa, Homo sapiens [sequence omitted]

SEQ ID NO: 631 - protein, 11 aa, Homo sapiens [sequence omitted]

SEQ ID NO: 632 - protein, 11 aa, Homo sapiens [sequence omitted]

SEQ ID NO: 633 - protein, 11 aa, Homo sapiens
CMQGLKIRRTF

SEQ ID NO: 634 - protein, 11 aa, Homo sapiens
CQQVDTYPLTF

SEQ ID NO: 635 - protein, 11 aa, Homo sapiens [sequence omitted]

SEQ ID NO: 636 - protein, 12 aa, Homo sapiens
CQQYHSYSGYTF

SEQ ID NO: 637 - protein, 11 aa, Homo sapiens
CQQYSKSPATF

SEQ ID NO: 638 - protein, 11 aa, Homo sapiens [sequence omitted]

SEQ ID NO: 639 - protein, 11 aa, Homo sapiens
CQQSKSFPPTF

SEQ ID NO: 640 - protein, 11 aa, Homo sapiens
CQQYGGSGLTF

SEQ ID NO: 641 - protein, 11 aa, Homo sapiens
CQQYFGSPYTF

SEQ ID NO: 642 - protein, 11 aa, Homo sapiens
CQQSANSPHTF

SEQ ID NO: 643 - protein, 11 aa, Homo sapiens [sequence omitted]

SEQ ID NO: 644 - protein, 11 aa, Homo sapiens
CQHSYNTPYTF

SEQ ID NO: 645 - protein, 12 aa, Homo sapiens [sequence omitted]

SEQ ID NO: 646 - protein, 11 aa, Homo sapiens
CLQHNISPYTF

SEQ ID NO: 647 - protein, 11 aa, Homo sapiens
CQQFFRSPFTF

SEQ ID NO: 648 - protein, 11 aa, Homo sapiens [sequence omitted]

SEQ ID NO: 649 - protein, 11 aa, Homo sapiens [sequence omitted]

SEQ ID NO: 650 - protein, 12 aa, Homo sapiens [sequence omitted]

SEQ ID NO: 651 - protein, 11 aa, Homo sapiens [sequence omitted]

SEQ ID NO: 652 - protein, 10 aa, Homo sapiens [sequence omitted]

SEQ ID NO: 653 - protein, 12 aa, Homo sapiens
CQQSYSYRALTF

SEQ ID NO: 654 - protein, 13 aa, Homo sapiens [sequence omitted]

SEQ ID NO: 655 - protein, 11 aa, Homo sapiens
CQQYDFLPYTF

SEQ ID NO: 656 - protein, 13 aa, Homo sapiens
CQHYVNLPPSFTF

SEQ ID NO: 657 - protein, 11 aa, Homo sapiens [sequence omitted]

SEQ ID NO: 658 - protein, 11 aa, Homo sapiens
CQQYYQTPLTF

SEQ ID NO: 659 - protein, 11 aa, Homo sapiens [sequence omitted]

SEQ ID NO: 660 - protein, 10 aa, Homo sapiens
CQQYKNDWTF

SEQ ID NO: 661 - protein, 11 aa, Homo sapiens
CQHSYSTRFTF

SEQ ID NO: 662 - protein, 11 aa, Homo sapiens [sequence omitted]

SEQ ID NO: 663 - protein, 11 aa, Homo sapiens [sequence omitted]

SEQ ID NO: 664 - protein, 11 aa, Homo sapiens [sequence omitted]

SEQ ID NO: 665 - protein, 11 aa, Homo sapiens [sequence omitted]

SEQ ID NO: 666 - protein, 11 aa, Homo sapiens [sequence omitted]

SEQ ID NO: 667 - protein, 11 aa, Homo sapiens [sequence omitted]

SEQ ID NO: 668 - protein, 11 aa, Homo sapiens [sequence omitted]

SEQ ID NO: 669 - protein, 11 aa, Homo sapiens [sequence omitted]

SEQ ID NO: 670 - protein, 11 aa, Homo sapiens
CQQFHSTPRTF

SEQ ID NO: 671 - protein, 11 aa, Homo sapiens
CQQYNSFSFTF

SEQ ID NO: 672 - protein, 11 aa, Homo sapiens [sequence omitted]

SEQ ID NO: 673 - protein, 11 aa, Homo sapiens [sequence omitted]

SEQ ID NO: 674 - protein, 11 aa, Homo sapiens [sequence omitted]

SEQ ID NO: 675 - protein, 11 aa, Homo sapiens [sequence omitted]

SEQ ID NO: 676 - protein, 11 aa, Homo sapiens [sequence omitted]

SEQ ID NO: 677 - protein, 11 aa, Homo sapiens
CQQYNIYSPTF

SEQ ID NO: 678 - protein, 11 aa, Homo sapiens [sequence omitted]

SEQ ID NO: 679 - protein, 11 aa, Homo sapiens
CLQYHYLPYTF

SEQ ID NO: 680 - protein, 11 aa, Homo sapiens [sequence omitted]

SEQ ID NO: 681 - protein, 11 aa, Homo sapiens
CQQADTFPFTF

SEQ ID NO: 682 - protein, 11 aa, Homo sapiens [sequence omitted]

SEQ ID NO: 683 - protein, 11 aa, Homo sapiens [sequence omitted]

SEQ ID NO: 684 - protein, 11 aa, Homo sapiens
CQHFANLPYTF

SEQ ID NO: 685 - protein, 11 aa, Homo sapiens [sequence omitted]

SEQ ID NO: 686 - protein, 12 aa, Homo sapiens [sequence omitted]

SEQ ID NO: 687 - protein, 10 aa, Homo sapiens [sequence omitted]

SEQ ID NO: 688 - protein, 11 aa, Homo sapiens [sequence omitted]

SEQ ID NO: 689 - protein, 11 aa, Homo sapiens
CQHFANLPYTF

SEQ ID NO: 690 - protein, 12 aa, Homo sapiens
CQEYNNWPLLTF

SEQ ID NO: 691 - protein, 11 aa, Homo sapiens [sequence omitted]

SEQ ID NO: 692 - protein, 12 aa, Homo sapiens
CQQSYSTPIYTF

SEQ ID NO: 693 - protein, 11 aa, Homo sapiens [sequence omitted]

SEQ ID NO: 694 - protein, 11 aa, Homo sapiens [sequence omitted]

SEQ ID NO: 695 - protein, 11 aa, Homo sapiens
CQQYNNWPQTF

SEQ ID NO: 696 - protein, 11 aa, Homo sapiens [sequence omitted]

SEQ ID NO: 697 - protein, 12 aa, Homo sapiens
CQQSYSVPRLTF

SEQ ID NO: 698 - protein, 10 aa, Homo sapiens
CQESFSSSTF

SEQ ID NO: 699 - protein, 10 aa, Homo sapiens
CQHRRSWPTF

SEQ ID NO: 700 - protein, 12 aa, Homo sapiens
CQQYNMWPPWTF

SEQ ID NO: 701 - protein, 11 aa, Homo sapiens [sequence omitted]

SEQ ID NO: 702 - protein, 11 aa, Homo sapiens
CMQSLQTSITF

SEQ ID NO: 703 - protein, 11 aa, Homo sapiens [sequence omitted]

SEQ ID NO: 704 - protein, 11 aa, Homo sapiens
CQQGHSTPYTF

SEQ ID NO: 705 - protein, 11 aa, Homo sapiens [sequence omitted]

SEQ ID NO: 706 - protein, 11 aa, Homo sapiens [sequence omitted]

SEQ ID NO: 707 - protein, 10 aa, Homo sapiens
CLQATQFLTF

SEQ ID NO: 708 - protein, 11 aa, Homo sapiens
CQQYDKWPETF

SEQ ID NO: 709 - protein, 11 aa, Homo sapiens
CQQYDNWLPTF

SEQ ID NO: 710 - protein, 11 aa, Homo sapiens [sequence omitted]

SEQ ID NO: 711 - protein, 298 aa, respiratory syncytial virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDHKPQTTKPKEVPTTKPTEEPTINTTKTNIITTLLTNNTTGNPKLTSQMETFHSTSSEGNLSPSQVSTTSEHPSQPSSPPNTTRQ

SEQ ID NO: 712 - protein, 292 aa, respiratory syncytial virus
MSKHKNQRTARTLEKTWDTLNHLIVISSCLYRLNLKSIAQIALSVLAMIISTSLIIAAIIFIISANHKVTLTTVTVQTIKNHTEKNISTYLTQVPPERVNSSKQPTTTSPIHTNSATISPNTKSETHHTTAQTKGRITTSTQTNKPSTKSRSKNPPKKPKDDYHFEVFNFVPCSICGNNQLCKSICKTIPSNKPKKKPTIKPTNKPTTKTTNKRDPKTPAKMPKKEIITNPAKKPTLKTTERDTSISQSTVLDTITPKYTIQQQSLHSTTSENTPSSTQIPTASEPSTLNPN

SEQ ID NO: 713 - protein, 77 aa, respiratory syncytial virus
QPTTVKTKNTTTTQTQPSKPTTKQRQNKPPNKPNNDFHFEVFNFVPCSICSNNPTCWAICKRIPNKKPGKKTTTKPT

SEQ ID NO: 714 - protein, 77 aa, respiratory syncytial virus
HHTTAQTKGRITTSTQTNKPSTKSRSKNPPKKPKDDYHFEVFNFVPCSICGNNQLCKSICKTIPSNKPKKKPTIKPT